SEQUENCE LISTING (142 sequences)

SEQ ID NO: 1 - DNA, 1635 bp, Cannabis sativa
atgatgatgcggtggaagaggtgggatactttgttcgtttctaaaaaaattattgggatcaactttagttttcaccttaactaacctgttaaaatttttaccaaaatacttttcaccccaaatacgtgcttgtgtgtaattattaggactcgcatgattagtttttcctaaatcaaggtccctataattgagatacgccaatcttggattttgggacacataaggagtcgtaaaattataaacacttcgaacccagtttatatgcttttcattatcttcttgcttctcccaggaagcagtgtaccaaagttcatacattattccagctcgatgagggaatggaattgctgattctgaaatctcctccattataccaccgtaagggtacaacacatacatcccagctcctacatcttcttcatataatttttccaaaattttgaccattgcagtttctggaattggtttcttaacatagtctaacttaattgagaaagccgtcttcttcccagctgatctatcaagcaaaatttcctttttaaaattagcagtgttaaaatttacaacaccactgtagaagatggttgtatcaatccagctaaattctttgcaatcagtttttttaatacccaactcacgaaagctcttgttcatcaagtcgactagactatccactccaccatgaaaaattgaagagaagtaaccatgtactgtagtcttattcttcccatgattatctgtaatattctttgttatgaagtgagtcatgagtactaaatctttgtcatacttgtaagcaatattttgccatttgttaaataacttgacaagcccatgtatctccatgttctttttaacactgaatatagtagactttgatgggacagcaaccagtttgattttccatgctgcaatgattccaaagttttctcctccaccaccacgtatagcccaaaacagatcttctcccatggattttcgatctagaacttttccatcaacattgactaagtgtgcatcaataatattatcagccgcaaggccataatttcgcatcaatgctccatagcctcctccactaaagtgtccacctacgccaacagtagggcaatacccaccaggaaaactaagattctcattcttctcattgatccaataataaacttctccaagggtagctccggcttcaacccacgcagtttggctatgaacatctattttgatcgaatgcatgtttctcaagtctactacaacaaatgggacttgagatatgtaggacataccctcagcatcatggccaccgcttcgagttcgaatctgcaagccaactttcttagagcataaaatagttgcttggatatgggagttatttgaaggagtgacaataacgagtggttttggggttgtatcagagatgaatctaagattttgtattgtcgaattcaggatagacatatacaattggtcgtgttgagtgtatacgagttttggatttgctacattgttgggaatatgttttgagaagcatttaaggaagttttctcgaggattagctattgaaatttggatatggaatgagagaaagaaaaatattattttgcaaacaaaccaaaaggaaaatgctgagcaattcat

SEQ ID NO: 2 - protein, 545 aa, Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 3 - DNA, 1634 bp, Cannabis sativa
tgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcgtgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcat

SEQ ID NO: 4 - DNA, 1634 bp, Cannabis sativa
atgatgatgcggtggaagaggtgggatactttgttcgtttctaaaaaaattattgggatcaactttagttttcaccttaactaacctgttaaaatttttaccaaaatacttttcaccccaaatacgtgcttgtgtgtaattattaggactcgcatgattagtttttcctaaatcaaggtccctataattgagatacgccaatcttggattttgggacacataaggagtcgtaaaattataaacacttcgaacccagtttatatgcttttcattatcttcttgcttctcccaggaagcagtgtaccaaagttcatacattattccagctcgatgagggaatggaattgctgattctgaaatctcctccattataccaccgtaagggtacaacacatacatcccagctcctacatcttcttcatataatttttccaaaattttgaccattgcagtttctggaattggtttcttaacatagtctaacttaattgagaaagccgtcttcttcccagctgatctatcaagcaaaatttcctttttaaaattagcagtgttgtaatttacaacaccactgtagaagatggttgtatcaatccagctcaattctttgcaatcagtttttttaatacccaactcacgaaagctcttgttcatcaagtcgactagactatccactccaccatgaaaaattgaagagaagtaaccatgtactgtagtcttattcttcccatgattatctgtaatattctttgttatgaagtgagtcatgagtactaaatctttgtcatacttgtaagcaatattttgccatttgttaaataacttgacaagcccatgtatctccatgttctttttaacactgaatatagtagactttgatgggacagcaaccagtttgattttccatgctgcaatgattccaaagttttctcctccaccaccacgtatagcccaaaacagatcttctcccatggattttcgatctagaacttttccatcaacattgactaagtgtgcatcaatgatattatcagccgcaaggccataatttcgcatcaatgctccatagcctcctccactaaagtgtccacctacgccaacagtagggcaatacccaccaggaaaactaagattctcattcttctcattgatccaataataaacttctccaagggtagctccggcttcaacccacgcagtttggctatgaacatctattttgatcgaatgcatgtttctcaagtctactacaacaaatgggacttgagatatgtaggacataccctcagcatcatggccaccgcttcgagttcgaatctgcaagccaactttcttagagcataaaatagttgcttggatatgggagttatttgaaggagtgacaataacgagtggttttggggttgtatcagagatgaatctaagattttgtattgtcgaattcaggatagacatatacaattggtcgtgttgagtgtatacgagttttggatttgctacattgttgggaatatgttttgagaagcatttaaggaagttttctcgaggattagctattgaaatttggatatggaatgagagaaagaaaaatattattttgcaaacaaaccaaaaggaaaatgctgagcaattca

SEQ ID NO: 5 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 6 - DNA, 1635 bp, Cannabis sativa; identical to SEQ ID NO: 1

SEQ ID NO: 7 - DNA, 1634 bp, Cannabis sativa
atgatgacgcggtggaagaggtgggatactttgttcgtttctaaaaaaattattgggatcagctttggttttcaccttaactaacctgttaaaatttttaccaaaatacttttcaccccaaatacgtgcttgtgtgtaattattaggactctcaggattagtttttcctaaatcaaggtccctataattgagatacgccaatcttggattttgggacacataaggagttgtgaaattataaacacttcgaacccagtttatatgcttttcgttatcttcttgcttctcccaggtagcagtgtaccaaagttcatacattattccagctcgatgagggaatggaattgctgattctgaaatctcatccattataccaccgtaagggtacaacacatacatcccaactcctacctcttcttcatataatttttccaaaattttgaccattacagtttcaggtattagtttcttaacatagtctaacttaattgagaaagccgtcttcttcccagctgatctatcaagcaaaatttcctttttaaaattagcagtgttgtaatttacaacaccactgtagaagatggttgtatcaatccagctcaattctttgcaatcagtttttttaatacccaactcaggaaagctcttgttcatcaagtcaactagactatccactccaccaagaaaaatggaagagaagtaaccatgtactgtagtcttattcttcccatgattatctgtaatattcctagtttctgaagtgagtcgtgagcattaaatctttgtcatacttgtaagcaatattttgccatttgttaaataacttgacaagcccatgtatctccatgttctttttaacactgaatatagtagcctttgatgggacaacaacaagtttgattttccatgctgcaatgattccaaagttttctcctcctccaccacgtatagcccaaaatagatcttctccatggattttcgatctagaacttttccatcaacattgactaagtgtgcatcaatgatattatcagccgcaaggccataatttcgcatcaatgctccatagcctcctccactaaagtgtccacctacgccaacagtagggcaatacccaccaggaaaactaaaattctcattcatctcattgatccaataataaacttctccaagggtagctccggcttcaacccacgcagtttggctatgaatatctactttgaccgtatgcatgttttcaagtctactatagcaaatgggacttgagatatgtaggacaaaccctcagcatcatggccaccgcttcgagttcgaatctgcaaaccaactttcttggagcagagaatactggcctggatatgggagacatttgaaggagtgacaataacgagtggttttggggttgtatcagaggtgaatctaagattttgtattgtcgaattcaggacagacatatacaattggtcgtgttgagtgtatatgaattttggatttgctggattgttaggaatatattccgagaagcatttaaggaagttttcttgaggattagctattgaaatttggatattgaatgagagaaagaaaaatattattttgcaaacaaaccaaaaggagaatgttgagcaattcat

SEQ ID NO: 8 - DNA, 1635 bp, Cannabis sativa
atgatgacgcggtggaagaggtgggatactttgttcgtttctaaaaaaattattgggatcagctttggttttcaccttaactaacctgttaaaatttttaccaaaatacttttcaccccaaatacgtgcttgtgtgtaattattaggactctcaggattagtttttcctaaatcaaggtccctataattgagatacgccaatcttggattttgggacacataaggagttgtgaaattataaacacttcgaacccagtttatatgcttttcgttatcttcttgcttctcccaggtagcagtgtaccaaagttcatacattattccagctcgatgagggaatggaattgctgattctgaaatctcatccattataccaccgtaagggtacaacacatacatcccaactcctacctcttcttcatataatttttccaaaattttgaccattgcagtttcaggtattagtttcttaacatagtctaacttaattgagaaagccgtcttcttcccagctgatctatcaagcaaaatttcctttttaaaattagcagtgttgtaatttacaacaccactgtagaagatggttgtatcaatccagctcaattctttgcaatcagtttttttaatacccaactcaggaaagctcttgttcatcaagtcaactagactatccactccaccaagaaaaatggaagagaagtaaccatgtactgtagtcttattcttcccatgattatctgtaatattcctagttctgaagtgagtcgtgagcattaaatctttgtcatacttgtaagcaatattttgccatttgttaaataacttgacaagcccatgtatctccatgttctttttaacactgaatatagtagcctttgatgggacaacaacaagtttgattttccatgctgcaatgattccaaagttttctcctcctccaccacgtatagcccaaaatagatcttctcccatggattttcgatctagaacttttccatcaacattgactaagtgtgcatcaatgatattatcagccgcaaggccataatttcgcatcaatgctccatagcctcctccactaaagtgtccacctacgccaacagtagggcaatacccaccaggaaaactaaaattctcattcatctcattgatccaataataaacttctccaagggtagctccggcttcaacccacgcagtttggctatgaatatctactttgaccgtatgcatgtttctcaagtctactatagcaaatgggacttgagatatgtaggacaaaccctcagcatcatggccaccgcttcgagttcgaatctgcaaaccaactttcttggagcagagaatactggcctggatatgggagacatttgaaggagtgacaataacgagtggttttggggttgtatcagaggtgaatctaagattttgtattgtcgaattcaggacagacatatacaattggtcgtgttgagtgtatatgaattttggatttgctggattgttaggaatatattccgagaagcatttaaggaagttttcttgaggattagctattgaaatttggatattgaatgagagaaagaaaaatattattttgcaaacaaaccaaaaggagaatgttgagcaattcat

SEQ ID NO: 9 - DNA, 1631 bp, Cannabis sativa
atgatgacgcggtggaagaggtgggatactttgttcgtttctaaaaaaattattggatcagctttggtttcacctaactaacctgttaaaatttttaccaaaatacttttcaccccaaatacgtgcttgtgtgtaattattaggactctcaggattagtttttcctaaatcaaggtccctataattgagatacgccaatcttggattttgggacacataaggagttgtgaaattaataaacacttcgaaccagtttatatgcttttcgttatcttcttgctctcccaggtagcagtgtaccaaagttcatacattattccagctcgatgagggaatggaattgctgattctgaaatctcatccattataccaccgtaagggtacaacacatacatcccaactcctacctcttcttcatataatttttccaaaattttgaccattgcagtttcaggtattagtttcttaacatagtctaacttaattgagaaagccgtcttcttcccagctgatctatcaagcaaaatttcctttttaaaattagcagtgttgtaatttacaacaccactgtagaagatggttgtatcaatccagctcaattctttgcaatcagtttttttaatacccaactcaggaaagctcttgttcatcaagtcaactagactatccactccaccaagaaaaatggaagagaagtaaccatgtactgtagtcttattcttcccatgattatctgtaatattcctagttctgaagtgagtcgtgagcattaaatctttgtcatacttgtaagcaatattttgccatttgttaaataacttgacaagcccatgtatctccatgttctttttaacactgaatatagtagcctttgatgggacaacaacaagtttgattttccatgctgcaatgattccaaagttttctcctcctccaccacgtatagcccaaaatagatcttctcccatggattttcgatctagaacttttccatcaacattgactaagtgtgcatcaatgatattatcagccgcaaggccataatttcgcatcaatgctccatagcctcctccactaaagtgtccacctacgccaacagtagggcaatacccaccaggaaaactaaaattctcattcatctcattgatccaataataaacttctccaagggtagctccggcttcaacccacgcagtttggctatgaatatctactttgaccgtatgcatgtttctcaagtctactatagcaaatgggacttgagatatgtaggacaaaccctcagcatcatggccaccgcttcgagttcgaatctgcaaaccaactttcttggagcagagaatactggcctggatatgggagacatttgaaggagtgacaataacgagtggttttggggttgtatcagaggtgaatctaagattttgtattgtcgaattcaggacagacatatacaattggtcgtgttgagtgtatatgaattttggatttgctggattgttaggaatatattccgagaagcatttaaggaagttttcttgaggattagctattgaaatttggatattgaatgagagaaagaaaaatattattttgcaaacaaaccaaaaggagaatgttgagcaattcat

SEQ ID NO: 10 - DNA, 1629 bp, Cannabis sativa
atgatgacgcggtggaagaggtgggatactttgttcgtttctaaaaaaattattgggatcagctttggttttcaccttaactaacctgttaaaatttttaccaaaatacttttcaccccaaatacgtgcttgtgtgtaattattaggactctcaggattagttttcctaaatcaaggtccctataattgagatacgccaatcttggattttgggacacataaggagttgtgaaattataaacacttcgaacccagtttatatgcttttcgttatcttcttgcttctcccaggtagcagtgtaccaaagttcatacattattccagctcgatgagggaatggaattgctgattctgaaatctcatccattataccacgtaagggtacaacacatacatcccaactcctacctcttcttcatataatttttccaaaattttgaccattgcagtttcaggtattagtttcttaacatagtctaacttaattgagaaagccgtcttcttcccagctgatctatcaagaaaatttcctttttaaaattagcagtgttgtaatttacaacaccactgtagaagatggttgtatcaatccagctcaattctttgcaatcagtttttttaatacccaactcaggaaagctcttgttcatcaagtcaactagactatccactccaccaagaaaaatggaagagaagtaaccatgtactgtagtcttattcttcccatgattatctgtaatattcctagttctgaagtgagtcgtgagcattaaatctttgtcatacttgtaagcaatattttgccatttgttaaataacttgacaagcccatgtatctccatgttctttttaacactgaatatagtagcctttgatgggacaacaacaagtttgattttccatgctgcaatgattccaaagttttctcctcctccaccacgtatagcccaaaatagatcttctcccatggattttcgatctagaacttttccatcaacattgactaagtgtgcatcaatgatattatcagccgcaaggccataatttcgcatcaatgctccatagcctcctccactaaagtgtccacctacgccaacagtagggcaatacccaccaggaaaactaaaattctcattcatcttgatccaataataaacttctccaagggtagctccggcttcaacccacgcagtttggctatgaatatctactttgaccgtatgcatgtttctcaagtctactatagcaaatgggacttgagatatgtaggacaaaccctcagcatcatggccaccgcttcgagttcgaatctgcaaaccaactttcttggagcagagaatactggcctggatatgggagacatttgaaggagtgacaataacgagtggttttggggttgtatcagaggtgaatctaagattttgtattgtcgaattcaggacagacatatacaattggtcgtgttgagtgtatatgaattttggatttgctggattgttaggaatatattccgagaagcatttaaggaagttttcttgaggattagctattgaaatttggatattgaatgagagaaagaaaaatattattttgcaaacaaaccaaaaggagaatgttgagcaattcat

SEQ ID NO: 11 - protein, 533 aa, Cannabis sativa
CKIIFFFLSFNIQISIANPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAAWKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRH

SEQ ID NO: 12 - protein, 545 aa, Cannabis sativa
MNCSTFSFWFVCKIIFFFLSFNIQISIANPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAAWKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 13 - protein, 545 aa, Cannabis sativa; identical to SEQ ID NO: 12

SEQ ID NO: 14 - protein, 309 aa, Cannabis sativa
ENFGIIAAWKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 15 - protein, 534 aa, Cannabis sativa
CKIIFFFLSFNIQISIANPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAAWKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 16 - protein, 468 aa, Cannabis sativa
TPKPLVIITPLNVSHIQGTILCSKKVGLQIRTRSGGHDAEGMSYISQVPFVIVDLRNMHSVKIDVHSQTAWVEAGATLGEVYYWINENNENLSFPAGYCPTVGAGGHFSGGGYGALMRNYGLAADNIIDAHLVNVDGKVLDRKSMGEDLFWAIRGGGGENFGIIAAWKIRLDAVPSMSTIFSVKKNMEIHELVKLVNKWQNIAYMYEKELLLFTHFITRNITDNQGKNKTTIHSYFSSIFHGGVDSLVDLMNKSFPELGIKKTDCKQLSWIDTIIFYSGVVNYNTTNFKKEILLDRSGGRKAAFSIKLDYVKKPIPETAMVTILEKLYEEDVGVGMFVFYPYGGIMDEISESAIPFPHRAGIMYEIWYIASWEKQEDNEKHINWIRNVYNFTTPYVSQNPRMAYLNYRDLDLGKTNFESPNNYTQARIWGEKYFGKNFNRLVKVKTKVDHDNFFRNEQSIPPLPLRHH

SEQ ID NO: 17 - protein, 539 aa, Cannabis sativa
STFSFRFVYKIIFFFLSFNIKISIANPQENFLNCFSQYIHNNPANLKLVYTQHDQLYMSVLNLTIQNLRFTSDTTPKPLVIVTPSNVSHIQATILCSKKVGLQIRTRSGGHDAEGLSYTSQVPFVIVDLRNMHSVKIDIRSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIRLVAVPSRATIFSVKRNMEIHGLVKLFNKWQNIAYKYDKDLLLMTHFITRNIIDNQGKNKTTVHGYFSCIFHGGVDSLVNLMNKSFPELGIKKTDCKELSWIDTTIFYSGVVNYNTTNFQKEILLDRSAGQKVAFSIKLDYVKKPIPETAIVKILEKLYEEDVGVGVYVLYPYGGIMDKISESTIPFPHRAGIMYEVWYAATWEKQEDNEKHINWVRSVYNFMTPYVSQNPRMAYLNYRDLDLGKTDPKSPNNYTQARIWGEKYFGKNFDKLVKVKTKVDPNNFFRNEQSIPPLPP

SEQ ID NO: 18 - protein, 490 aa, Cannabis sativa
KYSTFCFWYVCKIIFFFLSFNIQISIANPQENFLKCFSQYIPTNVTNAKLVYTQHDQFYMSILNSTIQNLRFTSETTPKPLVIITPLNVSHIQGTILCSKKVGLQIRTRSGGHDAEGMSYISQVPFVIVDLRNMHSVKIDVHSQTAWVEAGATLGEVYYWINENNENLSFPAGYCPTVGAGGHFSGGGYGALMRNYGLAADNIIDAHLVNVDGKVLDRKSMGEDLFWAIRGGGGENFGIIAAWKIRLVAVPSMSTIFSVKKNMEIHELVKLVNKWQNIAYMYEKELLLFTHFITRNITDNQGKNKTTIHSYFSSIFHGGVDSLVDLMNKSFPELGIKKTDCKQLSWIDTIIFYSGVVNYNTTNFKKEILLDRSGGRKAAFSIKLDYVKKPIPETAMVTILEKLYEEDVGVGMFVFYPYGGIMDEISESAIPFPHRAGIMYEIWYIASWEKQEDNEKHINWIRNVYNFTTPYVSQNPRMAYLNYRDLDLGK

SEQ ID NO: 19 - protein, 492 aa, Cannabis sativa
  MOD_RES (490)..(490): Any amino acid or absent
STFCFWYVCKIIFFFLSFNIQISIANPQENFLKCFSQYIPTNVTNAKLVYTQHDQFYMSILNSTIQNLRFTSETTPKPLVIITPLNVSHIQGTILCSKKVGLQIRTRSGGHDAEGMSYISQVPFVIVDLRNMHSVKIDVHSQTAWVEAGATLGEVYYWINENNENLSFPAGYCPTVGAGGHFSGGGYGALMRNYGLAADNIIDAHLVNVDGKVLDRKSMGEDLFWAIRGGGGENFGIIAAWKIRLVAVPSMSTIFSVKKNMEIHELVKLVNKWQNIAYMYEKELLLFTHFITRNITDNQGKNKTTIHSYFSSIFHGGVDSLVDLMNKSFPELGIKKTDCKQLSWIDTIIFYSGVVNYNTTNFKKEILLDRSGGRKAAFSIKLDYVKKPIPETAMVTILEKLYEEDVGVGMFVFYPYGGIMDEISESAIPFPHRAGIMYEIWYIASWEKQEDNEKHINWIRNVYNFTTPYVSQNPRMAYLNYRDLDLGKNXFR

SEQ ID NO: 20 - protein, 281 aa, Cannabis sativa
LKCFSQYIPTNVTNAKLVYTQHDQFYMSILNSTIQNLRFTSDTTPKPLVIITPLNVSHIQGTILCSKKVGLQIRTRSGGHDAEGMSYISQVPFVIVDLRNMHSVKIDVHSQTAWVEAGATLGEVYYWINENNENLSFPAGYCPTVGAGGHFSGGGYGALMRNYGLAADNIIDAHLVNVDGKVLDRKSMGEDLFWAIRGGGGENFGIIAAWKIRLVAVPSMSTIFSVKKNMEIHELVKLVNKWQNIAYMYEKELLLFTHFITRNITDNQGKNKTTIHSYFSS

SEQ ID NO: 21 - RNA, 20 nt, Artificial Sequence
  Description of Artificial Sequence Synthetic oligonucleotide
ggaauauuacagauaaucau

SEQ ID NO: 22 - RNA, 20 nt, Artificial Sequence
  Description of Artificial Sequence Synthetic oligonucleotide
ucauccauuauaccaccgua

SEQ ID NO: 23 - RNA, 20 nt, Artificial Sequence
  Description of Artificial Sequence Synthetic oligonucleotide
aaauuauaugaagaagaggu

SEQ ID NO: 24 - RNA, 20 nt, Artificial Sequence
  Description of Artificial Sequence Synthetic oligonucleotide
gaugacgcgguggaagaggu

SEQ ID NO: 25 - RNA, 20 nt, Artificial Sequence
  Description of Artificial Sequence Synthetic oligonucleotide
ucguuucuaaaaaaauuauu

SEQ ID NO: 26 - RNA, 20 nt, Artificial Sequence
  Description of Artificial Sequence Synthetic oligonucleotide
aaauuuuaacagguuaguua

SEQ ID NO: 27 - RNA, 20 nt, Artificial Sequence
  Description of Artificial Sequence Synthetic oligonucleotide
uacacacaagcacguauuug

SEQ ID NO: 28 - RNA, 20 nt, Artificial Sequence
  Description of Artificial Sequence Synthetic oligonucleotide
cuuggauuuugggacacaua

SEQ ID NO: 29 - RNA, 20 nt, Artificial Sequence
  Description of Artificial Sequence Synthetic oligonucleotide
guuaucuucuugcuucuccc

SEQ ID NO: 30 - RNA, 20 nt, Artificial Sequence
  Description of Artificial Sequence Synthetic oligonucleotide
uacauuauuccagcucgaug

SEQ ID NO: 31 - RNA, 20 nt, Artificial Sequence
  Description of Artificial Sequence Synthetic oligonucleotide
uacaacaccacuguagaaga

SEQ ID NO: 32 - RNA, 20 nt, Artificial Sequence
  Description of Artificial Sequence Synthetic oligonucleotide
caauuuaggaaauuuucuug

SEQ ID NO: 33 - RNA, 20 nt, Artificial Sequence
  Description of Artificial Sequence Synthetic oligonucleotide
gaaggagugacaauaacgag

SEQ ID NO: 34 - RNA, 20 nt, Artificial Sequence
  Description of Artificial Sequence Synthetic oligonucleotide
uugcagauucgaacucgaag

SEQ ID NO: 35 - protein, 545 aa, Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFRELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 36 - protein, 545 aa, Cannabis sativa; identical to SEQ ID NO: 12

SEQ ID NO: 37 - protein, 377 aa, Cannabis sativa
MNCSTFSFWFVCKIIFFFLSFNIQISIANPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWIKMNENFSFPGGYCPTVGVGGHFSGGGYGALMRNYGLAADNIIDAHLVNVDGKVLDRKSMGEDLFWAIRGGGGENFGIIAAWKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEIFLIDQLGRR

SEQ ID NO: 38 - protein, 420 aa, Cannabis sativa
  MOD_RES (162)..(162): Any amino acid or absent
PICYSRLENMHTVKVDIHSQTAWVEAGATLGEVYYWINEMNENFSFPGGYCPTVGVGGHFSGGGYGALMRNYGLAADNIIDAHLVNVDGKVLDRKSMEKIYFGLYVVEEEKTLESLQHGKSNLLLSHQRLLYSVLKRTWRYMGLSSYLTNGKILLTSMTKIXCSRLTSETRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETVMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 39 - protein, 450 aa, Cannabis sativa
MNCSTFSFWFVCKIIFFFLSFNIQISIANPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAAWKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWE

SEQ ID NO: 40 - protein, 542 aa, Cannabis sativa
  MOD_RES (369)..(369): Any amino acid or absent
STFCFWYVCKIIFFFLSFNIQISIANPQENFLKCFSQYIPTNVTNAKLVYTQHDQFYMSILNSTIQNLRFTSDTTPKPLVIITPLNVSHIQGTILCSKKVGLQIRTRSGGHDAEGMSYISQVPFVIVDLRNMHSVKIDVHSQTAWVEAGATLGEVYYWINENNENLSFPAGYCPTVGAGGHFSGGGYGALMRNYGLAADNIIDAHLVNVDGKVLDRKSMGEDLFWAIRGGGGENFGIIAAWKIRLVAVPSMSTIFSVKKNMEIHELVKLVNKWQNIAYMYEKELLLFTHFITRNITDNQGKNKTTIHCYFSSIFHGGLDSLVDLMNKSFPELGIKKTDCKQLSWIDTIIFNSGLVNYNTTNFKKEILLXRSGGRKAAFSIKLDYVKKPIPETAMVTILEKLYEEDVGVGMFVFYPYGGIMDEISESAIPFPHRAGIMYEIWYIASWEKQEDNEKHINWIRNVYNFTTPYVSQNPRMAYLNYRDLDLGKTNFESPNNYTQARIWGEKYFGKNFNRLVKVKTKVDPDNFFRNEQSIPPLPLRHH

SEQ ID NO: 41 - protein, 430 aa, Cannabis sativa
STFCFWYVCKIIFFFLSFNIQISIANPQENFLKCLSQYIPTNVTNAKLVYTQHDQFYMSILNSTIQNLRFTSDTTPKPLVIITPLNVSHIQGTILCSKKFGLQIRTRSGGHDAEGMSYISQVPFVIVDLRNMHSVKIDVHSQNAWVEAGATLGEVYYWINENNENLSFPAGYCPTVGACGHFSGGGYGALMRNYGLAADNIIDAHLVNVDGKVLDRKSMGEDLFWAIRGGGGENFGIIAAWKIRLVAVPSMSTIFSVKKNMEIHELVKLVNKWQNIAYMYEKELLLFTHFITRNITDNQGKNKTTIHSYFSSIFHGGVDSLVDLMNKSFPELGIKKRDCKQLSWIDTIIFYSGLVNYNTTNFKKEILLDRSGGRKAAFSIKLDYVKKPIPETAMVTILEKLYEEDVGVGMFVFYPYGGIMDEISESAIPF

SEQ ID NO: 42 - protein, 536 aa, Cannabis sativa
  MOD_RES (438)..(438): Any amino acid or absent
  MOD_RES (457)..(457): Any amino acid or absent
STFSFRFVYKIIFFFLSFNIKISIANPQENFLKCFSQYIHNNPANLKLVYTQHDQLYMSVLNLTIQNLRFTSDTTPKPLVIVTPSNVSHIQATILCSKKVGLQIRTRSGGHDAEGLSYTSQVPFVIVDLRNMHSVKIDIRSQIAWVEAGATLGEVYYWINENLSFPGGYCPTVGVGGHFSGGGYRALMRNYGLAADNIIDAHLVNVDGKVLDRKSMGEDLFWAIRGGGGENFGIIAAWKIRLVAVPSRATIFSVKRNMEIHGLVKLFNKWQNIAYKYDKDLLLMTHFITRNIIDNQGKNKTTVHGYFSCIFHGGVDSLVNLMNKSFPELGIKKTDCKELSWIDTTIFYSGVVNYNTTNFQKEILLDRSAGQKVAFSVKLDYVKKPIPETAIVKILEKLYEEDVGVGVYVLYPYGGIMDKISESTIPFPHRAGIMYEVXYAATWEKQEDNEKHINWVXSVYNFMTPYVSQNPRMAYLNYRDLDLGKTDPKSPNNYTQARIWGEKYFGKNFDKLVKVKTKVDPNNFFRNEQSIPPLPP

SEQ ID NO: 43 - protein, 543 aa, Cannabis sativa
  MOD_RES (105)..(105): Any amino acid or absent
  MOD_RES (285)..(285): Any amino acid or absent
  MOD_RES (385)..(385): Any amino acid or absent
KYSTFCFWYVCKIIFFFLSFNIQISIANPEGNFLKCFSQYIPTNVTNAKLVYTQHDQFYMSILNSTIQNLRFTFDTTPKPLVIITPLNVSHIQGTILCSKKVGLXIRTRSGGHDAEGMSYISQVPFVIVNLRNMHSVKIDVHSETAWVEAGATLGEVYYWINENNENLSFLAGYCPTVGAGGHFSGGGYGALMRNYGLAANNIIDAHLVNVDGKVLDRKSMGEDLFWAIRGGGENFGIIAAWKIRFVAVPSMSTIFSVKKNMEIHELVKLVNKWQNIAYMYEKEXLLFTHFITRNITDNQGKNKTTIHSYFSSIFYGGVDSLVDLMNKSFPELGIKKTDCKQLSWIDTIIFYSGLVNYNTTNFKKELLLDRSGGRKAAFSIKLDXVKKPIPETAMVTILEKLYEEDVGVGMFVFYPYGGIMDEISESAIPFPHRAGIMYEIWYIASWEKQEDNEKHINWIRNVYNFTTPYVSQNPRMAYLNYRDLDLGKTNFESPNNYTQARIWGEKYFGKNFNRLVKVKTKVDPDNFFRNEQSIPPLPLRHH

SEQ ID NO: 44 - protein, 516 aa, Cannabis sativa
NPRENFLKCFSQYIPNNATNLKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYWVNEKNESLSLAAGYCPTVCAGGHFGGGGYGPLMRSYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHHH

SEQ ID NO: 45 - protein, 463 aa, Cannabis sativa
NPQENFLKCFSQYIPTNVTNAKLVYTQHDQFYMSILNSTIQNLRFTSETTPKPLVIITPLNVSHIQGTILCSKKVGLQIRTRSGGHDAEGMSYISQVPFVIVDLRNMHSVKIDVHSQTAWVEAGATLGEVYYWINENNENLSFPAGYCPTVGAGGHFSGGGYGALMRNYGLAADNIIDAHLVNVDGKVLDRKSMGEDLFWAIRGGGGENFGIIAAWKIRLVAVPSMSTIFSVKKNMEIHELVKLVNKWQNIAYMYEKELLLFTHFITRNITDNQGKNKTTIHSYFSSIFHGGVDSLVDLMNKSFPELGIKKTDCKQLSWIDTIIFYSGVVNYNTTNFKKEILLDRSGGRKAAFSIKLDYVKKPIPETAMVTILEKLYEEDVGVGMFVFYPYGGIMDEISESAIPFPHRAGIMYEIWYIASWEKQEDNEKHINWIRNVYNFTTPYVSQNPRMAYLNYRDLDLGK

SEQ ID NO: 46 - protein, 545 aa, Cannabis sativa
  MOD_RES (545)..(545): Any amino acid or absent
MKYSTFSFWFVCKIIFFFFSFNIQTSIANPRENFLKCFSQYIPNNATNLKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYWVNEKNESLSLAAGYCPTVCAGGHFGGGGYGPLMRSYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHHHX

SEQ ID NO: 47 - protein, 544 aa, Cannabis sativa
  MOD_RES (27)..(27): Any amino acid or absent
  MOD_RES (34)..(34): Any amino acid or absent
  MOD_RES (70)..(70): Any amino acid or absent
  MOD_RES (74)..(74): Any amino acid or absent
  MOD_RES (113)..(113): Any amino acid or absent
  MOD_RES (115)..(115): Any amino acid or absent
  MOD_RES (142)..(142): Any amino acid or absent
  MOD_RES (148)..(148): Any amino acid or absent
  MOD_RES (162)..(162): Any amino acid or absent
  MOD_RES (165)..(165): Any amino acid or absent
  MOD_RES (168)..(168): Any amino acid or absent
  MOD_RES (184)..(184): Any amino acid or absent
  MOD_RES (200)..(201): Any amino acid or absent
  MOD_RES (204)..(204): Any amino acid or absent
  MOD_RES (211)..(211): Any amino acid or absent
  MOD_RES (245)..(245): Any amino acid or absent
  MOD_RES (259)..(259): Any amino acid or absent
  MOD_RES (266)..(266): Any amino acid or absent
  MOD_RES (272)..(272): Any amino acid or absent
  MOD_RES (282)..(282): Any amino acid or absent
  MOD_RES (299)..(299): Any amino acid or absent
  MOD_RES (304)..(304): Any amino acid or absent
  MOD_RES (544)..(544): Any amino acid or absent
MKYSTFCFWYVCKIIFSFSHSISKFQXLILKKTXMLLTIYSHQCNKCKTRIHSTRPILYVYPKFDHTKSXIYLXHNPKTTCYHHSFKCLPYPRHYSMLQESWLADSNSKRWSXCXGHVLHISSPICYSRLEKHAFGQNRCSXPNCMGXSRSYPWRSLLLDQXEQXESXFSCWVLPYCWRGWTLXWRRLWSIDAKLWPRGXXYHXCALSQCXWKSFRSKIHGGRFVLGYTWWWRRKLWNHCSVENXTCCCPINVYYIQCXKEHGDTXACQVSXQMAKYCLHVXKRIITLYSLYNQEYYRXSREEXDNNTQLLLLIFHGGVDSLVDLMNKSFPELGIKKTDCKQLSWIDTIIFYSGVVNYNTTNFKKEILLDRSGGRKAAFSIKLDYVKKPIPETAMVTILEKLYEEDVGVGMFVFYPYGGIMDEISESAIPFPHRAGIMYEIWYIASWEKQEDNEKHINWIRNVYNFTTPYVSQNPRMAYLNYRDLDLGKTNFESPNNYTQARIWGEKYFGKNFNRLVKVKTKVDPDNFFRNEQSIPPLPLRHHX

SEQ ID NO: 48 - protein, 546 aa, Cannabis sativa
  MOD_RES (493)..(493): Any amino acid or absent
  MOD_RES (498)..(499): Any amino acid or absent
  MOD_RES (508)..(508): Any amino acid or absent
  MOD_RES (513)..(513): Any amino acid or absent
  MOD_RES (516)..(517): Any amino acid or absent
  MOD_RES (526)..(526): Any amino acid or absent
  MOD_RES (529)..(529): Any amino acid or absent
  MOD_RES (532)..(532): Any amino acid or absent
MKYSTFCFWYVCKIIFFFLSFNIQISIANPQENFLKCFSQYIPTNVTNAKLVYTQHDQFYMSILNSTIQNLRFTSETTPKPLVIITPLNVSHIQGTILCSKKVGLQIRTRSGGHDAEGMSYISQVPFVIVDLRNMHSVKIDVHSQTAWVEAGATLGEVYYWINENNENLSFPAGYCPTVGAGGHFSGGGYGALMRNYGLAADNIIDAHLVNVDGKVLDRKSMGEDLFWAIRGGGGENFGIIAAWKIRLVAVPSMSTIFSVKKNMEIHELVKLVNKWQNIAYMYEKELLLFTHFITRNITDNQGKNKTTIHSYFSSIFHGGVDSLVDLMNKSFPELGIKKTDCKQLSWIDTIIFYSGVVNYNTTNFKKEILLDRSGGRKAAFSIKLDYVKKPIPETAMVTILEKLYEEDVGVGMFVFYPYGGIMDEISESAIPFPHRAGIMYEIWYIASWEKQEDNEKHINWIRNVYNFTTPYVSQNPRMAYLNYRDLDLGKNXFRESXXLHTSTYLGXKVFWXKFXXVSKSKNQGXSRXFLXKRTKHPTSSPASSL

SEQ ID NO: 49 - protein, 546 aa, Cannabis sativa; identical to SEQ ID NO: 48
  MOD_RES (493)..(493): Any amino acid or absent
  MOD_RES (498)..(499): Any amino acid or absent
  MOD_RES (508)..(508): Any amino acid or absent
  MOD_RES (513)..(513): Any amino acid or absent
  MOD_RES (516)..(517): Any amino acid or absent
  MOD_RES (526)..(526): Any amino acid or absent
  MOD_RES (529)..(529): Any amino acid or absent
  MOD_RES (532)..(532): Any amino acid or absent

SEQ ID NO: 50 - protein, 545 aa, Cannabis sativa
  MOD_RES (88)..(88): Any amino acid or absent
  MOD_RES (129)..(130): Any amino acid or absent
  MOD_RES (132)..(132): Any amino acid or absent
  MOD_RES (140)..(140): Any amino acid or absent
  MOD_RES (193)..(193): Any amino acid or absent
  MOD_RES (209)..(209): Any amino acid or absent
  MOD_RES (217)..(217): Any amino acid or absent
  MOD_RES (272)..(272): Any amino acid or absent
  MOD_RES (294)..(294): Any amino acid or absent
  MOD_RES (324)..(324): Any amino acid or absent
  MOD_RES (327)..(328): Any amino acid or absent
  MOD_RES (345)..(345): Any amino acid or absent
  MOD_RES (358)..(358): Any amino acid or absent
  MOD_RES (385)..(385): Any amino acid or absent
  MOD_RES (409)..(409): Any amino acid or absent
  MOD_RES (422)..(422): Any amino acid or absent
  MOD_RES (446)..(446): Any amino acid or absent
  MOD_RES (459)..(459): Any amino acid or absent
  MOD_RES (489)..(489): Any amino acid or absent
  MOD_RES (518)..(519): Any amino acid or absent
  MOD_RES (521)..(521): Any amino acid or absent
MKYSTFCFWYVCKIIFFFLSFNIQISIANPQENFLKCFSQYIPTNVTNAKLVYTQHDQFYMSILNSTIQNLRFTSEQPQNHLLSSLLXMSPISKALFYAPRKLACRFELEAVVMMLRACPTYLKSHLLXXTXETCIRSKXMFIAKLHGLKPELPLEKFIIGSMRTMRILVFLLGTALLLARVDTLVEEAMEHXCEIMASRLIISLMRTXSMLMEKFXIENPWGKICFGLYVVVEEKTLESLQRGKLDLMLSHQCLLYSVLKRTWRYMSLSSXLTNGKILLTCMKKNYYSLLTLXPGILQIIKGRIRQQYTVTSPPFSMVEWIVXSTXXTRAFLNWVLKKQIANSXAGLILSSSTVVLXITTQLILKKKFCLIDQVGGRRLSRLSXTMLRNRFQKPQWSQFWKNYMKKMXELGCLCFTLMVVXWMRFQNQQFHSLIELESCMKFGTXLHGRSKKIMKSIXTGFGMFIISRLLMCPKIQEWRISIIGTLIXEKLISRVLIITHKHVFGVKSILVKILIGXXKXKPRLITIISLETNKASHLFPCVII

SEQ ID NO: 51 - protein, 545 aa, Cannabis sativa; identical to SEQ ID NO: 50
  MOD_RES (88)..(88): Any amino acid or absent
  MOD_RES (129)..(130): Any amino acid or absent
  MOD_RES (132)..(132): Any amino acid or absent
  MOD_RES (140)..(140): Any amino acid or absent
  MOD_RES (193)..(193): Any amino acid or absent
  MOD_RES (209)..(209): Any amino acid or absent
  MOD_RES (217)..(217): Any amino acid or absent
  MOD_RES (272)..(272): Any amino acid or absent
  MOD_RES (294)..(294): Any amino acid or absent
  MOD_RES (324)..(324): Any amino acid or absent
  MOD_RES (327)..(328): Any amino acid or absent
  MOD_RES (345)..(345): Any amino acid or absent
  MOD_RES (358)..(358): Any amino acid or absent
  MOD_RES (385)..(385): Any amino acid or absent
  MOD_RES (409)..(409): Any amino acid or absent
  MOD_RES (422)..(422): Any amino acid or absent
  MOD_RES (446)..(446): Any amino acid or absent
  MOD_RES (459)..(459): Any amino acid or absent
  MOD_RES (489)..(489): Any amino acid or absent
  MOD_RES (518)..(519): Any amino acid or absent
  MOD_RES (521)..(521): Any amino acid or absent

SEQ ID NO: 52 - protein, 358 aa, Cannabis sativa
  MOD_RES (173)..(173): Any amino acid or absent
  MOD_RES (248)..(248): Any amino acid or absent
  MOD_RES (337)..(337): Any amino acid or absent
SKKIGLQIRTRSGGHDSEDMSYISQVPFVIVDLRNMHSINIDVHSQIARVEAGATLGEVYYWVNEKNENLSLAAGYCPTVSAAGHFGGGGYGPLMQNYGLAADNIVDAHLVNVDAKVLDRKSMGEDLFWAIRGGGGESFGIIVAWKIRLVAVPTKSTMFSVKKIMEIHELVKXVNKWQNIAYKYDKDLLLMTHFITRNITNNHGKNKTTIHTYFSSVFLGGVDSLVDLMNKSFPELGIKKTDCKQLSXIDIIIFYSGVVNYGTDNFNKEILLDRSAGQNGSLKIKLDYVKKPIPESAFVKILEKLYEEDEGAGMYALYPYGGIMDEISESAIPFPHXAGIMYELWYICSWEKHEDNEK

SEQ ID NO: 53 - protein, 505 aa, Cannabis sativa
  MOD_RES (112)..(112): Any amino acid or absent
  MOD_RES (114)..(114): Any amino acid or absent
  MOD_RES (166)..(166): Any amino acid or absent
  MOD_RES (191)..(191): Any amino acid or absent
MKYSTFSFWFVCKIIFFFLSFNIQPSIANPRENFLKCFSQYIPTNVTNLKLTPKTTLYMPVQNSTIHNLRFTSNTTPKLLVIVTLHMSLISKALFYVQENWFANSNSKRWSXFXRHVPHISSPICYSRLEKHAFNQKMFIAKSQGLKPELPLEKFIIGLMRKMRSXFGCWYCPTVSAAGHFGGGGYGPLMXNYGLADDNIVDAHLVNVDGKVLDRKSMGQDLFWAIRGGGRESFRIIVAWKIRLVAVPTKSTMFSVKKIKEIHELVKLVNKWQNISYKYDIDLLLMTHFITRNITDNQGKNKTTIHTYFSLVFLGGVDSLVDLMNKSFPEFGIKKIDCKQLSWIDTIIFYSGVVNYGTDNFNNQISLVRSAGQNGAFKIKLDYVKKPIPESAFVKILEKLYEEDKGVGMYALYPYGCLMDEISESAIPFPHRVGIMYELWYICSWEKHEDKEKYLNWIRNVDNFMTPYVSQNPRLTYLNYRHLDIGINDPKSQNNYTEACIWGEK

SEQ ID NO: 54 - protein, 541 aa, Cannabis sativa
  MOD_RES (372)..(372): Any amino acid or absent
MKYSTFCFWYVCKIIFFFLSFNIQISIANPQENFLKCFSQYIPTNVTNAKLVYTQHDQFYMSILNSTIQNLRFTSDTTPKPLVIITPLNVSHIQGTILCSKKVGLQIRTRSGGHDAEGMSYISQVPFVIVDLRNMHSVKIDVHSQTAWVEAGATLGEVYYWINENNENLSFPAGYCPTVGAGGHFSGGGYGALMRNYGLAADNIIDAHLVNVDGKVLDRKSMGEDLFWAIRGGGGENFGIIAAWKIRLVAVPSMSTIFSVKKNMEIHELVKLVNKWQNIAYMYEKELLLFTHFITRNITDNQGKNKTTIHCYFSSIFHGGLDSLVDLMNKSFPELGIKKTDCKQLSWIDTIIFNSGLVNYNTTNFKKEILLXRSGGRKAAFSIKLDYVKKPIPETAMVTILEKLYEEDVGVGMFVFYPYGGIMDEISESAIPFPHRAGIMYEIWYIASWEKQEDNEKHINWIRNVYNFTTPYVSQNPRMAYLNYRDLDLGKTNFESPNNYTQARIWGEKYFGKNFNRLVKVKTKVDPDNFFRNEQSIPPLP

SEQ ID NO: 55 - protein, 545 aa, Cannabis sativa; identical to SEQ ID NO: 35

SEQ ID NO: 56 - protein, 433 aa, Cannabis sativa
MKYSTFCFWYVCKIIFFFLSFNIQISIANPQENFLKCLSQYIPTNVTNAKLVYTQHDQFYMSILNSTIQNLRFTSDTTPKPLVIITPLNVSHIQGTILCSKKFGLQIRTRSGGHDAEGMSYISQVPFVIVDLRNMHSVKIDVHSQNAWVEAGATLGEVYYWINENNENLSFPAGYCPTVGACGHFSGGGYGALMRNYGLAADNIIDAHLVNVDGKVLDRKSMGEDLFWAIRGGGGENFGIIAAWKIRLVAVPSMSTIFSVKKNMEIHELVKLVNKWQNIAYMYEKELLLFTHFITRNITDNQGKNKTTIHSYFSSIFHGGVDSLVDLMNKSFPELGIKKRDCKQLSWIDTIIFYSGLVNYNTTNFKKEILLDRSGGRKAAFSIKLDYVKKPIPETAMVTILEKLYEEDVGVGMFVFYPYGGIMDEISESAIPF

SEQ ID NO: 57 - protein, 433 aa, Cannabis sativa; identical to SEQ ID NO: 56

SEQ ID NO: 58 - protein, 490 aa, Cannabis sativa
  MOD_RES (78)..(78): Any amino acid or absent
  MOD_RES (232)..(232): Any amino acid or absent
  MOD_RES (332)..(332): Any amino acid or absent
NPEGNFLKCFSQYIPTNVTNAKLVYTQHDQFYMSILNSTIQNLRFTFDTTPKPLVIITPLNVSHIQGTILCSKKVGLXIRTRSGGHDAEGMSYISQVPFVIVNLRNMHSVKIDVHSETAWVEAGATLGEVYYWINENNENLSFLAGYCPTVGAGGHFSGGGYGALMRNYGLAANNIIDAHENFGIIAAWKIRFVAVPSMSTIFSVKKNMEIHELVKLVNKWQNIAYMYEKEXLLFTHFITRNITDNQGKNKTTIHSYFSSIFYGGVDSLVDLMNKSFPELGIKKTDCKQLSWIDTIIFYSGLVNYNTTNFKKELLLDRSGGRKAAFSIKLDXVKKPIPETAMVTILEKLYEEDVGVGMFVFYPYGGIMDEISESAIPFPHRAGIMYEIWYIASWEKQEDNEKHINWIRNVYNFTTPYVSQNPRMAYLNYRDLDLGKTNFESPNNYTQARIWGEKYFGKNFNRLVKVKTKVDPDNFFRNEQSIPPLPLRHH

SEQ ID NO: 59 - protein, 541 aa, Cannabis sativa
MNCSTFSFWFVCKIIFFFLSFNIQISIANPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAAWKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLP

SEQ ID NO: 60 - protein, 416 aa, Cannabis sativa
  MOD_RES (162)..(162): Any amino acid or absent
PICYSRLENMHTVKVDIHSQTAWVEAGATLGEVYYWINEMNENFSFPGGYCPTVGVGGHFSGGGYGALMRNYGLAADNIIDAHLVNVDGKVLDRKSMEKIYFGLYVVEEEKTLESLQHGKSNLLLSHQRLLYSVLKRTWRYMGLSSYLTNGKILLTSMTKIXCSRLTSETRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETVMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLP

SEQ ID NO: 61 - protein, 377 aa, Cannabis sativa; identical to SEQ ID NO: 37

SEQ ID NO: 62 - protein, 539 aa, Cannabis sativa
  MOD_RES (438)..(438): Any amino acid or absent
  MOD_RES (457)..(457): Any amino acid or absent
STFSFRFVYKIIFFFLSFNIKISIANPQENFLKCFSQYIHNNPANLKLVYTQHDQLYMSVLNLTIQNLRFTSDTTPKPLVIVTPSNVSHIQATILCSKKVGLQIRTRSGGHDAEGLSYTSQVPFVIVDLRNMHSVKIDIRSQIAWVEAGATLGEVYYWINENLSFPGGYCPTVGVGGHFSGGGYRALMRNYGLAADNIIDAHLVNVDGKVLDRKSMGEDLFWAIRGGGGENFGIIAAWKIRLVAVPSRATIFSVKRNMEIHGLVKLFNKWQNIAYKYDKDLLLMTHFITRNIIDNQGKNKTTVHGYFSCIFHGGVDSLVNLMNKSFPELGIKKTDCKELSWIDTTIFYSGVVNYNTTNFQKEILLDRSAGQKVAFSVKLDYVKKPIPETAIVKILEKLYEEDVGVGVYVLYPYGGIMDKISESTIPFPHRAGIMYEVXYAATWEKQEDNEKHINWVXSVYNFMTPYVSQNPRMAYLNYRDLDLGKTDPKSPNNYTQARIWGEKYFGKNFDKLVKVKTKVDPNNFFRNEQSIPPLPPRRH

SEQ ID NO: 63 - protein, 450 aa, Cannabis sativa; identical to SEQ ID NO: 39

SEQ ID NO: 64 - DNA, 1635 bp, Cannabis sativa
atgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctctcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatttctcaagtcccatttgttgtagtagacttgaggaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattcctgaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcat

SEQ ID NO: 65 - DNA, 1635 bp, Cannabis sativa
atgaattgctcagcattttccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcatcatcat

SEQ ID NO: 66 - DNA, 1635 bp, Cannabis sativa
atgaattgctcagcattttccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcaacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcatcatcat

SEQ ID NO: 67 - DNA, 1635 bp, Cannabis sativa
atgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcat

SEQ ID NO: 68 - DNA, 1635 bp, Cannabis sativa
atgaattgctcagcattttccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgattcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtcaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaagtgaaaaccaaagctgatcccaataatttttttagaaacgaacaaagtatcccacctcttccaccgcatcatcat

SEQ ID NO: 69 - DNA, 1635 bp, Cannabis sativa; identical to SEQ ID NO: 67

SEQ ID NO: 70 - DNA, 1635 bp, Cannabis sativa; identical to SEQ ID NO: 67

SEQ ID NO: 71 - DNA, 1635 bp, Cannabis sativa
atgaattgctcagcattttccttttggtttgtttgcaaaataatatttttctttctctcattcaatatccaaatttcattagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacgtgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttacaatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcatcatcat

SEQ ID NO: 72 - DNA, 1635 bp, Cannabis sativa; identical to SEQ ID NO: 67

SEQ ID NO: 73 - DNA, 1635 bp, Cannabis sativa; identical to SEQ ID NO: 67

SEQ ID NO: 74 - DNA, 1635 bp, Cannabis sativa
atgaattgctcagcattttccttttggtttgtttgcaaaataataattttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcatcatcat

SEQ ID NO: 75 - DNA, 1635 bp, Cannabis sativa
atgaattgctcagcattttccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatgcaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcatcatcat

SEQ ID NO: 76 - DNA, 1635 bp, Cannabis sativa
atgaattgctcagcattttccttttggtttgtttgcaaaataatatttttctttctctcattcaatatccaaatttcattagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaacgccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcatcatcat

SEQ ID NO: 77 - DNA, 20 bp, Cannabis sativa
cgagaaaacttccttaaatg

SEQ ID NO: 78 - DNA, 20 bp, Cannabis sativa
caaaaccactcgttattgtc

SEQ ID NO: 79 - DNA, 20 bp, Cannabis sativa
ctcgttattgtcactccttc

SEQ ID NO: 80 - DNA, 20 bp, Cannabis sativa
aacgtctaagcttgagcttc

SEQ ID NO: 81 - DNA, 20 bp, Cannabis sativa
gtctaagcttgagcttcgcc

SEQ ID NO: 82 - DNA, 20 bp, Cannabis sativa
tgatgctgagggtatgtcct

SEQ ID NO: 83 - DNA, 20 bp, Cannabis sativa
tcgccaccggtactacgact

SEQ ID NO: 84 - DNA, 20 bp, Cannabis sativa
acaagtatcggtttgacgca

SEQ ID NO: 85 - DNA, 20 bp, Cannabis sativa
ggtgggtattgccctactgt

SEQ ID NO: 86 - DNA, 20 bp, Cannabis sativa
catccacctgtgaaatcacc

SEQ ID NO: 87 - RNA, 96 nt, Artificial Sequence
  Description of Artificial Sequence Synthetic oligonucleotide
cauuuaaggaaguuuucucgguuuuagagcuagaaauagcaaguuaaaauaaggcuaguccguuaucaacuugaaaaaguggcaccgagucggugc

SEQ ID NO: 88 - RNA, 96 nt, Artificial Sequence
  Description of Artificial Sequence Synthetic oligonucleotide
gacaauaacgagugguuuugguuuuagagcuagaaauagcaaguuaaaauaaggcuaguccguuaucaacuugaaaaaguggcaccgagucggugc

SEQ ID NO: 89 - RNA, 96 nt, Artificial Sequence
  Description of Artificial Sequence Synthetic oligonucleotide
gaaggagugacaauaacgagguuuuagagcuagaaauagcaaguuaaaauaaggcuaguccguuaucaacuugaaaaaguggcaccgagucggugc

SEQ ID NO: 90 - RNA, 96 nt, Artificial Sequence
  Description of Artificial Sequence Synthetic oligonucleotide
cagauucgaacucgaagcggguuuuagagcuagaaauagcaaguuaaaauaaggcuaguccguuaucaacuugaaaaaguggcaccgagucggugc

SEQ ID NO: 91 - RNA, 96 nt, Artificial Sequence
  Description of Artificial Sequence Synthetic oligonucleotide
aggacauacccucagcaucaguuuuagagcuagaaauagcaaguuaaaauaaggcuaguccguuaucaacuugaaaaaguggcaccgagucggugc

SEQ ID NO: 92 - RNA, 96 nt, Artificial Sequence
  Description of Artificial Sequence Synthetic oligonucleotide
agcgguggccaugaugcugaguuuuagagcuagaaauagcaaguuaaaauaaggcuaguccguuaucaacuugaaaaaguggcaccgagucggugc

SEQ ID NO: 93 - RNA, 96 nt, Artificial Sequence
  Description of Artificial Sequence Synthetic oligonucleotide
uguucauagccaaacugcguguuuuagagcuagaaauagcaaguuaaaauaaggcuaguccguuaucaacuugaaaaaguggcaccgagucggugc

SEQ ID NO: 94 - RNA, 96 nt, Artificial Sequence
  Description of Artificial Sequence Synthetic oligonucleotide
acaguagggcaauacccaccguuuuagagcuagaaauagcaaguuaaaauaaggcuaguccguuaucaacuugaaaaaguggcaccgagucggugc

SEQ ID NO: 95 - RNA, 96 nt, Artificial Sequence
  Description of Artificial Sequence Synthetic oligonucleotide
guagguggacacuuuaguggguuuuagagcuagaaauagcaaguuaaaauaaggcuaguccguuaucaacuugaaaaaguggcaccgagucggugc

SEQ ID NO: 96 - DNA, 13490 bp, Artificial Sequence
  Description of Artificial Sequence Synthetic polynucleotide
agcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcaggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctgcaagcaagaattcaagcttggagccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgatgacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtaatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagactatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagattatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtctccacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtttcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaaggatctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctactaggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgcttgcctctgttccccagagggcagttttagagctagaaatagcaagttaaaataaggctagtccgttatcaacttgaaaaagtggcaccgagtcggtgctttttttctagacccagctttcttgtacaaagttggcattacgctttacgaattcccatggggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgcttgctgttccccagagggcaggggttttagagctagaaatagcaagttaaaataaggctagtccgttatcaacttgaaaaagtggcaccgagtcggtgctttttttctagacccagctttcttgtacaaagttggcattacgctcagagaattcgcatgcggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgcttgaacctcaagcacgagaacttgttttagagctagaaatagcaagttaaaataaggctagtccgttatcaacttgaaaaagtggcaccgagtcggtgctttttttctagacccagctttcttgtacaaagttggcattacgcttgtgtgagaccgaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagcgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccattacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactctgtgggtctcatgccgaattcggatccggaggaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaagtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtcaagactggcgaacagttcatacagagtcttttacgactcaatgacaagaagaaaatcttcgtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatgaaaa

SEQ ID NO: 97 - DNA, 13377 bp, Artificial Sequence
  Description of Artificial Sequence Synthetic polynucleotide
ctcgagcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttcttgactcttcgcgatgtacgggccagatatgtcgaccgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcgcgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctcgcggcctggccggcgagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggcgagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagttagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcacctctagatggcaggatatattgtggtgtaaacagtttaaacagtgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcgaacgtccggagttatcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccattgttggtacccagcttgggtctagtcgtattaagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccattacataggcccatcggagctaacgcagtgaattcagaaatctcaaaattccggcagaacaattttgaatctcgatccgtagaaacgagacggtcattgttttagttccaccacgattatatttgaaatttacgtgagtgtgagtgagacttgcataagaaaataaaatctttagttgggaaaaaattcaataatataaatgggcttgagaaggaagcgagggataggcctttttctaaaataggcccatttaagctattaacaatcttcaaaagtaccacagcgcttaggtaaagaaagcagctgagtttatatatggttagagacgaagtagtgattggatggcaggtggaagaatggacacctgcgagagttttagagctagaaatagcaagttaaaataaggctagtccgttatcaacttgaaaaagtggcaccgagtcggtgctttttttacagtgaaagcttactgcgttagctccgatgggcctatgtaatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctcttaatacgactcactatagggagacccaagctggctagcaacaatggataagaagtactctatcggactcgatatcggaactaactctgttggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagattttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagggtgaacaccgagatcaccaaggctccactttctgcttctatgatcaagagatacgatgagcaccaccaggatctcacacttctcaaggctcttgttagacagcagctcccagagaagtacaaagaaatcttcttcgatcagtctaagaacggatacgctggttacatcgatggtggtgcatctcaagaagagttctacaagttcatcaagccaatcttggagaagatggatggaaccgaggaactcctcgtgaagctcaatagagaggatctccttaggaagcagaggaccttcgataacggatctatccctcatcagatccacctcggagagttgcacgctatccttagaaggcaagaggatttctacccattcctcaaggataacagagagaagattgagaagatcctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttcctaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagtggaaatctctggtgttgaggataggttcaacgcatctctcggaacctaccacgatctcctcaagatcattaaggataaggatttcttggataacgaggaaaacgaggatatcttggaggatatcgttcttaccctcaccctcttcgaggatagagagatgatagaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatcccctgcaatcaagaagggaatcctccagactgtgaagattgtggatgagttggtgaaggtgatgggacacaagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaacgagaagctctacctctactacttgcagaacggaagggatatgtatgtggatcaagagcttgatattaacaggctctctgattacgatgttgatcatatcgtgccacagtcttttatcaaagatgattctatcgataacaaggtgctcactaggtctgataagaacaggggtaagagtgataacgtgccaagtgaagaggttgtgaagaaaatgaagaactattggaggcagctcctcaacgctaagctcatcactcagagaaagttcgataacttgaccaaggctgagaggggaggactctctgaattggataaggcaggattcatcaagagacagctcgtggaaaccaggcagatcaccaaacatgtggcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatccaaagttggagtctgagttcgtgtacggtgattataaggtgtacgatgtgaggaagatgatcgctaagtctgagcaagagatcggaaaggctaccgctaagtatttcttctactctaacatcatgaatttcttcaagaccgagatcactctcgctaacggtgagatcagaaagaggccactcatcgagacaaacggtgaaacaggtgagatcgtgtgggataagggaagggatttcgctaccgttagaaaggtgctctctatgcctcaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttccttgaggctaagggatacaaagaggttaagaaggatctcatcatcaagctcccaaagtactcacttttcgagttggagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatcagtgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagcttggaggtgatcctaagaagaaaagaaaggttagatcttgatgacccgggtctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaaggccttgacaggatatattggcgggtaaacctaagagaaaagagcgtttattagaataacggatatttaaaactcgag

SEQ ID NO: 98 - DNA, 11850 bp, Artificial Sequence
  Description of Artificial Sequence Synthetic polynucleotide
gatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgactcgtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactcgaagtcacagccaaaagccagacagagtctgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttcagctgtctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaagctagccaccaccaccaccaccacgtgtgaattacaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttagcttcatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcttgaccatggta

SEQ ID NO: 99 - DNA, 17981 bp, Artificial Sequence
  Description of Artificial Sequence Synthetic polynucleotide
tgagcgtcgcaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggcccggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctaatcaaacaagtttgtacaaaaaagctgaacgagaaacgtaaaatgatgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcattaaaatatattgatatttatatcattttacgtttctcgttcagctttcttgtacaaagtggttcgatctagaggatccatggtgagcaagggcgaggagctgttcaccggggtggtgcccatcctggtcgagctggacggcgacgtgaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaaagcggcccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattagcttcatcaacgcaagacatgcgcacgaccgtctgacaggagaggaatttccgacgagcacagaaaggacttgctcttggacgtaggcctatttctcaggcacatgtatcaagtgttcggacgtgggttttcgatggtgtatcagccgccgccaactgggagatgaggaggctttcttggggggcagtcagcagttcatttcacaagacagaggaacttgtaaggagatgcactgatttatcttggcgcaaaccagcaggacgaattagtgggaatagcccgcgaatatctaagttatgcctgtcggcatgagcagaaacttccaattcgaaacagtttggagaggttgtttttgggcataccttttgttagtcagcctctcgattgctcatcgtcattacacagtaccgaagtttgatcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcgacgcactccttctttactccaccatctcgtccttattgaaaacgtgggtagcaccaaaacgaatcaagtcgctggaactgaagttaccaatcacgctggatgatttgccagttggattaatcttgcctttccccgcatgaataatattgatgaatgcatgcgtgaggggtatttcgattttggcaatagctgcaattgccgcgacatcctccaacgagcataattcttcagaaaaatagcgatgttccatgttgtcagggcatgcatgatgcacgttatgaggtgacggtgctaggcagtattccctcaaagtttcatagtcagtatcatattcatcattgcattcctgcaagagagaattgagacgcaatccacacgctgcggcaaccttccggcgttcgtggtctatttgctcttggacgttgcaaacgtaagtgttggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatcggggtcgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgacggatctctaggacgcgtcctagaagctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatatcaactcccctatccattgctcaccgaatggtacaggtcggggacccgaagttccgactgtcggcctgatgcatccccggctgatcgaccccagatctggggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtgtctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccgg

SEQ ID NO: 100 - DNA, 17978 bp, Artificial Sequence
  Description of Artificial Sequence Synthetic polynucleotide
tgagcgtcgcaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggcccggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctaatcaaacaagtttgtacaaaaaagctgaacgagaaacgtaaaatgatgaagtactcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcagctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagagtcttagtttggctgctgggtattgccctactgtttgcgcaggtggacactttggtggaggaggctatggaccattgatgagaagctatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttctgaatcagcaattccattccctcatcgagctggaatcttgtatgagttatggtacatatgtagctgggagaagcaagaagataacgaaaagcatctaaactggattagaaatatttataacttcatgactccttatgtgtcccaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataatttttttagaaacgaacaaagcatcccacctcttccacggcatcatcattaaaatatattgatatttatatcattttacgtttctcgttcagctttcttgtacaaagtggttcgatctagaggatccatggtgagcaagggcgaggagctgttcaccggggtggtgcccatcctggtcgagctggacggcgacgtgaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaaagcggcccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattagcttcatcaacgcaagacatgcgcacgaccgtctgacaggagaggaatttccgacgagcacagaaaggacttgctcttggacgtaggcctatttctcaggcacatgtatcaagtgttcggacgtgggttttcgatggtgtatcagccgccgccaactgggagatgaggaggctttcttggggggcagtcagcagttcatttcacaagacagaggaacttgtaaggagatgcactgatttatcttggcgcaaaccagcaggacgaattagtgggaatagcccgcgaatatctaagttatgcctgtcggcatgagcagaaacttccaattcgaaacagtttggagaggttgtttttgggcataccttttgttagtcagcctctcgattgctcatcgtcattacacagtaccgaagtttgatcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcgacgcactccttctttactccaccatctcgtccttattgaaaacgtgggtagcaccaaaacgaatcaagtcgctggaactgaagttaccaatcacgctggatgatttgccagttggattaatcttgcctttccccgcatgaataatattgatgaatgcatgcgtgaggggtatttcgattttggcaatagctgcaattgccgcgacatcctccaacgagcataattcttcagaaaaatagcgatgttccatgttgtcagggcatgcatgatgcacgttatgaggtgacggtgctaggcagtattccctcaaagtttcatagtcagtatcatattcatcattgcattcctgcaagagagaattgagacgcaatccacacgctgcggcaaccttccggcgttcgtggtctatttgctcttggacgttgcaaacgtaagtgttggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatcggggtcgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgacggatctctaggacgcgtcctagaagctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatatcaactcccctatccattgctcaccgaatggtacaggtcggggacccgaagttccgactgtcggcctgatgcatccccggctgatcgaccccagatctggggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtgtctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccgg

SEQ ID NO: 101 - DNA, 56 bp, Artificial Sequence
  Description of Artificial Sequence Synthetic primer
  modified_base (15)..(33): a, c, t, g, unknown or other
tgtggtctcaattgnnnnnnnnnnnnnnnnnnngttttagagctagaaatagcaag

SEQ ID NO: 102 - DNA, 23 bp, Artificial Sequence
  Description of Artificial Sequence Synthetic oligonucleotide
gttttagagctagaaatagcaag

SEQ ID NO: 103 - DNA, 31 bp, Artificial Sequence
  Description of Artificial Sequence Synthetic primer
tgtggtctcaagcgtaatgccaactttgtac

SEQ ID NO: 104 - DNA, 17 bp, Artificial Sequence
  Description of Artificial Sequence Synthetic oligonucleotide
taatgccaactttgtac

SEQ ID NO: 105 - DNA, 1075 bp, Cannabis sativa
tcaacattctcctttaggtttgtttacaaaattatatttttctttctctcattcaatatcaaaatttcaatagctaatcctcaagaaaatttcctaaattgcttctcccaatatattcataacaatccagcaaatctaaaactcgtatacactcaacacgaccaattgtatatgtctgtcctgaatttgacaatacaaaatcttagatttacctctgatacaaccccaaaaccactcgttattgtcactccttcaaatgtctcccatatccaagccactattctatgctccaagaaagttggcttgcagattcgaactcgaagcggtggccatgatgctgagggtttgtcctacacatctcaagtcccatttgttatagtagacttgagaaacatgcattcggtgaaaatagatattcgtagccaaactgcgtgggttgaagccggagctacccttggagaagtttattattggattaatgagaagaatgagaatcttagttttcctggtgggtattgccctactgttggcgtaggtggacactttagtggaggaggctatggagcattaatgcgaaattatggcctcgcagctgataatatcattgatgcacacttagtcaatgttgatggaaaagttctagatcgaaaatccatgggggaagatctattttgggctatacgtggtggtggaggtgaaaactttggaatcattgcagcgtggaaaattagactggttgctgtcccatcaagggctactatattcagtgttaaaaggaatatggagatacatgggcttgtcaagttatttaacaaatggcaaaatattgcttacaagtatgacaaagatttattactcatgactcacttcataaccaggaatattatagataatcaaggaaagaataagactacagtacacggttacttctcttgcattttccatggtggagtggatagtctagtcaacttgatgaacaagagctttcctgagttgggtattaaaaaaactgattgcaaagaattgagctggattgatactaccatcttctacagtggtgttgtaaattataaca

SEQ ID NO: 106 - DNA, 1051 bp, Cannabis sativa
tgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaaca

SEQ ID NO: 107 - DNA, 1084 bp, Cannabis sativa
atgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaaca

SEQ ID NO: 108 - DNA, 1051 bp, Cannabis sativa; identical to SEQ ID NO: 106

SEQ ID NO: 109 - DNA, 1084 bp, Cannabis sativa; identical to SEQ ID NO: 107

SEQ ID NO: 110 - DNA, 1086 bp, Cannabis sativa
atgaattgctcaacattctccttttggtttgtttgcaaaataatatttttctttctctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaaca

SEQ ID NO: 111 - DNA, 1083 bp, Cannabis sativa
atgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggagagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaaca

SEQ ID NO: 112 - DNA, 855 bp, Cannabis sativa
caaccccaaaaccacttgttatcatcactcctttaaatgtctcccatatccaaggcactattctatgctccaagaaagttggcttgcagattcgaactcgaagcggtggtcatgatgctgagggcatgtcctacatatctcaagtcccatttgttatagtagacttgagaaacatgcattcggtcaaaatagatgttcatagccaaactgcatgggttgaagccggagctacccttggagaagtttattattggatcaatgagaacaatgagaatcttagttttcctgctgggtactgccctactgttggcgcgggtggacactttagtggaggaggctatggagcattgatgcgaaattatggcctcgcggctgataatatcattgatgcgcacttagtcaatgttgatggaaaagttttagatcgaaaatccatgggggaagatttgttttgggctatacgtggtggtggaggagaaaactttggaatcattgcagcgtggaaaattagacttgatgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacacagttacttctcctccattttccatggtggagtggatagtctagtcgacttgatgaacaagagctttcctgaattgggtattaaaaaaacagattgcaaacagttgagctggattgatactatcatcttctacagtggtgttgtaaattacaaca

SEQ ID NO: 113 - DNA, 1069 bp, Cannabis sativa
tcaacattctgtttttggtatgtttgcaagataatattttctttctctcattcaatatccaaatttcaatagctaatcctcaagaaaacttaaatgcttctcacaatatattcccaccaatgtaacaaatgcaaaactcgtatacactcaacacgaccaattttatatgtctatcctaaattcgaccatacaaaatcttagatttacctctgacacaaccccaaaaccacttgttatcatcactcctttaaatgtctcccatatccaaggcactattctatgctccaagaaagttggcttgcagattcgaactcgaagcggtggtcatgatgctgagggcatgtcctacatatctcaagtcccatttgttatagtagacttgagaaacatgcattcggtcaaaatagatgttcatagccaaactgcatgggttgaagccggagctacccttggagaagtttattattggatcaatgagaacaatgagaatcttagttttcctgctgggtactgccctactgttggcgcgggtggacactttagtggaggaggctatggagcattgatgcgaaattatggcctcgcggctgataatatcattgatgcgcacttagtcaatgttgatggaaaagttttagatcgaaaatccatgggggaagatttgttttgggctatacgtggtggtggaggagaaaactttggaatcattgcagcgtggaaaattagacttgttgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacacagttacttctcctcattttccatggtggagtggatagtctagtcgacttgatgaacaagagctttcctgaattgggtattaaaaaaacagattgcaaacagttgagctggattgatactatcatcttctacagtggtgttgtaaattacaaca

SEQ ID NO: 114 - DNA, 1083 bp, Cannabis sativa
tgaagtactcaacattctgtttttggtatgtttgcaagataatatttttctttctctcattcaatatccaaatttcaatagctaatcctcaagaaaacttccttaaatgcttctcacaatatattcccaccaatgtaacaaatgcaaaactcgtatacactcaacacgaccaattttatatgtctatcctaaattcgaccatacaaaatcttagatttacctctgaaacaaccccaaaaccacttgttatcatcactcctttaaatgtctcccatatccaaggcactattctatgctccaagaaagttggcttgcagattcgaactcgaagcggtggtcatgatgctgagggcatgtcctacatatctcaagtcccatttgttatagtagacttgagaaacatgcattcggtcaaaatagatgttcatagccaaactgcatgggttgaagccggagctacccttggagaagtttattattggatcaatgagaacaatgagaatcttagttttcctgctgggtactgccctactgttggcgcgggtggacactttagtggaggaggctatggagcattgatgcgaaattatggcctcgcggctgataatatcattgatgcgcacttagtcaatgttgatggaaaagttttagatcgaaaatccatgggggaagatttgttttgggctatacgtggtggtggaggagaaaactttggaatcattgcagcgtggaaaattagacttgatgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacacagttacttctcctccattttccatggtggagtggatagtctagtcgacttgatgaacaagagctttcctgaattgggtattaaaaaaacagattgcaaacagttgagctggattgatactatcatcttctacagtggtgttgtaaattacaaca

SEQ ID NO: 115 - DNA, 1075 bp, Cannabis sativa
tcaacattctgtttttggtatgtttgcaagataatatttttctttctctcattcaatatccaaatttcaatagctaatcctcaagaaaacttccttaaatgcttctcacaatatattcccaccaatgtaacaaatgcaaaactcgtatacactcaacacgaccaattttatatgtctatcctaaattcgaccatacaaaatcttagatttacctctgaaacaaccccaaaaccacttgttatcatcactcctttaaatgtctcccatatccaaggcactattctatgctccaagaaagttggcttgcagattcgaactcgaagcggtggtcatgatgctgagggcatgtcctacatatctcaagtcccatttgttatagtagacttgagaaacatgcattcggtcaaaatagatgttcatagccaaactgcatgggttgaagccggagctacccttggagaagtttattattggatcaatgagaacaatgagaatcttagttttcctgctgggtactgccctactgttggcgcgggtggacactttagtggaggaggctatggagcattgatgcgaaattatggcctcgcggctgataatatcattgatgcgcacttagtcaatgttgatggaaaagttttagatcgaaaatccatgggggaagatttgttttgggctatacgtggtggtggaggagaaaactttggaatcattgcagcgtggaaaattagacttgttgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacacagttacttctcctccattttccatggtggagtggatagtctagtcgacttgatgaacaagagctttcctgaattgggtattaaaaaaacagattgcaaacagttgagctggattgatactatcatcttctacagtggtgttgtaaattacaaca

SEQ ID NO: 116 - DNA, 1601 bp, Cannabis sativa
tgatgacgcggtggaagaggtgggatactttgttcgtttctaaaaaaattattgggatcagctttggttttcaccttaactaacctgttaaaatttttaccaaaatacttttcaccccaaatacgtgcttgtgtgtaattattaggactctcaggattagtttttcctaaatcaaggtccctataattgagatacgccaatcttggattttgggacacataaggagttgtgaaattataaacacttcgaacccagtttatatgcttttcgttatcttcttgcttctcccaggtagcagtgtaccaaagttcatacattattccagctcgatgagggaatggaattgctgattctgaaatctcatccattataccaccgtaagggtacaacacatacatcccaactcctacctcttcttcatataatttttccaaaattttgaccattgcagtttcaggtattagtttcttaacatagtctaacttaattgagaaagccgtcttcttcccagctgatctatcaagcaaaatttcctttttaaaattagcagtgttgtaatttacaacaccactgtagaagatggttgtatcaatccagctcaattctttgcaatcagtttttttaatacccaactcaggaaagctcttgttcatcaagtcaactagactatccactccaccaagaaaaatggaagagaagtaaccatgtactgtagtcttattcttcccatgattatctgtaatattcctagttctgaagtgagtcgtgagcattaaatctttgtcatacttgtaagcaatattttgccatttgttaaataacttgacaagcccatgtatctccatgttctttttaacactgaatatagtagcctttgatgggacaacaacaagtttgattttccatgctgcaatgattccaaagttttctcctcctccaccacgtatagcccaaaatagatcttctcccatggattttcgatctagaacttttccatcaacattgactaagtgtgcatcaatgatattatcagccgcaaggccataatttcgcatcaatgctccatagcctcctccactaaagtgtccacctacgccaacagtagggcaatacccaccaggaaaactaaaattctcattcatctcattgatccaataataaacttctccaagggtagctccggcttcaacccacgcagtttggctatgaatatctactttgaccgtatgcatgtttctcaagtctactatagcaaatgggacttgagatatgtaggacaaaccctcagcatcatggccaccgcttcgagttcgaatctgcaaaccaactttcttggagcagagaatactggcctggatatgggagacatttgaaggagtgacaataacgagtggttttggggttgtatcagaggtgaatctaagattttgtattgtcgaattcaggacagacatatacaattggtcgtgttgagtgtatatgaattttggatttgctggattgttaggaatatattccgagaagcatttaaggaagttttcttgaggattagctattgaaatttggatattgaatgagagaaagaaaaatattattttgca

SEQ ID NO: 117 - DNA, 1635 bp, Cannabis sativa; identical to SEQ ID NO: 8

SEQ ID NO: 118 - DNA, 1635 bp, Cannabis sativa; identical to SEQ ID NO: 8

SEQ ID NO: 119 - DNA, 1617 bp, Cannabis sativa
tcaacattctcctttaggtttgtttacaaaattatatttttctttctctcattcaatatcaaaatttcaatagctaatcctcaagaaaatttcctaaattgcttctcccaatatattcataacaatccagcaaatctaaaactcgtatacactcaacacgaccaattgtatatgtctgtcctgaatttgacaatacaaaatcttagatttacctctgatacaaccccaaaaccactcgttattgtcactccttcaaatgtctcccatatccaagccactattctatgctccaagaaagttggcttgcagattcgaactcgaagcggtggccatgatgctgagggtttgtcctacacatctcaagtcccatttgttatagtagacttgagaaacatgcattcggtgaaaatagatattcgtagccaaactgcgtgggttgaagccggagctacccttggagaagtttattattggattaatgagaagaatgagaatcttagttttcctggtgggtattgccctactgttggcgtaggtggacactttagtggaggaggctatggagcattaatgcgaaattatggcctcgcagctgataatatcattgatgcacacttagtcaatgttgatggaaaagttctagatcgaaaatccatgggggaagatctattttgggctatacgtggtggtggaggtgaaaactttggaatcattgcagcgtggaaaattagactggttgctgtcccatcaagggctactatattcagtgttaaaaggaatatggagatacatgggcttgtcaagttatttaacaaatggcaaaatattgcttacaagtatgacaaagatttattactcatgactcacttcataaccaggaatattatagataatcaaggaaagaataagactacagtacacggttacttctcttgcattttccatggtggagtggatagtctagtcaacttgatgaacaagagctttcctgagttgggtattaaaaaaactgattgcaaagaattgagctggattgatactaccatcttctacagtggtgttgtaaattataacactactaattttcaaaaggaaattttgcttgatagatcagctgggcagaaagtagctttctcaattaagttagactacgttaagaaaccaattccagaaactgcaattgtcaaaattttggagaaattgtatgaagaagatgtaggagttggagtgtatgtattgtacccttacggtggtataatggacaagatctcagaatcaacaattcctttccctcatcgagctggaatcatgtacgaagtttggtacgcagctacctgggagaagcaagaagataatgaaaagcatataaactgggttcgaagtgtttataatttcatgacgccttatgtgtcccaaaatccaagaatggcgtatctcaattatagggaccttgatttaggaaaaactgatcccaagagtcctaataattacacccaagcacgtatctggggtgaaaagtactttggtaaaaactttgacaagttagttaaggtgaaaaccaaagttgatcccaataatttttttagaaacgagcaaagcatcccacctcttccgcca

SEQ ID NO: 120 - DNA, 1637 bp, Cannabis sativa
atgaattgctcaacattctccttttggtttgtttgcaaaataatatttttctttctctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaaataatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcat

SEQ ID NO: 121 - DNA, 1602 bp, Cannabis sativa
tgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcat

SEQ ID NO: 122 - DNA, 1626 bp, Cannabis sativa
tcaacattctgtttttggtatgtttgcaagataatatttttctttctctcattcaatatccaaatttcaatagctaatcctcaagaaaacttccttaaatgcttctcacaatatattcccaccaatgtaacaaatgcaaaactcgtatacactcaacacgaccaattttatatgtctatcctaaattcgaccatacaaaatcttagatttacctctgacacaaccccaaaaccacttgttatcatcactcctttaaatgtctcccatatccaaggcactattctatgctccaagaaagttggcttgcagattcgaactcgaagcggtggtcatgatgctgagggcatgtcctacatatctcaagtcccatttgttatagtagacttgagaaacatgcattcggtcaaaatagatgttcatagccaaactgcatgggttgaagccggagctacccttggagaagtttattattggatcaatgagaacaatgagaatcttagttttcctgctgggtactgccctactgttggcgcgggtggacactttagtggaggaggctatggagcattgatgcgaaattatggcctcgcggctgataatatcattgatgcgcacttagtcaatgttgatggaaaagttttagatcgaaaatccatgggggaagatttgttttgggctatacgtggtggtggaggagaaaactttggaatcattgcagcgtggaaaattagacttgttgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacactgttacttctcctccattttccatggtggactggatagtctagtcgacttgatgaacaagagctttcctgaattgggtattaaaaaaacagattgcaaacagttgagctggattgatactatcatcttcaacagtggtcttgtaaattacaacactactaattttaaaaaagaaattttgctttaaagatcaggtgggcggaaggcggctttctcaattaagttagactatgttaagaaaccgattccagaaaccgcaatggtcacaattttggaaaaattatatgaagaagatgtaggagttgggatgtttgtgttttacccttatggtggtataatggatgagatttcagaatcagcaattccattccctcatcgagctggaatcatgtatgaaatttggtacatagcttcatgggagaagcaagaagataatgaaaagcatataaactggattcggaatgtttataatttcacgactccttatgtgtcccaaaatccaagaatggcgtatctcaattatagggaccttgatttaggaaaaactaatttcgagagtcctaataattacacacaagcacgtatttggggtgaaaagtattttggtaaaaattttaataggttagtaaaagtaaaaaccaaggttgatcccgataatttctttagaaacgaacaaagcatcccacctcttcccctgcgtcatcat

SEQ ID NO: 123 - DNA, 1625 bp, Cannabis sativa
tcaacattctgtttttggtatgtttgcaagataatatttttctttctctcattcaatatccaaatttcaatagctaatcctcaagaaaacttccttaaatgcctctcacaatatattcccaccaatgtaacaaatgcaaaactcgtatacactcaacacgaccaattttatatgtctatcttaaattcgaccatacaaaatcttagatttacctctgacacaaccccaaaaccacttgttatcatcactcctttaaatgtctcccatatccaaggcactattctatgctccaagaaatttggcttgcagattcgaactcgaagcggtggtcatgatgctgagggcatgtcctacatatctcaagtcccatttgttatagtagacttgagaaacatgcattcggtcaaaatagatgttcatagccaaaatgcatgggttgaagccggagctacccttggagaagtttattattggatcaatgagaacaatgagaatcttagttttcctgctgggtactgccctactgttggcgcttgtggacactttagtggaggaggctatggagcattgatgcgaaattatggcctcgcggctgataatatcattgatgcacacttagtcaatgttgatggaaaagttttagatcgaaaatccatgggggaagatttgttttgggctatacgtggtggtggaggagaaaactttggaatcattgcagcgtggaaaattagacttgttgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacacagttacttctcctccattttccatggtggagtggatagtctagtcgacttgatgaacaagagctttcctgaattgggtattaaaaaaagagattgcaaacagttgagctggattgatactatcatcttctacagtggtcttgtaaattacaacacaactaattttaaaaaagaaattttgcttgatagatcaggtgggcggaaggcggctttctcgattaagttagactatgttaagaaaccgattccagaaaccgcaatggtcacaattttggaaaaattatatgaagaagatgtaggagttgggatgtttgtgttttacccttatggtggtataatggatgagatttcagaatcagcaattccattcctcatcgagctggaatcatgtatgaaatttggtacatagcttcatgggagaagcaagaagataatgaaaagcatataaactggattcggaatgtttataatttcacgactccttatgtgtcccaaaatccaagaatggcgtatctcaattatagggaccttgatttaggaaaaactaatttcgagagtcctaataattacacacaagcacgtatttggggtgaaaagtattttggtaaaaattttaataggttagtaaaagtaaaaaccaaggttgatcccgataatttctttagaaacgaacaaagcatcccacctcttcccctgcgtcatcat

SEQ ID NO: 124 - DNA, 1631 bp, Cannabis sativa
tgaagtactcaacattctgtttttggtatgtttgcaagataatatttttctttctctcattcaatatccaaatttcaatagctaatcctgaaggaaacttccttaaatgcttctcacaatatattcccaccaatgtaacaaatgcaaaactcgtatacactcaacacgaccaattttatatgtctatcctaaattcgaccatacaaaatcttagatttacctttgacacaaccccaaaaccacttgttatcatcactcctttaaatgtctcccatatccaaggcactattctatgctccaagaaagttggcttgtagattcgaactcgaagcggtggtcatgatgctgagggcatgtcctacatatctcaagtcccatttgttatagtaaacttgagaaacatgcattcggtcaaaatagatgttcatagcgaaactgcatgggttgaagccggagctacccttggagaagtttattattggatcaatgagaacaatgagaatcttagttttcttgctgggtactgccctactgttggcgcgggtggacactttagtggaggaggctatggagcattgatgcgaaattatggcctcgcggctaataacatcattgatgcgcacttagtcaatgttgatggaaaagttttagatcgaaaatccatgggggaagatttgttttgggctatacgtggtggaggagaaaactttggaatcattgcagcgtggaaaattagatttgttgctgtcccatcaatgtctactatattcagtgttaaaaagaacatggagatacatgagcttgtcaagttagttaacaaatggcaaaatattgcttacatgtatgaaaaagaatgattactctttactcactttataaccaggaatattacagataatcaagggaagaataagacaacaatacacagttacttctcctccattttctatggtggagtggatagtctagtcgacttgatgaacaagagctttcctgaattgggtattaaaaaaacagattgcaaacagttgagctggattgatactatcatcttctacagtggtcttgtaaattacaacactactaattttaaaaaagaacttttgcttgatagatcaggtgggcggaaggcggctttctcgattaagttagactaagttaagaaaccgattccagaaaccgcaatggtcacaattttggaaaaattatatgaagaagatgtaggagttgggatgtttgtgttttacccttatggtggtataatggatgagatttcagaatcagcaattccattccctcatcgagctggaatcatgtatgaaatttggtacatagcttcatgggagaagcaagaagataatgaaaagcatataaactggattcggaatgtttacaatttcacgactccttatgtgtcccaaaatccaagaatggcgtatctcaattatagggaccttgatttaggaaaaactaatttcgagagtcctaataattacacacaagcacgtatttggggtgaaaagtattttggtaaaaattttaataggttagtaaaagtaaaaaccaaggttgatcccgataatttctttagaaacgaacaaagcatcccacctcttcccctacgtcatcat

SEQ ID NO: 125 - DNA, 1635 bp, Cannabis sativa
atgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcgtgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcat

SEQ ID NO: 126 - DNA, 1634 bp, Cannabis sativa
atgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgaaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgtaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcat

SEQ ID NO: 127 - DNA, 1635 bp, Cannabis sativa
atgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcat

SEQ ID NO: 128 - DNA, 1629 bp, Cannabis sativa
atgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacgtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcat

SEQ ID NO: 129 - DNA, 1631 bp, Cannabis sativa
atgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagagcaagaagataacgaaaagcatataaactggttcgaagtgtttattaatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaggtgaaaccaaagctgatccaataatttttttagaaacgaacaaagtatcccacctcttccaccgcgtcatcat

SEQ ID NO: 130 - DNA, 1608 bp, Cannabis sativa
tcaacattctcctttaggtttgtttacaaaattatatttttctttctctcattcaatatcaaaatttcaatagctaatcctcaagaaaatttcctaaaatgcttctcccaatatattcataacaatccagcaaatctaaaactcgtatacactcaacacgaccaattgtatatgtctgtcctgaatttgacaatacaaaatcttagatttacctctgatacaaccccaaaaccactcgttattgtcactccttcaaatgtctcccatatccaagccactattctatgctccaagaaagtgggcttgcaaattcgaactcgaagcggtggccatgatgctgagggtttgtcctacacatctcaagtcccatttgttatagtagacttgagaaacatgcattcggtgaaaatagatattcgtagccaaattgcgtgggttgaagccggagctacccttggagaagtttattattggattaatgagaatcttagttttcctggtgggtattgccctactgttggcgtaggtggacactttagtggaggaggctatagagcattaatgcgaaattatggcctcgcagctgataatatcattgatgcacacttagtcaatgttgatggaaaagttctagatcgaaaatccatgggggaagatctattttgggctatacgtggtggtggaggtgaaaactttggaatcattgcagcgtggaaaattagactggttgctgtcccatcaagggctactatattcagtgttaaaaggaatatggagatacatgggcttgtcaagttatttaataaatggcaaaatattgcttacaagtatgacaaagatttattactcatgactcacttcataaccaggaatattatagataatcaaggaaagaataagactacagtacacggttacttctcttgcattttccatggtggagtagatagtctagtcaacttgatgaacaagagctttcctgagttgggtattaaaaaaactgattgcaaagaattgagctggattgatactaccatcttctacagtggtgttgtaaattataacactactaattttcaaaaggaaattttgcttgatagatcagctgggcagaaagtagctttctcagttaagttagactacgttaagaaaccaattccagaaactgcaattgtcaaaattttggagaaattgtatgaagaagatgtaggagttggggtgtatgtattgtacccttacggtggtataatggacaagatctcagaatcaacaattcctttccctcatcgagctggaatcatgtacgaagtttgatacgcagctacctgggagaagcaagaagataatgaaaagcatataaactgggtttgaagtgtttataatttcatgacgccttatgtgtcccaaaatccaagaatggcgtatctcaattatagggaccttgatttaggaaaaactgatcccaagagtcctaataattacacccaagcacgtatctggggtgaaaagtactttggtaaaaactttgacaagttagttaaggtgaaaaccaaagttgatcccaataatttttttagaaacgagcaaagcatcccacctcttccgcca

SEQ ID NO: 131 - DNA, 1550 bp, Cannabis sativa
ctaatcctcgagaaaacttccttaaatgcttctcgcaatatattcccaataatgcaacaaatctaaaactcgtatacactcaaaacaacccattgtatatgtctgtcctaaattcgacaatacacaatcttagattcagctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagagtcttagtttggctgctgggtattgccctactgtttgcgcaggtggacactttggtggaggaggctatggaccattgatgagaagctatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttctgaatcagcaattccattccctcatcgagctggaatcttgtatgagttatggtacatatgtagctgggagaagcaagaagataacgaaaagcatctaaactggattagaaatatttataacttcatgactccttatgtgtcccaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataatttttttagaaacgaacaaagcatcccacctcttccacggcatcatcat

SEQ ID NO: 132 - DNA, 1394 bp, Cannabis sativa
ctaatcctcaagaaaacttccttaaatgcttctcacaatatattcccaccaatgtaacaaatgcaaaactcgtatacactcaacacgaccaattttatatgtctatcctaaattcgaccatacaaaatcttagatttacctctgaaacaaccccaaaaccacttgttatcatcactcctttaaatgtctcccatatccaaggcactattctatgctccaagaaagttggcttgcagattcgaactcgaagcggtggtcatgatgctgagggcatgtcctacatatctcaagtcccatttgttatagtagacttgagaaacatgcattcggtcaaaatagatgttcatagccaaactgcatgggttgaagccggagctacccttggagaagtttattattggatcaatgagaacaatgagaatcttagttttcctgctgggtactgccctactgttggcgcgggtggacactttagtggaggaggctatggagcattgatgcgaaattatggcctcgcggctgataatatcattgatgcgcacttagtcaatgttgatggaaaagttttagatcgaaaatccatgggggaagatttgttttgggctatacgtggtggtggaggagaaaactttggaatcattgcagcgtggaaaattagacttgttgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacacagttacttctcctccattttccatggtggagtggatagtctagtcgacttgatgaacaagagctttcctgaattgggtattaaaaaaacagattgcaaacagttgagctggattgatactatcatcttctacagtggtgttgtaaattacaacacaactaattttaaaaaagaaattttgcttgatagatcaggtgggcggaaggcggctttctcgattaagttagactatgttaagaaaccgattccagaaaccgcaatggtcacaattttggaaaaattatatgaagaagatgtaggagttgggatgtttgtgttttacccttatggtggtataatggatgagatttcagaatcagcaattccattccctcatcgagctggaatcatgtatgaaatttggtacatagcttcatgggagaagcaagaagataatgaaaagcatataaactggattcggaatgtttataatttcacgactccttatgtgtcccaaaatccaagaatggcgtatctcaattatagggaccttgatttaggaaaaaac

SEQ ID NO: 133 - DNA, 1631 bp, Cannabis sativa
atgaagtactcaacattctccttttggtttgtttgcaagataatatttttctttctctcattcaatatccaaacttcaattgctaatcctcgagaaaacttccttaaatgcttctcgcaatatattcccaccaatgtaacaaatctaaaacttacacccaaaacaaccaattgtatatgcctgtccaaaattcgacaatacacaatcttagattcacctctaacacaaccccaaaactacttgttatcgtcactccttcacatgtctctcatatccaaggcactattctatgtccaagaaaattggtttgcaaattcgaactcgaagcggtggtcatgattctgaagacatgtcctacatatctcaagtcccatttgttatagtagacttgagaaacatgcattcaatcaacatagatgttcatagccaaatcgcaagggttgaagccggagctacccttggagaagtttattattgggttaatgagaaaaatgagaatcttagtttggctgctgggtattgccctactgttagcgcagctggacactttggtggaggaggatatggaccattgatgcaaaattatggcctcgcggctgataatatcgttgatgcacacttagtcaacgttgatgcaaaagtgctagatcgaaaatctatgggggaagatctcttttgggctatacgtggtggtggaggagaaagcttcggaatcattgtagcatggaaaattagactggttgctgtcccaacaaagtctactatgtttagtgttaaaaagatcatggagatacatgagcttgtcaagtgagttaacaaatggcaaaatattgcttacaagtatgacaaagatttattactcatgactcacttcataactaggaatattacaaataatcatgggaagaataagacaacaatacacacttacttctcttcagttttccttggtggagtggatagtctagtcgacttgatgaataagagttttcctgagttgggtattaaaaaaacagattgcaaacaattgagctagattgatattatcatcttttatagcggtgttgtaaattacggcactgataattttaataaggaaattttgcttgatagatcagctgggcagaacggttctttaaagattaagttagactacgttaagaaaccaattccagaatctgcgtttgtcaaaattttggaaaaattatatgaagaagatgaaggagctgggatgtatgcgttgtacccttacggtggtataatggatgagatttcagaatcagcaattccattccctcattgagctggaatcatgtatgaattatggtacatatgtagctgggagaagcacgaagataacgaaaaagcatctaaactggattcgaaatgtttatagcttcattactccttatgtgtcctaaaatccaagattggcatatctcaattatagagaccttgatactggaataaatgatcccaagagtccaaataattacacacaagaaagtatttggggtgagaagtattttggtaaaaattttgacagggtagtaaaagtgaaaaccctggttgatcccaataatttttttagaaatgaacaaagcatcccacctcttccaccgcatcgtcat

SEQ ID NO: 134 - DNA, 1622 bp, Cannabis sativa
atgaagtactcaacattctccttttggtttgtttgcaagataatatttttctttctctcattcaatatccaaccttcaattgctaatcctcgagaaaacttccttaaatgcttctcgcaatatattcccaccaatgtaacaaatctaaaacttacacccaaaacaacattgtatatgcctgtccaaaattcgacaatacacaatcttagattcacctctaacacaaccccaaaactacttgttatcgtcactcttcacatgtctctcatatccaaggcactattctatgtccaagaaaattggtttgcaaattcaaactcgaaacggtggtcatgattctgaaggcatgtcccacatatctcaagtcccatttgttatagtagacttgagaaacatgcattcaatcaaaagatgttcatagccaaatcgcaagggttgaagccggagctacccttggagaagtttattattgggttaatgagaaaaatgagatcttagtttggctgctggtattgccctactgttagcgcagctggacactttggtggaggaggctatggaccattgatgtgaaattatggcctcgcggatgataatatcgttgatgcacacttagtcaacgttgatggaaaagtactagatcgaaaatctatgggacaagatctcttttgggctatacgtggtggtggaagagaaagcttcagaatcattgtagcatggaaaattagactggttgctgtcccaacaaagtctactatgtttagtgttaaaaagatcaaggagatacatgagcttgtgaagttagttaacaagtggcaaaatatttcttacaagtatgacatagatttattactcatgactcacttcataactaggaatattacagataatcaagggaagaataagacaacaatacacacttacttctctttagttttccttggtggagtggatagtctagtcgacttaatgaacaagagttttcctgagtttggtattaaaaaaatagattgcaaacaattgagctggattgatactatcatcttctatagcggtgttgtaaattacggcactgataattttaataaccaaatttcgcttgttagatcagctgggcagaacggtgctttcaagattaagttagactatgttaagaaaccaattccagaatctgcatttgtcaaaattttggaaaaattatatgaagaagataaaggagttgggatgtatgcgttgtacccttacggttgtctaatggatgagatttcagaatcagcaattccattccctcatcgagttggaatcatgtatgaattatggtacatatgtagctgggagaagcacgaagataaagaaaagtatctaaactggattcgaaatgttgataacttcatgactccttatgtgtcccaaaatccaagattgacatatctcaattatagacatcttgatataggaataaatgatcccaagagtcaaaataattacacagaagcatgtatttggggtgagaaatctttggtaaaaattttgacaggctagtaaaagtgaaaaccctggttgatttcaataatctttttagaaatgaacaaagcatcccacctcttccaccgcatcgtcat

SEQ ID NO: 135 - DNA, 1623 bp, Cannabis sativa
atgaagtactcaacattctgtttttggtatgtttgcaagataatatttttctttctctcattcaatatccaaatttcaatagctaatcctcaagaaaacttccttaaatgcttctcacaatatattcccaccaatgtaacaaatgcaaaactcgtatacactcaacacgaccaattttatatgtctatcctaaattcgaccatacaaaatcttagatttacctctgacacaaccccaaaaccacttgttatcatcactcctttaaatgtctcccatatccaaggcactattctatgctccaagaaagttggcttgcagattcgaactcgaagcggtggtcatgatgctgagggcatgtcctacatatctcaagtcccatttgttatagtagacttgagaaacatgcattcggtcaaaatagatgttcatagccaaactgcatgggttgaagccggagctacccttggagaagtttattattggatcaatgagaacaatgagaatcttagttttcctgctgggtactgccctactgttggcgcgggtggacactttagtggaggaggctatggagcattgatgcgaaattatggcctcgcggctgataatatcattgatgcgcacttagtcaatgttgatggaaaagttttagatcgaaaatccatgggggaagatttgttttgggctatacgtggtggtggaggagaaaactttggaatcattgcagcgtggaaaattagacttgttgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacactgttacttctcctccattttccatggtggactggatagtctagtcgacttgatgaacaagagctttcctgaattgggtattaaaaaaacagattgcaaacagttgagctggattgatactatcatcttcaacagtggtcttgtaaattacaacactactaattttaaaaaagaaattttgctttaaagatcaggtgggcggaaggcggctttctcaattaagttagactatgttaagaaaccgattccagaaaccgcaatggtcacaattttggaaaaattatatgaagaagatgtaggagttgggatgtttgtgttttacccttatggtggtataatggatgagatttcagaatcagcaattccattccctcatcgagctggaatcatgtatgaaatttggtacatagcttcatgggagaagcaagaagataatgaaaagcatataaactggattcggaatgtttataatttcacgactccttatgtgtcccaaaatccaagaatggcgtatctcaattatagggaccttgatttaggaaaaactaatttcgagagtcctaataattacacacaagcacgtatttggggtgaaaagtattttggtaaaaattttaataggttagtaaaagtaaaaaccaaggttgatcccgataatttctttagaaacgaacaaagcatcccacctcttccc

SEQ ID NO: 136 - DNA, 1622 bp, Cannabis sativa
atgaagtactcaacattctgtttttggtatgtttgcaagataatatttttctttctctcattcaatatccaaatttcaatagctaatcctcaagaaaacttccttaaatgcctctcacaatatattcccaccaatgtaacaaatgcaaaactcgtatacactcaacacgaccaattttatatgtctatcttaaattcgaccatacaaaatcttagatttacctctgacacaaccccaaaaccacttgttatcatcactcctttaaatgtctcccatatccaaggcactattctatgctccaagaaatttggcttgcagattcgaactcgaagcggtggtcatgatgctgagggcatgtcctacatatctcaagtcccatttgttatagtagacttgagaaacatgcattcggtcaaaatagatgttcatagccaaaatgcatgggttgaagccggagctacccttggagaagtttattattggatcaatgagaacaatgagaatcttagttttcctgctgggtactgccctactgttggcgcttgtggacactttagtggaggaggctatggagcattgatgcgaaattatggcctcgcggctgataatatcattgatgcacacttagtcaatgttgatggaaaagttttagatcgaaaatccatgggggaagatttgttttgggctatacgtggtggtggaggagaaaactttggaatcattgcagcgtggaaaattagacttgttgctgtcccatcaatgtctactatattcagtgttaaaaagaacatggagatacatgagcttgtcaagttagttaacaaatggcaaaatattgcttacatgtatgaaaaagaattattactctttactcactttataaccaggaatattacagataatcaagggaagaataagacaacaatacacagttacttctcctccattttccatggtggagtggatagtctagtcgacttgatgaacaagagctttcctgaattgggtattaaaaaaagagattgcaaacagttgagctggattgatactatcatcttctacagtggtcttgtaaattacaacacaactaattttaaaaaagaaattttgcttgatagatcaggtgggcggaaggcggctttctcgattaagttagactatgttaagaaaccgattccagaaaccgcaatggtcacaattttggaaaaattatatgaagaagatgtaggagttgggatgtttgtgttttacccttatggtggtataatggatgagatttcagaatcagcaattccattcctcatcgagctggaatcatgtatgaaatttggtacatagcttcatgggagaagcaagaagataatgaaaagcatataaactggattcggaatgtttataatttcacgactccttatgtgtcccaaaatccaagaatggcgtatctcaattatagggaccttgatttaggaaaaactaatttcgagagtcctaataattacacacaagcacgtatttggggtgaaaagtattttggtaaaaattttaataggttagtaaaagtaaaaaccaaggttgatcccgataatttctttagaaacgaacaaagcatcccacctcttccc

SEQ ID NO: 137 - DNA, 1550 bp, Cannabis sativa
ctaatcctgaaggaaacttccttaaatgcttctcacaatatattcccaccaatgtaacaaatgcaaaactcgtatacactcaacacgaccaattttatatgtctatcctaaattcgaccatacaaaatcttagatttacctttgacacaaccccaaaaccacttgttatcatcactcctttaaatgtctcccatatccaaggcactattctatgctccaagaaagttggcttgtagattcgaactcgaagcggtggtcatgatgctgagggcatgtcctacatatctcaagtcccatttgttatagtaaacttgagaaacatgcattcggtcaaaatagatgttcatagcgaaactgcatgggttgaagccggagctacccttggagaagtttattattggatcaatgagaacaatgagaatcttagttttcttgctgggtactgccctactgttggcgcgggtggacactttagtggaggaggctatggagcattgatgcgaaattatggcctcgcggctaataacatcattgatgcgcacttagtcaatgttgatggaaaagttttagatcgaaaatccatgggggaagatttgttttgggctatacgtggtggaggagaaaactttggaatcattgcagcgtggaaaattagatttgttgctgtcccatcaatgtctactatattcagtgttaaaaagaacatggagatacatgagcttgtcaagttagttaacaaatggcaaaatattgcttacatgtatgaaaaagaatgattactctttactcactttataaccaggaatattacagataatcaagggaagaataagacaacaatacacagttacttctcctccattttctatggtggagtggatagtctagtcgacttgatgaacaagagctttcctgaattgggtattaaaaaaacagattgcaaacagttgagctggattgatactatcatcttctacagtggtcttgtaaattacaacactactaattttaaaaaagaacttttgcttgatagatcaggtgggcggaaggcggctttctcgattaagttagactaagttaagaaaccgattccagaaaccgcaatggtcacaattttggaaaaattatatgaagaagatgtaggagttgggatgtttgtgttttacccttatggtggtataatggatgagatttcagaatcagcaattccattccctcatcgagctggaatcatgtatgaaatttggtacatagcttcatgggagaagcaagaagataatgaaaagcatataaactggattcggaatgtttacaatttcacgactccttatgtgtcccaaaatccaagaatggcgtatctcaattatagggaccttgatttaggaaaaactaatttcgagagtcctaataattacacacaagcacgtatttggggtgaaaagtattttggtaaaaattttaataggttagtaaaagtaaaaaccaaggttgatcccgataatttctttagaaacgaacaaagcatcccacctcttcccctacgtcatcat

SEQ ID NO: 138 - DNA, 1622 bp, Cannabis sativa
atgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgaaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgtaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttcca

SEQ ID NO: 139 - DNA, 1623 bp, Cannabis sativa
atgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttcca

SEQ ID NO: 140 - DNA, 1617 bp, Cannabis sativa
atgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacgtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttcca

SEQ ID NO: 141 - DNA, 1619 bp, Cannabis sativa
atgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggccagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttgacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagagcaagaagataacgaaaagcatataaactggttcgaagtgtttattaatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaggtgaaaccaaagctgatccaataatttttttagaaacgaacaaagtatcccacctcttcca

SEQ ID NO: 142 - DNA, 1617 bp, Cannabis sativa
tcaacattctcctttaggtttgtttacaaaattatatttttctttctctcattcaatatcaaaatttcaatagctaatcctcaagaaaatttcctaaaatgcttctcccaatatattcataacaatccagcaaatctaaaactcgtatacactcaacacgaccaattgtatatgtctgtcctgaatttgacaatacaaaatcttagatttacctctgatacaaccccaaaaccactcgttattgtcactccttcaaatgtctcccatatccaagccactattctatgctccaagaaagtgggcttgcaaattcgaactcgaagcggtggccatgatgctgagggtttgtcctacacatctcaagtcccatttgttatagtagacttgagaaacatgcattcggtgaaaatagatattcgtagccaaattgcgtgggttgaagccggagctacccttggagaagtttattattggattaatgagaatcttagttttcctggtgggtattgccctactgttggcgtaggtggacactttagtggaggaggctatagagcattaatgcgaaattatggcctcgcagctgataatatcattgatgcacacttagtcaatgttgatggaaaagttctagatcgaaaatccatgggggaagatctattttgggctatacgtggtggtggaggtgaaaactttggaatcattgcagcgtggaaaattagactggttgctgtcccatcaagggctactatattcagtgttaaaaggaatatggagatacatgggcttgtcaagttatttaataaatggcaaaatattgcttacaagtatgacaaagatttattactcatgactcacttcataaccaggaatattatagataatcaaggaaagaataagactacagtacacggttacttctcttgcattttccatggtggagtagatagtctagtcaacttgatgaacaagagctttcctgagttgggtattaaaaaaactgattgcaaagaattgagctggattgatactaccatcttctacagtggtgttgtaaattataacactactaattttcaaaaggaaattttgcttgatagatcagctgggcagaaagtagctttctcagttaagttagactacgttaagaaaccaattccagaaactgcaattgtcaaaattttggagaaattgtatgaagaagatgtaggagttggggtgtatgtattgtacccttacggtggtataatggacaagatctcagaatcaacaattcctttccctcatcgagctggaatcatgtacgaagtttgatacgcagctacctgggagaagcaagaagataatgaaaagcatataaactgggtttgaagtgtttataatttcatgacgccttatgtgtcccaaaatccaagaatggcgtatctcaattatagggaccttgatttaggaaaaactgatcccaagagtcctaataattacacccaagcacgtatctggggtgaaaagtactttggtaaaaactttgacaagttagttaaggtgaaaaccaaagttgatcccaataatttttttagaaacgagcaaagcatcccacctcttccaccacgacgtcat

(19) United States
(12) Patent Application Publication
(10) Pub. No.: US 2022/0298523 A1
(43) Pub. Date: Sep. 22, 2022

(54) GENETICALLY MODIFIED PLANTS AND METHODS OF MAKING THE SAME

(71) Applicant: EMPYREAN NEUROSCIENCE, INC., New York, NY (US)

(72) Inventors: Thomas HENLEY, New York, NY (US); Modassir CHOUDHRY, New York, NY (US); Jose FERNANDEZ-GOMEZ, New York, NY (US)

(21) Appl. No.: 17/711,206

(22) Filed: Apr. 1, 2022

Related U.S. Application Data
(63) Continuation of application No. PCT/US2020/053865, filed on Oct. 1, 2020.

(51) Int. Cl. C12N 15/82; C07K 14/415; C12N 9/02; C12N 9/22

(52) U.S. Cl. CPC C12N 15/8243; C07K 14/415; C12N 9/0004; C12N 9/22

(57) ABSTRACT

Provided herein are compositions comprising genetically modified cells, organisms, or plants described herein or extracts and products thereof and methods for making and using the same. Also provided are therapeutics derived from genetically modified cells, organisms, or plants described herein or extracts and products thereof for use in preventing, treating, or stabilizing disease and conditions.

### CROSS REFERENCE

This application is a continuation of International Application No. PCT/US2020/53865, filed Oct. 1, 2020, which claims the benefit of U.S. Provisional Patent Application No. 62/909,074, filed Oct. 1, 2019, which is entirely incorporated herein by reference.

### SEQUENCE LISTING

The instant application contains a Sequence Listing which has been submitted electronically in ASCII format and is hereby incorporated by reference in its entirety. Said ASCII copy, created on Mar. 22, 2022, is named 200021_705301_SL.txt and is 411,971 bytes in size.

### BACKGROUND

Naturally occurring components in Cannabis may impact the efficacy of therapy and any potential side effects. Accordingly, Cannabis plants having a modified therapeutic component(s) profile may be useful in the production of Cannabis and/or may also be useful in the production of genetically modified Cannabis providing a desired drug profile.

### SUMMARY

Provided herein is a transgenic plant that comprises an endonuclease-mediated stably inherited genomic modification of a tetrahydrocannabinol acid synthase (THCAS) gene. In some cases, a modification can result in increased cannabidiol (CBD) as compared to a comparable control plant without a modification and wherein the transgenic plant comprises less than 1% of tetrahydrocannabinol (THC) as measured by dry weight. Provided herein is also a transgenic plant comprising an endonuclease mediated genetic modification of a tetrahydrocannabinol acid synthase (THCAS) gene that results in a cannabidiol (CBD) to tetrahydrocannabinol (THC) ratio in the transgenic plant of at least 25: 1 as measured by dry weight. In some cases, a modification reduces or suppresses expression of a THCAS gene.

In some cases, a transgenic plant described herein comprises a modification that completely reduces or suppresses a CBDAS gene. In some cases, a transgenic plant with increased CBDAS production, comprises an unmodified CBDAS gene. In some cases, a transgenic plant comprises an unmodified endogenous cannabidiolic acid synthase (CBDAS) gene. In some cases, a transgenic plant comprises at least 25% more CBD as measured by dry weight as compared to a comparable control plant without a modification. In some cases, a transgenic plant comprises at least 50% more CBD as measured by dry weight as compared to a comparable control plant without a modification.

In some instances, a transgenic plant, described herein, contains less than 0.05% of THC as measured by dry weight. In some cases, a transgenic plant comprises a CBD to THC ratio of at least 25:1, 26:1, 27:1, 28:1, 29:1, 30:1, 31:1, 32:1, 33:1, 34:1, 35:1, 40:1, 45:1, or up to about 50:1 as measured by dry weight. In some cases, a transgenic plant comprises 0% THC or an untraceable amount of THC as measured by dry weight as compared to a comparable control plant without a modification.

In some cases, a transgenic plant as described herein is modified by use of an endonuclease wherein the endonuclease comprises a clustered regularly interspaced short palindromic repeats (CRISPR) enzyme, transcription activator-like effector (TALE)-Nuclease, transposon-based nuclease, Zinc finger nuclease, argonaute, meganuclease, or Mega-TAL. In some cases, an endonuclease can be a CRISPR enzyme or argonuate enzyme which can complex with a guide polynucleotide. In some cases, a guide polynucleotide can be a guide RNA or guide DNA. In some cases, a gRNA or gDNA can comprise a sequence that is complementary to a target sequence, or a sequence on a complementary strand to a target sequence in a THCAS gene. In some cases, a guide polynucleotide binds a THCAS gene sequence. In some cases, a CRISPR enzyme complexed with a guide polynucleotide can be introduced into a transgenic plant as a ribonuclear protein (RNP). In some cases, a guide polynucleotide can be chemically modified. In some cases, a CRISPR enzyme and a guide polynucleotide can be introduced into a transgenic plant by a vector comprising a nucleic acid encoding a CRISPR enzyme and a guide polynucleotide. In some cases, a vector can be a binary vector or a Ti plasmid. In some cases, a vector further comprises a selection marker or a reporter. In some cases, an RNP or vector can be introduced into a transgenic plant via electroporation, agrobacterium mediated transformation, biolistic particle bombardment, or protoplast transformation.

In some cases, a transgenic plant or cell thereof further comprises a donor polynucleotide. In some cases, a donor polynucleotide comprises homology to sequences flanking a target sequence. In some cases, a donor polynucleotide introduces a stop codon into a THCAS gene. In some cases, a donor polynucleotide comprises a barcode, a reporter, or a selection marker. In some instances, a guide polynucleotide is a single guide RNA (sgRNA). In some cases, a guide polynucleotide can be a chimeric single guide comprising RNA and DNA. In some embodiments, a target sequence can be at least 18 nucleotides, at least 19 nucleotides, at least 20 nucleotides, at least 21 nucleotides, or at least 22 nucleotides in length. In some cases, a target sequence can be at most 17 nucleotides in length. In some cases, a CRISPR enzyme is Cas9. In some cases, Cas9 recognizes a canonical PAM. In some cases, Cas9 recognizes a non-canonical PAM. In some cases, a guide polynucleotide binds a target sequence from 3-10 nucleotides from a protospacer adjacent motif (PAM). In some cases, a target sequence comprises a sequence complementary to a sequence selected from the group consisting of SEQ ID NOs: 24-34. In some cases, a guide polynucleotide comprises a sequence that comprises at least 50%, 60%, 70%, 80%, 85%, 90%, 95%, 96%, 97%, 98%, 99%, or up to about 100% identity to a sequence selected from the group consisting of SEQ ID NOs 21-34. In some cases, a modification comprises an insertion, a deletion, a substitution, or a frameshift. In some cases, a modification is in a coding region of a THCAS gene. In some cases, a modification can be in a regulatory region of a THCAS gene. In some instances, a plant is a Cannabis plant. In some instances, a modification results in up to about 50% of indel formation. In some cases, a modification results in less than or up to about 25%, less than or up to about 15%, less than or up to about 10%, or less than or up to about 1% of indel formation.

Provided herein is a method for generating a transgenic plant, the method comprising (a) contacting a plant cell comprising a tetrahydrocannabinol acid synthase (THCAS) gene with an endonuclease or a polynucleotide encoding the endonuclease, wherein the endonuclease introduces a stably inherited genomic modification in the THCAS gene; (b) culturing the plant cell with a modification in THCAS gene thereby generating a transgenic plant, wherein the modification results in increased cannabidiol (CBD) as compared to a comparable control plant without the modification and less than 1% of tetrahydrocannabinol (THC) in the transgenic plant as measured by dry weight. Provided herein is also a method for generating a transgenic plant, the method comprising (a) contacting a plant cell comprising a THCAS gene with an endonuclease or a polynucleotide encoding the endonuclease, wherein the endonuclease introduces a genetic modification in the tetrahydrocannabinol acid synthase (THCAS) gene; (b) culturing the plant cell with a modification in THCAS gene thereby generating a transgenic plant, wherein the modification results in a cannabidiol (CBD) to tetrahydrocannabinol (THC) ratio in the transgenic plant of at least 25:1 as measured by dry weight. In some cases, contacting can be via electroporation, agrobacterium mediated transformation, biolistic particle bombardment, or protoplast transformation. In some aspects, a method further comprises culturing a plant cell in with a modification in THCAS gene to generate a callus, a cotyledon, a root, a leaf, or a fraction thereof of the transgenic plant. In some cases, a modification reduces or suppresses expression of a THCAS gene. In some cases, a modification does not alter a cannabidiolic acid synthase (CBDAS) gene in a transgenic plant. In some cases, a modification results in at least 25% more CBD measured by dry weight in a transgenic plant as compared to a comparable control plant without a modification. In some aspects, a modification results in at least 50% more CBD as measured by dry weight in a transgenic plant as compared to a comparable control plant without a modification. In some aspects, a modification results in less than 0.05% of THC in a transgenic plant as measured by dry weight. In some cases, a modification results in a CBD to THC ratio of at least 25:1, 26:1, 27:1, 28:1, 29:1, 30:1, 31:1, 32:1, 33:1, 34:1, 35:1, 40:1, 45:1, or up to about 50:1 as measured by dry weight. In some instances, a transgenic plant an contain 0% THC or an untraceable amount of THC as measured by dry weight as compared to a comparable control plant without a modification. In some cases, an endonuclease comprises a clustered regularly interspaced short palindromic repeats (CRISPR) enzyme, transcription activator-like effector (TALE)-nuclease, transposon-based nuclease, Zinc finger nuclease, meganuclease, argonaute, or Mega-TAL. In some cases, an endonuclease can be a CRISPR enzyme or argonaute enzyme complexed with a guide polynucleotide that can be complementary to a target sequence in a THCAS gene. In some cases, a CRISPR enzyme complexed with a guide polynucleotide (RNP) or a CRISPR enzyme and a guide polynucleotide can be contacted with a plant cell. In some instances, a guide polynucleotide can be chemically modified. In some instances, a CRISPR enzyme complexed with a guide polynucleotide can be contacted with a plant cell. In other instances, a plant cell is contacted with a vector comprising a nucleic acid encoding a CRISPR enzyme and a guide polynucleotide. In some cases, a vector can be a binary vector or a Ti plasmid. In some cases, a vector further comprises a selection marker or a reporter. In some cases, a method further comprises contacting a plant cell with a donor polynucleotide. In some cases, a donor polynucleotide comprises homology to sequences flanking a target sequence. In some aspects, a donor polynucleotide introduces a stop codon into a THCAS gene. In some cases, a donor polynucleotide comprises a barcode, a reporter, or a selection marker. In some cases, a guide polynucleotide can be a single guide RNA (sgRNA). In some cases, a guide polynucleotide can be a chimeric single guide comprising RNA and DNA. In some cases, a target sequence can be at least 18 nucleotides, at least 19 nucleotides, at least 20 nucleotides, at least 21 nucleotides, or at least 22 nucleotides in length. In some cases, a target sequence can be at most 17 nucleotides in length. In some cases, a CRISPR enzyme can be Cas9. In some instances, Cas9 recognizes a canonical protospacer adjacent motif (PAM). In some instances, Cas9 recognizes a non-canonical PAM. In some cases, a guide polynucleotide binds a target sequence from 3-10 nucleotides from a PAM. In some instances, a target sequence comprises a sequence complementary to a sequence selected from the group consisting of SEQ ID NOs 21-34. In some instances, a guide polynucleotide comprises a sequence that comprises at least 50%, 60%, 70%, 80%, 85%, 90%, 95%, 96%, 97%, 98%, 99%, or up to about 100% identity to a sequence selected from the group consisting of SEQ ID NOs 21-34. In some cases, a modification comprises an insertion, a deletion, a substitution, or a frameshift. In some cases, a modification is in a coding region of the THCAS gene. In some cases, a modification is in a regulatory region of the THCAS gene. In some cases, a plant is a Cannabis plant. In some cases, a modification results in at least or up to about 50% of indel formation. In some cases, a modification results in less than or up to about 25%, less than or up to about 15%, less than or up to about 10%, or less than or up to about 1% of indel formation.

Provided herein is a genetically modified cell comprising an endonuclease mediated modification in a tetrahydrocannabinol acid synthase (THCAS) gene, wherein a cell comprises an unmodified cannabidiolic acid synthase (CBDAS) gene, and wherein a cell produces an enhanced amount of CBD as compared to a comparable control cell without a modification. In some cases, the modification reduces or suppresses expression of a THCAS gene. In some cases, a modified cell comprises an unmodified amount of CBD as compared to a comparable control cell without a modification. In some cases, a genetically modified cell comprises at least 25% more CBD as compared to a comparable control cell without a modification. In some cases, a genetically modified cell comprises at least 50% more CBD measured by dry weight as compared to a cell from a comparable control plant without a modification. In some cases, a genetically modified cell comprises a modification that results in at least 99% reduction of tetrahydrocannabinol (THC) as compared to a comparable control cell without a modification. In some cases, a modification results in at least 99.9% reduction of THC as compared to a comparable control cell without a modification. In some cases, a modified cell comprises a CBD to THC ratio of at least 25:1, 26:1, 27:1, 28:1, 29:1, 30:1, 31:1, 32:1, 33:1, 34:1, 35:1, 40:1, 45:1, or up to about 50:1. In some cases, a genetically modified cell is a plant cell, an agrobacterium cell, a E. coli cell, or a yeast cell. In some instances, a genetically modified cell is a plant cell. In some instances, a genetically modified cell is a Cannabis plant cell. In some cases, a genetically modified cell is a callus cell, a protoplast, an embryonic cell, a leaf cell, a seed cell, a stem cell, or a root cell. In some cases, a modification is integrated in the genome of a cell. In some cases, a THCAS gene and/or a CBDAS gene is endogenous to a cell. In some cases, an endonuclease comprises a clustered regularly interspaced short palindromic repeats (CRISPR) enzyme, transcription activator-like effector (TALE)-nuclease, transposon-based nuclease, Zinc finger nuclease, argonaute, meganuclease, or Mega-TAL In some cases, an endonuclease can be a CRISPR enzyme or argonaute enzyme or a CRISPR enzyme that can complex with a guide polynucleotide or an argonaute enzyme that can complex with a guide polynucleotide, wherein the guide polynucleotide comprises a sequence that binds a target sequence within or adjacent to a THCAS gene. In some cases, a guide polynucleotide binds a portion of a THCAS sequence. In some cases, a guide polynucleotide comprises a sequence that binds a THCAS gene sequence. In some cases, a CRISPR enzyme complexed with a guide polynucleotide forms an RNP and is introduced into a genetically modified cell. In some cases, a guide polynucleotide is a chemically modified. In some cases, a CRISPR enzyme and a guide polynucleotide are introduced into a cell by a vector comprising a nucleic acid encoding a CRISPR enzyme and a guide polynucleotide. In an aspect, a vector is a binary vector or a Ti plasmid. In an aspect, a vector further comprises a selection marker or a reporter. In an aspect, an RNP or vector is introduced into a cell via electroporation, agrobacterium mediated transformation, biolistic particle bombardment, or protoplast transformation. In an aspect, a cell further comprises a donor polynucleotide. In some cases, a donor polynucleotide comprises homology to sequences flanking the target sequence. In some cases, a donor polynucleotide introduces a stop codon into the THCAS gene. In some cases, a donor polynucleotide comprises a barcode, a reporter, or a selection marker. In some cases, a guide polynucleotide can be a single guide RNA (sgRNA). In some cases, a guide polynucleotide is a chimeric single guide comprising RNA and DNA. In some cases, a target sequence is at least 18 nucleotides, at least 19 nucleotides, at least 20 nucleotides, at least 21 nucleotides, or at least 22 nucleotides in length. In some cases, a target sequence is at most 17 nucleotides in length. In some cases, a CRISPR enzyme can be a Cas9. In an aspect, Cas9 recognizes a canonical protospacer adjacent motif (PAM). In an aspect, Cas9 recognizes a non-canonical PAM. In some cases, a guide polynucleotide binds a target sequence 3-10 nucleotides from PAM. In some cases, a guide polynucleotide hybridizes with a target sequence within the THCAS gene selected from the group consisting of SEQ ID NOs 21-34 or a complementary thereof. In some cases, a guide polynucleotide comprises a sequence that comprises at least 50%, 60%, 70%, 80%, 85%, 90%, 95%, 96%, 97%, 98%, 99%, or up to about 100% identity to a sequence selected from the group consisting of SEQ ID NOs 21-34. In some cases, a modification comprises an insertion, a deletion, a substitution, or a frameshift. In some cases, a modification is in a coding region of the THCAS gene. In some cases, a modification is in a regulatory region of the THCAS gene. In some cases, a modification results in at least or up to about 50% of indel formation. In some cases, a modification results in less than or up to about 25%, less than or up to about 15%, less than or up to about 10%, or less than or up to about 1% of indel formation.

Provided herein is a tissue comprising the genetically modified cell of any one of the claims 78-119. In an aspect, a tissue is a Cannabis plant tissue. In an aspect, a tissue is a callus tissue. In an aspect, a tissue contains less than 1% of THC. In an aspect, a tissue contains less than 0.05% of THC. In an aspect, a tissue contains 0% THC or an untraceable amount thereof. In some cases, a tissue comprises at least 25% more CBD measured by dry weight as compared to a comparable control tissue without a modification. In some cases, a tissue comprises at least 50% more CBD measured by dry weight as compared to a comparable control tissue without a modification.

Provided herein is a plant comprising a tissue. In some cases, a plant comprises at least 25% more CBD measured by dry weight as compared to a comparable control plant without a modification. In some cases, a plant comprises at least 50% more CBD measured by dry weight as compared to a comparable control plant without a modification. In some cases, a plant is a Cannabis plant.

Provided herein is a method for increasing cannabidiol (CBD) production in a plant cell, the method comprising introducing an endonuclease mediated genomic modification into a tetrahydrocannabinol acid synthase (THCAS) gene of the plant cell, thereby minimizing THCAS expression and increasing CBD production of the plant cell as compared to a comparable control cell without the modification. In some cases, a modification reduces or suppresses expression of a THCAS gene. In some cases, a plant comprises an unmodified endogenous CBDAS gene. In some cases, a modification results in at least 25% more CBD in a plant cell as compared to a comparable control cell without a modification. In some cases, a modification results in a CBD to THC ratio of at least 25:1, 26:1, 27:1, 28:1, 29:1, 30:1, 31:1, 32:1, 33:1, 34:1, 35:1, 40:1, 45:1, or up to about 50:1 in a plant cell. In some cases, a modification results in at least 99% reduction of THC in a plant cell as compared to a comparable control cell without a modification. In some cases, a modification results in at least 99.9% reduction of THC in a plant cell as compared to a comparable control cell without a modification. In an aspect, an endonuclease comprises a clustered regularly interspaced short palindromic repeats (CRISPR) enzyme, transcription activator-like effector (TALE)-nuclease, transposon-based nuclease, Zinc finger nuclease, argonaute, meganuclease, or Mega-TAL. In an aspect, an endonuclease is a CRISPR enzyme or argonaute enzyme complexed with a guide polynucleotide that comprises a sequence that binds a target sequence within or adjacent to a THCAS gene. In some cases, a guide polynucleotide binds a portion of a THCAS sequence. In some cases, a guide polynucleotide comprises a sequence that binds a THCAS gene sequence. In some cases, a CRISPR enzyme complexed with a guide polynucleotide forms an RNP that can be introduced into a plant cell. In some cases, a guide polynucleotide is a chemically modified. In some cases, a CRISPR enzyme and a guide polynucleotide are introduced into a plant cell by a vector comprising a nucleic acid encoding a CRISPR enzyme and a guide polynucleotide. In some cases, a vector is a binary vector or a Ti plasmid. In some cases, a vector further comprises a selection marker or a reporter. In an aspect, an RNP or vector can be introduced into a plant cell via electroporation, agrobacterium mediated transformation, biolistic particle bombardment, or protoplast transformation. In some cases, a method further comprises introducing a donor polynucleotide into a plant cell. In an aspect, a donor polynucleotide comprises homology to sequences flanking a target sequence. In some cases, a donor polynucleotide introduces a stop codon into a THCAS gene. In some cases, a donor polynucleotide comprises a barcode, a reporter, or a selection marker. In some cases, a guide polynucleotide is a single guide RNA (sgRNA). In an aspect, a guide polynucleotide is a chimeric single guide comprising RNA and DNA. In some cases, a target sequence is at least 18 nucleotides, at least 19 nucleotides, at least 20 nucleotides, at least 21 nucleotides, or at least 22 nucleotides in length. In some cases, a target sequence is at most 17 nucleotides in length. In some cases, a CRISPR enzyme can be a Cas9. In some cases, Cas9 recognizes a canonical PAM. In some cases, Cas9 recognizes a non-canonical PAM. In some cases, a guide polynucleotide binds a target sequence from 3-10 nucleotides from a PAM. In some cases, a guide polynucleotide binds a target sequence within a THCAS gene, or binds a sequence complementary to a target sequence within a THCAS gene. In some cases, a guide polynucleotide comprises a sequence comprising from about 50%, 60%, 70%, 80%, 85%, 90%, 95%, 96%, 97%, 98%, 99%, or up to about 100% identity to a sequence selected from the group consisting of SEQ ID NOs 21-34. In an aspect, a modification comprises an insertion, a deletion, a substitution, or a frameshift. In an aspect, a modification is in a coding region of the THCAS gene. In an aspect, a modification is in a regulatory region of the THCAS gene. In an aspect, a plant cell is a Cannabis plant cell. In some cases, a method further comprises culturing a plant cell to generate a plant tissue. In some cases, a method further comprises culturing a plant tissue to generate a plant. In some cases, a plant contains less than 0.01% of THC measured by dry weight. In some cases, a plant comprises a ratio of CBD to THC of at least 25:1 measured by dry weight. In some cases, a plant comprises at least 25% more CBD measured by dry weight as compared to a comparable control plant without a modification. In some cases, a modification results in at least or up to about 50% of indel formation. In an aspect, a modification results in less than or up to about 25%, less than or up to about 15%, less than or up to about 10%, or less than or up to about 1% of indel formation.

Provided herein is a composition comprising an endonuclease or a polynucleotide encoding an endonuclease, wherein an endonuclease preferentially binds a tetrahydrocannabinol acid synthase (THCAS) gene over a cannabidiolic acid synthase (CBDAS) gene and is capable of introducing a modification into a THCAS gene, wherein a modification reduces or abrogates expression of a THCAS gene. In some cases, a modification reduces or suppresses expression of the THCAS gene. In an aspect, a modification comprises an insertion, a deletion, a substitution, or a frameshift. In an aspect, a modification is in a coding region of the THCAS gene. In some cases, a modification is in a regulatory region of the THCAS gene. In some cases, an endonuclease comprises a clustered regularly interspaced short palindromic repeats (CRISPR) enzyme, transcription activator-like effector (TALE)-nuclease, transposon-based nuclease, Zinc finger nuclease, argonaute, meganuclease, or Mega-TAL. In some cases, an endonuclease is a CRISPR enzyme or argonaute enzyme complexed with a guide polynucleotide that comprises a sequence that binds a target sequence within or adjacent to a THCAS gene. In some cases, a guide polynucleotide binds a portion of a THCAS sequence. In some cases, a guide polynucleotide comprises less than 50% identity to a CBDAS gene. In some cases, a CRISPR enzyme complexed with a guide polynucleotide forms a ribonuclear protein (RNP). In some cases, a guide polynucleotide is chemically modified. In some cases, a CRISPR enzyme complexed with a guide polynucleotide are encoded by a vector. A vector can be a binary vector or a Ti plasmid. In some instances, a vector further comprises a selection marker or a reporter. In some instances, an RNP or vector can be introduced into a plant cell provided herein via electroporation, agrobacterium mediated transformation, biolistic particle bombardment, or protoplast transformation. In some cases, composition provided herein further comprises a donor polynucleotide. In some cases, a donor polynucleotide comprises homology to sequences flanking the target sequence. In some cases, a donor polynucleotide introduces a stop codon into a THCAS gene. In some cases, a donor polynucleotide comprises a barcode, a reporter, or a selection marker. In some cases, a guide polynucleotide is a single guide RNA (sgRNA). In some cases, a guide polynucleotide is a chimeric single guide comprising RNA and DNA. In some cases, a target sequence is at least 18 nucleotides, at least 19 nucleotides, at least 20 nucleotides, at least 21 nucleotides, or at least 22 nucleotides in length. In some cases, a target sequence is at most 17 nucleotides in length. In an aspect, a CRISPR enzyme can be Cas9. In some cases, Cas9 recognizes a canonical PAM. In some cases, Cas9 recognizes a non-canonical PAM. In some cases, a guide polynucleotide binds a target sequence from 3-10 nucleotides from a PAM. A target sequence can comprise a sequence complementary to a sequence selected from the group consisting of SEQ ID NOs 21-34. In some cases, a guide polynucleotide comprises a sequence comprising from about 50%, 60%, 70%, 80%, 85%, 90%, 95%, 96%, 97%, 98%, 99%, or up to about 100% identity to a sequence selected from the group consisting of SEQ ID NOs 21-34. In some cases, a modification comprises an insertion, a deletion, a substitution, or a frameshift. In some cases, a modification is in a coding region of the THCAS gene. In some cases, a modification is in a regulatory region of the THCAS gene.

Provided herein is a kit for genome editing comprising a composition provided herein.

Provided herein is a cell comprising a composition provided herein. A cell can be a plant cell, an agrobacterium cell, a E. coli cell, or a yeast cell. In some cases, a cell is a plant cell. In some cases, a cell is a Cannabis plant cell. In some cases, a cell is a callus cell, a protoplast, an embryonic cell, a leaf cell, a seed cell, a stem cell, or a root cell.

Provided herein is a plant comprising a cell provided herein.

Provided herein is a pharmaceutical composition comprising a transgenic plant or a derivative or extract thereof. Also provided herein is a genetically modified cell and/or a tissue. In some cases, a pharmaceutical composition further comprises a pharmaceutically acceptable excipient, diluent, or carrier. A pharmaceutically acceptable excipient can be a lipid.

Provided herein is a nutraceutical composition comprising a transgenic plant or a derivative or extract thereof. Provided herein is also a nutraceutical composition comprising a genetically modified cell or a tissue.

Provided herein is a food supplement comprising a transgenic plant or a derivative or extract thereof. Provided herein is also a genetically modified cell or a tissue. In some aspects a nutraceutical composition or a food supplement can be in an oral form, a transdermal form, an oil formulation, an edible food, or a food substrate, an aqueous dispersion, an emulsion, a solution, a suspension, an elixir, a gel, a syrup, an aerosol, a mist, a powder, a tablet, a lozenge, a gel, a lotion, a paste, a formulated stick, a balm, a cream, or an ointment.

Provided herein is a method of treating a disease or condition comprising administering a pharmaceutical composition, a nutraceutical composition, or a food supplement to a subject in need thereof. In some cases, a disease or condition is selected from the group consisting of anorexia, emesis, pain, inflammation, multiple sclerosis, Parkinson's disease, Huntington's disease, Tourette's syndrome, Alzheimer's disease, epilepsy, glaucoma, osteoporosis, schizophrenia, cardiovascular disorders, cancer, and obesity.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows an exemplary portion of the THCAS gene (SEQ ID NO: 1) that can be targeted using methods provided herein, such as CRISPR. THCAS in PK (CM010797.2, start 28650052, end 28651687) annotated with SNPs (in green) from likely PK CBCAS (AGQN03005496.1). Shown are guides with 1 bp difference (pink), guides with 2 bp difference (purple), guides with 3 bp or more difference (orange).

FIG. 2 shows nucleotide alignment of THCAS hits in Finola at 85% stringency (SEQ ID NOS 105-115, respectively, in order of appearance).

FIG. 3 shows clustal alignment of THCAS in Finola (SEQ ID NOS 116-121, respectively, in order of appearance). Shown are all the THCAS annotated hits with guides annotated. Shared nucleotides are marked with a star, regions of high similarity or difference were used for designing the three groups of guides. QKVJ02004887.1_13942_15577 chrnan and CM011610.1_22244180_22245797 chr:6.0 were used for guide design in Benchling

FIG. 4 shows nucleotide alignment of THCAS hits in purple kush at 85% stringency (SEQ ID NOS 122-130, respectively, in order of appearance).

FIG. 5 shows nucleotide alignment of CBDAS in Finola at 85% stringency (SEQ ID NOS 131 and 132, respectively, in order of appearance).

FIG. 6 shows multiple sequence alignments of the identified genomics sequences mapping to the THCAS gene in Purple Kush Cannabis genome (SEQ ID NOS 133-137, 125, 138-142, respectively, in order of appearance).

FIGS. 7A and 7B show agrobacterium mediated transformation in callus cell from Finola plants resulting in expression of a representative transgene, namely GUS (blue with arrow pointed to). In some embodiments, the callus cells may be transformed with agrobacterium resulting in expression of THCAS transgene.

FIGS. 8A-8C show cotyledon inoculated with agrobacterium carrying an exemplary transgene GUS expression vector pCambia1301. FIGS. 8A and 8B show that GUS expression (blue; indicated by an arrow) is observed in cotyledon proximal site where callus regeneration occurs. In some embodiments, THCAS expression may be observed in cotyledon proximal sites where callus regeneration occurs when cotyledon is inoculated with agrobacterium carrying THCAS transgene. FIG. 8C shows that explant regenerated from primordia cells showing random GUS expression in regenerated explant. In some embodiments, an explant regenerated from primordia cells may display random THCAS gene.

FIGS. 9A-9D show that hypocotyls inoculated with pCambia:1301:GUS showed blue stain in regenerative tissues (b and d), and in regenerated explant (a and c) after 5 days on selection media.

FIG. 10 shows that Hemp isolated protoplasts were transfected with GUS expressing plasmid pCambia1301. GUS assay was conducted 72 hrs after transfection. Blue nuclei indicate GUS expression (indicated by black arrow).

FIG. 11 shows that Hemp Floral dipping was conducted by submerging female floral organs into Agrobacterium immersion solution for 10 min. Process was repeated 48 hrs later and inoculated plants were ready to be crossed with male pollen donors 24 hrs after the last inoculation.

FIGS. 12A-12C show that Cotyledon regeneration was achieved from a diversity of tissues. Primordia cells regenerate a long strong shoot (black arrow shown in FIG. 12A). In addition, callus regeneration from cotyledon proximal side also regenerate random numbers of shoots (white arrows shown in FIGS. 12B and 12C).

FIG. 13 shows that hypocotyl Regeneration showed high efficiency. Hypocotyl produced shoots and roots on plates and then were transferred to bigger pots where they could develop further. Once plants have developed strong roots, and the shoot is elongated, plantlets are transferred to compost for further growth.

FIG. 14 shows that agroinfiltration of hemp Finola leaves. Agrobacterium carrying the representative transgene GUS expression vector pCambia1302 was injected into the adaxial side of leaves using a 1 ml syringe. After 72 hrs, GUS assay was performed, and blues was observed in infiltrated leaves (indicated by black arrows).

FIGS. 15A-15C show maps of vectors disclosed herein.

### DETAILED DESCRIPTION

As used in the specification and claims, the singular forms “a,” “an,” and “the” include plural references unless the context clearly dictates otherwise. For example, the term “a chimeric transmembrane receptor polypeptide” includes a plurality of chimeric transmembrane receptor polypeptides.

The term “about” or “approximately” means within an acceptable error range for the particular value as determined by one of ordinary skill in the art, which can depend in part on how the value can be measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an algal cell, seaweeds, a fungal cell, an animal cell, a cell from an invertebrate animal, a cell from a vertebrate animal, a cell from a mammal, and the like. Sometimes a cell is not originating from a natural organism (e.g. a cell can be a synthetically made, sometimes termed an artificial cell).

The term “gene,” as used herein, refers to a nucleic acid (e.g., DNA such as genomic DNA and cDNA) and its corresponding nucleotide sequence that can be involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region can contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism but which can be introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence).

The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS] dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide can be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides can include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif.; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The term “percent (%) identity,” as used herein, can refer to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment, for purposes of determining percent identity, can be achieved in various ways that are within the skill in the art, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

As used herein, the term “plant” includes a whole plant and any descendant, cell, tissue, or part of a plant. A class of plant that can be used in the present disclosure can be generally as broad as the class of higher and lower plants amenable to mutagenesis including angiosperms (monocotyledonous and dicotyledonous plants), gymnosperms, ferns and multicellular algae. Thus, “plant” includes dicot and monocot plants. The term “plant parts” include any part(s) of a plant, including, for example and without limitation: seed (including mature seed and immature seed); a plant cutting; a plant cell; a plant cell culture; a plant organ (e.g., pollen, embryos, flowers, fruits, shoots, leaves, roots, stems, and explants). A plant tissue or plant organ may be a seed, protoplast, callus, or any other group of plant cells that can be organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks.

As used herein, the term “tetrahydrocannabinolic acid (THCA) synthase inhibitory compound” refers to a compound that suppresses or reduces an activity of THCA synthase enzyme activity, or expression of THCA synthase enzyme, such as for example synthesis of mRNA encoding a THCA synthase enzyme (transcription) and/or synthesis of a THCA synthase polypeptide from THCA synthase mRNA (translation). In some embodiments the selective THCA synthase inhibitory compound specifically inhibits a THCA synthase that decreases formation of delta-9-tetrahydrocannabinol (THC) and/or increases cannabidiol (CBD).

As used herein, the term “transgene” refers to a segment of DNA which has been incorporated into a host genome or is capable of autonomous replication in a host cell and is capable of causing the expression of one or more coding sequences. Exemplary transgenes will provide the host cell, or plants regenerated therefrom, with a novel phenotype relative to the corresponding non-transformed cell or plant. Transgenes may be directly introduced into a plant by genetic transformation or may be inherited from a plant of any previous generation which was transformed with the DNA segment. In some cases, a transgene can be a barcode. In some cases, a transgene can be a marker.

As used herein, the term “transgenic plant” refers to a plant or progeny plant of any subsequent generation derived therefrom, wherein the DNA of the plant or progeny thereof contains an introduced exogenous DNA segment not naturally present in a non-transgenic plant of the same strain. The transgenic plant may additionally contain sequences which are native to the plant being transformed, but wherein the “exogenous” gene has been altered in order to alter the level or pattern of expression of the gene, for example, by use of one or more heterologous regulatory or other elements.

A vector can be a polynucleotide (e.g., DNA or RNA) used as a vehicle to artificially carry genetic material into a cell, where it can be replicated and/or expressed. Such a polynucleotide can be in the form of a plasmid, YAC, cosmid, phagemid, BAC, virus, or linear DNA (e.g., linear PCR product), for example, or any other type of construct useful for transferring a polynucleotide sequence into another cell. A vector (or portion thereof) can exist transiently (i.e., not integrated into the genome) or stably (i.e., integrated into the genome) in the target cell.

The practice of some methods disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

### Genetically Modified Plants and Portions Thereof

Described are genetically modified Cannabis and/or hemp plants, portions of plants, and Cannabis and/or hemp plant derived products as well as expression cassettes, vectors, compositions, and materials and methods for producing the same. Cannabis contains many chemically distinct components, many of which have therapeutic properties that can be altered. Therapeutic components of medical Cannabis are delta-9-tetrahydrocannabinol (THC) and cannabidiol (CBD). Provided herein are genetically modified Cannabis having substantially low levels of tetrahydrocannabinol (THC), substantially high levels of cannabidiol (CBD), or combinations thereof. Provided herein are also methods of making genetically modified Cannabis utilizing Clustered Regularly Interspaced Short Palindromic Repeats (CRISPR) technology and reagents for generating the genetically modified Cannabis. Compositions and methods provided herein can be utilized for the generation of a substantially CBD-only plant strain. Compositions provided herein can also be utilized for various uses including but not limited to therapeutic uses, preventative uses, palliative uses, and recreational uses.

C. sativa has been intensively bred, resulting in extensive variation in morphology and chemical composition. It is perhaps best known for producing cannabinoids, a unique class of compounds that may function in chemical defense, but also have pharmaceutical and psychoactive properties. Heat converts the cannabinoid acids (e.g. tetrahydrocannabinolic acid, THCA) to neutral molecules (e.g. (−)-trans-Δ 9 50-tetrahydrocannabinol, THC) that bind to endocannabinoid receptors. This pharmacological activity leads to analgesic, antiemetic, and appetite-stimulating effects and may alleviate symptoms of neurological disorders including epilepsy (Devinsky et al. 2014) and multiple sclerosis (van Amerongen et al. 2017). There are over 113 known cannabinoids (Elsohly and Slade 2005), but the two most abundant natural derivatives are THC and cannabidiol (CBD). THCA and CBDA are both synthesized from cannabigerolic acid by the related enzymes THCA synthase (THCAS) and CBDA synthase (CBDAS), respectively (Sirikantaramas et al. 2004; 66 Taura et al. 2007). Expression of THCAS and CBDAS appear to be the major factor determining cannabinoid content.

THC is responsible for the well-known psychoactive effects of Cannabis and/or hemp consumption, but CBD, while non-intoxicating, also has therapeutic properties, and is specifically being investigated as a treatment for both schizophrenia (Osborne et al. 2017) and Alzheimer's disease (Watt and Karl 2017). Cannabis has traditionally been classified as having a drug (“marijuana”) or hemp chemotype based on the relative proportion of THC to CBD, but types grown for psychoactive use produce relatively large amounts of both. Cannabis containing high levels of CBD is increasingly grown for medical use. Examples of cannabinoids comprise compounds belonging to any of the following classes of molecules, their derivatives, salts, or analogs: Tetrahydrocannabinol (THC), Tetrahydrocannabivarin (THCV), Cannabichromene (CBC), Cannabichromanon (CBCN), Cannabidiol (CBD), Cannabielsoin (CBE), Cannabidivarin (CBDV), Cannbifuran (CBF), Cannabigerol (CBG), Cannabicyclol (CBL), Cannabinol (CBN), Cannabinodiol (CBND), Cannabitriol (CBT), Cannabivarin (CBV), cannabigerovarin (CGGV), cannabichromevarin (CBCV), cannabigerol monomethyl ether (CBGM), and Isocanabinoids.

In some aspects, a gene or portion thereof associated with THC production may be disrupted. In other aspects, a gene or portion thereof associated with THC production of Cannabis may be down regulated. The DNA sequences encoding the THCA synthase gene in Cannabis and Hemp plants is mapped and annotated using the published genome sequence of Cannabis sativa and Hemp (Finola).

In some aspects, low THC hemp and high CBD strains of Cannabis will be genomically engineered. In some aspects, genetically modified plants or portions thereof, such as transgenic F1 plants, can be used to establish clonal strains in which the THC synthase inactivating mutations have been stably transmitted. In an aspect, a transgenic plant provided herein can comprise an endonuclease mediated stably inherited genomic modification. A stably inherited genomic modification can be in a THCAS gene or portion thereof. In some cases, a donor sequence may also be introduced into the genetically modified plants, such as a barcode sequence. A donor sequence may be inserted into a safe harbor locus or intergenic region of a sequence.

In some aspects, a sequence that can be modified is listed in Table 1, Table 2, Table 3, or Table 7. A sequence that can be modified can be or can be about 70%, 75%, 80%, 85%, 90%, 95%, 98%, 99%, or 100% identical to SEQ ID NO: 1, SEQ ID NO: 2, SEQ ID NO: 3, SEQ ID NO: 4, SEQ ID NO: 5, SEQ ID NO: 6-10, and/or SEQ ID NO: 64-76. In some aspects, a gene sequence or a portion thereof such as sequences listed in SEQ ID NO: 1-5, SEQ ID NO: 6-10, and/or SEQ ID NO: 64-76 can be disrupted or modified with an efficiency from about 5%, 10%, 15%, 20%, 25%, 30%, 35%, 40%, 45%, 50%, 55%, 60%, 65%, 70%, 75%, 80%, 85%, 90%, 95%, 98%, or up to about 100%. In some cases, a polypeptide provided herein comprises a modification as compared to a comparable wildtype or unmodified polypeptide. Modified polypeptides can be from about 50%, 60%, 65%, 70%, 75%, 80%, 85%, 90%, 95%, 98%, 99%, or 100% percent identical to any one of SEQ ID NO: 52-63; SEQ ID NO: 44-51, SEQ ID NO: 11-20, and/or SEQ ID NO: 35-43.

In an aspect, a genomic modification can result in a transgenic plant, portion of a plant, and/or plastid of a plant having less than about 5%, 4%, 3%, 2%, 1%, 1.75%, 1.5%, 1.25%, 1.1%, 0.5%, 0.25%, 0.05%, 0.02%, 0.01%, or 0% of THC as measured by dry weight. In another aspect, a transgenic plant or portion of a plant comprising an endonuclease mediated genetic modification of a THCAS gene or portion thereof can result in a CBD to THC ratio in said plant of at least about 25:1, 26:1, 27:1, 28:1, 29:1, 30:1, 31:1, 32:1, 33:1, 34:1, 35:1, 40:1, 45:1, or up to about 50:1, 100: 50, 75: 25, 50: 12.5, 25: 6.25, 12.5: 3.1, 25: 3, 25: 2, 25: 1, 25: 0.5, 25: 0.25, or 25:0.

TABLE 1
Tetrahdrocannabinolic acid synthase
gene sequence and peptide sequence

| SEQ ID | Sequence |
|---|---|
| 1 | atgatgatgcggtggaagaggtggg |
| (CM010797. | atactttgttcgtttctaaaaaaat |
| 2_28650052_ | tattgggatcaactttagttttcac |
| 28651687 | cttaactaacctgttaaaattttta |
| CHR:7.0) | ccaaaatacttttcaccccaaatac |
| | gtgcttgtgtgtaattattaggact |
| | cgcatgattagtttttcctaaatca |
| | aggtccctataattgagatacgcca |
| | atcttggattttgggacacataagg |
| | agtcgtaaaattataaacacttcga |
| | acccagtttatatgcttttcattat |
| | cttcttgcttctcccaggaagcagt |
| | gtaccaaagttcatacattattcca |
| | gctcgatgagggaatggaattgctg |
| | attctgaaatctcctccattatacc |
| | accgtaagggtacaacacatacatc |
| | ccagctcctacatcttcttcatata |
| | atttttccaaaattttgaccattgc |
| | agtttctggaattggtttcttaaca |
| | tagtctaacttaattgagaaagccg |
| | tcttcttcccagctgatctatcaag |
| | caaaatttcctttttaaaattagca |
| | gtgttaaaatttacaacaccactgt |
| | agaagatggttgtatcaatccagct |
| | aaattctttgcaatcagttttttta |
| | atacccaactcacgaaagctcttgt |
| | tcatcaagtcgactagactatccac |
| | tccaccatgaaaaattgaagagaag |
| | taaccatgtactgtagtcttattct |
| | tcccatgattatctgtaatattctt |
| | tgttatgaagtgagtcatgagtact |
| | aaatctttgtcatacttgtaagcaa |
| | tattttgccatttgttaaataactt |
| | gacaagcccatgtatctccatgttc |
| | tttttaacactgaatatagtagact |
| | ttgatgggacagcaaccagtttgat |
| | tttccatgctgcaatgattccaaag |
| | ttttctcctccaccaccacgtatag |
| | cccaaaacagatcttctcccatgga |
| | ttttcgatctagaacttttccatca |
| | acattgactaagtgtgcatcaataa |
| | tattatcagccgcaaggccataatt |
| | tcgcatcaatgctccatagcctcct |
| | ccactaaagtgtccacctacgccaa |
| | cagtagggcaatacccaccaggaaa |
| | actaagattctcattcttctcattg |
| | atccaataataaacttctccaaggg |
| | tagctccggcttcaacccacgcagt |
| | ttggctatgaacatctattttgatc |
| | gaatgcatgtttctcaagtctacta |
| | caacaaatgggacttgagatatgta |
| | ggacataccctcagcatcatggcca |
| | ccgcttcgagttcgaatctgcaagc |
| | caactttcttagagcataaaatagt |
| | tgcttggatatgggagttatttgaa |
| | ggagtgacaataacgagtggttttg |
| | gggttgtatcagagatgaatctaag |
| | attttgtattgtcgaattcaggata |
| | gacatatacaattggtcgtgttgag |
| | tgtatacgagttttggatttgctac |
| | attgttgggaatatgttttgagaag |
| | catttaaggaagttttctcgaggat |
| | tagctattgaaatttggatatggaa |
| | tgagagaaagaaaaatattattttg |
| | caaacaaaccaaaaggaaaatgctg |
| | agcaattcat |
| 2 | MNCSAFSFWFVCKIIFFFLSFHIQI |
| | SIANPRENFLKCFSKHIPNNVANPK |
| | LVYTQHDQLYMSILNSTIQNLRFIS |
| | DTTPKPLVIVTPSNNSHIQATILCS |
| | KKVGLQIRTRSGGHDAEGMSYISQV |
| | PFVVVDLRNMHSIKIDVHSQTAWVE |
| | AGATLGEVYYWINEKNENLSFPGGY |
| | CPTVGVGGHFSGGGYGALMRNYGLA |
| | ADNIIDAHLVNVDGKVLDRKSMGED |
| | LFWAIRGGGGENFGIIAAWKIKLVA |
| | VPSKSTIFSVKKNMEIHGLVKLFNK |
| | WQNIAYKYDKDLVLMTHFITKNITD |
| | NHGKNKTTVHGYFSSIFHGGVDSLV |
| | DLMNKSFPELGIKKTDCKEFSWIDT |
| | TIFYSGVVNFNTANFKKEILLDRSA |
| | GKKTAFSIKLDYVKKPIPETAMVKI |
| | LEKLYEEDVGAGMYVLYPYGGIMEE |
| | ISESAIPFPHRAGIMYELWYTASWE |
| | KQEDNEKHINWVRSVYNFTTPYVSQ |
| | NPRLAYLNYRDLDLGKTNHASPNNY |
| | TQARIWGEKYFGKNFNRLVKVKTKV |
| | DPNNFFRNEQSIPPLPPHHH |

TABLE 2
Tetrahydrocannabinolic acid synthase gene
sequence negative strand and reverse
complement

| SEQ ID | Sequence |
|---|---|
| 3 | tgaattgctcagcattttccttttg |
| | gtttgtttgcaaaataatatttttc |
| | tttctctcattccatatccaaattt |
| | caatagctaatcctcgagaaaactt |
| | ccttaaatgcttctcaaaacatatt |
| | cccaacaatgtagcaaatccaaaac |
| | tcgtatacactcaacacgaccaatt |
| | gtatatgtctatcctgaattcgaca |
| | atacaaaatcttagattcatctctg |
| | atacaaccccaaaaccactcgttat |
| | tgtcactccttcaaataactcccat |
| | atccaagcaactattttatgctcta |
| | agaaagttggcttgcagattcgaac |
| | tcgaagcggtggccatgatgctgag |
| | ggtatgtcctacatatctcaagtcc |
| | catttgttgtagtagacttgagaaa |
| | catgcattcgatcaaaatagatgtt |
| | catagccaaactgcgtgggttgaag |
| | ccggagctacccttggagaagttta |
| | ttattggatcaatgagaagaatgag |
| | aatcttagttttcctggtgggtatt |
| | gccctactgttggcgtaggtggaca |
| | ctttagtggaggaggctatggagca |
| | ttgatgcgaaattatggccttgcgg |
| | ctgataatatcattgatgcacactt |
| | agtcaatgttgatggaaaagttcta |
| | gatcgaaaatccatgggagaagatc |
| | tgttttgggctatacgtggtggtgg |
| | aggagaaaactttggaatcattgca |
| | gcatggaaaatcaaactggttgctg |
| | tcccatcaaagtctactatattcag |
| | tgttaaaaagaacatggagatacat |
| | gggcttgtcaagttatttaacaaat |
| | ggcaaaatattgcttacaagtatga |
| | caaagatttagtactcatgactcac |
| | ttcataacaaagaatattacagata |
| | atcatgggaagaataagactacagt |
| | acatggttacttctcttcaattttt |
| | catggtggagtggatagtctagtcg |
| | acttgatgaacaagagctttcgtga |
| | gtt |
| | gggtattaaaaaaactgattgcaaa |
| | gaattgagctggattgatacaacca |
| | tcttctacagtggtgttgtaaatta |
| | caacactgctaattttaaaaaggaa |
| | atutgcUgatagatcagctgggaag |
| | aagacggctUctcaattaagttaga |
| | ctatgttaagaaaccaaitccagaa |
| | actgcaatggtcaaaattttggaaa |
| | aattatatgaagaagatgtaggagc |
| | tgggatgtatgtgUgtacccttacg |
| | gtggtataatggaggagatttcaga |
| | atcagcaattccattccctcatcga |
| | gctggaataatgtatgaactttggt |
| | acactgcttcctgggagaagcaaga |
| | agataatgaaaagcatataaactgg |
| | gUcgaagtgtttataattttacgac |
| | tccttatgtgtcccaaaatccaaga |
| | ttggcgtatctcaattatagggacc |
| | ttgatttaggaaaaactaatcatgc |
| | gagtcctaataattacacacaagca |
| | cgtantggggtgaaaagtattttgg |
| | taaaaattttaacaggttaguaagg |
| | tgaaaactaaagttgatcccaataa |
| | tttttttagaaacgaacaaagtatc |
| | ccacctcttccaccgcatcatcat |
| 4 | atgatgatgcggtggaagaggtggg |
| | atactttgttcgtttctaaaaaaat |
| | tattgggatcaactttagttttcac |
| | cttaactaacctgttaaaattttta |
| | ccaaaatacttttcaccccaaatac |
| | gtgcttgtgtgtaattattaggact |
| | cgcatgattagtttttcctaaatca |
| | aggtccctataattgagatacgcca |
| | atcttggattttgggacacataagg |
| | agtcgtaaaattataaacacttcga |
| | acccagtttatatgctutcattatc |
| | ttcttgcttctcccaggaagcagtg |
| | taccaaagttcatacattattccag |
| | ctcgatgagggaatggaattgctga |
| | ttctgaaatctcctccattatacca |
| | ccgtaagggtacaacacatacatcc |
| | cagctcctacatcttcttcatataa |
| | tUUccaaaattttgaccattgcagU |
| | tctggaattggtttcttaacatagt |
| | ctaacUaattgagaaagccgtcttc |
| | ttcccagctgatctatcaagcaaaa |
| | tttcctttttaaaattagcagtgtt |
| | gtaatttacaacaccactgtagaag |
| | atggttgtatcaatccagctcaatt |
| | ctttgcaatcagtttttttaatacc |
| | caactcacgaaagctcttgttcatc |
| | aagtcgactagactatccactccac |
| | catgaaaaattgaagagaagtaacc |
| | atgtactgtagtcttattcttccca |
| | tgattatctgtaatattctttgtta |
| | tgaagtgagtcatgagtactaaatc |
| | tttgtcatacttgtaagcaatattt |
| | tgccatttgttaaataacttgacaa |
| | gcccatgtatctccatgttcttttt |
| | aacactgaatatagtagactttgat |
| | gggacagcaaccagtttgattttcc |
| | atgctgcaatgattccaaagttttc |
| | tcctccaccaccacgtatagcccaa |
| | aacagatcttctcccatggattttc |
| | gatctagaacttttccatcaacatt |
| | gactaagtgtgcatcaatgatatta |
| | tcagccgcaaggccataatttcgca |
| | tcaatgctccatagcctcctccact |
| | aaagtgtccacctacgccaacagta |
| | gggcaatacccaccaggaaaactaa |
| | gaUctcattcttctcattgatccaa |
| | taataaacttctccaagggtagctc |
| | cggcttcaacccacgcagtttggct |
| | atgaacatctattttgatcgaatgc |
| | atgtttctcaagtctactacaacaa |
| | atgggacttgagatatgtaggacat |
| | accctcagcatcatggccaccgctt |
| | cgagttcgaatctgcaagccaactt |
| | tcttagagcataaaatagttgcttg |
| | gatatgggagttatugaaggagtga |
| | caataacgagtggttttggggttgt |
| | atcagagatgaatctaagattUgta |
| | Ugtcgaattcaggatagacatatac |
| | aattggtcgtgttgagtgtatacga |
| | gtUtggatttgctacattgUgggaa |
| | tatgttttgagaagcaUtaaggaag |
| | ttttctcgaggattagctattgaaa |
| | tttggatatggaatgagagaaagaa |
| | aaatattattttgcaaacaaaccaa |
| | aaggaaaatgctgagcaattca |

TABLE 3
Cannabidiolic acid synthase peptide sequence

| SEQ ID | Sequence |
|---|---|
| 5 | MKCSTFSFWFVCKIIFFFFSFNIQTS |
| | IANPRENFLKCFSQYIPNNATNLKL |
| | VYTQNNPLYMSVLNSTIHNLRFTSD |
| | TTPKPLVIVTPSHVSHIQGTILCSK |
| | KVGLQIRTRSGGHDSEGMSYISQVP |
| | FVIVDLRNMRSIKIDVHSQTAWVEA |
| | GATLGEVYYWVNEKNENLSLAAGYC |
| | PTVCAGGHFGGGGYGPLMRNYGLAA |
| | DNIIDAHLVNVHGKVLDRKSMGEDL |
| | FWALRGGGAESFGIIVAWKIRLVAV |
| | PKSTMFSVKKIMEIHELVKLVNKWQ |
| | NIAYKYDKDLLLMTHFITRNITDNQ |
| | GKNKTAIHTYFSSVFLGGVDSLVDL |
| | MNKSFPELGIKKTDCRQLSWIDTII |
| | FYSGVVNYDTDNFNKEILLDRSAGQ |
| | NGAFKIKLDYVKKPIPESVFVQILE |
| | KLYEEDIGAGMYALYPYGGIMDEIS |
| | ESAIPFPHRAGILYELWYICSWEKQ |
| | EDNEKHLNWIRNIYNFMTPYVSKNP |
| | RLAYLNYRDLDIGINDPKNPNNYTQ |
| | ARIWGEKYFGKNFDRLVKVKTLVDP |
| | NNFFRNEQSIPPLPRHRH |

In specific embodiments, there are provided Cannabis and/or hemp plants and/or cells having enhanced production of CBD and/or cannabichromene and downregulated expression and/or activity of THCA synthase. In another aspect, a modification reduces, suppresses, or completely represses expression of a THCAS gene in a plant or plastid of a plant. In some cases, a transgenic plant comprises an unmodified endogenous CBDAS gene. In some cases, a transgenic plant with increased CBDAS production, comprises an unmodified CBDAS gene. In some cases, a transgenic plant provided herein can contain increased levels of CBDAS as compared to a comparable plant that is absent the genomic modification. In some cases, a transgenic plant provided herein can contain from about 5%, 10%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 125%, 150%, 175%, 200%, 225%, 250%, 275% or up to about 300% more CBD as measured by dry weight as compared to a comparable control plant without the genomic modification. In some cases, a transgenic plant provided herein can contain from about 1 fold, 2 fold, 3 fold, 4 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, 100 fold, 150 fold, 200 fold, 250 fold, 300 fold, 350 fold, 400 fold, or up to about 500 fold more CBD as measured by dry weight as compared to a comparable control plant without the genomic modification. In some cases, a transgenic plant provided herein can comprise a CBD to THC ratio of at least: 25:1, 26:1, 27:1, 28:1, 29:1, 30:1, 31:1, 32:1, 33:1, 34:1, 35:1, 40:1, 45:1, 50:1, 5:1, 10:1, 20:1, 30:1, 40:1, 50:1, 60:1, 70:1, 80:1, 90:1, 100:1, 120:1, 130:1, 140:1, 150:1, 160:1, 180:1, 200:1, 220:1, 240:1, 260:1, 280:1, or up to about 300:1 as measured by dry weight.

In some aspects, the efficiency of genomic disruption of a Cannabis and/or hemp plants or any part thereof, including but not limited to a cell, with any of the nucleic acid delivery platforms described herein, can result in disruption of a gene or portion thereof at about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or up to about 100% as measured by nucleic acid or protein analysis.

In one embodiment, the Cannabis cultivar produces an assayable combined cannabidiolic acid and cannabidiol concentration of about 18% to about 60% by weight. In one embodiment, the Cannabis cultivar produces an assayable combined cannabidiolic acid and cannabidiol concentration of about 20% to about 40% by weight. In one embodiment, the Cannabis cultivar produces an assayable combined cannabidiolic acid and cannabidiol concentration of about 20% to about 30% by weight. In one embodiment, the Cannabis cultivar produces an assayable combined cannabidiolic acid and cannabidiol concentration of about 25% to about 35% by weight. It should be understood that any subvalue or subrange from within the values described above are contemplated for use with the embodiments described herein.

In some cases, included are methods for producing a medical Cannabis composition, the method comprising obtaining a Cannabis and/or hemp plant, growing the Cannabis and/or hemp plant under plant growth conditions to produce plant tissue from the Cannabis and/or hemp plant, and preparing a medical Cannabis composition from the plant tissue or a portion thereof. In one aspect, described herein is a Cannabis plant that can be a Cannabis cultivar that produces substantially high levels of CBD (and/or CBDA) and substantially low levels of THC (and/or THCA) as compared to an unmodified comparable Cannabis plant and/or Cannabis cell.

### Genetic Engineering

Provided herein can be systems of genomic engineering. Systems of genomic engineering can include any one of clustered regularly interspaced short palindromic repeats (CRISPR) enzyme, transcription activator-like effector (TALE)-nuclease, transposon-based nuclease, Zinc finger nuclease, meganuclease, argonaute, or Mega-TAL.

### I. Clustered Regularly Interspaced Short Palindromic Repeats (CRISPR)

In some cases, genetic engineering can be performed using a CRISPR system or portion thereof. A CRISPR system can be a multicomponent system comprising a guide polynucleotide or a nucleic acid encoding the guide polynucleotide and a CRISPR enzyme or a nucleic acid encoding the CRISPR enzyme. A CRISPR system can also comprise any modification of the CRISPR components or any portions of any of the CRISPR components.

Methods described herein can take advantage of a CRISPR system. There are at least five types of CRISPR systems which all incorporate guide RNAs and Cas proteins and encoding polynucleic acids. The general mechanism and recent advances of CRISPR system is discussed in Cong, L. et al., “Multiplex genome engineering using CRISPR systems,” Science, 339(6121): 819-823 (2013); Fu, Y. et al., “High-frequency off-target mutagenesis induced by CRISPR-Cas nucleases in human cells,” Nature Biotechnology, 31, 822-826 (2013); Chu, V T et al. “Increasing the efficiency of homology-directed repair for CRISPR-Cas9-induced precise gene editing in mammalian cells,” Nature Biotechnology 33, 543-548 (2015); Shmakov, S. et al., “Discovery and functional characterization of diverse Class 2 CRISPR-Cas systems,” Molecular Cell, 60, 1-13 (2015); Makarova, K S et al., “An updated evolutionary classification of CRISPR-Cas systems,”, Nature Reviews Microbiology, 13, 1-15 (2015). Site-specific cleavage of a target DNA occurs at locations determined by both 1) base-pairing complementarity between the guide RNA and the target DNA (also called a protospacer) and 2) a short motif in the target DNA referred to as the protospacer adjacent motif (PAM). In an aspect, a PAM can be a canonical PAM or a non-canonical PAM. For example, an engineered cell, such as a plant cell, can be generated using a CRISPR system, e.g., a type II CRISPR system. In other aspects, a CRISPR system may be used to modify a agrobacterium cell, a E. coli cell, or a yeast cell. A Cas enzyme used in the methods disclosed herein can be Cas9, which catalyzes DNA cleavage. In an aspect, a Cas provided herein can be codon optimized for use in a plant, for example Cannabis and/or hemp. In another aspect, a plant codon optimized Cas can be used in a hemp or Cannabis plant provided herein. A plant codon optimized sequence can be from a closely related species, such as flax. Enzymatic action by Cas9 derived from Streptococcus pyogenes or any closely related Cas9 can generate double stranded breaks at target site sequences which hybridize to about 20 nucleotides of a guide sequence and that have a protospacer-adjacent motif (PAM) following the about 20 nucleotides of the target sequence. In some aspects, less than 20 nucleotides can be hybridized. In some aspects, more than 20 nucleotides can be hybridized. Provided herein can be genomically disrupting activity of a THCA synthase comprising introducing into a Cannabis and/or hemp plant or a cell thereof at least one RNA-guided endonuclease comprising at least one nuclear localization signal or nucleic acid encoding at least one RNA-guided endonuclease comprising at least one nuclear localization signal, at least one guiding nucleic acid encoding at least one guide RNA. In some aspects, a modified plant or portion thereof can be cultured.

### Clustered Regularly Interspaced Short Palindromic Repeats (CRISPR) Enzyme

A CRISPR enzyme can comprise or can be a Cas enzyme. In some aspects, a nucleic acid that encodes a Cas protein or portion thereof can be utilized in embodiments provided herein. Non-limiting examples of Cas enzymes can include Cas1, Cas1B, Cas2, Cas3, Cas4, Cas5, Cas6, Cas7, Cas8, Cas9 (also known as Csn1 or Csx12), Cas10, Csy1 , Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof, or modified versions thereof. In some cases, a catalytically dead Cas protein can be used, for example a dCas9. An unmodified CRISPR enzyme can have DNA cleavage activity, such as Cas9. A CRISPR enzyme can direct cleavage of one or both strands at a target sequence, such as within a target sequence and/or within a complement of a target sequence. In some aspects, a target sequence can be found within an intron or exon of a gene. In some cases, a CRISPR system can target an exon of a THCAS gene. For example, a CRISPR enzyme can direct cleavage of one or both strands within or within about 1, 2, 3, 4, 5, 6, 7, 8, 9, 10, 15, 20, 25, 50, 100, 200, 500, or more base pairs from the first or last nucleotide of a target sequence. For example, a CRISPR enzyme can direct cleavage of one or both strands within or within about 1, 2, 3, 4, 5, 6, 7, 8, 9, 10, 15, 20, 25, 50, 100, 200, 500, or more base pairs from a PAM sequence. A vector that encodes a CRISPR enzyme that is mutated with respect to a corresponding wild-type enzyme such that the mutated CRISPR enzyme lacks the ability to cleave one or both strands of a target polynucleotide containing a target sequence can be used. A Cas protein can be a high-fidelity Cas protein such as Cas9HiFi. In some cases, a Cas protein can be modified. For example, a Cas protein modification can comprise N7-Methyl-Gppp (2′-O-Methyl-A).

Cas9 can refer to a polypeptide with at least or at least about 50%, 60%, 70%, 80%, 90%, 100% sequence identity and/or sequence similarity to a wild type exemplary Cas9 polypeptide (e.g., Cas9 from S. pyogenes). Cas9 can refer to a polypeptide with at most or at most about 50%, 60%, 70%, 80%, 90%, 100% sequence identity and/or sequence similarity to a wild type exemplary Cas9 polypeptide (e.g., from S. pyogenes). Cas9 can refer to the wild type or a modified form of the Cas9 protein that can comprise an amino acid change such as a deletion, insertion, frameshift, substitution, variant, mutation, fusion, chimera, or any combination thereof.

A polynucleotide encoding an endonuclease (e.g., a Cas protein such as Cas9) can be codon optimized for expression in particular cells, such as a plant cell, agrobacterium cell, a E. coli cell, or a yeast cell. This type of optimization can entail the mutation of foreign-derived (e.g., recombinant) DNA to mimic the codon preferences of the intended host organism or cell while encoding the same protein.

In some cases, synthetic SpCas9-derived variants with non-NGG PAM sequences may be used. Additionally, other Cas9 orthologues from various species have been identified and these “non-SpCas9s” bind a variety of PAM sequences that could also be useful for the present disclosure. For example, the relatively large size of SpCas9 (approximately 4 kb coding sequence) means that plasmids carrying the SpCas9 cDNA may not be efficiently expressed in a cell. Conversely, the coding sequence for Staphylococcus aureus Cas9 (SaCas9) is approximately 1 kilobase shorter than SpCas9, possibly allowing it to be efficiently expressed in a cell.

Alternatives to S. pyogenes Cas9 may include RNA-guided endonucleases from the Cpf1 family. Unlike Cas9 nucleases, the result of Cpf1-mediated DNA cleavage is a double-strand break with a short 3′ overhang. Cpf1's staggered cleavage pattern may open up the possibility of directional gene transfer, analogous to traditional restriction enzyme cloning, which may increase the efficiency of gene editing. Like the Cas9 variants and orthologues described above, Cpf1 may also expand the number of sites that can be targeted by CRISPR to AT-rich regions or AT-rich genomes that lack the NGG PAM sites favored by SpCas9.

In some aspects Cas sequence can contain a nuclear localization sequence (NLS). A nuclear localization sequence can be from SV40. An NLS can be from at least one of: SV40, nucleoplasmin, importin alpha, C-myc, EGL-13, TUS, hnRNPA1, Mata2, or PY-NLS. An NLS can be on a C-terminus or an N-terminus of a Cas protein. In some cases, a Cas protein may contain from 1 to 5 NLS sequences. A Cas protein can contain 1, 2, 3, 4, 5, 6, 7, 8, 9, or up to 10 NLS sequences. A Cas protein, such as Cas9, may contain two NLS sequences. A Cas protein may contain a SV40 and nuceloplasmin NLS sequence. A Cas protein may also contain at least one untranslated region.

In some aspects, a vector that encodes a CRISPR enzyme can contain a nuclear localization sequences (NLS) sequence. In some cases, a vector can comprise one or more NLSs. In some cases, a vector can contain about 1, 2, 3, 4, 5, 6, 7, 8, 9, or 10 NLSs. For example, a CRISPR enzyme can comprise more than or more than about 1, 2, 3, 4, 5, 6, 7, 8, 9, 10 NLSs at or near the ammo-terminus, more than or more than about 1, 2, 3, 4, 5, 6, 7, 8, 9, 10, NLSs at or near the carboxyl-terminus, or any combination of these (e.g., one or more NLS at the ammo-terminus and one or more NLS at the carboxyl terminus). When more than one NLS is present, each can be selected independently of others, such that a single NLS can be present in more than one copy and/or in combination with one or more other NLSs present in one or more copies.

An NLS can be monopartite or bipartite. In some cases, a bipartite NLS can have a spacer sequence as opposed to a monopartite NLS. An NLS can be from at least one of: SV40, nucleoplasmin, importin alpha, C-myc, EGL-13, TUS, hnRNPA1, Mata2, or PY-NLS. An NLS can be located anywhere within the polypeptide chain, e.g., near the N- or C-terminus. For example, the NLS can be within or within about 1, 2, 3, 4, 5, 10, 15, 20, 25, 30, 40, 50 amino acids along a polypeptide chain from the N- or C-terminus. Sometimes the NLS can be within or within about 50 amino acids or more, e.g., 100, 200, 300, 400, 500, 600, 700, 800, 900, or 1000 amino acids from the N- or C-terminus.

Any functional concentration of Cas protein can be introduced to a cell. For example, 15 micrograms of Cas mRNA can be introduced to a cell. In other cases, a Cas mRNA can be introduced from 0.5 micrograms to 100 micrograms. A Cas mRNA can be introduced from 0.5, 5, 10, 15, 20, 25, 30, 35, 40, 45, 50, 55, 60, 65, 70, 75, 80, 85, 90, 95, or 100 micrograms.

In some cases, a dual nickase approach may be used to introduce a double stranded break or a genomic break. Cas proteins can be mutated at known amino acids within either nuclease domains, thereby deleting activity of one nuclease domain and generating a nickase Cas protein capable of generating a single strand break. A nickase along with two distinct guide RNAs targeting opposite strands may be utilized to generate a double stranded break (DSB) within a target site (often referred to as a “double nick” or “dual nickase” CRISPR system). This approach may dramatically increase target specificity, since it is unlikely that two off-target nicks will be generated within close enough proximity to cause a DSB.

A nuclease, such as Cas9, can be tested for identity and potency prior to use. For example, identity and potency can be determined using at least one of spectrophotometric analysis, RNA agarose gel analysis, LC-MS, endotoxin analysis, and sterility testing. In some cases, a nuclease sequence, such as a Cas9 sequence can be sequenced to confirm its identity. In some cases, a Cas protein, such as a Cas9 protein, can be sequenced prior to clinical or therapeutic use. For example, a purified in vitro transcription product can be assessed by polyacrylamide gel electrophoresis to verify no other mRNA species exist or substantially no other mRNA species exist within a clinical product other than Cas9. Additionally, purified mRNA encoding a Cas protein, such as Cas9, can undergo validation by reverse-transcription followed by a sequencing step to verify identity at a nucleotide level. A purified in vitro transcription product can be assessed by polyacrylamide gel electrophoresis (PAGE) to verify that an mRNA is the size expected for Cas9 and substantially no other mRNA species exist within a clinical or therapeutic product.

In some cases, an endotoxin level of a nuclease, such as Cas9, can be determined. A clinically/therapeutically acceptable level of an endotoxin can be less than 3 EU/mL. A clinically/therapeutically acceptable level of an endotoxin can be less than 2 EU/mL. A clinically/therapeutically acceptable level of an endotoxin can be less than 1 EU/mL. A clinically/therapeutically acceptable level of an endotoxin can be less than 0.5 EU/mL.

In some cases a nuclease, such as Cas9, can undergo sterility testing. A clinically/therapeutically acceptable level of a sterility testing can be 0 or denoted by no growth on a culture. A clinically/therapeutically acceptable level of a sterility testing can be less than 0.5%, 0.3%, 0.1%, or 0.05% growth.

### Guiding Polynucleic Acid

A guiding polynucleic acid can be DNA or RNA. A guiding polynucleic acid can be single stranded or double stranded. In some cases, a guiding polynucleic acid can contains regions of single stranded areas and double stranded areas. A guiding polynucleic acid can also form secondary structures. As used herein, the term “guide RNA (gRNA),” and its grammatical equivalents can refer to an RNA which can be specific for a target DNA and can form a complex with a Cas protein. A guide RNA can comprise a guide sequence, or spacer sequence, that specifies a target site and guides an RNA/Cas complex to a specified target DNA for cleavage. For example, a guide RNA can target a CRISPR complex to a target gene or portion thereof and perform a targeted double strand break. Site-specific cleavage of a target DNA occurs at locations determined by both 1) base-pairing complementarity between a guide RNA and a target DNA (also called a protospacer) and 2) a PAM. In an aspect, a PAM can be a canonical PAM or a non-canonical PAM. In some cases, gRNAs can be designed using an algorithm which can identify gRNAs located in early exons within commonly expressed transcripts.

Functional gene copies, gene variants and pseudogenes are mapped and aligned to produce a sequence template for CRISPR design. In some instances, a non-functional copy of a gene may be targeted. Non-functional copies of genes can be referred to a pseudogenes. Pseudogenes may arise due to gene duplication during evolution and may show the characteristics of sharing a significant degree of identity with a functional copy, for example CBDAS.

In some aspects, a gRNA can be designed to bind a target sequence in a coding region or in a non-coding region. In some cases, a gRNA can be designed to bind a target sequence in a regulatory region. In some cases, a gRNA can be designed to target at exon of a THCAS gene or portion thereof. In some cases, gRNAs can be designed to disrupt an early coding sequence. In some cases, a gRNA can be selected based on the pattern of indels it inserts into a target gene. Any number of indels may be observed at a modified site, for example from about 20%, 30%, 40%, 50%, 60%, 70%, 80%, 90%, or 100% indels may be observed. In an aspect, a modification results in less than or up to about: 50%, 40%, 30%, 25%, 15%, 10%, 1% of indel formation. Candidate gRNAs can be ranked by off-target potential using a scoring system that can take into account: (a) the total number of mismatches between the gRNA sequence and any closely matching genomic sequences; (b) the mismatch position(s) relative to the PAM site which correlate with a negative effect on activity for mismatches falling close to the PAM site; (c) the distance between mismatches to account for the cumulative effect of neighboring mismatches in disrupting guide-DNA interactions; and any combination thereof. In some cases, a greater number of mismatches between a gRNA and a genomic target site can yield a lower potential for CRISPR-mediated cleavage of that site. In some cases, a mismatch position is directly adjacent to a PAM site. In other cases, a mismatch position can be from 1 nucleotide up to 100 kilobases away from a PAM site. Candidate gRNAs comprising mismatches may not be adjacent to a PAM in some cases. In other cases, at least two candidate gRNAs comprising mismatches may bind a genome from 1 nucleotide up to 100 kilobases away from each other. A mismatch can be a substitution of a nucleotide. For example, in some cases a G will be substituted for a T. Mismatches between a gRNA and a genome may allow for reduced fidelity of CRISPR gene editing. In some cases, a positive scoring gRNA can be about 110 nucleotides in length and may contain no mismatches to a complementary genome sequence. In other cases, a positive scoring gRNA can be about 110 nucleotides in length and may contain up to 3 mismatches to a complementary genome sequence. In other cases, a positive scoring gRNA can be about 110 nucleotides in length and may contain up to 20 mismatches to a complementary genome sequence. In some cases, a guiding polynucleic acid can contain internucleotide linkages that can be phosphorothioates. Any number of phosphorothioates can exist. For example, from 1 to about 100 phosphorothioates can exist in a guiding polynucleic acid sequence. In some cases, from 1 to 10 phosphorothioates are present. In some cases, 8 phosphorothioates exist in a guiding polynucleic acid sequence.

In some cases, top scoring gRNAs can be designed and selected and an on-target editing efficiency of each can be assessed experimentally in plant cells, bacterial cells, yeast cells, agrobacterium cells. In some cases, an editing efficiency as determined by TiDE analysis can exceed at least about 20%. In other cases, editing efficiency can be from about 20% to from about 50%, from about 50% to from about 80%, from about 80% to from about 100%. In some cases, a percent indel can be determined in a trial GMP run. For example, a final cellular product can be analyzed for on-target indel formation by Sanger sequencing and TIDE analysis. Genomic DNA can be extracted from about 1×10⁶ cells from both a control and experimental sample and subjected to PCR using primers flanking a gene that has been disrupted, such as THCAS. Sanger sequencing chromatograms can be analyzed using a TIDE software program that can quantify indel frequency and size distribution of indels by comparison of control and knockout samples.

A method disclosed herein also can comprise introducing into a cell or plant embryo at least one guide RNA or nucleic acid, e.g., DNA encoding at least one guide RNA. A guide RNA can interact with a RNA-guided endonuclease to direct the endonuclease to a specific target site, at which site the 5′ end of the guide RNA base pairs with a specific protospacer sequence in a chromosomal sequence.

A guide RNA can comprise two RNAs, e.g., CRISPR RNA (crRNA) and transactivating crRNA (tracrRNA). A guide RNA can sometimes comprise a single-guide RNA (sgRNA) formed by fusion of a portion (e.g., a functional portion) of crRNA and tracrRNA. A guide RNA can also be a dual RNA comprising a crRNA and a tracrRNA. A guide RNA can comprise a crRNA and lack a tracrRNA. Furthermore, a crRNA can hybridize with a target DNA or protospacer sequence.

As discussed above, a guide RNA can be an expression product. For example, a DNA that encodes a guide RNA can be a vector comprising a sequence coding for the guide RNA. A guide RNA can be transferred into a cell or organism by transfecting the cell or plant embryo with an isolated guide RNA or plasmid DNA comprising a sequence coding for the guide RNA and a promoter. In some aspects, a promoter can be selected from the group consisting of a leaf-specific promoter, a flower-specific promoter, a THCA synthase promoter, a CaMV35S promoter, a FMV35S promoter, and a tCUP promoter. A guide RNA can also be transferred into a cell or plant embryo in other way, such as using particle bombardment.

A guide RNA can be isolated. For example, a guide RNA can be transfected in the form of an isolated RNA into a cell or plant embryo. A guide RNA can be prepared by in vitro transcription using any in vitro transcription system. A guide RNA can be transferred to a cell in the form of isolated RNA rather than in the form of plasmid comprising encoding sequence for a guide RNA.

A guide RNA can comprise a DNA-targeting segment and a protein binding segment. A DNA-targeting segment (or DNA-targeting sequence, or spacer sequence) comprises a nucleotide sequence that can be complementary to a specific sequence within a target DNA (e.g., a protospacer). A protein-binding segment (or protein-binding sequence) can interact with a site-directed modifying polypeptide, e.g. an RNA-guided endonuclease such as a Cas protein. By “segment” it is meant a segment/section/region of a molecule, e.g., a contiguous stretch of nucleotides in an RNA. A segment can also mean a region/section of a complex such that a segment may comprise regions of more than one molecule. For example, in some cases a protein-binding segment of a DNA-targeting RNA is one RNA molecule and the protein-binding segment therefore comprises a region of that RNA molecule. In other cases, the protein-binding segment of a DNA-targeting RNA comprises two separate molecules that are hybridized along a region of complementarity.

A guide RNA can comprise two separate RNA molecules or a single RNA molecule. An exemplary single molecule guide RNA comprises both a DNA-targeting segment and a protein-binding segment.

An exemplary two-molecule DNA-targeting RNA can comprise a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA”) molecule. A first RNA molecule can be a crRNA-like molecule (targeter-RNA), that can comprise a DNA-targeting segment (e.g., spacer) and a stretch of nucleotides that can form one half of a double-stranded RNA (dsRNA) duplex comprising the protein-binding segment of a guide RNA. A second RNA molecule can be a corresponding tracrRNA-like molecule (activator-RNA) that can comprise a stretch of nucleotides that can form the other half of a dsRNA duplex of a protein-binding segment of a guide RNA. In other words, a stretch of nucleotides of a crRNA-like molecule can be complementary to and can hybridize with a stretch of nucleotides of a tracrRNA-like molecule to form a dsRNA duplex of a protein-binding domain of a guide RNA. As such, each crRNA-like molecule can be said to have a corresponding tracrRNA-like molecule. A crRNA-like molecule additionally can provide a single stranded DNA-targeting segment, or spacer sequence. Thus, a crRNA-like and a tracrRNA-like molecule (as a corresponding pair) can hybridize to form a guide RNA. A subject two-molecule guide RNA can comprise any corresponding crRNA and tracrRNA pair.

A DNA-targeting segment or spacer sequence of a guide RNA can be complementary to sequence at a target site in a chromosomal sequence, e.g., protospacer sequence such that the DNA-targeting segment of the guide RNA can base pair with the target site or protospacer. In some cases, a DNA-targeting segment of a guide RNA can comprise from or from about 10 nucleotides to from or from about 25 nucleotides or more. For example, a region of base pairing between a first region of a guide RNA and a target site in a chromosomal sequence can be or can be about 10, 11, 12, 13, 14, 15, 16, 17, 18, 19, 20, 22, 23, 24, 25, or more than 25 nucleotides in length. Sometimes, a first region of a guide RNA can be or can be about 19, 20, or 21 nucleotides in length.

A guide RNA can target a nucleic acid sequence of or of about 20 nucleotides. A target nucleic acid can be less than or less than about 20 nucleotides. A target nucleic acid can be at least or at least about 5, 10, 15, 16, 17, 18, 19, 20, 21, 22, 23, 24, 25, 30 or more nucleotides. A target nucleic acid can be at most or at most about 5, 10, 15, 16, 17, 18, 19, 20, 21, 22, 23, 24, 25, 30 or more nucleotides in length. A target nucleic acid sequence can be or can be about 20 bases immediately 5′ of the first nucleotide of the PAM. A guide RNA can target the nucleic acid sequence. A guiding polynucleic acid, such as a guide RNA, can bind to a genomic sequence with at least or at least about 50%, 60%, 65%, 70%, 75%, 80%, 85%, 90%, 95%, 98%, or up to about 100% sequence identity and/or sequence similarity to any of the sequences of Table 6. In some cases, a guiding polynucleic acid, such as a guide RNA, can bind a genomic region from about 1 base pair to about 20 base pairs away from a PAM. A guide can bind a genomic region from about 1, 2, 3, 4,5 6, 7, 8, 9, 10, 11, 12, 13, 14, 15, 16, 17, 18, 19, or up to about 20 base pairs away from a PAM. A guide polynucleotide can comprise less than about 70%, 60%, 50%, 40%, 30%, 20%, 10%, 5%, 2.5%, or 1% identity to an endogenous CBDAS gene or portion thereof. In some cases, a gRNA or gDNA can target a gene that is not CBDAS to generate a transgenic plant that exhibits increased CBDAS production.

A guide nucleic acid, for example, a guide RNA, can refer to a nucleic acid that can hybridize to another nucleic acid, for example, the target nucleic acid or protospacer in a genome of a cell. A guide nucleic acid can be RNA. A guide nucleic acid can be DNA. The guide nucleic acid can be programmed or designed to bind to a sequence of nucleic acid site-specifically. A guide nucleic acid can comprise a polynucleotide chain and can be called a single guide nucleic acid. A guide nucleic acid can comprise two polynucleotide chains and can be called a double guide nucleic acid.

A guide nucleic acid can comprise one or more modifications to provide a nucleic acid with a new or enhanced feature. A guide nucleic acid can comprise a nucleic acid affinity tag. A guide nucleic acid can comprise synthetic nucleotide, synthetic nucleotide analog, nucleotide derivatives, and/or modified nucleotides.

A guide nucleic acid can comprise a nucleotide sequence (e.g., a spacer), for example, at or near the 5′ end or 3′ end, that can hybridize to a sequence in a target nucleic acid (e.g., a protospacer). A spacer of a guide nucleic acid can interact with a target nucleic acid in a sequence-specific manner via hybridization (i.e., base pairing). A spacer sequence can hybridize to a target nucleic acid that is located 5′ or 3′ of a protospacer adjacent motif (PAM). The length of a spacer sequence can be at least or at least about 5, 10, 15, 16, 17, 18, 19, 20, 21, 22, 23, 24, 25, 30 or more nucleotides. The length of a spacer sequence can be at most or at most about 5, 10, 15, 16, 17, 18, 19, 20, 21, 22, 23, 24, 25, 30 or more nucleotides.

A guide RNA can also comprise a dsRNA duplex region that forms a secondary structure. For example, a secondary structure formed by a guide RNA can comprise a stem (or hairpin) and a loop. A length of a loop and a stem can vary. For example, a loop can range from about 3 to about 10 nucleotides in length, and a stem can range from about 6 to about 20 base pairs in length. A stem can comprise one or more bulges of 1 to about 10 nucleotides. The overall length of a second region can range from about 16 to about 60 nucleotides in length. For example, a loop can be or can be about 4 nucleotides in length and a stem can be or can be about 12 base pairs. A dsRNA duplex region can comprise a protein-binding segment that can form a complex with an RNA-binding protein, such as an RNA-guided endonuclease, e.g. Cas protein.

A guide RNA can also comprise a tail region at the 5′ or 3′ end that can be essentially single-stranded. For example, a tail region is sometimes not complementarity to any chromosomal sequence in a cell of interest and is sometimes not complementarity to the rest of a guide RNA. Further, the length of a tail region can vary. A tail region can be more than or more than about 4 nucleotides in length. For example, the length of a tail region can range from or from about 5 to from or from about 60 nucleotides in length.

A guide RNA can be introduced into a cell or embryo as an RNA molecule. For example, a RNA molecule can be transcribed in vitro and/or can be chemically synthesized. A guide RNA can then be introduced into a cell or embryo as an RNA molecule. A guide RNA can also be introduced into a cell or embryo in the form of a non-RNA nucleic acid molecule, e.g., DNA molecule. For example, a DNA encoding a guide RNA can be operably linked to promoter control sequence for expression of the guide RNA in a cell or embryo of interest. A RNA coding sequence can be operably linked to a promoter sequence that is recognized by RNA polymerase III (Pol III).

A DNA molecule encoding a guide RNA can also be linear. A DNA molecule encoding a guide RNA can also be circular. A DNA sequence encoding a guide RNA can also be part of a vector. Some examples of vectors can include plasmid vectors, phagemids, cosmids, artificial/mini-chromosomes, transposons, and viral vectors. For example, a DNA encoding a RNA-guided endonuclease is present in a plasmid vector. Other non-limiting examples of suitable plasmid vectors include pUC, pBR322, pET, pBluescript, and variants thereof. Further, a vector can comprise additional expression control sequences (e g., enhancer sequences, Kozak sequences, polyadenylation sequences, transcriptional termination sequences, etc.), selectable marker sequences (e.g., antibiotic resistance genes), origins of replication, and the like.

When both a RNA-guided endonuclease and a guide RNA are introduced into a cell as DNA molecules, each can be part of a separate molecule (e.g., one vector containing fusion protein coding sequence and a second vector containing guide RNA coding sequence) or both can be part of a same molecule (e.g., one vector containing coding (and regulatory) sequence for both a fusion protein and a guide RNA). For example, in some cases, a CRISPR enzyme complexed with a guide polynucleotide can be introduced into a plant by a vector comprising a nucleic acid encoding a CRISPR enzyme and a guide polynucleotide. In some cases, a vector is a binary vector or a Ti plasmid. In some aspects, a vector can further comprise a selection marker or a reporter, or portion thereof.

A Cas protein, such as a Cas9 protein or any derivative thereof, can be pre-complexed with a guide RNA to form a ribonucleoprotein (RNP) complex. The RNP complex can be introduced into plant cells. Introduction of the RNP complex can be timed. The cell can be synchronized with other cells at G1, S, and/or M phases of the cell cycle. The RNP complex can be delivered at a cell phase such that HDR is enhanced. The RNP complex can facilitate homology directed repair. In some cases, a CRISPR enzyme can be complexed with a guide polynucleotide and introduced into a plant via RNP to generate a transgenic plant.

A guide RNA can also be modified. The modifications can comprise chemical alterations, synthetic modifications, nucleotide additions, and/or nucleotide subtractions. The modifications can also enhance CRISPR genome engineering. A modification can alter chirality of a gRNA. In some cases, chirality may be uniform or stereopure after a modification. A guide RNA can be synthesized. The synthesized guide RNA can enhance CRISPR genome engineering. A guide RNA can also be truncated. Truncation can be used to reduce undesired off-target mutagenesis. The truncation can comprise any number of nucleotide deletions. For example, the truncation can comprise 1, 2, 3, 4, 5, 10, 15, 20, 25, 30, 40, 50 or more nucleotides. A guide RNA can comprise a region of target complementarity of any length. For example, a region of target complementarity can be less than 20 nucleotides in length. A region of target complementarity can be more than 20 nucleotides in length. A region of target complementarity can target from about 5 bp to about 20 bp directly adjacent to a PAM sequence. A region of target complementarity can target about 13 bp directly adjacent to a PAM sequence. The polynucleic acids as described herein can be modified. A modification can be made at any location of a polynucleic acid. More than one modification can be made to a single polynucleic acid. A polynucleic acid can undergo quality control after a modification. In some cases, quality control may include PAGE, HPLC, MS, or any combination thereof. A modification can be a substitution, insertion, frameshift, deletion, chemical modification, physical modification, stabilization, purification, or any combination thereof. A polynucleic acid can also be modified by 5′adenylate, 5′ guanosine-triphosphate cap, 5′N⁷-Methylguanosine-triphosphate cap, 5′triphosphate cap, 3′phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′ deoxyribonucleoside analog purine, 2′ deoxyribonucleoside analog pyrimidine, ribonucleoside analog, 2′-O-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, or any combination thereof. In some cases, a modification can be permanent. In other cases, a modification can be transient. In some cases, multiple modifications are made to a polynucleic acid. A polynucleic acid modification may alter physio-chemical properties of a nucleotide, such as their conformation, polarity, hydrophobicity, chemical reactivity, base-pairing interactions, or any combination thereof. In some aspects a gRNA can be modified. In some cases, a modification is on a 5′ end, a 3′ end, from a 5′ end to a 3′ end, a single base modification, a 2′-ribose modification, or any combination thereof. A modification can be selected from a group consisting of base substitutions, insertions, deletions, chemical modifications, physical modifications, stabilization, purification, and any combination thereof. In some cases, a modification is a chemical modification.

In some cases, a modification is a 2-O-methyl 3 phosphorothioate addition denoted as “m”. A phosphothioate backbone can be denoted as “(ps).” A 2-0-methyl 3 phosphorothioate addition can be performed from 1 base to 150 bases. A 2-O-methyl 3 phosphorothioate addition can be performed from 1 base to 4 bases. A 2-O-methyl 3 phosphorothioate addition can be performed on 2 bases. A 2-O-methyl 3 phosphorothioate addition can be performed on 4 bases. A modification can also be a truncation. A truncation can be a 5-base truncation. In some cases, a modification may be at C terminus and N terminus nucleotides.

A modification can also be a phosphorothioate substitute. In some cases, a natural phosphodiester bond may be susceptible to rapid degradation by cellular nucleases and; a modification of internucleotide linkage using phosphorothioate (PS) bond substitutes can be more stable towards hydrolysis by cellular degradation. A modification can increase stability in a polynucleic acid. A modification can also enhance biological activity. In some cases, a phosphorothioate enhanced RNA polynucleic acid can inhibit RNase A, RNase T1, calf serum nucleases, or any combinations thereof. These properties can allow the use of PS-RNA polynucleic acids to be used in applications where exposure to nucleases is of high probability in vivo or in vitro. For example, phosphorothioate (PS) bonds can be introduced between the last 3-5 nucleotides at the 5′- or 3′-end of a polynucleic acid which can inhibit exonuclease degradation. In some cases, phosphorothioate bonds can be added throughout an entire polynucleic acid to reduce attack by endonucleases.

In another embodiment, down-regulating the activity of a THCA synthase or portion thereof comprises introducing into a transgenic plant such as a Cannabis and/or hemp plant or a cell thereof (i) at least one RNA-guided endonuclease comprising at least one nuclear localization signal or nucleic acid encoding at least one RNA-guided endonuclease comprising at least one nuclear localization signal, (ii) at least one guide RNA or DNA encoding at least one guide RNA, and, optionally, (iii) at least one donor polynucleotide such as a barcode; and culturing the Cannabis and/or hemp plant or cell thereof such that each guide RNA directs an RNA-guided endonuclease to a targeted site in the chromosomal sequence where the RNA-guided endonuclease introduces a double-stranded break in the targeted site, and the double-stranded break is repaired by a DNA repair process such that the chromosomal sequence is modified, wherein the targeted site is located in the THCA synthase gene and the chromosomal modification interrupts or interferes with transcription and/or translation of the THCA synthase gene. In an aspect, a donor polynucleotide comprises homology to sequences flanking a target sequence, for example a THCAS gene or portion thereof.

In some cases, a GUIDE-Seq analysis can be performed to determine the specificity of engineered guide RNAs. The general mechanism and protocol of GUIDE-Seq profiling of off-target cleavage by CRISPR system nucleases is discussed in Tsai, S. et al., “GUIDE-Seq enables genome-wide profiling of off-target cleavage by CRISPR system nucleases,” Nature, 33: 187-197 (2015). To assess off-target frequencies by next generation sequencing cells can be transfected with Cas9 mRNA and a guiding RNA, such as anti-THCAS gRNA. Genomic DNA can be isolated from transfected cells from about 72 hours post transfection and PCR amplified at potential off-target sites. A potential off-target site can be predicted using the Wellcome Trust Sanger Institute Genome Editing database (WGE) algorithm. Candidate off-target sites can be chosen based on sequence homology to an on-target site. In some cases, sites with about 4 or less mismatches between a gRNA and a genomic target site can be utilized. For each candidate off-target site, two primer pairs can be designed. PCR amplicons can be obtained from both untreated (control) and Cas9/gRNA-treated cells. PCR amplicons can be pooled. NGS libraries can be prepared using TruSeq Nano DNA library preparation kit (Illumina). Samples can be analyzed on an Illumina HiSeq machine using a 250 bp paired-end workflow. In some cases, from about 40 million mappable NGS reads per gRNA library can be acquired. This can equate to an average number of about 450,000 reads for each candidate off-target site of a gRNA. In some cases, detection of CRISPR-mediated disruption can be at a frequency as low as 0.1% at any genomic locus.

Computational predictions can be used to select candidate gRNAs likely to be the safest choice for a targeted gene, such as THCAS functional disruption. Candidate gRNAs can then tested empirically using a focused approach steered by computational predictions of potential off-target sites. In some cases, an assessment of gRNA off-target safety can employ a next-generation deep sequencing approach to analyze the potential off-target sites predicted by the CRISPR design tool for each gRNA. In some cases, gRNAs can be selected with fewer than 3 mismatches to any sequence in the genome (other than the perfect matching intended target). In some cases, a gRNA can be selected with fewer than 50, 40, 30, 20, 10, 5, 4, 3, 2, or 1 mismatch(es) to any sequence in a genome. In some cases, a computer system or software can be utilized to provide recommendations of candidate gRNAs with predictions of low off-target potential.

In some cases, potential off-target sites can be identified with at least one of: GUIDE-Seq and targeted PCR amplification, and next generation sequencing. In addition, modified cells, such as Cas9/gRNA-treated cells can be subjected to karyotyping to identify any chromosomal re-arrangements or translocations.

A gRNA can be introduced at any functional concentration. For example, a gRNA can be introduced to a cell at 10 micrograms. In other cases, a gRNA can be introduced from 0.5 micrograms to 100 micrograms. A gRNA can be introduced from 0.5, 5, 10, 15, 20, 25, 30, 35, 40, 45, 50, 55, 60, 65, 70, 75, 80, 85, 90, 95, or 100 micrograms.

A guiding polynucleic acid can have any frequency of bases. For example, a guiding polynucleic acid can have 29 As, 17 Cs, 23 Gs, 23 Us, 3 mGs, 1 mCs, and 4 mUs. A guiding polynucleic acid can have from about 1 to about 100 nucleotides. A guiding polynucleic acid can have from about 1 to 30 of a single polynucleotide. A guiding polynucleic acid can have from about 1 to 10, 10 to 20, or from 20 to 30 of a single nucleotide.

A guiding polynucleic acid can be tested for identity and potency prior to use. For example, identity and potency can be determined using at least one of spectrophotometric analysis, RNA agarose gel analysis, LC-MS, endotoxin analysis, and sterility testing. In some cases, identity testing can determine an acceptable level for clinical/therapeutic use. For example, an acceptable spectrophotometric analysis result can be 14±2μL/vial at 5.0±0.5 mg/mL. an acceptable spectrophotometric analysis result can also be from about 10-20±2μL/vial at 5.0±0.5 mg/mL or from about 10-20±2μL/vial at about 3.0 to 7.0±0.5 mg/mL. An acceptable clinical/therapeutic size of a guiding polynucleic acid can be about 100 bases. A clinical/therapeutic size of a guiding polynucleic acid can be from about 5 bases to about 150 bases. A clinical/therapeutic size of a guiding polynucleic acid can be from about 20 bases to about 150 bases. A clinical/therapeutic size of a guiding polynucleic acid can be from about 40 bases to about 150 bases. A clinical/therapeutic size of a guiding polynucleic acid can be from about 60 bases to about 150 bases. A clinical/therapeutic size of a guiding polynucleic acid can be from about 80 bases to about 150 bases. A clinical/therapeutic size of a guiding polynucleic acid can be from about 100 bases to about 150 bases. A clinical/therapeutic size of a guiding polynucleic acid can be from about 110 bases to about 150 bases. A clinical/therapeutic size of a guiding polynucleic acid can be from about 120 bases to about 150 bases.

In some cases, a mass of a guiding polynucleic acid can be determined. A mass can be determined by LC-MS assay. A mass can be about 32,461.0 amu. A guiding polynucleic acid can have a mass from about 30,000 amu to about 50,000 amu. A guiding polynucleic acid can have a mass from about 30,000 amu to 40,000 amu, from about 40,000 amu to about 50,000 amu. A mass can be of a sodium salt of a guiding polynucleic acid.

In some cases, a guiding polynucleic acid can go sterility testing. A clinically/therapeutically acceptable level of a sterility testing can be 0 or denoted by no growth on a culture. A clinically/therapeutically acceptable level of a sterility testing can be less than 0.5% growth.

Guiding polynucleic acids can be assembled by a variety of methods, e.g., by automated solid-phase synthesis. A polynucleic acid can be constructed using standard solid-phase DNA/RNA synthesis. A polynucleic acid can also be constructed using a synthetic procedure. A polynucleic acid can also be synthesized either manually or in a fully automated fashion. In some cases, a synthetic procedure may comprise 5′-hydroxyl oligonucleotides can be initially transformed into corresponding 5′-H-phosphonate mono esters, subsequently oxidized in the presence of imidazole to activated 5′-phosphorimidazolidates, and finally reacted with pyrophosphate on a solid support. This procedure may include a purification step after the synthesis such as PAGE, HPLC, MS, or any combination thereof.

In some cases, a genomic disruption can be performed by a system selected from: CRISPR, TALEN, transposon-based nuclease, argonaute, sleeping beauty, ZEN, meganuclease, or Mega-TAL. In some cases, a genomic editing system can be complexed with a guide polynucleotide that is complementary to a target sequence in a THCAS gene or portion thereof. In some aspects, a gRNA or gDNA comprises a sequence that binds a target sequence within or adjacent to a THCAS gene. In some cases, a guide polynucleotide binds a portion of a THCAS sequence. A target sequence can contain mismatches and still allow for binding and functionality of a gene editing system. Donor sequences

In some cases, a donor polynucleotide or nucleic acid encoding a donor may be introduced to a Cannabis and/or hemp plant or portion thereof. In some cases, a donor can be a barcode. A barcode can comprise a non-natural sequence. In some aspects, a barcode contains natural sequences. In some aspects, a barcode can be utilized to allow for identification of transgenic plants via genotyping. Barcode sequences can be introduced as exogenous DNA, inserted into predetermined sites and can serve as unique identifiers whose sequence. A barcode can be useful if modified plants provided herein are distributed and need to be controlled and tracked. A barcode sequence can be any unique string of DNA which can be easily amplified and sequenced by standard methods and complex enough to not occur naturally or be easily discovered.

In another aspect, an alternative approach to a barcode which does not rely on the insertion of foreign DNA, can be to engineer an additional CRISPR-mediated indel into the genome of a plant at a precise location. A genomic region can be selected that is absent of any genes (gene desert), or a safe harbor-locus. In some cases, a gRNA or multiple gRNAs are designed to target close positions to that precise location and can be selected such that the gRNA or gRNAs introduce a known and consistent pattern of indels at that precise location (such as series of +1 insertions, or small deletions). This becomes a unique mutational fingerprint that does not occur naturally and that can identify a modified plant.

In an aspect, a donor sequence that can be introduced into a genome of a plant, for example Cannabis and/or hemp can be a promoter or portion thereof. Promoters can be full length gene promoters, portions of full-length gene promoters, cis-acting promoters, or partial sequences comprising cis-acting promoter elements. In an aspect, a promoter or portion thereof can drive enhanced gene transcription of a sequence of interest or target sequence. A sequence of interest can be a CBDAS. In some cases, donor sequences can comprise a full length CBDAS coding sequence and a strong promoter sequence, to add extra copies of the gene to enable elevated constitutive expression of the gene. Single or multiple copies can be added to tune the expression to engineer plants with varying levels of CBD. For example, from about 1 ,2, 3, 4, 5, 6, 7, 8, 9, or 10 copies of a sequence of interest, such as a gene or portion thereof, may be introduced to a plant.

In some aspects, a donor sequence can be a marker. Selectable marker genes can include, for example, photosynthesis (atpB, tscA, psaA/B, petB, petA, ycf3, rpoA, rbcL), antibiotic resistance (rrnS, rrnL, aadA, nptll, aphA-6), herbicide resistance (psbA, bar, AHAS (ALS), EPSPS, HPPD, sul) and metabolism (BADH, codA, ARG8, ASA2) genes. The sul gene from bacteria has herbicidal sulfonamide-insensitive dihydropteroate synthase activity and can be used as a selectable marker when the protein product is targeted to plant mitochondria (U.S. Pat. No. 6,121,513). In some embodiments, the sequence encoding the marker may be incorporated into the genome of the Cannabis and/or hemp. In some embodiments, the incorporated sequence encoding the marker may by subsequently removed from the transformed Cannabis and/or hemp genome. Removal of a sequence encoding a marker may be facilitated by the presence of direct repeats before and after the region encoding the marker. Removal of the sequence encoding the marker can occur via the endogenous homologous recombination system of the organelle or by use of a site-specific recombinase system such as cre-lox or FLP/FRT.

In some cases, a marker can refer to a label capable of detection, such as, for example, a radioisotope, fluorescent compound, bioluminescent compound, a chemiluminescent compound, metal chelator, or enzyme. Examples of detectable markers include, but are not limited to, the following: fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags).

Selectable or detectable markers normally comprise DNA segments that allow a cell, or a molecule marked with a “tag” inside a cell of interest, to be identified, often under specific conditions. Such markers can encode an activity, selected from, but not limited to, the production of RNA, peptides, or proteins, or the marker can provide a bonding site for RNA, peptides, proteins, inorganic and organic compounds or composites, etc. By way of example, selectable markers comprise, without being limited thereto, DNA segments that comprise restriction enzyme cleavage points, DNA segments comprising a fluorescent probe, DNA segments that encode products that provide resistance to otherwise toxic compounds, comprising antibiotics, e.g. spectinomycin, ampicillin, kanamycin, tetracycline, BASTA, neomycin-phosphotransferase II (NEO) and hygromycin-phosphotransferase (HPT), DNA segments that encode products that a plant target cell of interest would not have under natural conditions, e.g. tRNA genes, auxotrophic markers and the like, DNA segments that encode products that can be readily identified, in particular optically observable markers, e.g. phenotype markers such as -galactosidases, GUS, fluorescent proteins, e.g. green fluorescent protein (GFP) and other fluorescent proteins, e.g. blue (CFP), yellow (YFP) or red (RFP) fluorescent proteins, and surface proteins, wherein those fluorescent proteins that exhibit a high fluorescence intensity are of particular interest, because these proteins can also be identified in deeper tissue layers if, instead of a single cell, a complex plant target structure or a plant material or a plant comprising numerous types of tissues or cells can be to be analyzed, new primer sites for PCR, the recording of DNA sequences that cannot be modified in accordance with the present disclosure by restriction endonucleases or other DNA modified enzymes or effector domains, DNA sequences that are used for specific modifications, e.g. epigenetic modifications, e.g. methylations, and DNA sequences that carry a PAM motif, which can be identified by a suitable CRISPR system in accordance with the present disclosure, and also DNA sequences that do not have a PAM motif, such as can be naturally present in an endogenous plant genome sequence.

In one embodiment, a donor comprises a selectable, screenable, or scoreable marker gene or portion thereof. In some cases, a marker serves as a selection or screening device may function in a regenerable plant tissue to produce a compound that would confer upon the plant tissue resistance to an otherwise toxic compound. Genes of interest for use as a selectable, screenable, or scoreable marker would include but are not limited to gus, green fluorescent protein (gfp), luciferase (lux), genes conferring tolerance to antibiotics like kanamycin (Dekeyser et al., 1989) or spectinomycin (e.g. spectinomycin aminoglycoside adenyltransferase (aadA), genes that encode enzymes that give tolerance to herbicides like glyphosate (e.g. 5-enolpyruvylshikimate-3-phosphate synthase (EPSPS); glyphosate oxidoreductase (GOX); glyphosate decarboxylase; or glyphosate N-acetyltransferase (GAT), dalapon (e.g. dehI encoding 2,2-dichloropropionic acid dehalogenase conferring tolerance to 2,2-dichloropropionic acid, bromoxynil (haloarylnitrilase (Bxn) for conferring tolerance to bromoxynil, sulfonyl herbicides (e.g. acetohydroxyacid synthase or acetolactate synthase conferring tolerance to acetolactate synthase inhibitors such as sulfonylurea, imidazolinone, triazolopyrimidine, pyrimidyloxybenzoates and phthalide; encoding ALS, GST-II), bialaphos or phosphinothricin or derivatives (e.g. phosphinothricin acetyltransferase (bar) conferring tolerance to phosphinothricin or glufosinate, atrazine (encoding GST-III), dicamba (dicamba monooxygenase), or sethoxydim (modified acetyl-coenzyme A carboxylase for conferring tolerance to cyclohexanedione (sethoxydim) and aryloxyphenoxypropionate (haloxyfop), among others. Other selection procedures can also be implemented including positive selection mechanisms (e.g. use of the manA gene of E. coli, allowing growth in the presence of mannose), and dual selection (e.g. simultaneously using 75-100 ppm spectinomycin and 3-10 ppm glufosinate, or 75 ppm spectinomycin and 0.2-0.25 ppm dicamba). Use of spectinomycin at a concentration of about 25-1000 ppm, such as at about 150 ppm, can be also contemplated. In an embodiment, a detectable marker can be attached by spacer arms of various lengths to reduce potential steric hindrance.

In an aspect, a donor provided herein comprises homology to sequences flanking a target sequence, for example a THCAS gene or portion thereof. In an aspect, a donor polynucleotide can result in decreased or abrogated activity or expression of a THCAS gene. For example, a donor may introduce a stop codon into a THCAS gene. In another aspect, a donor can introduce an inactivating mutation within a critical and/or catalytic region of a gene to have the similar effects as inactivating the gene, either by preventing gene or protein expression and/or by rendering the expressed protein unable to produce THCA. For example, a donor may introduce a nonsense mutation, a missense mutation, a premature stop codon, a frameshift, or an aberrant splicing site.

### Transformation

Appropriate transformation techniques can include but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium tumeficiens mediated transformation. Transformation means introducing a nucleotide sequence, such as a CRISPR system, into a plant in a manner to cause stable or transient expression of the sequence.

Following transformation, plants may be selected using a dominant selectable marker incorporated into the transformation vector. In certain embodiments, such marker confers antibiotic or herbicide resistance on the transformed plants, and selection of transformants can be accomplished by exposing the plants to appropriate concentrations of the antibiotic or herbicide. After transformed plants are selected and grown to maturity, those plants showing a modified trait are identified. The modified trait can be any of those traits described above. Additionally, expression levels or activity of the polypeptide or polynucleotide of the disclosure can be determined by analyzing mRNA expression using Northern blots, RT-PCR, RNA seq or microarrays, or protein expression using immunoblots or Western blots or gel shift assays.

Suitable methods for transformation of plant or other cells for use with the current disclosure are believed to include virtually any method by which DNA can be introduced into a cell, such as by direct delivery of DNA such as by PEG-mediated transformation of protoplasts, by desiccation/inhibition-mediated DNA uptake, by electroporation, by agitation with silicon carbide fibers, by Agrobacterium-mediated transformation and by acceleration of DNA coated particles. Through the application of techniques such as these, the cells of virtually any plant species may be stably transformed, and these cells developed into transgenic plants.

### Agrobacterium Mediated Transformation

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA, for example a CRISPR system or donor, into plant cells is also provided herein.

Agrobacterium-mediated transformation can be efficient in dicotyledonous plants and can be used for the transformation of dicots, including Arabidopsis, tobacco, tomato, alfalfa and potato. Indeed, while Agrobacterium-mediated transformation has been routinely used with dicotyledonous plants for a number of years. In some cases, agrobacterium-mediated transformation can be used in monocotyledonous plants. For example, Agrobacterium-mediated transformation techniques have now been applied to rice, wheat, barley, alfalfa and maize. In some aspects, Agrobacterium-Mediated Transformation can be used to transform a Cannabis and/or hemp plant or cell thereof.

Modern Agrobacterium transformation vectors are capable of replication in E. coli as well as Agrobacterium, allowing for convenient manipulations as described. Moreover, recent technological advances in vectors for Agrobacterium-mediated gene transfer have improved the arrangement of genes and restriction sites in the vectors to facilitate the construction of vectors capable of expressing various polypeptide coding genes. In some aspects, a vector can have convenient multi-linker regions flanked by a promoter and a polyadenylation site for direct expression of inserted polypeptide coding genes and are suitable for purposes described herein. In addition, Agrobacterium containing both armed and disarmed Ti genes can be used for the transformations.

### Electroporation

In some aspects, a Cannabis and/or hemp plant or cell thereof may be modified using electroporation. To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells, such as Cannabis and/or hemp cells, by exposing them to pectin-degrading enzymes (pectolyases) or mechanically wounding in a controlled manner

Any transfection system can be utilized. In some cases, a Neon transfection system may be utilized. A Neon system can be a three-component electroporation apparatus comprising a central control module, an electroporation chamber that can be connected to a central control module by a 3-foot-long electrical cord, and a specialized pipette. In some cases, a specialized pipette can be fitted with exchangeable and/or disposable sterile tips. In some cases, an electroporation chamber can be fitted with exchangeable/disposable sterile electroporation cuvettes. In some cases, standard electroporation buffers supplied by a manufacturer of a system, such as a Neon system, can be replaced with GMP qualified solutions and buffers. In some cases, a standard electroporation buffer can be replaced with GMP grade phosphate buffered saline (PBS). A self-diagnostic system check can be performed on a control module prior to initiation of sample electroporation to ensure the Neon system is properly functioning. In some cases, a transfection can be performed in a class 1,000 biosafety cabinet within a class 10,000 clean room in a cGMP facility. In some cases, electroporation pulse voltage may be varied to optimize transfection efficiency and/or cell viability. In some cases, electroporation pulse width may be varied to optimize transfection efficiency and/or cell viability. In some cases, the number of electroporation pulses may be varied to optimize transfection efficiency and/or cell viability. In some cases, electroporation may comprise a single pulse. In some cases, electroporation may comprise more than one pulse. In some cases, electroporation may comprise 2 pulses, 3 pulses, 4 pulses, 5 pulses 6 pulses, 7 pulses, 8 pulses, 9 pulses, or 10 or more pulses.

In some aspects, protoplasts of plants may be used for electroporation transformation.

### Microprojectile Bombardment

Another method for delivering transforming DNA segments to plant cells in accordance with the disclosure is microprojectile bombardment. In this method, particles may be coated with nucleic acids and delivered into cells by a propelling force. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. In some aspects, DNA-coated particles may increase the level of DNA delivery via particle bombardment. For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a filter surface covered with monocot plant cells cultured in suspension. The screen disperses the particles so that they are not delivered to the recipient cells in large aggregates.

### Other Transformation Methods

Additional transformation methods include but are not limited to calcium phosphate precipitation, polyethylene glycol treatment, electroporation, and combinations of these treatments.

To transform plant strains that cannot be successfully regenerated from protoplasts, other ways to introduce DNA into intact cells or tissues can be utilized. For example, regeneration of plants from immature embryos or explants can be affected as described. Also, silicon carbide fiber-mediated transformation may be used with or without protoplasting. Transformation with this technique can be accomplished by agitating silicon carbide fibers together with cells in a DNA solution. DNA passively enters as the cells are punctured.

In some cases, a starting cell density for genomic editing may be varied to optimize editing efficiency and/or cell viability. In some cases, the starting cell density for genomic editing may be less than about 1×10⁵ cells. In some cases, the starting cell density for electroporation may be at least about 1×10⁵ cells, at least about 2×10⁵ cells, at least about 3×10⁵ cells, at least about 4×10⁵ cells, at least about 5×10⁵ cells, at least about 6×10⁵ cells, at least about 7×10⁵ cells, at least about 8×10⁵ cells, at least about 9×10⁵ cells, at least about 1×10⁶ cells, at least about 1.5×10⁶ cells, at least about 2×10⁶ cells, at least about 2.5×10⁶ cells, at least about 3×10⁶ cells, at least about 3.5×10⁶ cells, at least about 4×10⁶ cells, at least about 4.5×10⁶ cells, at least about 5×10⁶ cells, at least about 5.5×10⁶ cells, at least about 6×10⁶ cells, at least about 6.5×10⁶ cells, at least about 7×10⁶ cells, at least about 7.5×10⁶ cells, at least about 8×10⁶ cells, at least about 8.5×10⁶ cells, at least about 9×10⁶ cells, at least about 9.5×10⁶ cells, at least about 1×10⁷ cells, at least about 1.2×10⁷ cells, at least about 1.4×10⁷ cells, at least about 1.6×10⁷ cells, at least about 1.8×10⁷ cells, at least about 2×10⁷ cells, at least about 2.2×10⁷ cells, at least about 2.4×10⁷ cells, at least about 2.6×10⁷ cells, at least about 2.8×10⁷ cells, at least about 3×10⁷ cells, at least about 3.2×10⁷ cells, at least about 3.4×10⁷ cells, at least about 3.6×10⁷ cells, at least about 3.8×10⁷ cells, at least about 4×10⁷ cells, at least about 4.2×10⁷ cells, at least about 4.4×10⁷ cells, at least about 4.6×10⁷ cells, at least about 4.8×10⁷ cells, or at least about 5×10⁷ cells.

The efficiency of genomic disruption of plants or any part thereof, including but not limited to a cell, with any of the nucleic acid delivery platforms described herein, can result in disruption of a gene or portion thereof at about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or up to about 100% as measured by nucleic acid or protein analysis.

In an aspect, provided herein can be engineering of a plant cell with a CRISPR system followed by genotypic analysis, and quantification of cannabinoid content. In an aspect, a CRISPR system can be used to disrupt THC in the plant cell. In some cases, a barcode is introduced into the plant cell. Quantification of cannabinoid content can be performed using various methods for instance, qPCR, western blot, sequencing, and/or metabolic analysis.

### Pharmaceutical Compositions and Methods

Provided herein can be pharmaceutical compositions comprising genetically modified cells, organisms, or plants described herein or an extract or product thereof. Provided herein can also be pharmaceutical reagents, methods of using the same, and method of making pharmaceutical compositions comprising genetically modified cells, organisms, or plants described herein or an extract or product thereof. Provided herein are also pharmaceutically and nutraceutical-suitable cells, organisms, or plants described herein or an extract or product thereof.

In some cases, a genetically modified cells, organisms, or plants described herein or an extract or product thereof can be used as a pharmaceutical or nutraceutical agent. In some cases, a composition comprising such a pharmaceutical or nutraceutical agents can be used for treating conditions such as glaucoma, Parkinson's disease, Huntington's disease, migraines, inflammation, epilepsy, fibromyalgia, AIDS, HIV, bipolar disorder, Crohn's disease, dystonia, rheumatoid arthritis, dementia, emesis due to chemotherapy, inflammatory bowel disease, atherosclerosis, posttraumatic stress disorder (PTSD), cardiac reperfusion injury, cancer, and Alzheimer's disease. In some cases, cells, organisms, or plants described herein or an extract or product thereof may also be useful for treating conditions such as Severe debilitating epileptic conditions, Glaucoma, Cachexia, seizures, Hepatitis C, Amyotrophic lateral sclerosis/Lou Gehrig's disease, Agitation of Alzheimer's disease, Tourette's Syndrome, Ulcerative colitis, Anorexia, Spasticity, Multiple sclerosis, Sickle Cell Disease, Post Laminectomy Syndrome with Chronic Radiculopathy, severe Psoriasis and Psoriatic Arthritis, Complex Regional Pain Syndrome, Cerebral palsy, Cystic fibrosis, Muscular dystrophy, and Post Herpetic Neuralgia. Cannabis and/or hemp may also be useful for treating conditions such as Osteogenesis Imperfecta, Decompensated cirrhosis, Autism, mitochondrial disease, epidermolysis bullosa, Lupus, Arnold-Chiari malformation, Interstitial cystitis, Myasthenia gravis, nail-patella syndrome, Sjogren's syndrome, Spinocerebellar ataxia, Syringomyelia, Tarlov cysts, Lennox-Gestaut syndrome, Dravet syndrome, chronic pancreatitis, and/or Idiopathic Pulmonary Fibrosis.

In some aspects, cells, organisms, or plants described herein or an extract or product thereof can be used to treat particular symptoms. For example, pain, nausea, weight loss, wasting, multiple sclerosis, allergies, infection, vasoconstrictor, depression, migraine, hypertension, post-stroke neuroprotection, as well as inhibition of tumor growth, inhibition of angiogenesis, and inhibition of metastasis, antioxidant, and neuroprotectant. In some aspects, cells, organisms, or plants described herein or an extract or product thereof can be used to treat additional symptoms. For instance, persistent muscle spasms, including those that are characteristic of multiple sclerosis, severe arthritis, peripheral neuropathy, intractable pain, migraines, terminal illness requiring end of life care, Hydrocephalus with intractable headaches, Intractable headache syndromes, neuropathic facial pain, shingles, chronic nonmalignant pain, causalgia, chronic inflammatory demyelinating polyneuropathy, bladder pain, myoclonus, post-concussion syndrome, residual limb pain, obstructive sleep apnea, traumatic brain injury (TBI), elevated intraocular pressure, opioids or opiates withdrawal, and/or appetite loss.

In some cases, cells, organisms, or plants described herein or an extract or product thereof may also comprise other pharmaceutically relevant compounds, including flavonoids and phytosterols (e.g., apigenin, quercetin, cannflavin A, beta-sitosterol and the like).

While a wide range of medical uses has been identified, the benefits achieved by cannabinoids for a particular disease or condition are believed to be attributable to a subgroup of cannabinoids or to individual cannabinoids. That is to say that different subgroups or single cannabinoids have beneficial effects on certain conditions, while other subgroups or individual cannabinoids have beneficial effects on other conditions. For example, THC is the main psychoactive cannabinoid produced by Cannabis and is well-characterized for its biological activity and potential therapeutic application in a broad spectrum of diseases. CBD, another major cannabinoid constituent of Cannabis, acts as an inverse agonist of the CB1 and CB2 cannabinoid receptors. Unlike THC, CBD does nor or can have substantially lower levels of psychoactive effects in humans. In some aspects, CBD can exert analgesic, antioxidant, anti-inflammatory, and immunomodulatory effects.

Provided herein are also extracts from cells, organisms, or plants described herein. Kief can refer to trichomes collected from Cannabis. The trichomes of Cannabis are the areas of cannabinoid and terpene accumulation. Kief can be gathered from containers where Cannabis flowers have been handled. It can he obtained from mechanical separation of the trichomes from inflorescence tissue through methods such as grinding flowers or collecting and sifting through dust after manicuring or handling Cannabis. Kief can be pressed into hashish for convenience or storage. Hash—sometimes known as hashish, is often composed of preparations of Cannabis trichomes. Hash pressed from kief is often solid. Bubble Hash—sometimes called bubble melt hash can take on paste-like properties with varying hardness and pliability. Bubble hash is usually made via water separation in which Cannabis material is placed in a cold-water bath and stirred for a long time (around 1 hour). Once the mixture settles it can be sifted to collect the hash. Solvent reduced oils—also sometimes known as hash oil, honey oil, or full melt hash among other names. This type of Cannabis oil is made by soaking plant material in a chemical solvent. After separating plant material, the solvent can be boiled or evaporated off, leaving the oil behind. Butane Hash Oil is produced by passing butane over Cannabis and then letting the butane evaporate. Budder or Wax is produced through isopropyl extraction of Cannabis. The resulting substance is a wax like golden brown paste. Another common extraction solvent for creating Cannabis oil is C02. Persons having skill in the art will be familiar with C02 extraction techniques and devices, including those disclosed in US 20160279183, US 2015/01505455, U.S. Pat. No. 9,730,911, and US 2018/0000857. Tinctures—are alcoholic extracts of Cannabis. These are usually made by mixing Cannabis material with high proof ethanol and separating out plant material. E-juice—are Cannabis extracts dissolved in either propylene glycol, vegetable glycerin, or a combination of both. Some E-juice formulations will also include polyethylene glycol and flavorings. E-juice tends to be less viscous than solvent reduced oils and is commonly consumed on e-cigarettes or pen vaporizers. Rick Simpson Oil (ethanol extractions)—are extracts produced by contacting Cannabis with ethanol and later evaporating the vast majority of ethanol away to create a cannabinoid paste. In some embodiments, the extract produced from contacting the Cannabis with ethanol is heated so as to decarboxylate the extract. While these types of extracts have become a popular form of consuming Cannabis, the extraction methods often lead to material with little or no Terpene Profile. That is, the harvest, storage, handling, and extraction methods produce an extract that is rich in cannabinoids, but often devoid of terpenes.

In some embodiments, cells, organisms, or plants described herein or an extract or product thereof can be subject to methods comprising extractions that preserve the cannabinoids and terpenes. In other embodiments, said methods can be used with any Cannabis plants. The extracts of the present disclosure are designed to produce products for human or animal consumption via inhalation (via combustion, vaporization and nebulization), buccal absorption within the mouth, oral administration, and topical application delivery methods. The present disclosure teaches an optimized method at which we extract compounds of interest, by extracting at the point when the drying harvested plant has reached 15% water weight, which minimizes the loss of terpenes and plant volatiles of interest. Stems are typically still ‘cool’ and ‘rubbery’ from evaporation taking place. This timeframe (or if frozen at this point in process) allow extractor to minimize terpene loss to evaporation. There is a direct correlation between cool/slow, -'dry and preservation of essential oils. Thus, there is a direct correlation to EO loss in flowers that dry too fast, or too hot conditions or simply dry out too much (<10% H20). The chemical extraction of cells, organisms, or plants described herein or an extract or product thereof can be accomplished employing polar and non-polar solvents m various phases at varying pressures and temperatures to selectively or comprehensively extract terpenes, cannabinoids and other compounds of flavor, fragrance or pharmacological value for use individually or combination in the formulation of our products. The extractions can be shaped and formed into single or multiple dose packages, e.g., dabs, pellets and loads. The solvents employed for selective extraction of our cultivars may include water, carbon dioxide, 1,1,1,2-tetrafluoroethane, butane, propane, ethanol, isopropyl alcohol, hexane, and limonene, in combination or series. We can also extract compounds of interest mechanically by sieving the plant parts that produce those compounds. Measuring the plant part i.e. trichome gland head, to be sieved via optical or electron microscopy can aid the selection of the optimal sieve pore size, ranging from 30 to 130 microns, to capture the plant part of interest. The chemical and mechanical extraction methods of the present disclosure can be used to produce products that combine chemical extractions with plant parts containing compounds of interest. The extracts of the present disclosure may also be combined with pure compounds of interest to the extractions, e.g. cannabinoids or terpenes to further enhance or modify the resulting formulation's fragrance, flavor or pharmacology. In some embodiments, the extractions are supplemented with terpenes or cannabinoids to adjust for any loss of those compounds during extraction processes. In some embodiments, the Cannabis extracts of the present disclosure mimic the chemistry of the Cannabis flower material. In some embodiments, the Cannabis extracts of the present disclosure will contain about the same cannabinoid and Terpene Profile of the dried flowers of the cells, organisms, or plants described herein or an extract or product thereof.

In some aspects, extracts of the present disclosure can be used for vaporization, production of e-juice or tincture for e-cigarettes, or for the production of other consumable products such as edibles, balms, or topical spreads. In an aspect, a modified composition provided herein can be used as a supplement, for example a food supplement. Cannabis edibles such as candy, brownies, and other foods are a popular method of consuming Cannabis for medicinal and recreational purposes. In some embodiments, the cells, organisms, or plants described herein or an extract or product thereof can be used to make edibles. Edible recipes can begin with the extraction of cannabinoids and terpenes, which are then used as an ingredient in various edible recipes. In one embodiment, the Cannabis extract used to make edibles out of the Specialty Cannabis of the present disclosure is Cannabis butter. Cannabis butter is made by melting butter in a container with Cannabis and letting it simmer for about half an hour, or until the butter turns green. The butter is then chilled and used in normal recipes. Other extraction methods for edibles include extraction into cooking oil, milk, cream, balms, flour (grinding Cannabis and blending with flour for baking). Lipid rich extraction mediums/edibles are believed to facilitate absorption of cannabinoids into the blood stream. Lipids may be utilized as excipients in combination with the various compositions provided herein. THC absorbed by the body is converted by the liver into 11-hydroxy-THC. This modification increases the ability of the THC molecule to bind to the CB1 receptor and also facilitates crossing of the brain blood barrier thereby increasing the potency and duration of its effects. In other aspects, pharmaceutical compositions provided herein can comprise: oral forms, a transdermal forms, an oil formulation, an edible food, or a food substrate, an aqueous dispersion, an emulsion, a solution, a suspension, an elixir, a gel, a syrup, an aerosol, a mist, a powder, a tablet, a lozenge, a gel, a lotion, a paste, a formulated stick, a balm, a cream, or an ointment.

Provided herein are also kits comprising compositions provided herein. Kits can include packaging, instructions, and various compositions provided herein. In some aspects, kits can also contain additional compositions used to generate the various plants and portions of plants provided herein such as pots, soil, fertilizers, water, and culturing tools.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EXAMPLES

### Example 1

### Target Identification for Gene Editing of Cannabis

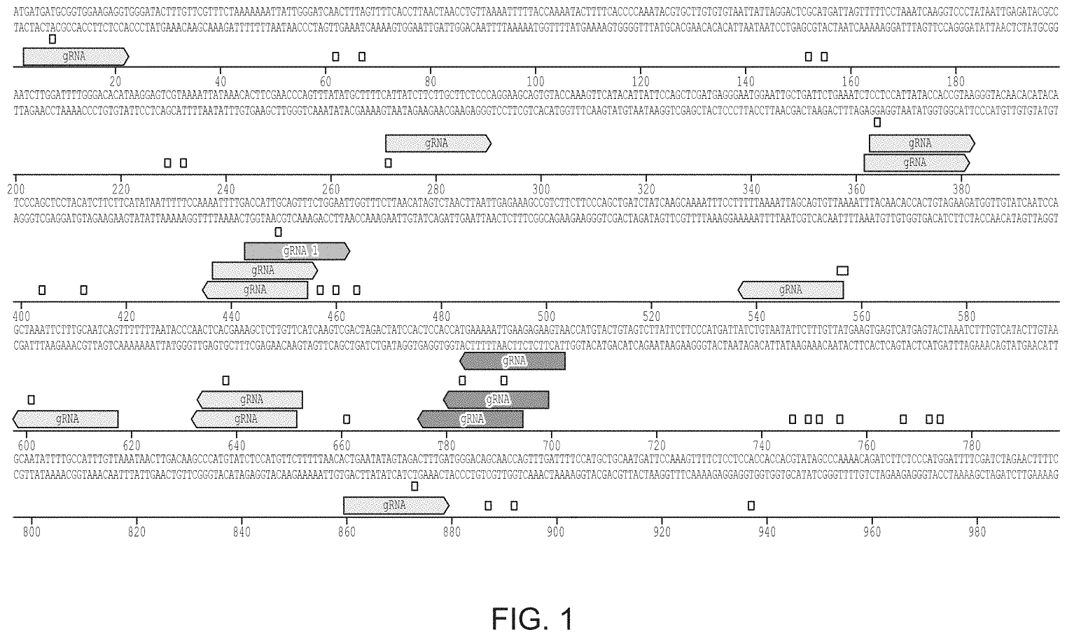

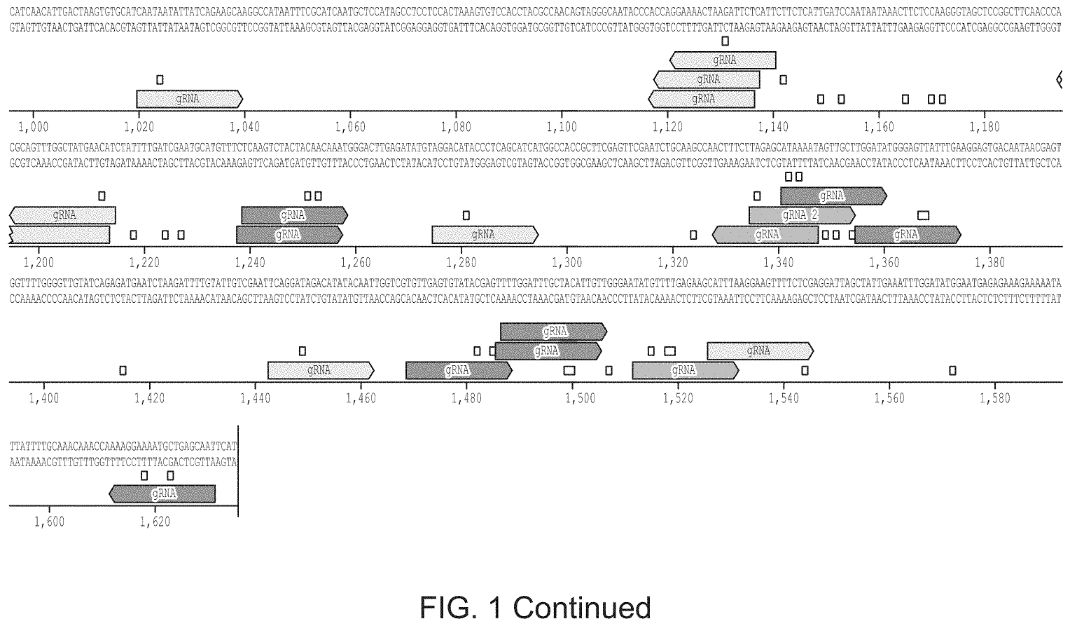

Amplify and sequence the whole gDNA sequence of the gene of interest to design targets on the variety to be used. Target will be selected using the N/G20NGG rule. Software Deskgene or rgenome.net will be used to confirm targets. An exemplary genomic sequence that can be genomicaly editing using methods provided herein is shown in FIG. 1.

### MICAS Mapping

The THCAS protein sequence is obtained from UNIPROT and is used as a reference for retrieving THCAS locus from C. sativa genome. Using BLAT the coordinates of the THCAS gene in Purple Kush genome is obtained. The results were further filtered using a python script blat.ipynb.

TABLE 4
THCAS mapping results at 90% stringency. Associated
nucleic acid sequences shown in Table 7.

| Chromosome | Start | End | Id | Gene |
|---|---|---|---|---|
| CM010797.2 | 28650052 | 28651687 | +99% | THCAS |
| AGQN03005496.1 | 2986 | 4620 | 92% | Likely |
| | | | | CBCAS |
| CM010797.2 | 46549881 | 46551515 | 91% | Likely |
| | | | | pseudo CBDAS |
| AGQN03010271.1 | 2976 | 12143 | 92% | |
| AGQN03006963.1 | 14287 | 35513 | 90% | |

TABLE 5
THCAS mapping results at 85% stringency. Associated
nucleic acid sequences shown in Table 7.

| Homology | Length | Scaffold | Start | End | Chromosome |
|---|---|---|---|---|---|
| 99.816514 | 1635 | CM010797.2 | 28650052 | 28651687 | 7 |
| 92.844037 | 1635 | AGQN03005496.1 | 2985 | 4620 | NaN |
| 92.110092 | 1629 | AGQN03010271.1 | 2976 | 4605 | NaN |
| 91.926606 | 1634 | CM010797.2 | 46549881 | 46551515 | 7 |
| 90.458716 | 1631 | AGQN03006963.1 | 14287 | 15918 | NaN |
| 88.256881 | 1626 | CM010796.2 | 62089462 | 62091088 | 6 |
| 87.706422 | 1625 | AGQN03001397.1 | 578 | 2203 | NaN |
| 86.972477 | 1608 | AGQN03001586.1 | 35792 | 37400 | NaN |
| 86.606 | 1631 | AGQN03001397.1 | 88111 | 89742 | NaN |

The CBDAS genome was blasted against purple kush genome

TABLE 6
Results of BLAST of CBDAS against the purple kush genome

| Chromosome | Start | End | Identity | Gene |
|---|---|---|---|---|
| CM010792.2 | 58200739 | 73430137 | 90% | None |

TABLE 7
THCAS nucleic acid sequences of individual
hits located in different loci of the purple
kush genome using mapping at 90% and 85%
stringency described in Table 4 and Table 5.

| SEQ | | |
|---|---|---|
| ID | | |
| NO | Name | Sequence |
| 6 | (CM01079 | atgatgatgcggtggaagaggtggg |
| | 7.2_28650 | atactttgttcgtttctaaaaaaat |
| | 052_28651 | tattgggatcaactttagttttcac |
| | 687 | cttaactaacctgttaaaattttta |
| | CHR:7.0) | ccaaaatacttttcaccccaaatac |
| | | gtgcttgtgtgtaattattaggact |
| | | cgcatgattagtttttcctaaatca |
| | | aggtccctataattgagatacgcca |
| | | atcttggattttgggacacataagg |
| | | agtcgtaaaattataaacacttcga |
| | | acccagtttatatgcttttcattat |
| | | cttcttgcttctcccaggaagcagt |
| | | gtaccaaagttcatacattattcca |
| | | gctcgatgagggaatggaattgctg |
| | | attctgaaatctcctccattatacc |
| | | accgtaagggtacaacacatacatc |
| | | ccagctcctacatcttcttcatata |
| | | atttttccaaaattttgaccattgc |
| | | agtttctggaattggtttcttaaca |
| | | tagtctaacttaattgagaaagccg |
| | | tcttcttcccagctgatctatcaag |
| | | caaaatttcctttttaaaattagca |
| | | gtgttaaaatttacaacaccactgt |
| | | agaagatggttgtatcaatccagct |
| | | aaattctttgcaatcagttttttta |
| | | atacccaactcacgaaagctcttgt |
| | | tcatcaagtcgactagactatccac |
| | | tccaccatgaaaaattgaagagaag |
| | | taaccatgtactgtagtcttattct |
| | | tcccatgattatctgtaatattctt |
| | | tgttatgaagtgagtcatgagtact |
| | | aaatctttgtcatacttgtaagcaa |
| | | tattttgccatttgttaaataactt |
| | | gacaagcccatgtatctccatgttc |
| | | tttttaacactgaatatagtagact |
| | | ttgatgggacagcaaccagtttgat |
| | | tttccatgctgcaatgattccaaag |
| | | ttttctcctccaccaccacgtatag |
| | | cccaaaacagatcttctcccatgga |
| | | ttttcgatctagaacttttccatca |
| | | acattgactaagtgtgcatcaataa |
| | | tattatcagccgcaaggccataatt |
| | | tcgcatcaatgctccatagcctcct |
| | | ccactaaagtgtccacctacgccaa |
| | | cagtagggcaatacccaccaggaaa |
| | | actaagattctcattcttctcattg |
| | | atccaataataaacttctccaaggg |
| | | tagctccggcttcaacccacgcagt |
| | | ttggctatgaacatctattttgatc |
| | | gaatgcatgtttctcaagtctacta |
| | | caacaaatgggacttgagatatgta |
| | | ggacatacccttcagcatcatggcc |
| | | accgcttcgagttcgaatctgcaag |
| | | ccaactttcttagagcataaaatag |
| | | ttgcttggatatgggagttatttga |
| | | aggagtgacaataacgagtggtttt |
| | | ggggttgtatcagagatgaatctaa |
| | | gattttgtattgtcgaattcaggat |
| | | agacatatacaattggtcgtgttga |
| | | gtgtatacgagttttggatttgcta |
| | | cattgttgggaatatgttttgagaa |
| | | gcatttaaggaagttttctcgagga |
| | | ttagctattgaaatttggatatgga |
| | | atgagagaaagaaaaatattatttt |
| | | gcaaacaaaccaaaaggaaaatgct |
| | | tgagcaattcat |
| 7 | (CM01079 | atgatgacgcggtggaagaggtggg |
| | 7.2_46549 | atactttgttcgtttctaaaaaaat |
| | 881_46551 | tattgggatcagctttggttttcac |
| | 515 | cttaactaacctgttaaaattttta |
| | CHR:7.0) | ccaaaatacttttcaccccaaatac |
| | | gtgcttgtgtgtaattattaggact |
| | | ctcaggattagtttttcctaaatca |
| | | aggtccctataattgagatacgcca |
| | | atcttggattttgggacacataagg |
| | | agttgtgaaattataaacacttcga |
| | | acccagtttatatgcttttcgttat |
| | | cttcttgcttctcccaggtagcagt |
| | | gtaccaaagttcatacattattcca |
| | | gctcgatgagggaatggaatttgct |
| | | gattctgaaatctcatccatttata |
| | | ccaccgtaagggtacaacacataca |
| | | tcccaactcctacctcttcttcata |
| | | taatttttccaaaattttgaccatt |
| | | acagtttcaggtattagtttcttaa |
| | | catagtctaacttaattgagaaagc |
| | | cgtcttcttcccagctgatctatca |
| | | agcaaaatttcctttttaaaattag |
| | | cagtgttgtaatttacaacaccact |
| | | gtagaagatggttgtatcaatccag |
| | | ctcaattctttgcaatcagtttttt |
| | | taatacccaactcaggaaagctctt |
| | | gttcatcaagtcaactagactatcc |
| | | actccaccaagaaaaatggaagaga |
| | | agtaaccatgtactgtagtcttatt |
| | | cttcccatgattatctgtaatattc |
| | | ctagtttctgaagtgagtcgtgagc |
| | | attaaatctttgtcatacttgtaag |
| | | caatattttgccatttgttaaataa |
| | | cttgacaagcccatgtatctccatg |
| | | ttctttttaacactgaatatagtag |
| | | cctttgatgggacaacaacaagttt |
| | | gattttccatgctgcaatgattcca |
| | | aagttttctcctcctccaccacgta |
| | | tagcccaaaatagatcttctccatg |
| | | gattttcgatctagaacttttccat |
| | | caacattgactaagtgtgcatcaat |
| | | gatattatcagccgcaaggccataa |
| | | tttcgcatcaatgctccatagcctc |
| | | ctccactaaagtgtccacctacgcc |
| | | aacagtagggcaatacccaccagga |
| | | aaactaaaattctcattcatctcat |
| | | tgatccaataataaacttctccaag |
| | | ggtagctccggcttcaacccacgca |
| | | gtttggctatgaatatctactttga |
| | | ccgtatgcatgttttcaagtctact |
| | | atagcaaatgggacttgagatatgt |
| | | aggacaaaccctcagcatcatggcc |
| | | accgcttcgagttcgaatctgcaaa |
| | | ccaactttcttggagcagagaatac |
| | | tggcctggatatgggagacatttga |
| | | aggagtgacaataacgagtggtttt |
| | | ggggttgtatcagaggtgaatctaa |
| | | gattttgtattgtcgaattcaggac |
| | | agacatatacaattggtcgtgttga |
| | | gtgtatatgaattttggatttgctg |
| | | gattgttaggaatatattccgagaa |
| | | gcatttaaggaagttttcttgagga |
| | | ttagctattgaaatttggatattga |
| | | atgagagaaagaaaaatattatttt |
| | | gcaaacaaaccaaaaggagaatgtt |
| | | gagcaattcat |
| 8 | (AGQN03 | atgatgacgcggtggaagaggtggg |
| | 005496.1_ | atactttgttcgtttctaaaaaaat |
| | 2986_4620 | tattgggatcagctttggttttcac |
| | CHR:NAN) | cttaactaacctgttaaaattttta |
| | | ccaaaatacttttcaccccaaatac |
| | | gtgcttgtgtgtaattattaggact |
| | | ctcaggattagtttttcctaaatca |
| | | aggtccctataattgagatacgcca |
| | | atcttggattttgggacacataagg |
| | | agttgtgaaattataaacacttcga |
| | | acccagtttatatgcttttcgttat |
| | | cttcttgcttctcccaggtagcagt |
| | | gtaccaaagttcatacattattcca |
| | | gctcgatgagggaatggaattgctg |
| | | attctgaaatctcatccattatacc |
| | | accgtaagggtacaacacatacatc |
| | | ccaactcctacctcttcttcatata |
| | | atttttccaaaattttgaccattgc |
| | | agtttcaggtattagtttcttaaca |
| | | tagtctaacttaattgagaaagccg |
| | | tcttcttcccagctgatctatcaag |
| | | caaaatttccttttttaaaattagc |
| | | agtgttgtaatttacaacaccactg |
| | | tagaagatggttgtatcaatccagc |
| | | tcaattctttgcaatcagttttttt |
| | | aatacccaactcaggaaagctcttg |
| | | ttcatcaagtcaactagactatcca |
| | | ctccaccaagaaaaatggaagagaa |
| | | gtaaccatgtactgtagtcttattc |
| | | ttcccatgattatctgtaatattcc |
| | | tagttctgaagtgagtcgtgagcat |
| | | taaatctttgtcatacttgtaagca |
| | | atattttgccatttgttaaataact |
| | | tgacaagcccatgtatctccatgtt |
| | | ctttttaacactgaatatagtagcc |
| | | tttgatgggacaacaacaagtttga |
| | | ttttccatgctgcaatgattccaaa |
| | | gttttctcctcctccaccacgtata |
| | | gcccaaaatagatcttctcccatgg |
| | | attttcgatctagaacttttccatc |
| | | aacattgactaagtgtgcatcaatg |
| | | atattatcagccgcaaggccataat |
| | | ttcgcatcaatgctccatagcctcc |
| | | tccactaaagtgtccacctacgcca |
| | | acagtagggcaatacccaccaggaa |
| | | aactaaaattctcattcatctcatt |
| | | gatccaataataaacttctccaagg |
| | | gtagctccggcttcaacccacgcag |
| | | tttggctatgaatatctactttgac |
| | | cgtatgcatgttttctcaagtctac |
| | | tatagcaaatgggacttgagatatg |
| | | taggacaaaccctcagcatcatggc |
| | | caccgcttcgagttcgaatctgcaa |
| | | accaactttcttggagcagagaata |
| | | ctggcctggatatgggagacatttg |
| | | aaggagtgacaataacgagtggttt |
| | | tggggttgtatcagaggtgaatcta |
| | | agattttgtattgtcgaattcagga |
| | | cagacatatacaattggtcgtgttg |
| | | agtgtatatgaattttggatttgct |
| | | ggattgttaggaatatattccgaga |
| | | agcatttaaggaagttttcttgagg |
| | | attagctattgaaatttggatattg |
| | | aatgagagaaagaaaaatattattt |
| | | tgcaaacaaaccaaaaggagaatgt |
| | | tgagcaattcat |
| 9 | (AGQN03 | atgatgacgcggtggaagaggtggg |
| | 006963.1_ | atactttgttcgtttctaaaaaaat |
| | 14287_159 | tattggatcagctttggtttcacct |
| | 18 CHR: | aactaacctgttaaaatttttacca |
| | NAN) | aaatacttttcaccccaaatacgtg |
| | | cttgtgtgtaattattaggactctc |
| | | aggattagtttttcctaaatcaagg |
| | | tccctataattgagatacgccaatc |
| | | ttggattttgggacacataaggagt |
| | | tgtgaaattaataaacacttcgaac |
| | | cagtttatatgcttttcgttatctt |
| | | cttgctctcccaggtagcagtgtac |
| | | caaagttcatacattattccagctc |
| | | gatgagggaatggaattgctgattc |
| | | tgaaatctcatccattataccaccg |
| | | taagggtacaacacatacatcccaa |
| | | ctcctacctcttcttcatataattt |
| | | ttccaaaattttgaccattgcagtt |
| | | tcaggtattagtttcttaacatagt |
| | | ctaacttaattgagaaagccgtctt |
| | | cttcccagctgatctatcaagcaaa |
| | | atttcctttttaaaattagcagtgt |
| | | tgtaatttacaacaccactgtagaa |
| | | gatggttgtatcaatccagctcaat |
| | | tctttgcaatcagtttttttaatac |
| | | ccaactcaggaaagctcttgttcat |
| | | caagtcaactagactatccactcca |
| | | ccaagaaaaatggaagagaagtaac |
| | | catgtactgtagtcttattcttccc |
| | | atgattatctgtaatattcctagtt |
| | | ctgaagtgagtcgtgagcattaaat |
| | | ctttgtcatacttgtaagcaatatt |
| | | ttgccatttgttaaataacttgaca |
| | | agcccatgtatctccatgttctttt |
| | | taacactgaatatagtagcctttga |
| | | tgggacaacaacaagtttgattttc |
| | | catgctgcaatgattccaaagtttt |
| | | ctcctcctccaccacgtatagccca |
| | | aaatagatcttctcccatggatttt |
| | | cgatctagaacttttccatcaacat |
| | | tgactaagtgtgcatcaatgatatt |
| | | atcagccgcaaggccataatttcgc |
| | | atcaatgctccatagcctcctccac |
| | | taaagtgtccacctacgccaacagt |
| | | agggcaatacccaccaggaaaacta |
| | | aaattctcattcatctcattgatcc |
| | | aataataaacttctccaagggtagc |
| | | tccggcttcaacccacgcagtttgg |
| | | ctatgaatatctactttgaccgtat |
| | | gcatgtttctcaagtctactatagc |
| | | aaatgggacttgagatatgtaggac |
| | | aaaccctcagcatcatggccaccgc |
| | | ttcgagttcgaatctgcaaaccaac |
| | | tttcttggagcagagaatactggcc |
| | | tggatatgggagacatttgaaggag |
| | | tgacaataacgagtggttttggggt |
| | | tgtatcagaggtgaatctaagattt |
| | | tgtattgtcgaattcaggacagaca |
| | | tatacaattggtcgtgttgagtgta |
| | | tatgaattttggatttgctggattg |
| | | ttaggaatatattccgagaagcatt |
| | | taaggaagttttcttgaggattagc |
| | | tattgaaatttggatattgaatgag |
| | | agaaagaaaaatattattttgcaaa |
| | | caaaccaaaaggagaatgttgagca |
| | | attcat |
| 10 | (AGQN03 | atgatgacgcggtggaagaggtggg |
| | 010271.1_ | atactttgttcgtttctaaaaaaat |
| | 2976_4605 | tattgggatcagctttggttttcac |
| | CHR: | cttaactaacctgttaaaattttta |
| | NAN) | ccaaaatacttttcaccccaaatac |
| | | gtgcttgtgtgtaattattaggact |
| | | ctcaggattagttttcctaaatcaa |
| | | ggtccctataattgagatacgccaa |
| | | tcttggattttgggacacataagga |
| | | gttgtgaaattataaacacttcgaa |
| | | cccagtttatatgcttttcgttatc |
| | | ttcttgcttctcccaggtagcagtg |
| | | taccaaagttcatacattattccag |
| | | ctcgatgagggaatggaattgctga |
| | | ttctgaaatctcatccattatacca |
| | | cgtaagggtacaacacatacatccc |
| | | aactcctacctcttcttcatataat |
| | | ttttccaaaattttgaccattgcag |
| | | tttcaggtattagtttcttaacata |
| | | gtctaacttaattgagaaagccgtc |
| | | ttcttcccagctgatctatcaagaa |
| | | aatttcctttttaaaattagcagtg |
| | | ttgtaatttacaacaccactgtaga |
| | | agatggttgtatcaatccagctcaa |
| | | ttctttgcaatcagtttttttaata |
| | | cccaactcaggaaagctcttgttca |
| | | tcaagtcaactagactatccactcc |
| | | accaagaaaaatggaagagaagtaa |
| | | ccatgtactgtagtcttattcttcc |
| | | catgattatctgtaatattcctagt |
| | | tctgaagtgagtcgtgagcattaaa |
| | | tctttgtcatacttgtaagcaatat |
| | | tttgccatttgttaaataacttgac |
| | | aagcccattgttatctccatgttct |
| | | tttttaacactgaatatagtagcct |
| | | tttgattgggacaacaacaagtttg |
| | | atttttccatgctgcaatgattcca |
| | | aagttttctcctcctccaccacgta |
| | | tagcccaaaatagatcttctcccat |
| | | ggattttcgatctagaacttttcca |
| | | tcaacattgactaagtgtgcatcaa |
| | | tgatattatcagccgcaaggccata |
| | | atttcgcatcaatgctccatagcct |
| | | cctccactaaagtgtccacctacgc |
| | | caacagtagggcaatacccaccagg |
| | | aaaactaaaattctcattcatcttg |
| | | atccaataataaacttctccaaggg |
| | | tagctccggcttcaacccacgcagt |
| | | ttggctatgaatatctactttgacc |
| | | gtatgcatgtttctcaagtctacta |
| | | tagcaaatgggacttgagatatgta |
| | | ggacaaaccctcagcatcatggcca |
| | | ccgcttcgagttcgaatctgcaaac |
| | | caactttcttggagcagagaatact |
| | | ggcctggatatgggagacatttgaa |
| | | ggagtgacaataacgagtggttttg |
| | | gggttgtatcagaggtgaatctaag |
| | | attttgtattgtcgaattcaggaca |
| | | gacatatacaattggtcgtgttgag |
| | | tgtatatgaattttggatttgctgg |
| | | attgttaggaatatattccgagaag |
| | | catttaaggaagttttcttgaggat |
| | | tagctattgaaattttggatattga |
| | | atgagagaaagaaaaatattatttt |
| | | gcaaacaaaccaaaaggagaatgtt |
| | | gagcaattcat |

sgRNA preparation

The forward primer for the sgRNA preparation is: tgtggtctcaattgnnnnnnnnnnnnnn nnnnnghttagagctagaaatagcaag (SEQ ID NO: 101) (The BsaI recognition site is: ggtctc; the four base pair overhang produced by digestion with BsaI is ATTG—this fuses to the last four base pairs of the AtU6-26 promoter in plasmid pICSL90002; the 20 bp target sequence is GN NN; the portion of the oligonucleotide that anneals to the sgRNA template is gttttagagctagaaatagcaag (SEQ ID NO: 102))

The following reverse primer will be used in combination with the forward primer to amplify a PCR product using the plasmid pICSL90002 as template: tgtggtctcaagcgtaatgccaactttgtac (SEQ ID NO: 103)

(The BsaI recognition site is ggtctc; the four base pair overhang produced by digestion with BsaI is AGCG—this fused to the Level 1 acceptor plasmid; the portion of the oligonucleotide that anneals to the sgRNA template is taatgccaactttgtac (SEQ ID NO: 104))

After quantification the appropriate amount of DNA obtained from the PCR reaction (1), and after its purification, a Level 1 assembly reaction is set up using the following plasmids: three targets can be simultaneously used, therefore, three independent acceptor reaction are needed

TABLE 8
Plasmids for target identification

| Plasmid | Insert |
|---|---|
| pICSL90002* (AddGene | Promoter, U6-26 (Arabidopsis thaliand) |
| #68261) | |
| n/a | PCR amplicons from sgRNA PCR template |
| | (amplified from Addgene#46966 |
| | (pICSL90002) with primers described |
| | above) |
| pICH47751 (AddGene | Level 1, position 3 acceptor |
| #48002) | |
| pICH47761 (AddGene | Level 1, position 4 acceptor |
| #48003) | |
| pICH47772 (AddGene | Level 1, position 5 acceptor |
| #48004) | |

### Assembly of Level 1 Transcriptional Units

Level 1 assembly reactions contained 100-200 ng of the Level 1 acceptor plasmid (pICH477751 or 47761 or 47772) as well as 100-200 ng of Level 1 plasmids containing the U6-26 promoter (pICSL90002) and the sgRNA amplicon (amplified in 1) at a molar ratio to the acceptor 2:1. The reaction mix includes 10 units of BsaI (NEB), 2 uL of 10× BSA, 400 units of T4 DNA ligase (NEB) and 2 uL of T4 ligase buffer (provided with T4 ligase). Reaction volumes were made up to 20 uL using sterile distilled water. The reaction incubated in a thermocycler as follows: 26 cycles of 37° C. for 3 min/16° C. for 4 min followed by 50° C. for 5 min and finally 80° C. for 5 min. Transformation was done at a total of 2 uL of each reaction into chemically competent E. coli cells (Invitrogen). Cells were spread on LB agar plates containing 100 mg/L Ampicillin (Melford), 25 mg/L IPTG (Melford) and 40 mg/L Xgal (Melford). White colonies were selected, and the fidelity was confirmed of the clone utilizing restriction digest analysis and Sanger sequencing.

Assembly of Level M Binary Vectors with Multiple sgRNAs

Level 1 constructs were combined and assembled into Level M acceptor plasmids to make the final binary vectors delivered to plants. The following Level 1 constructs, end-linkers and Level M acceptors are used.

TABLE 9
Level 1 constructs, end-linkers, and level M acceptors

| Plasmid | Insert |
|---|---|
| pICSL11055* | Plant selection cassette; Kan |
| (AddGene #68252) | |
| pICSL11060* | Cas9 cassette |
| (AddGene #68264) | |
| pICLS47751 | sgRNA cassette 1 (from 3) |
| pICLS47761 | sgRNA cassette 2 (from 3) |
| pICSL47772 | sgRNA cassette 3 (from 3) |
| pICH50914 | Position 5 end linker |
| pAGM8031 | Binary Vector Backbone; Level M acceptor |
| (Addgene #48037) | |

The Level M assembly reaction contains 100-200 ng of the Level M acceptor plasmid (pAGM8031) as well as Level 1 plasmids containing each of the three targets to be included in the acceptor backbone at a 2:1 molar ratio to the acceptor. In addition, Level 1 vectors containing 100-200 ng of the plant selection cassette, (pICSL11055; Kan), and the Cas9 cassette (pICSL11060) are added. The reaction mix includes 20 units of BpiI ThermoFisher), 2 uL of 10× BSA, 400 units of T4 DNA ligase (NEB) and 2 uL of T4 ligase buffer (provided with T4 ligase). Reaction volumes are made up to 20 uL using sterile distilled water. Reactions are incubated in a thermocycler as follows: 26 cycles of 37° C. for 3 min/16° C. for 4 min followed by 50° C. for 5 min and finally 80° C. for 5 min.

2 uL of each reaction are transformed into chemically competent E. coli cells (Invitrogen). Cells are spread on LB agar plates containing 100 mg/L Spectinomycin (Sigma), 25 mg/L IPTG (Melford) and 40 mg/L Xgal (Melford). White colonies are selected and used to confirm the fidelity of the clone by restriction digest analysis and Sanger sequencing. The destination vector (pAGM8031) is sequenced and confirmed, and plasmid is electroporated in Agrobacterium. Positive colonies are selected for glycerol stock preparation (20% glycerol) and placed at −80C.

### Example 2

### Bioinformatic Analysis of THCAS in Hemp (Finola)

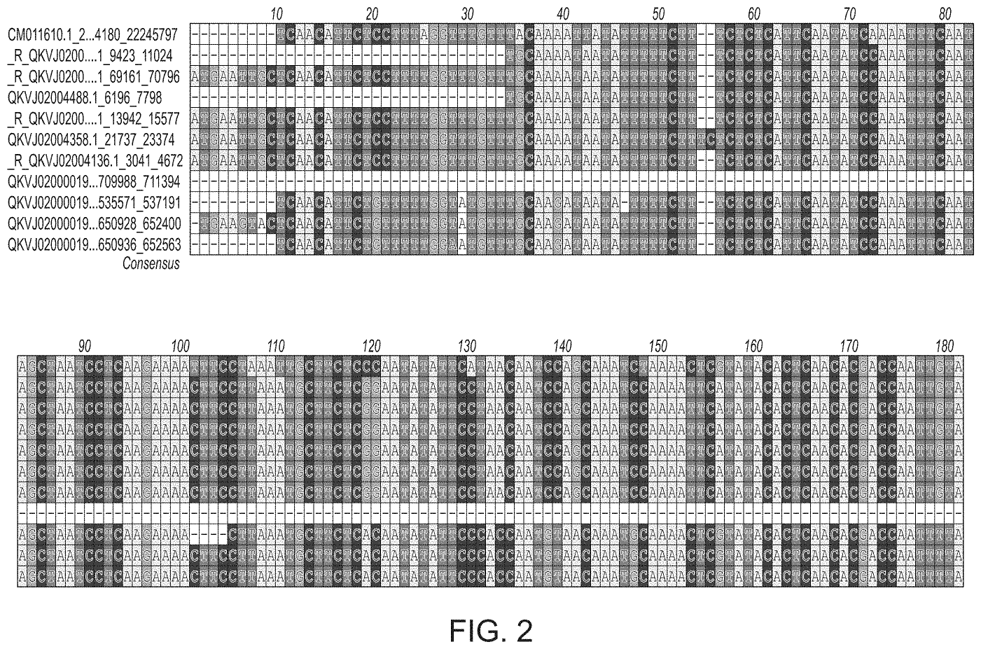

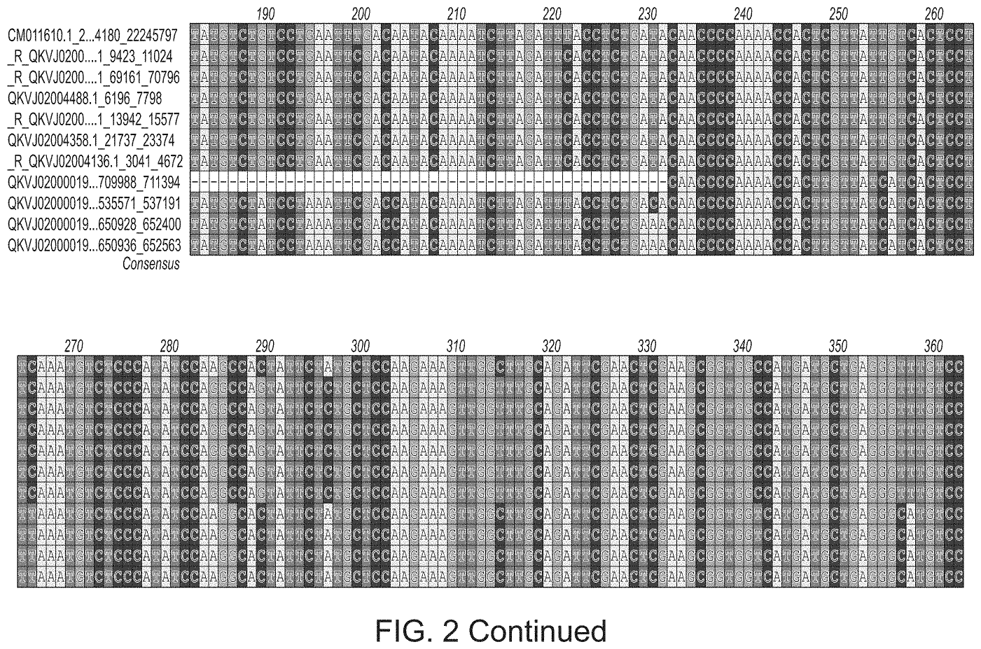

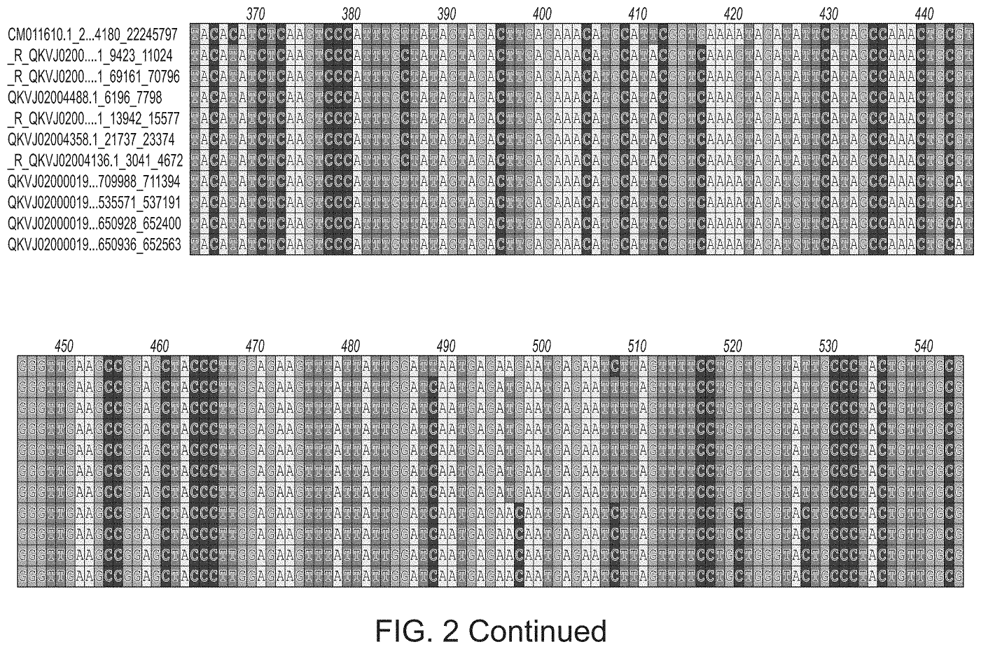

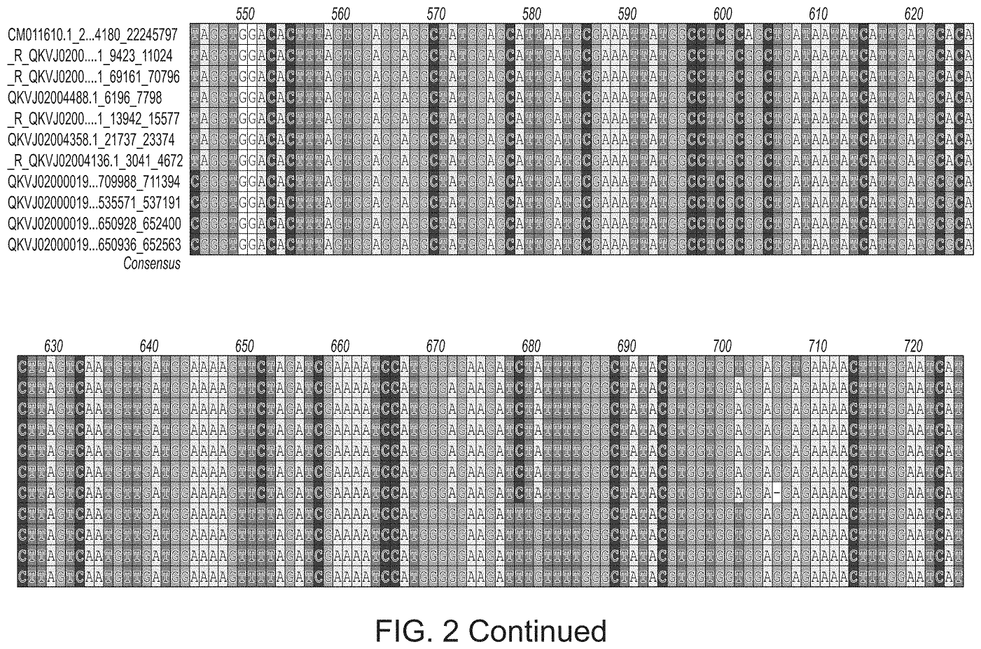

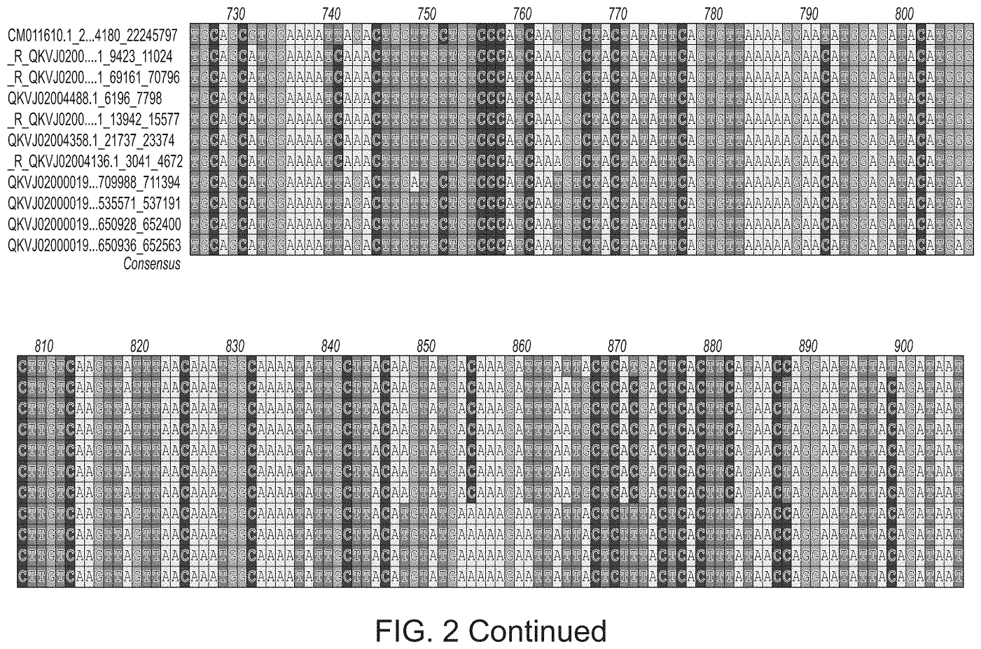

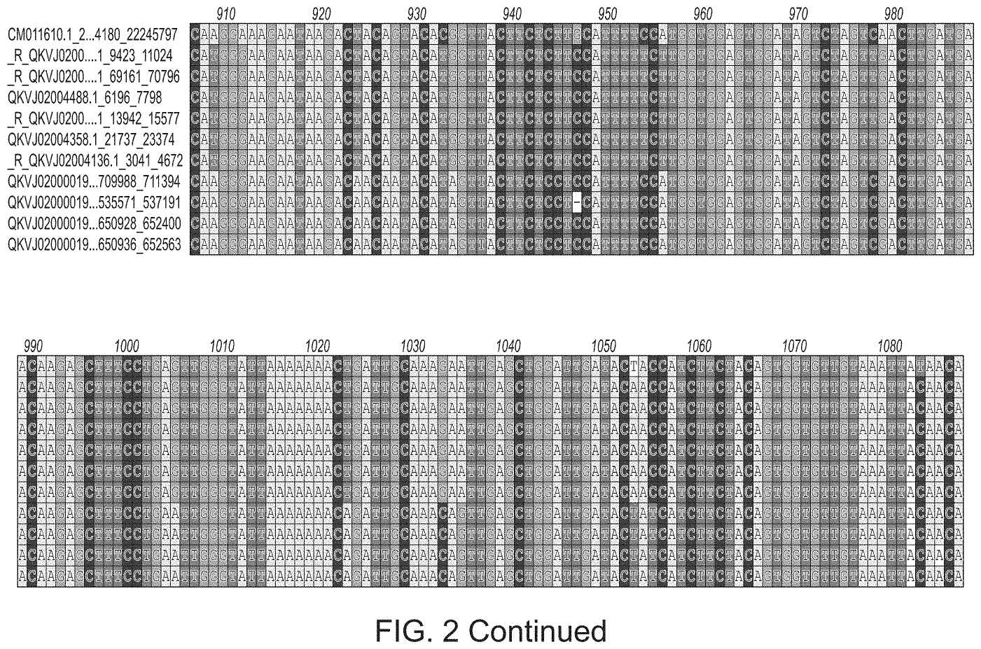

THCAS in Finola Hemp was analyzed at 85% stringency, Table 10. The nucleotide alignment of THCAS hits in Finola is shown in FIG. 2.

TABLE 10
THCAS in Finola (85% stringency). Hit numbers 1, 2, 3, 4, 5, 6 and
8 group together on the alignments. 7. 9.10 and 11 group together.

| | | | | | | | BLAST | BLASTx |
|---|---|---|---|---|---|---|---|---|
| | | | | | | | search of | search |
| Hit | | | | | | | nucleotide | (nucleotide |
| number | chromosome | end | homology | length | scaffold | start | sequence | to aa search) |
| 1 | NaN | 11024 | 92.884 | 1601 | QKVJ02001794.1 | 9423 | THCAS | THCAS 100% |
| | | | | | | | (BLAST | identity with |
| | | | | | | | search = | AJB28532.1 |
| | | | | | | | THCAS/ | |
| | | | | | | | THCA2) | |
| | | | | | | | High similarity | |
| | | | | | | | (99-100% | |
| | | | | | | | Identity) in | |
| | | | | | | | what looks | |
| | | | | | | | like 7 | |
| | | | | | | | different | |
| | | | | | | | Cannabis | |
| | | | | | | | sativa | |
| | | | | | | | cultivars | |
| 2 | NaN | 70796 | 92.844037 | 1635 | QKVJ02001794.1 | 69161 | BLAST | THCAS 99.8% |
| | | | | | | | search = | identity with |
| | | | | | | | THCA2 | AJB28532.1 |
| 3 | NaN | 15577 | 92.844037 | 1635 | QKVJ02004887.1 | 13942 | BLAST | THCAS 99.82% |
| | | | | | | | search = | identity with |
| | | | | | | | THCA2 | AJB28532.1 |
| 4 | NaN | 23374 | 92.66055 | 1637 | QKVJ02004358.1 | 21737 | BLAST | THCAS 99.44% |
| | | | | | | | search = | identity with |
| | | | | | | | THCA2 | AJB28532.1 |
| 5 | NaN | 4672 | 92.477064 | 1631 | QKVJ02004136.1 | 3041 | BLAST search = | THCAS 99.68% |
| | | | | | | | THCA2 | identity with |
| | | | | | | | (~99.6% | AJB2853 |
| | | | | | | | identity) | 2.1 |
| 6 | NaN | 7798 | 91.009174 | 1602 | QKVJ02004488.1 | 6196 | BLAST | THCAS 100% |
| | | | | | | | search = | identity with |
| | | | | | | | THCA2 | AJB28532.1 |
| | | | | | | | (~99.8% | |
| | | | | | | | identity) | |
| 7 | NaN | 711394 | 89.979 | 1406 | QKVJ02000019.1 | 709988 | BLAST | CBDAS1 99.36% |
| | | | | | | | search = | identity with |
| | | | | | | | CBDAS2 (first | A6P6W0.1 |
| | | | | | | | hit at 99.79% | |
| | | | | | | | identity), | |
| | | | | | | | CBDAS3 (second | |
| | | | | | | | hit at 99.5% | |
| | | | | | | | identity) | |
| | | | | | | | Missing ~220 | |
| | | | | | | | bp from the | |
| | | | | | | | start, no | |
| | | | | | | | start codon | |
| 8 | 6 | 22245797 | 88.990826 | 1617 | CM011610.1 | 22244180 | No annotated | THCAS |
| | | | | | | | top BLAST | 90.72% |
| | | | | | | | hits THCAS | identity |
| | | | | | | | identified at | with |
| | | | | | | | 93% identity | AF124256.1 |
| | | | | | | | low down the | |
| | | | | | | | list | |
| 9 | NaN | 652400 | 88.798 | 1472 | QKVJ02000019.1 | 650928 | BLAST | CBDAS1 99.39% |
| | | | | | | | search = | identity with |
| | | | | | | | CBDAS2 (first | A6P6W0.1 |
| | | | | | | | hit at 99.73% | |
| | | | | | | | identity), | |
| | | | | | | | CBDAS3 (second | |
| | | | | | | | hit at 99.32% | |
| | | | | | | | identity), | |
| | | | | | | | third hit is | |
| | | | | | | | THCAS ~150 | |
| | | | | | | | bp shorter and | |
| | | | | | | | without a | |
| | | | | | | | stop codon | |
| 10 | NaN | 652563 | 88.623853 | 1627 | QKVJ02000019.1 | 650936 | BLAST | CBDAS1 98.98% |
| | | | | | | | search = | identity with |
| | | | | | | | CBDAS2 (first | A6P6W0.1 |
| | | | | | | | hit at 99.75% | |
| | | | | | | | identity), | |
| | | | | | | | CBDAS3 (second | |
| | | | | | | | hit at 99.32% | |
| | | | | | | | identity), | |
| | | | | | | | third hit is | |
| | | | | | | | THCAS | |
| 11 | NaN | 537191 | 87.522936 | 1620 | QKVJ02000019.1 | 535571 | BLAST search = | CBDAS1 100% |
| | | | | | | | CBDAS2 (first | identity with |
| | | | | | | | hit at 99.51% | A6P6W0.1 |
| | | | | | | | identity), | |
| | | | | | | | CBDAS3 (second | |
| | | | | | | | hit at 99.14% | |
| | | | | | | | identity), | |
| | | | | | | | third hit is | |
| | | | | | | | THCAS (92.3% | |
| | | | | | | | identity) | |

THCAS hits in Finola were translated to amino acid sequences using BlastX Amino acid sequences are shown in Table 11.

TABLE 11
Amino acid sequences of THCAS hits in Finola identified at 85% stringency as
described in Table 10.

| SEQ | | |
|---|---|---|
| ID | | |
| NO | Name | Sequence |
| 11 | >_R_QKVJ02001794.1_9423_11024 | CKIIFFFLSFNIQISIANPQENFLKCFSEYIPNNPANPKFIYTQHDQLYMSVLNSTI |
| | chr:nan | QNLRFTSDTTPKPLVIVTPSNVSHIQASILCSKKVGLQIRTRSGGHDAEGLSYISQ |
| | THCAS | VPFAIVDLRNMHTVKVDIHSQTAWVEAGATLGEVYYWINEMNENFSFPGGYC |
| | | PTVGVGGHFSGGGYGALMRNYGLAADNIIDAHLVNVDGKVLDRKSMGEDLF |
| | | WAIRGGGGENFGIIAAWKIKLVVVPSKATIFSVKKNMEIHGLVKLFNKWQNIA |
| | | YKYDKDLMLTTHFRTRNITDNHGKNKTTVHGYFSSIFLGGVDSLVDLMNKSFP |
| | | ELGIKKTDCKELSWIDTTIFYSGVVNYNTANFKKEILLDRSAGKKTAFSIKLDY |
| | | VKKLIPETAMVKILEKLYEEEVGVGMYVLYPYGGIMDEISESAIPFPHRAGIMY |
| | | ELWYTATWEKQEDNEKHINWVRSVYNFTTPYVSQNPRLAYLNYRDLDLGKT |
| | | NPESPNNYTQARIWGEKYFGKNFNRLVKVKTKADPNNFFRNEQSIPPLPPRH |
| 12 | >_R_QKVJ02001794.1_69161_70796 | MNCSTFSFWFVCKIIFFFLSFNIQISIANPQENFLKCFSEYIPNNPANPKFIYTQHD |
| | chr:nan | QLYMSVLNSTIQNLRFTSDTTPKPLVIVTPSNVSHIQASILCSKKVGLQIRTRSG |
| | THCAS | GHDAEGLSYISQVPFAIVDLRNMHTVKVDIHSQTAWVEAGATLGEVYYWINE |
| | | MNENFSFPGGYCPTVGVGGHFSGGGYGALMRNYGLAADNIIDAHLVNVDGK |
| | | VLDRKSMGEDLFWAIRGGGGENFGIIAAWKIKLVVVPSKATIFSVKKNMEIHG |
| | | LVKLFNKWQNIAYKYDKDLMLTTHFRTRNITDNHGKNKTTVHGYFSSIFLGG |
| | | VDSLVDLMNKSFPELGIKKTDCKELSWIDTTIFYSGVVNYNTANFKKEILLDRS |
| | | AGKKTAFSIKLDYVKKLIPETAMVKILEKLYEEEVGVGMYVLYPYGGIMDEIS |
| | | ESAIPFPHRAGIMYELWYTATWEKQEDNEKHINWVRSVYNFTTPYVSQNPRLA |
| | | YLNYRDLDLGKTNPESPNNYTQARIWGEKYFGKNFNRLVKVKTKADPNNFFR |
| | | NEQSIPPLPPRHH |
| 13 | >_R_QKVJ02004887.1_13942_15577THCAS | MNCSTFSFWFVCKIIFFFLSFNIQISIANPQENFLKCFSEYIPNNPANPKFIYTQHD |
| | chr:nan | QLYMSVLNSTIQNLRFTSDTTPKPLVIVTPSNVSHIQASILCSKKVGLQIRTRSG |
| | | GHDAEGLSYISQVPFAIVDLRNMHTVKVDIHSQTAWVEAGATLGEVYYWINE |
| | | MNENFSFPGGYCPTVGVGGHFSGGGYGALMRNYGLAADNIIDAHLVNVDGK |
| | | VLDRKSMGEDLFWAIRGGGGENFGIIAAWKIKLVVVPSKATIFSVKKNMEIHG |
| | | LVKLFNKWQNIAYKYDKDLMLTTHFRTRNITDNHGKNKTTVHGYFSSIFLGG |
| | | VDSLVDLMNKSFPELGIKKTDCKELSWIDTTIFYSGVVNYNTANFKKEILLDRS |
| | | AGKKTAFSIKLDYVKKLIPETAMVKILEKLYEEEVGVGMYVLYPYGGIMDEIS |
| | | ESAIPFPHRAGIMYELWYTATWEKQEDNEKHINWVRSVYNFTTPYVSQNPRLA |
| | | YLNYRDLDLGKTNPESPNNYTQARIWGEKYFGKNFNRLVKVKTKADPNNFFR |
| | | NEQSIPPLPPRHH |
| 14 | >QKVJ02004358.1_21737_23374 | ENFGHAAWKIKLVVVPSKATIFSVKKNMEIHGLVKLFNKWQNIAYKYDKDLM |
| | chr:nan | LTTHFRTRNITDNHGKNKTTVHGYFSSIFLGGVDSLVDLMNKSFPELGIKKTDC |
| | THCAS | KELSWIDTTIFYSGVVNYNTANFKKEILLDRSAGKKTAFSIKLDYVKKLIPETA |
| | | MVKILEKLYEEEVGVGMYVLYPYGGIMDEISESAIPFPHRAGIMYELWYTATW |
| | | EKQ- |
| | | DNEKHINWVRSVYNFTTPYVSQNPRLAYLNYRDLDLGKTNPESPNNYTQARI |
| | | WGEKYFGKNFNRLVKVKTKADPNNFFRNEQSIPPLPPRHH |
| 15 | >QKVJ02004488.1_6196_7798THCAS | CKIIFFFLSFNIQISIANPQENFLKCFSEYIPNNPANPKFIYTQHDQLYMSVLNSTI |
| | chr:nan | QNLRFTSDTTPKPLVIVTPSNVSHIQASILCSKKVGLQIRTRSGGHDAEGLSYISQ |
| | | VPFAIVDLRNMHTVKVDIHSQTAWVEAGATLGEVYYWINEMNENFSFPGGYC |
| | | PTVGVGGHFSGGGYGALMRNYGLAADNIIDAHLVNVDGKVLDRKSMGEDLF |
| | | WAIRGGGGENFGIIAAWKIKLVVVPSKATIFSVKKNMEIHGLVKLFNKWQNIA |
| | | YKYDKDLMLTTHFRTRNITDNHGKNKTTVHGYFSSIFLGGVDSLVDLMNKSFP |
| | | ELGIKKTDCKELSWIDTTIFYSGVVNYNTANFKKEILLDRSAGKKTAFSIKLDY |
| | | VKKLIPETAMVKILEKLYEEEVGVGMYVLYPYGGIMDEISESAIPFPHRAGIMY |
| | | ELWYTATWEKQEDNEKHINWVRSVYNFTTPYVSQNPRLAYLNYRDLDLGKT |
| | | NPESPNNYTQARIWGEKYFGKNFNRLVKVKTKADPNNFFRNEQSIPPLPPRHH |
| 16 | >QKVJ02000019.1_709988_711394CBDAS1 | TPKPLVIITPLNVSHIQGTILCSKKVGLQIRTRSGGHDAEGMSYISQVPFVIVDLR |
| | chr:nan | NMHSVKIDVHSQTAWVEAGATLGEVYYWINENNENLSFPAGYCPTVGAGGH |
| | | FSGGGYGALMRNYGLAADNIIDAHLVNVDGKVLDRKSMGEDLFWAIRGGGG |
| | | ENFGHAAWKIRLDAVPSMSTIFSVKKNMEIHELVKLVNKWQNIAYMYEKELL |
| | | LFTHFITRNITDNQGKNKTTIHSYFSSIFHGGVDSLVDLMNKSFPELGIKKTDCK |
| | | QLSWIDTIIFYSGVVNYNTTNFKKEILLDRSGGRKAAFSIKLDYVKKPIPETAMV |
| | | TILEKLYEEDVGVGMFVFYPYGGIMDEISESAIPFPHRAGIMYEIWYIASWEKQ |
| | | EDNEKHINWIRNVYNFTTPYVSQNPRMAYLNYRDLDLGKTNFESPNNYTQARI |
| | | WGEKYFGKNFNRLVKVKTKVDHDNFFRNEQSIPPLPLRHH |
| 17 | >CM011610.1_22244180_22245797 | STFSFRFVYKIIFFFLSFNIKISIANPQENFLNCFSQYIHNNPANLKLVYTQHDQL |
| | chr:6.0 | YMSVLNLTIQNLRFTSDTTPKPLVIVTPSNVSHIQATILCSKKVGLQIRTRSGGH |
| | THCAS | DAEGLSYTSQVPFVIVDLRNMHSVKIDIRSQTAWVEAGATLGEVYYWINEKNE |
| | | NLSFPGGYCPTVGVGGHFSGGGYGALMRNYGLAADNIIDAHLVNVDGKVLD |
| | | RKSMGEDLFWAIRGGGGENFGIIAAWKIRLVAVPSRATIFSVKRNMEIHGLVK |
| | | LFNKWQNIAYKYDKDLLLMTHFITRNIIDNQGKNKTTVHGYFSCIFHGGVDSL |
| | | VNLMNKSFPELGIKKTDCKELSWIDTTIFYSGVVNYNTTNFQKEILLDRSAGQK |
| | | VAFSIKLDYVKKPIPETAIVKILEKLYEEDVGVGVYVLYPYGGIMDKISESTIPFP |
| | | HRAGIMYEVWYAATWEKQEDNEKHINWVRSVYNFMTPYVSQNPRMAYLNY |
| | | RDLDLGKTDPKSPNNYTQARIWGEKYFGKNFDKLVKVKTKVDPNNFFRNEQS |
| | | IPPLPP |
| 18 | >QKVJ02000019.1_650928_652400 | KYSTFCFWYVCKIIFFFLSFNIQISIANPQENFLKCFSQYIPTNVTNAKLVYTQHD |
| | chr:nanCBDAS1 | QFYMSILNSTIQNLRFTSETTPKPLVIITPLNVSHIQGTILCSKKVGLQIRTRSGGH |
| | | DAEGMSYISQVPFVIVDLRNMHSVKIDVHSQTAWVEAGATLGEVYYWINENN |
| | | ENLSFPAGYCPTVGAGGHFSGGGYGALMRNYGLAADNIIDAHLVNVDGKVLD |
| | | RKSMGEDLFWAIRGGGGENFGIIAAWKIRLVAVPSMSTIFSVKKNMEIHELVK |
| | | LVNKWQNIAYMYEKELLLFTHFITRNITDNQGKNKTTIHSYFSSIFHGGVDSLV |
| | | DLMNKSFPELGIKKTDCKQLSWIDTIIFYSGVVNYNTTNFKKEILLDRSGGRKA |
| | | AFSIKLDYVKKPIPETAMVTILEKLYEEDVGVGMFVFYPYGGIMDEISESAIPFP |
| | | HRAGIMYEIWYIASWEKQEDNEKHINWIRNVYNFTTPYVSQNPRMAYLNYRD |
| | | LDLGK |
| 19 | >QKVJ02000019.1_650936_652563 | STFCFWYVCKIIFFFLSFNIQISIANPQENFLKCFSQYIPTNVTNAKLVYTQHDQF |
| | chr:nanCBDAS1 | YMSILNSTIQNLRFTSETTPKPLVIITPLNVSHIQGTILCSKKVGLQIRTRSGGHD |
| | | AEGMSYISQVPFVIVDLRNMHSVKIDVHSQTAWVEAGATLGEVYYWINENNE |
| | | NLSFPAGYCPTVGAGGHFSGGGYGALMRNYGLAADNIIDAHLVNVDGKVLD |
| | | RKSMGEDLFWAIRGGGGENFGIIAAWKIRLVAVPSMSTIFSVKKNMEIHELVK |
| | | LVNKWQNIAYMYEKELLLFTHFITRNITDNQGKNKTTIHSYFSSIFHGGVDSLV |
| | | DLMNKSFPELGIKKTDCKQLSWIDTIIFYSGVVNYNTTNFKKEILLDRSGGRKA |
| | | AFSIKLDYVKKPIPETAMVTILEKLYEEDVGVGMFVFYPYGGIMDEISESAIPFP |
| | | HRAGIMYEIWYIASWEKQEDNEKHINWIRNVYNFTTPYVSQNPRMAYLNYRD |
| | | LDLGKN*FR |
| 20 | >QKVJ02000019.1_535571_537191CBDAS1 | LKCFSQYIPTNVTNAKLVYTQHDQFYMSILNSTIQNLRFTSDTTPKPLVIITPLN |
| | chr:nan | VSHIQGTILCSKKVGLQIRTRSGGHDAEGMSYISQVPFVIVDLRNMHSVKIDVH |
| | | SQTAWVEAGATLGEVYYWINENNENLSFPAGYCPTVGAGGHFSGGGYGALM |
| | | RNYGLAADNIIDAHLVNVDGKVLDRKSMGEDLFWAIRGGGGENFGHAAWKI |
| | | RLVAVPSMSTIFSVKKNMEIHELVKLVNKWQNIAYMYEKELLLFTHFITRNITD |
| | | NQGKNKTTIHSYFSS |

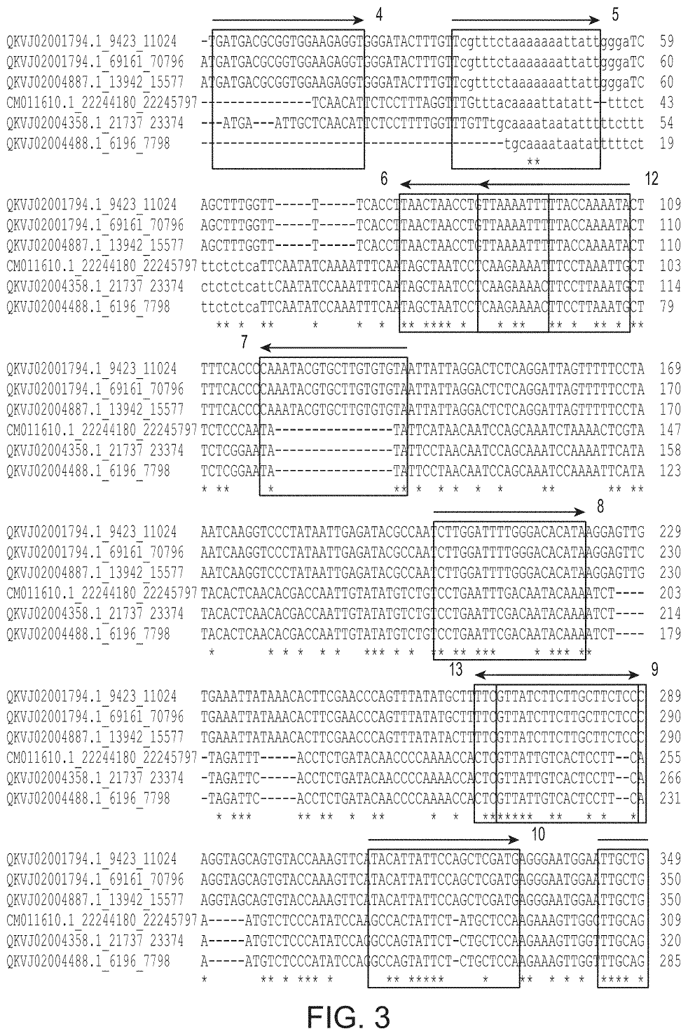

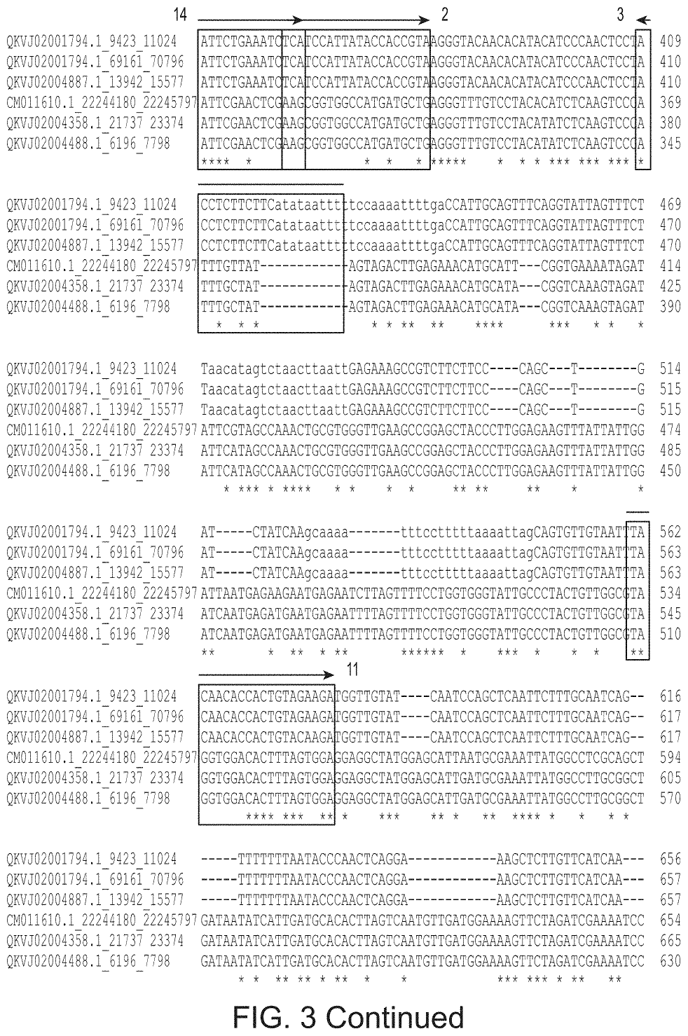

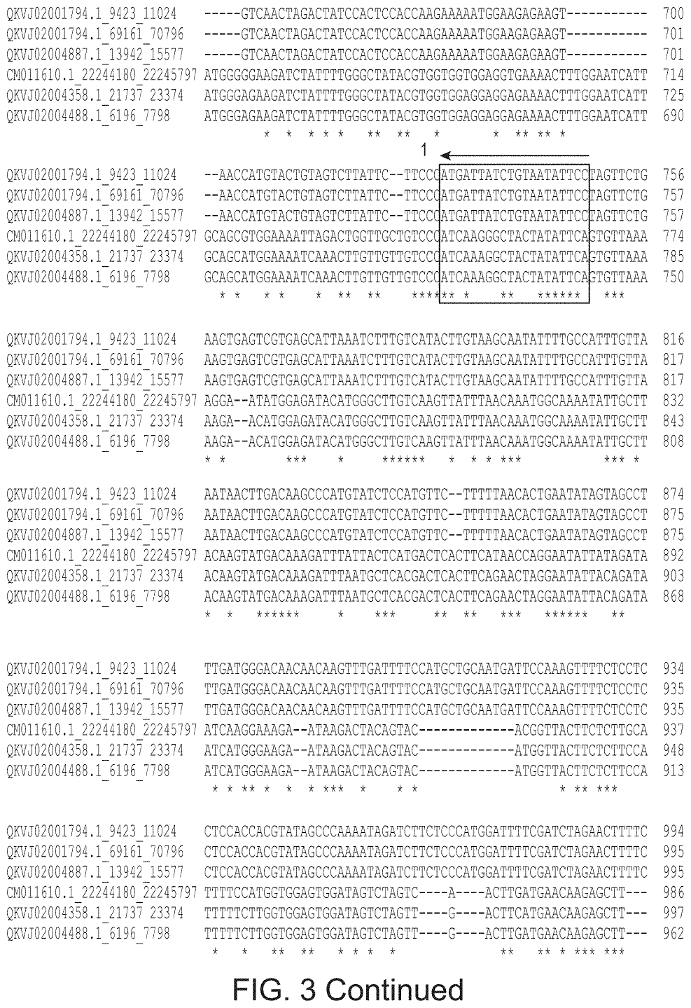

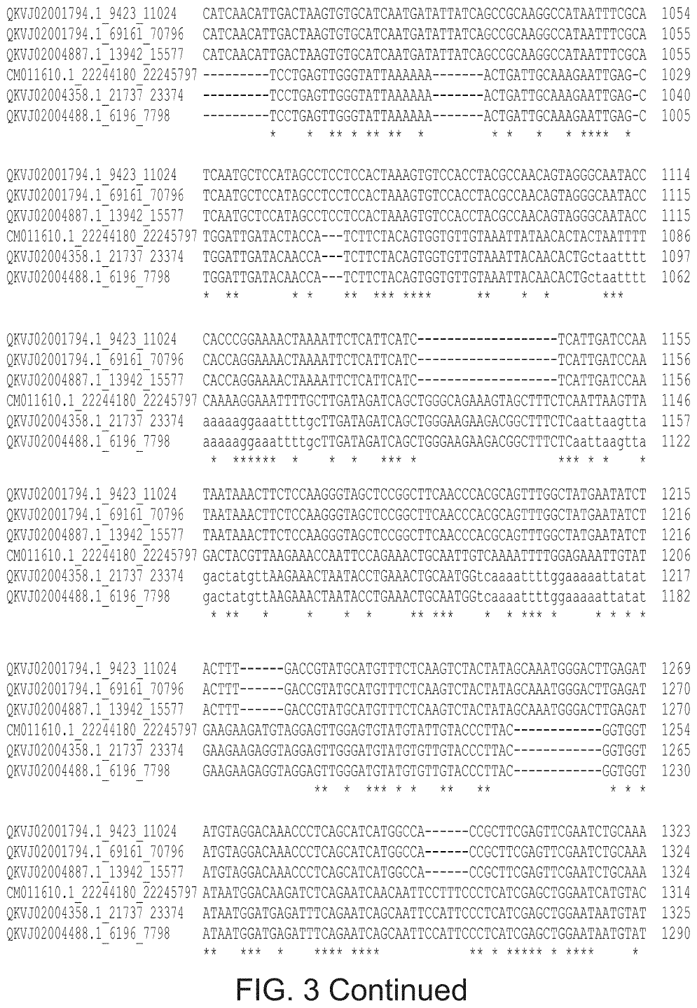

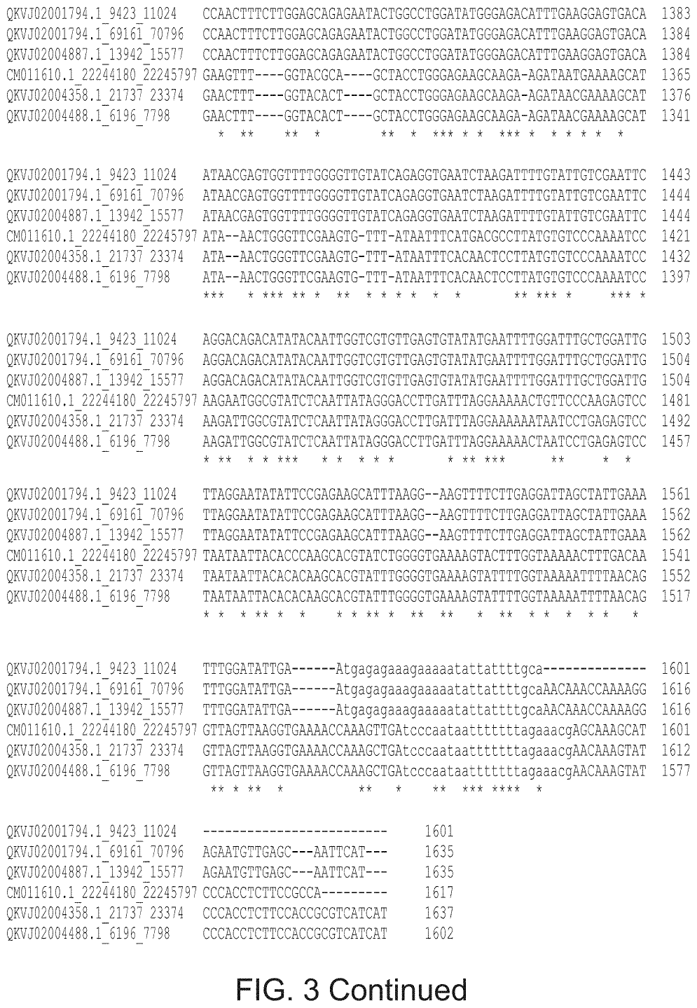

Six THCAS hits in Finola were aligned in clustal using their nucleotide sequences, FIG. 3. The alignment shows shared nucleotides are marked with a star. Whilst they do align, it is apparent that they group nicely into two groups of three. Therefore, the engineering strategy could be to target both groups individually (to study the effects on THC levels) and also to target them both together, either through guides that target all hits OR by using two guides designed for each group of hits. Therefore, three groups of guides have been designed, Table 12. QKVJ02004887.1_13942_15577 chrnan and CM011610.1_22244180_22245797 chr:6.0 were used for guide design in Benchling.

TABLE 12
THCAS hit references used for gRNA design in Finola

| | Tar- | Tar- | Tar- |
|---|---|---|---|
| | geted by | geted by | geted by |
| Hit reference used for | Group 1 | Group 2 | Group 3 |
| guide design | guides | guides | guides |
| QKVJ02001794.1_9423_11024 | Yes | Yes | No |
| chr: nan | | | |
| QKVJ02001794.1_69161_70796 | Yes | Yes | No |
| chr: nan | | | |
| QKVJ02004887.1_13942_15577 | Yes | Yes | No |
| chr: nan (used for guide design) | | | |
| CM011610.1_22244180_22245797 | Yes | No | Yes |
| chr: 6.0 (used for guide design) | | | |
| QKVJ02004358.1_21737_23374 | Yes | No | Yes |
| chr: nan | | | |
| QKVJ02004488.1_6196_7798 | Yes | No | Yes |
| chr: nan | | | |

gRNAs were designed using Benchling and the nucleotide alignments of the hits. In some instances, at least two gRNA may be selected to completely disrupt THCAS in Finola. In some instances, a gRNA from group 2 and a gRNA from group 3 may be selected.

TABLE 13
Setected gRNA binding region targeting THCAS in Finola. Off
target score from Benchling = Optimized score from Doench,
Fusi et at. (2016) optimized for 20 bp guides with NGG PAMs.
Score is from 0-100, higher is better. On target score from
Benchling = Specificity score from score is from 0-100. gRNA
sequence provided is written as 5′ to 3′ and is complementary
to the genomic sequence target.

| SEQ | | | | | On | |
|---|---|---|---|---|---|---|
| ID | | | | | Target | Off target |
| NO: | gRNA | Group | Sequence | Strand | score | score |
| 21 | FN | 1 | GGAAUAUUACAGAUAAUCAU | − | 56.8 | 94.2 |
| | THC 1 | | | | | |
| 22 | FN | 1 | UCAUCCAUUAUACCACCGUA | + | 52.6 | 98.8 |
| | THC 2 | | | | | |
| 23 | FN | 1 | AAAUUAUAUGAAGAAGAGGU | − | 54.3 | 84.4 |
| | THC 3 | | | | | |
| 24 | FN | 2 | GAUGACGCGGUGGAAGAGGU | + | — | 97.6 |
| | THC 4 | | | | | |
| 25 | FN | 2 | UCGUUUCUAAAAAAAUUAUU | + | 23.0 | 88.5 |
| | THC 5 | | | | | |
| 26 | FN | 2 | AAAUUUUAACAGGUUAGUUA | − | 35.6 | 93.8 |
| | THC 6 | | | | | |
| 27 | FN | 2 | UACACACAAGCACGUAUUUG | − | 52.5 | 99.1 |
| | THC 7 | | | | | |
| 28 | FN | 2 | CUUGGAUUUUGGGACACAUA | + | 45.6 | 89.9 |
| | THC 8 | | | | | |
| 29 | FN | 2 | GUUAUCUUCUUGCUUCUCCC | + | 49.5 | 95.0 |
| | THC 9 | | | | | |
| 30 | FN | 2 | UACAUUAUUCCAGCUCGAUG | − | 52.5 | 99.1 |
| | THC | | | | | |
| | 10 | | | | | |
| 31 | FN | 3 | UACAACACCACUGUAGAAGA | + | 53.1 | 98.3 |
| | THC | | | | | |
| | 11 | | | | | |
| 32 | FN | 3 | CAAUUUAGGAAAUUUUCUUG | − | 57.3 | 86.4 |
| | THC | | | | | |
| | 12 | | | | | |
| 33 | FN | 3 | GAAGGAGUGACAAUAACGAG | − | 66.5 | 98.5 |
| | THC | | | | | |
| | 13 | | | | | |
| 34 | FN | 3 | UUGCAGAUUCGAACUCGAAG | + | 68.6 | 98.9 |
| | THC | | | | | |
| | 14 | | | | | |

### Example 3

### Bioinformatic Analysis of THCAS in Cannabis (Purple Kush)

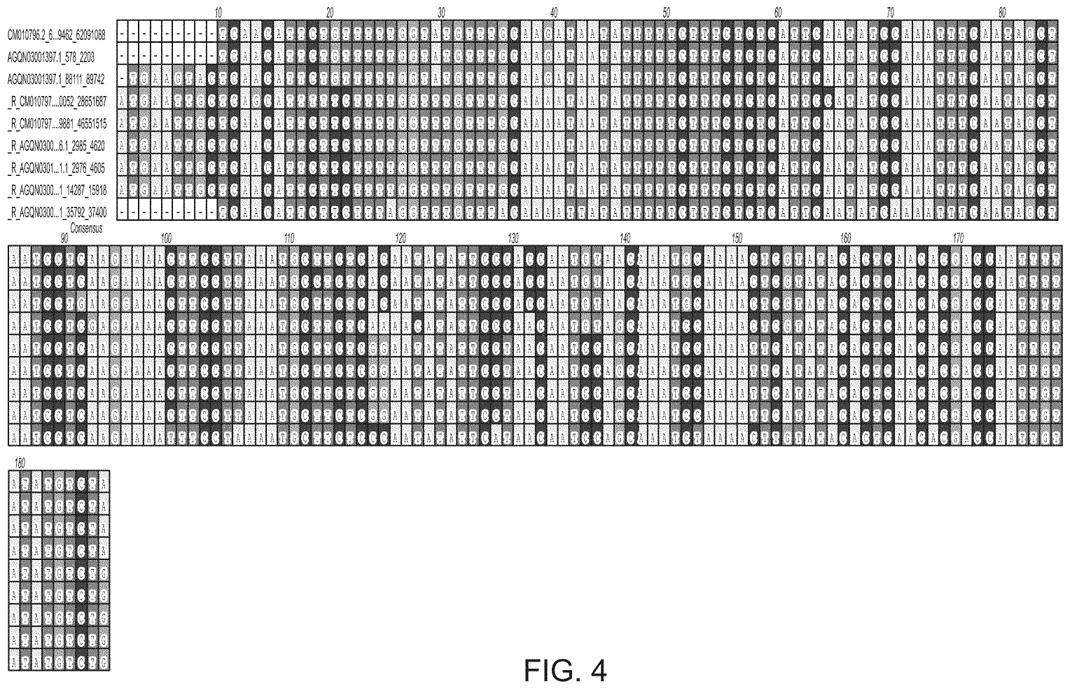

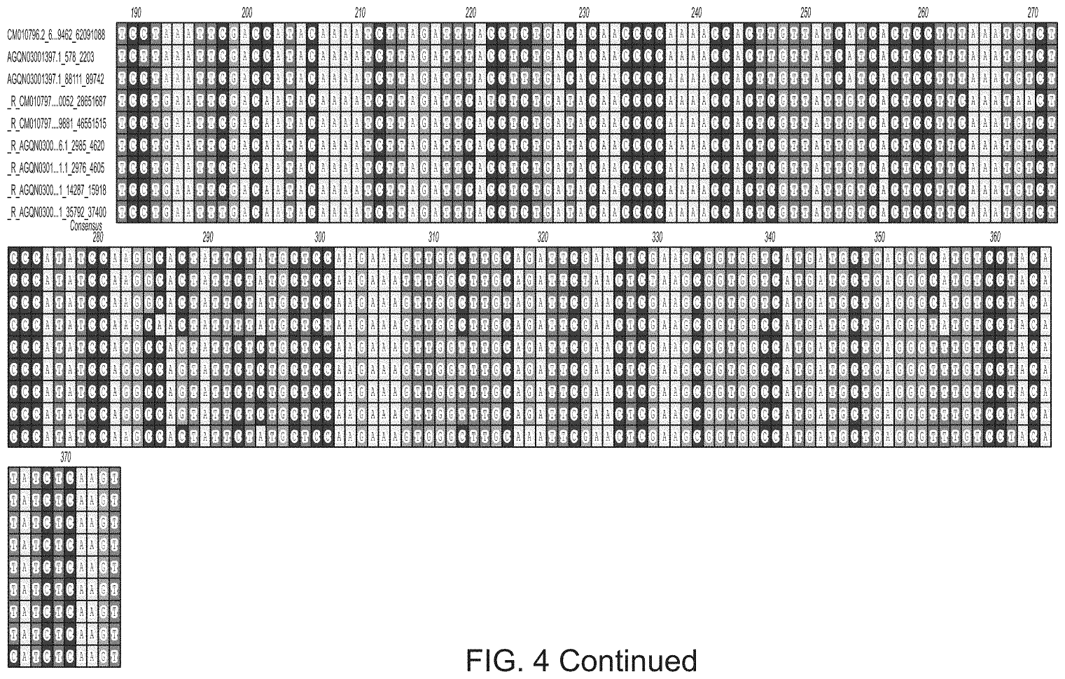

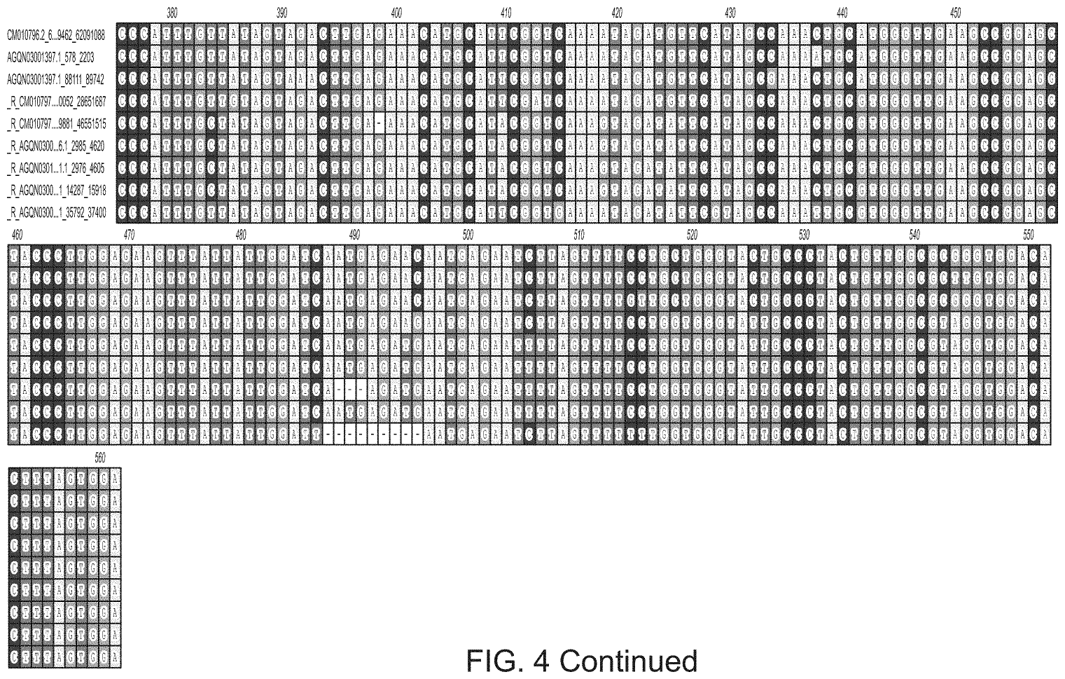

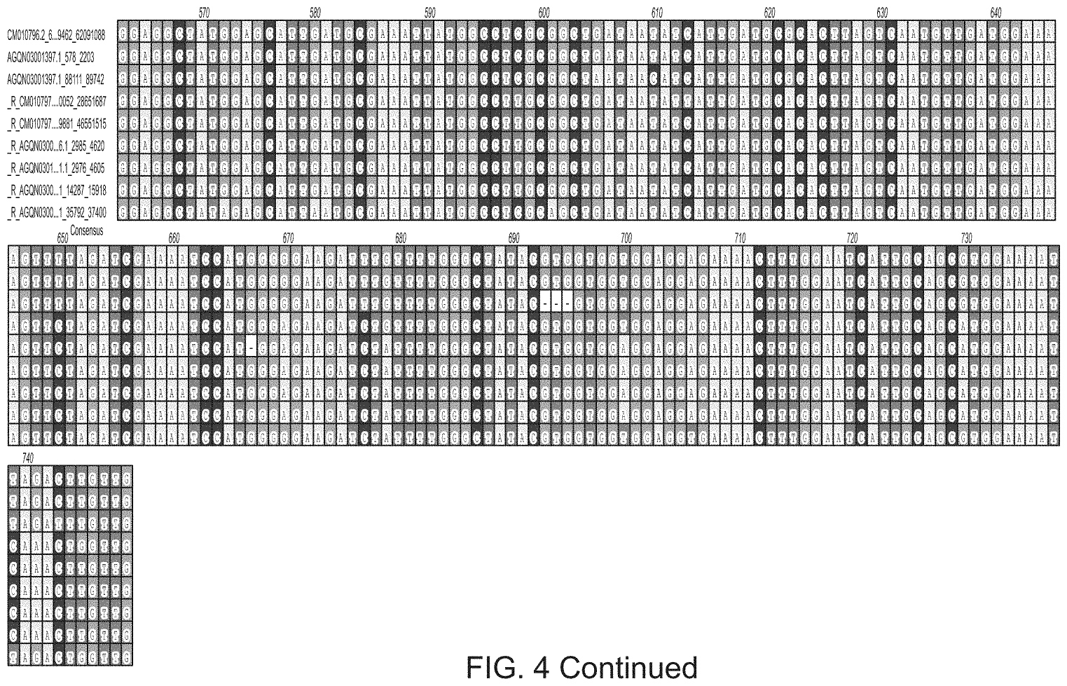

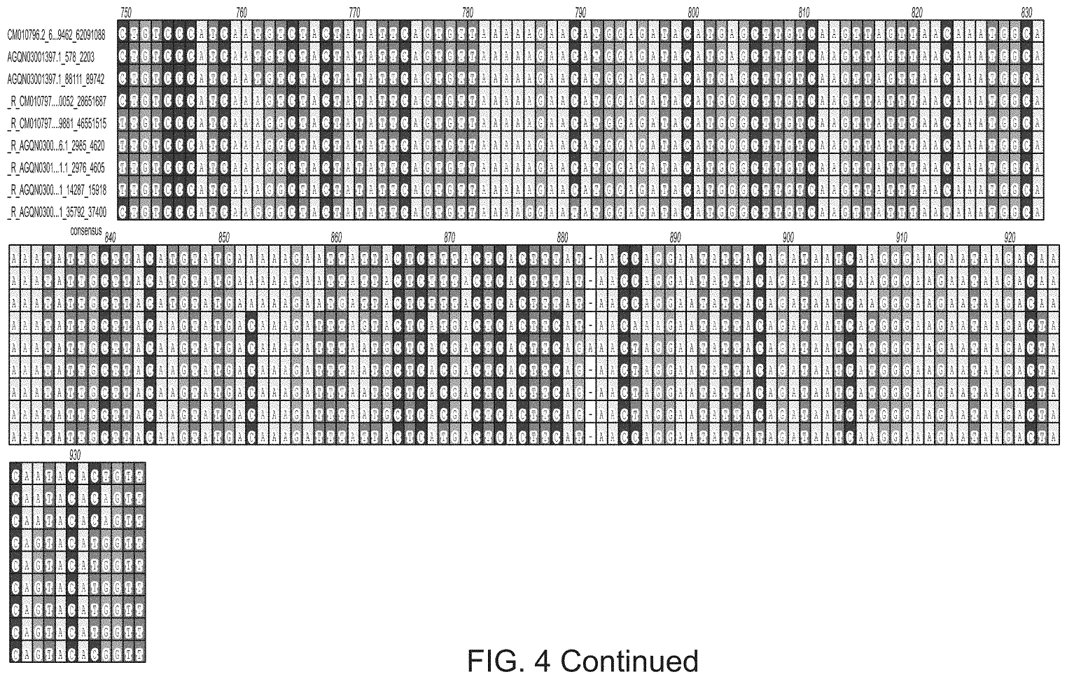

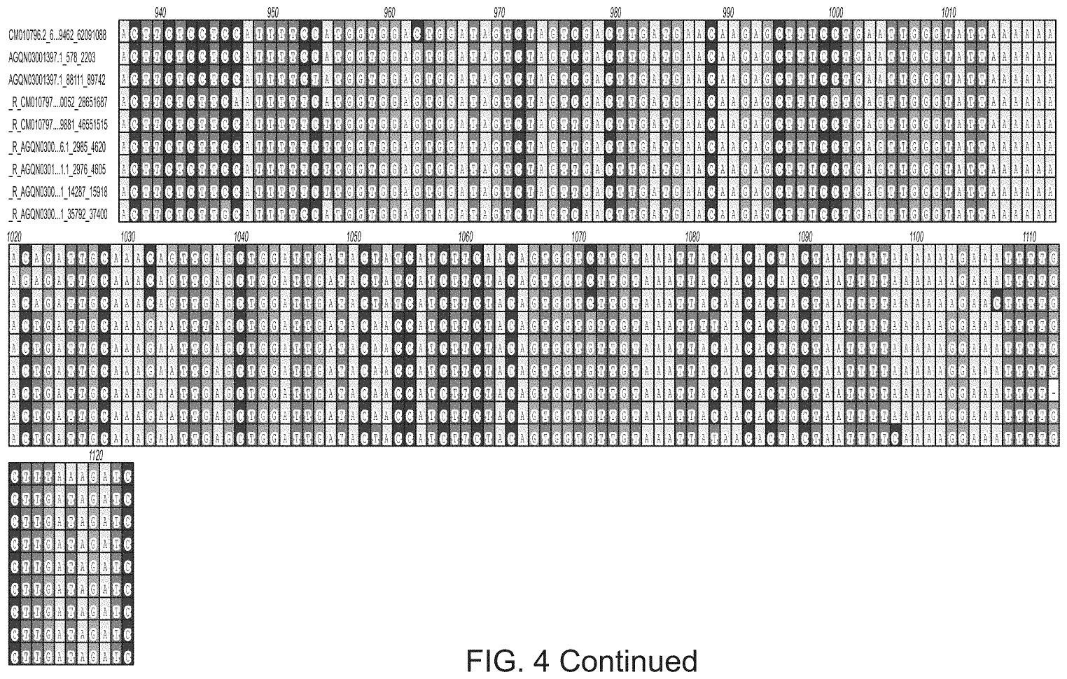

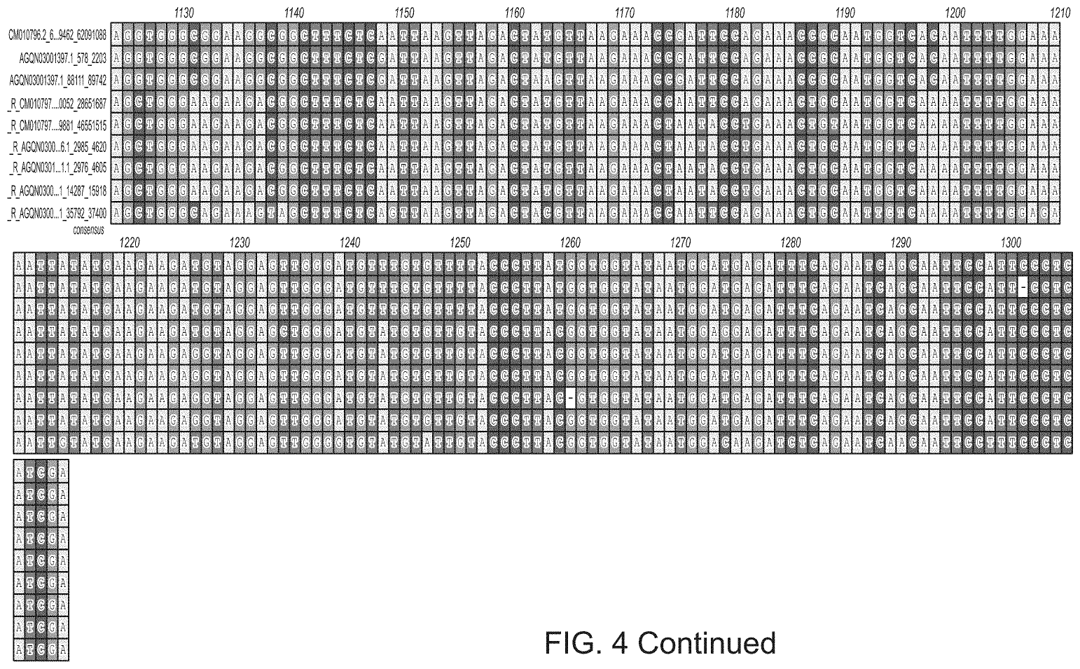

THCAS analysis in purple kush was performed to identify sequences of interest to design gRNA. Sequence alignments were performed to identify regions of interest in purple kush, Table 14 and FIG. 4.

TABLE 14
THCAS hits in purple kush (85% stringency) 4605

| | | | | | | | | BLAST | BLASTx |
|---|---|---|---|---|---|---|---|---|---|
| | | | | | | | | search of | search |
| | | | | | | | | nucleotide | (nucleotide |
| Hit | Chromosome | end | homology | length | scaffold | start | Comments | sequence | to aa search) |
| 1 | 7 | 28651687 | 99.816514 | 1635 | CM010797.2 | 28650052 | THCAS | Blast | THCAS 100% |
| | | | | | | | | hits = all | identity to |
| | | | | | | | | THCAS | AMQ48600.1 |
| 2 | NaN | 4620 | 92.844037 | 1635 | AGQN03005496.1 | 2985 | CBCAS | Blast | THCAS 99.82% |
| | | | | | | | | hits = all | identity to |
| | | | | | | | | THCAS | AJB28523.1 |
| 3 | NaN | 4605 | 92.110092 | 1629 | AGQN03010271.1 | 2976 | CBCAS | Blast | THCAS 97.35% |
| | | | | | | | | hits = all | identity to |
| | | | | | | | | THCAS | AYW35096.1 |
| 4 | 7 | 46551515 | 91.926606 | 1634 | CM010797.2 | 46549881 | pseudo | Blast | THCAS 82.86% |
| | | | | | | | CBDAS | hits all | identity to |
| | | | | | | | | THCAS | AJB28532.1 |
| 5 | NaN | 15918 | 90.458716 | 1631 | AGQN03006963.1 | 14287 | | Blast | THCAS 99.78% |
| | | | | | | | | hits = all | identity to |
| | | | | | | | | THCAS | AYW35096.1 |
| 6 | 6 | 62091088 | 88.256881 | 1626 | CM010796.2 | 62089462 | | BLAST | CBDAS 98.89% |
| | | | | | | | | search = | identity to |
| | | | | | | | | CBDAS2 (99.38% | A6P6W0.1 |
| | | | | | | | | identity to | 3^{rd} Hit = |
| | | | | | | | | AB292683.1) and | THCAS with |
| | | | | | | | | 2^{nd} hit | 88.93 |
| | | | | | | | | CBDAS3 (99.02% | identity to |
| | | | | | | | | identity to | AF124252.1 |
| | | | | | | | | AB292684.1). | STOP codon |
| | | | | | | | | Lower hits | in the middle |
| | | | | | | | | are THCAS | |
| 7 | NaN | 2203 | 87.706422 | 1625 | AGQN03001397.1 | 578 | | BLAST | CBDAS 98.37% |
| | | | | | | | | search = | identity to |
| | | | | | | | | CBDAS2 (99.26% | A6P6W0.1 |
| | | | | | | | | identity to | 3^{rd} Hit = |
| | | | | | | | | AB292683.1) and | THCAS with |
| | | | | | | | | 2^{nd} hit | 87.91% |
| | | | | | | | | CBDAS3 (99.14% | identity to |
| | | | | | | | | identity to | AF124253.1 |
| | | | | | | | | AB292684.1). | |
| | | | | | | | | Lower hits | |
| | | | | | | | | are THCAS | |
| 8 | NaN | 37400 | 86.972477 | 1608 | AGQN03001586.1 | 35792 | | BLAST | THCAS 89.42% |
| | | | | | | | | search = | identity to |
| | | | | | | | | THCAS but | AF124256.1 |
| | | | | | | | | lower down in | STOP codon |
| | | | | | | | | the hits | in the middle |
| | | | | | | | | (~92% | |
| | | | | | | | | identity) | |
| 9 | NaN | 89742 | 86.606 | 1631 | AGQN03001397.1 | 88111 | | BLAST | CBDAS 96.88 |
| | | | | | | | | search = | identity to |
| | | | | | | | | CBDAS2 (98.53% | A6P6W0.1 |
| | | | | | | | | identity to | 3^{rd} Hit = |
| | | | | | | | | AB292683.1) and | THCAS with |
| | | | | | | | | 2^{nd} hit | 86.9% |
| | | | | | | | | CBDAS3 (98.16% | identity to |
| | | | | | | | | identity to | AF124253.1 |
| | | | | | | | | AB292684.1). | 2 STOP codons |
| | | | | | | | | Lower hits | in the middle |
| | | | | | | | | are THCAS | |

THCAS hits in purple kush were translated to amino acid sequences using BlastX Amino acid sequences are shown in Table 15.

TABLE 15
Amino acid sequences of THCAS hits in purple kush identified at
85% stringency and described in Table 16.

| SEQ | | |
|---|---|---|
| ID | | |
| NO | Name | Sequence |
| 35 | >CM010797.2_28650052_28651687 | MNCSAFSFWFVCKIIFFFLSFHIQISIANPRENFLKCFSKHIPNNVANPKLVYTQH |
| | chr:7.0 | DQLYMSILNSTIQNLRFISDTTPKPLVIVTPSNNSHIQATILCSKKVGLQIRTRSG |
| | THCAS | GHDAEGMSYISQVPFVVVDLRNMHSIKIDVHSQTAWVEAGATLGEVYYWINE |
| | | KNENLSFPGGYCPTVGVGGHFSGGGYGALMRNYGLAADNIIDAHLVNVDGK |
| | | VLDRKSMGEDLFWAIRGGGGENFGIIAAWKIKLVAVPSKSTIFSVKKNMEIHG |
| | | LVKLFNKWQNIAYKYDKDLVLMTHFITKNITDNHGKNKTTVHGYFSSIFHGG |
| | | VDSLVDLMNKSFRELGIKKTDCKEFSWIDTTIFYSGVVNFNTANFKKEILLDRS |
| | | AGKKTAFSIKLDYVKKPIPETAMVKILEKLYEEDVGAGMYVLYPYGGIMEEIS |
| | | ESAIPFPHRAGIMYELWYTASWEKQEDNEKHINWVRSVYNFTTPYVSQNPRLA |
| | | YLNYRDLDLGKTNHASPNNYTQARIWGEKYFGKNFNRLVKVKTKVDPNNFF |
| | | RNEQSIPPLPPHHH |
| 36 | >AGQN03005496.1_2985_4620 | MNCSTFSFWFVCKIIFFFLSFNIQISIANPQENFLKCFSEYIPNNPANPKFIYTQHD |
| | chr:nan | QLYMSVLNSTIQNLRFTSDTTPKPLVIVTPSNVSHIQASILCSKKVGLQIRTRSG |
| | THCAS | GHDAEGLSYISQVPFAIVDLRNMHTVKVDIHSQTAWVEAGATLGEVYYWINE |
| | | MNENFSFPGGYCPTVGVGGHFSGGGYGALMRNYGLAADNIIDAHLVNVDGK |
| | | VLDRKSMGEDLFWAIRGGGGENFGHAAWKIKLVVVPSKATIFSVKKNMEIHG |
| | | LVKLFNKWQNIAYKYDKDLMLTTHFRTRNITDNHGKNKTTVHGYFSSIFLGG |
| | | VDSLVDLMNKSFPELGIKKTDCKELSWIDTTIFYSGVVNYNTANFKKEILLDRS |
| | | AGKKTAFSIKLDYVKKLIPETAMVKILEKLYEEEVGVGMYVLYPYGGIMDEIS |
| | | ESAIPFPHRAGIMYELWYTATWEKQEDNEKHINWVRSVYNFTTPYVSQNPRLA |
| | | YLNYRDLDLGKTNPESPNNYTQARIWGEKYFGKNFNRLVKVKTKADPNNFFR |
| | | NEQSIPPLPPRHH |
| 37 | >AGQN03010271.1_2976_4605 | MNCSTFSFWFVCKIIFFFLSFNIQISIANPQENFLKCFSEYIPNNPANPKFIYTQHD |
| | chr:nan | QLYMSVLNSTIQNLRFTSDTTPKPLVIVTPSNVSHIQASILCSKKVGLQIRTRSG |
| | THCAS | GHDAEGLSYISQVPFAIVDLRNMHTVKVDIHSQTAWVEAGATLGEVYYWIKM |
| | | NENFSFPGGYCPTVGVGGHFSGGGYGALMRNYGLAADNIIDAHLVNVDGKVL |
| | | DRKSMGEDLFWAIRGGGGENFGHAAWKIKLVVVPSKATIFSVKKNMEIHGLV |
| | | KLFNKWQNIAYKYDKDLMLTTHFRTRNITDNHGKNKTTVHGYFSSIFLGGVD |
| | | SLVDLMNKSFPELGIKKTDCKELSWIDTTIFYSGVVNYNTANFKKEIFLIDQLG |
| | | RR |
| 38 | >CM010797.2_46549881_46551515 | PICYSRLENMHTVKVDIHSQTAWVEAGATLGEVYYWINEMNENFSFPGGYCP |
| | chr:7.0 | TVGVGGHFSGGGYGALMRNYGLAADNIIDAHLVNVDGKVLDRKSMEKIYFG |
| | THCAS | LYVVEEEKTLESLQHGKSNLLLSHQRLLYSVLKRTWRYMGLSSYLTNGKILLT |
| | | SMTKI*CSRLTSETRNITDNHGKNKTTVHGYFSSIFLGGVDSLVDLMNKSFPEL |
| | | GIKKTDCKELSWIDTTIFYSGVVNYNTANFKKEILLDRSAGKKTAFSIKLDYVK |
| | | KLIPETVMVKILEKLYEEEVGVGMYVLYPYGGIMDEISESAIPFPHRAGIMYEL |
| | | WYTATWEKQEDNEKHINWVRSVYNFTTPYVSQNPRLAYLNYRDLDLGKTNP |
| | | ESPNNYTQARIWGEKYFGKNFNRLVKVKTKADPNNFFRNEQSIPPLPPRHH |
| 39 | >AGQN03006963.1_14287_15918 | MNCSTFSFWFVCKIIFFFLSFNIQISIANPQENFLKCFSEYIPNNPANPKFIYTQHD |
| | chr:nan | QLYMSVLNSTIQNLRFTSDTTPKPLVIVTPSNVSHIQASILCSKKVGLQIRTRSG |
| | THCAS | GHDAEGLSYISQVPFAIVDLRNMHTVKVDIHSQTAWVEAGATLGEVYYWINE |
| | | MNENFSFPGGYCPTVGVGGHFSGGGYGALMRNYGLAADNIIDAHLVNVDGK |
| | | VLDRKSMGEDLFWAIRGGGGENFGHAAWKIKLVVVPSKATIFSVKKNMEIHG |
| | | LVKLFNKWQNIAYKYDKDLMLTTHFRTRNITDNHGKNKTTVHGYFSSIFLGG |
| | | VDSLVDLMNKSFPELGIKKTDCKELSWIDTTIFYSGVVNYNTANFKKEILLDRS |
| | | AGKKTAFSIKLDYVKKLIPETAMVKILEKLYEEEVGVGMYVLYPYGGIMDEIS |
| | | ESAIPFPHRAGIMYELWYTATWE |
| 40 | >CM010796.2_62089462_62091088 | STFCFWYVCKIIFFFLSFNIQISIANPQENFLKCFSQYIPTNVTNAKLVYTQHDQF |
| | chr:6.0 | YMSILNSTIQNLRFTSDTTPKPLVIITPLNVSHIQGTILCSKKVGLQIRTRSGGHD |
| | CBDAS | AEGMSYISQVPFVIVDLRNMHSVKIDVHSQTAWVEAGATLGEVYYWINENNE |
| | | NLSFPAGYCPTVGAGGHFSGGGYGALMRNYGLAADNIIDAHLVNVDGKVLD |
| | | RKSMGEDLFWAIRGGGGENFGHAAWKIRLVAVPSMSTIFSVKKNMEIHELVK |
| | | LVNKWQNIAYMYEKELLLFTHFITRNITDNQGKNKTTIHCYFSSIFHGGLDSLV |
| | | DLMNKSFPELGIKKTDCKQLSWIDTHFNSGLVNYNTTNFKKEILL*RSGGRKA |
| | | AFSIKLDYVKKPIPETAMVTILEKLYEEDVGVGMFVFYPYGGIMDEISESAIPFP |
| | | HRAGIMYEIWYIASWEKQEDNEKHINWIRNVYNFTTPYVSQNPRMAYLNYRD |
| | | LDLGKTNFESPNNYTQARIWGEKYFGKNFNRLVKVKTKVDPDNFFRNEQSIPP |
| | | LPLRHH |
| 41 | >AGQN03001397.1_578_2203 | STFCFWYVCKIIFFFLSFNIQISIANPQENFLKCLSQYIPTNVTNAKLVYTQHDQF |
| | chr:nan | YMSILNSTIQNLRFTSDTTPKPLVIITPLNVSHIQGTILCSKKFGLQIRTRSGGHD |
| | THCAS | AEGMSYISQVPFVIVDLRNMHSVKIDVHSQNAWVEAGATLGEVYYWINENNE |
| | | NLSFPAGYCPTVGACGHFSGGGYGALMRNYGLAADNIIDAHLVNVDGKVLDR |
| | | KSMGEDLFWAIRGGGGENFGHAAWKIRLVAVPSMSTIFSVKKNMEIHELVKL |
| | | VNKWQNIAYMYEKELLLFTHFITRNITDNQGKNKTTIHSYFSSIFHGGVDSLVD |
| | | LMNKSFPELGIKKRDCKQLSWIDTIIFYSGLVNYNTTNFKKEILLDRSGGRKAA |
| | | FSIKLDYVKKPIPETAMVTILEKLYEEDVGVGMFVFYPYGGIMDEISESAIPF |
| 42 | >AGQN03001586.1_35792_37400 | STFSFRFVYKIIFFFLSFNIKISIANPQENFLKCFSQYIHNNPANLKLVYTQHDQL |
| | chr:nan | YMSVLNLTIQNLRFTSDTTPKPLVIVTPSNVSHIQATILCSKKVGLQIRTRSGGH |
| | THCAS | DAEGLSYTSQVPFVIVDLRNMHSVKIDIRSQIAWVEAGATLGEVYYWINENLS |
| | | FPGGYCPTVGVGGHFSGGGYRALMRNYGLAADNIIDAHLVNVDGKVLDRKS |
| | | MGEDLFWAIRGGGGENFGHAAWKIRLVAVPSRATIFSVKRNMEIHGLVKLFN |
| | | KWQNIAYKYDKDLLLMTHFITRNIIDNQGKNKTTVHGYFSCIFHGGVDSLVNL |
| | | MNKSFPELGIKKTDCKELSWIDTTIFYSGVVNYNTTNFQKEILLDRSAGQKVAF |
| | | SVKLDYVKKPIPETAIVKILEKLYEEDVGVGVYVLYPYGGIMDKISESTIPFPHR |
| | | AGIMYEV*YAATWEKQEDNEKHINWV*SVYNFMTPYVSQNPRMAYLNYRDL |
| | | DLGKTDPKSPNNYTQARIWGEKYFGKNFDKLVKVKTKVDPNNFFRNEQSIPPL |
| | | PP |
| 43 | >AGQN03001397.1_881112_89742 | KYSTFCFWYVCKIIFFFLSFNIQISIANPEGNFLKCFSQYIPTNVTNAKLVYTQHD |
| | chr:nan | QFYMSILNSTIQNLRFTFDTTPKPLVIITPLNVSHIQGTILCSKKVGL*IRTRSGGH |
| | CBDAS | DAEGMSYISQVPFVIVNLRNMHSVKIDVHSETAWVEAGATLGEVYYWINENN |
| | | ENLSFLAGYCPTVGAGGHFSGGGYGALMRNYGLAANNIIDAHLVNVDGKVL |
| | | DRKSMGEDLFWAIRGGGENFGHAAWKIRFVAVPSMSTIFSVKKNMEIHELVKL |
| | | VNKWQNIAYMYEKE*LLFTHFITRNITDNQGKNKTTIHSYFSSIFYGGVDSLVD |
| | | LMNKSFPELGIKKTDCKQLSWIDTIIFYSGLVNYNTTNFKKELLLDRSGGRKAA |
| | | FSIKLD*VKKPIPETAMVTILEKLYEEDVGVGMFVFYPYGGIMDEISESAIPFPH |
| | | RAGIMYEIWYIASWEKQEDNEKHINWIRNVYNFTTPYVSQNPRMAYLNYRDL |
| | | DLGKTNFESPNNYTQARIWGEKYFGKNFNRLVKVKTKVDPDNFFRNEQSIPPL |
| | | PLRHH |

### Example 4

### Bioinformatic Analysis of CBDAS in Finola

CBDAS analysis in finola was performed to identify sequences of interest to design gRNA. Sequence alignments were performed to identify regions of interest in purple kush, Table 16 and FIG. 5.

TABLE 16
CBDAS in Finola (85% stringency)

| | | | | | | | BLAST | BLASTx |
|---|---|---|---|---|---|---|---|---|
| | | | | | | | search of | search |
| | | | | | | | nucleotide | (nucleotide |
| Hit | Chromosome | end | homology | length | scaffold | start | sequence | to aa search) |
| 1 | 6 | 21838669 | 99.033 | 1550 | CM011610.1 | 21837119 | CBDAS | CBDAS 99.81% |
| | | | | | | | (BLAST | identity to |
| | | | | | | | accession | AJB28530.1 |
| | | | | | | | KJ469374.1) | |
| 2 | NaN | 652403 | 85.161 | 1394 | QKVJ02000019.1 | 651009 | BLAST | CBDAS 1 99.81% |
| | | | | | | | search = | identity to |
| | | | | | | | CBDAS2 (99.78% | A6P6W0.1 |
| | | | | | | | identity to | |
| | | | | | | | AB292683.1) | |
| | | | | | | | and 2^{nd}hit | |
| | | | | | | | CBDAS3 (99.35% | |
| | | | | | | | identity to | |
| | | | | | | | AB292684.1) | |

CBDAS hits in finola were translated to amino acid sequences using BlastX Amino acid sequences are shown in Table 17.

TABLE 17
Amino acid sequences of CBDAS hits in Finola identified at 85% stringency and
described in Table 16.

| SEQ | | |
|---|---|---|
| ID | | |
| NO | Name | Sequence |
| 44 | >CM011610.1_21837119_21838669 | NPRENFLKCFSQYIPNNATNLKLVYTQNNPLYMSVLNSTIHNLRFSSDTTPKPL |
| | chr:6.0 | VIVTPSHVSHIQGTILCSKKVGLQIRTRSGGHDSEGMSYISQVPFVIVDLRNMRS |
| | CBDAS | IKIDVHSQTAWVEAGATLGEVYYWVNEKNESLSLAAGYCPTVCAGGHFGGG |
| | | GYGPLMRSYGLAADNIIDAHLVNVHGKVLDRKSMGEDLFWALRGGGAESFGI |
| | | IVAWKIRLVAVPKSTMFSVKKIMEIHELVKLVNKWQNIAYKYDKDLLLMTHFI |
| | | TRNITDNQGKNKTAIHTYFSSVFLGGVDSLVDLMNKSFPELGIKKTDCRQLSWI |
| | | DTIIFYSGVVNYDTDNFNKEILLDRSAGQNGAFKIKLDYVKKPIPESVFVQILEK |
| | | LYEEDIGAGMYALYPYGGIMDEISESAIPFPHRAGILYELWYICSWEKQEDNEK |
| | | HLNWIRNIYNFMTPYVSQNPRLAYLNYRDLDIGINDPKNPNNYTQARIWGEKY |
| | | FGKNFDRLVKVKTLVDPNNFFRNEQSIPPLPRHHH |
| 45 | >QKVJ02000019.1_651009_652403 | NPQENFLKCFSQYIPTNVTNAKLVYTQHDQFYMSILNSTIQNLRFTSETTPKPLV |
| | chr:nan | IITPLNVSHIQGTILCSKKVGLQIRTRSGGHDAEGMSYISQVPFVIVDLRNMHSV |
| | CBDS1 | KIDVHSQTAWVEAGATLGEVYYWINENNENLSFPAGYCPTVGAGGHFSGGGY |
| | | GALMRNYGLAADNIIDAHLVNVDGKVLDRKSMGEDLFWAIRGGGGENFGHA |
| | | AWKIRLVAVPSMSTIFSVKKNMEIHELVKLVNKWQNIAYMYEKELLLFTHFIT |
| | | RNITDNQGKNKTTIHSYFSSIFHGGVDSLVDLMNKSFPELGIKKTDCKQLSWID |
| | | TIIFYSGVVNYNTTNFKKEILLDRSGGRKAAFSIKLDYVKKPIPETAMVTILEKL |
| | | YEEDVGVGMFVFYPYGGIMDEISESAIPFPHRAGIMYEIWYIASWEKQEDNEK |
| | | HINWIRNVYNFTTPYVSQNPRMAYLNYRDLDLGK |

Hits from the THCAS search that were annotated as CBDAS are shown in Table 18.

TABLE 18
CBDAS hits identified during THCAS search

| | | | | | | | BLAST | BLASTx |
|---|---|---|---|---|---|---|---|---|
| | | | | | | | search of | search |
| Hit | | | | | | | nucleotide | (nucleotide |
| number | chromosome | end | homology | length | scaffold | start | sequence | to aa search) |
| 7 | NaN | 711394 | 89.979 | 1406 | QKVJ02000019.1 | 709988 | BLAST | CBDAS1 99.36% |
| | | | | | | | search = | identity with |
| | | | | | | | CBDAS2 (first | A6P6W0.1 |
| | | | | | | | hit at 99.79% | |
| | | | | | | | identity), | |
| | | | | | | | CBDAS3 (second | |
| | | | | | | | hit at 99.5% | |
| | | | | | | | identity) | |
| | | | | | | | Missing ~220 | |
| | | | | | | | bp from the | |
| | | | | | | | start, no | |
| | | | | | | | start codon | |
| 9 | NaN | 652400 | 88.798 | 1472 | QKVJ02000019.1 | 650928 | BLAST | CBDAS 1 99.39% |
| | | | | | | | search = | identity with |
| | | | | | | | CBDAS2 (first | A6P6W0.1 |
| | | | | | | | hit at 99.73% | |
| | | | | | | | identity), | |
| | | | | | | | CBDAS3 (second | |
| | | | | | | | hit at 99.32% | |
| | | | | | | | identity), | |
| | | | | | | | third hit is | |
| | | | | | | | THCAS ~150 | |
| | | | | | | | bp shorter | |
| | | | | | | | and without | |
| | | | | | | | a stop codon | |
| 10 | NaN | 652563 | 88.623853 | 1627 | QKVJ02000019.1 | 650936 | BLAST | CBDAS 1 98.98% |
| | | | | | | | search = | identity with |
| | | | | | | | CBDAS2 (first | A6P6W0.1 |
| | | | | | | | hit at 99.75% | |
| | | | | | | | identity), | |
| | | | | | | | CBDAS3 (second | |
| | | | | | | | hit at 99.32% | |
| | | | | | | | identity), | |
| | | | | | | | third hit | |
| | | | | | | | is THCAS | |
| 11 | NaN | 537191 | 87.522936 | 1620 | QKVJ02000019.1 | 535571 | BLAST | CBDAS1 100% |
| | | | | | | | search = | identity with |
| | | | | | | | CBDAS2 (first | A6P6W0.1 |
| | | | | | | | hit at 99.51% | |
| | | | | | | | identity). | |
| | | | | | | | CBDAS3 (second | |
| | | | | | | | hit at 99.14% | |
| | | | | | | | identity), | |
| | | | | | | | third hit is | |
| | | | | | | | THCAS (92.3% | |
| | | | | | | | identity) | |

CBDAS hits were translated to amino acid sequences using BlastX Amino acid sequences are shown in Table 19.

TABLE 19
CBDAS amino acid sequences transtated directly from the nucleotide sequences
described in Table 20.

| SEQ | | |
|---|---|---|
| ID | | |
| NO | Name | Sequence |
| 46 | >CM011610.1_21836537_21839169 | MKYSTFSFWFVCKIIFFFFSFNIQTSIANPRENFLKCFSQYIPNNATNLKLVYTQN |
| | chr:6.0 | NPLYMSVLNSTIHNLRFSSDTTPKPLVIVTPSHVSHIQGTILCSKKVGLQIRTRSG |
| | CBDAS | GHDSEGMSYISQVPFVIVDLRNMRSIKIDVHSQTAWVEAGATLGEVYYWVNE |
| | (21837119) | KNESLSLAAGYCPTVCAGGHFGGGGYGPLMRSYGLAADNIIDAHLVNVHGKV |
| | | LDRKSMGEDLFWALRGGGAESFGIIVAWKIRLVAVPKSTMFSVKKIMEIHELV |
| | | KLVNKWQNIAYKYDKDLLLMTHFITRNITDNQGKNKTAIHTYFSSVFLGGVDS |
| | | LVDLMNKSFPELGIKKTDCRQLSWIDTIIFYSGVVNYDTDNFNKEILLDRSAGQ |
| | | NGAFKIKLDYVKKPIPESVFVQILEKLYEEDIGAGMYALYPYGGIMDEISESAIP |
| | | FPHRAGILYELWYICSWEKQEDNEKHLNWIRNIYNFMTPYVSQNPRLAYLNYR |
| | | DLDIGINDPKNPNNYTQARIWGEKYFGKNFDRLVKVKTLVDPNNFFRNEQSIPP |
| | | LPRHHH* |
| 47 | >QKVJ02000019.1_535062_537679 | MKYSTFCFWYVCKIIFSFSHSISKFQ*LILKKT*MLLTIYSHQCNKCKTRIHSTRPI |
| | chr:nan | LYVYPKFDHTKS*IYL*HNPKTTCYHHSFKCLPYPRHYSMLQESWLADSNSKR |
| | CBDAS | WS*C*GHVLHISSPICYSRLEKHAFGQNRCS*PNCMG*SRSYPWRSLLLDQ*EQ* |
| | (535571) | ES*FSCWVLPYCWRGWTL*WRRLWSIDAKLWPRG**YH*CALSQC*WKSFRS |
| | | KIHGGRFVLGYTWWWRRKLWNHCSVEN*TCCCPINVYYIQC*KEHGDT*ACQ |
| | | VS*QMAKYCLHV*KRIITLYSLYNQEYYR*SREE*DNNTQLLLLIFHGGVDSLV |
| | | DLMNKSFPELGIKKTDCKQLSWIDTIIFYSGVVNYNTTNFKKEILLDRSGGRKA |
| | | AFSIKLDYVKKPIPETAMVTILEKLYEEDVGVGMFVFYPYGGIMDEISESAIPFP |
| | | HRAGIMYEIWYIASWEKQEDNEKHINWIRNVYNFTTPYVSQNPRMAYLNYRD |
| | | LDLGKTNFESPNNYTQARIWGEKYFGKNFNRLVKVKTKVDPDNFFRNEQSIPP |
| | | LPLRHH* |
| 48 | >QKVJ02000019.1_650427_653051 | MKYSTFCFWYVCKIIFFFLSFNIQISIANPQENFLKCFSQYIPTNVTNAKLVYTQ |
| | chr:nan | HDQFYMSILNSTIQNLRFTSETTPKPLVIITPLNVSHIQGTILCSKKVGLQIRTRSG |
| | CBDAS | GHDAEGMSYISQVPFVIVDLRNMHSVKIDVHSQTAWVEAGATLGEVYYWINE |
| | (650928) | NNENLSFPAGYCPTVGAGGHFSGGGYGALMRNYGLAADNIIDAHLVNVDGK |
| | | VLDRKSMGEDLFWAIRGGGGENFGIIAAWKIRLVAVPSMSTIFSVKKNMEIHE |
| | | LVKLVNKWQNIAYMYEKELLLFTHFITRNITDNQGKNKTTIHSYFSSIFHGGVD |
| | | SLVDLMNKSFPELGIKKTDCKQLSWIDTIIFYSGVVNYNTTNFKKEILLDRSGG |
| | | RKAAFSIKLDYVKKPIPETAMVTILEKLYEEDVGVGMFVFYPYGGIMDEISESA |
| | | IPFPHRAGIMYEIWYIASWEKQEDNEKHINWIRNVYNFTTPYVSQNPRMAYLN |
| | | YRDLDLGKN*FRES**LHTSTYLG*KVFW*KF**VSKSKNQG*SR*FL*KRTKHP |
| | | TSSPASSL |
| 49 | >QKVJ02000019.1_650509_652903 | MKYSTFCFWYVCKIIFFFLSFNIQISIANPQENFLKCFSQYIPTNVTNAKLVYTQ |
| | chr:nan | HDQFYMSILNSTIQNLRFTSETTPKPLVIITPLNVSHIQGTILCSKKVGLQIRTRSG |
| | CBDAS | GHDAEGMSYISQVPFVIVDLRNMHSVKIDVHSQTAWVEAGATLGEVYYWINE |
| | (651009) | NNENLSFPAGYCPTVGAGGHFSGGGYGALMRNYGLAADNIIDAHLVNVDGK |
| | | VLDRKSMGEDLFWAIRGGGGENFGIIAAWKIRLVAVPSMSTIFSVKKNMEIHE |
| | | LVKLVNKWQNIAYMYEKELLLFTHFITRNITDNQGKNKTTIHSYFSSIFHGGVD |
| | | SLVDLMNKSFPELGIKKTDCKQLSWIDTIIFYSGVVNYNTTNFKKEILLDRSGG |
| | | RKAAFSIKLDYVKKPIPETAMVTILEKLYEEDVGVGMFVFYPYGGIMDEISESA |
| | | IPFPHRAGIMYEIWYIASWEKQEDNEKHINWIRNVYNFTTPYVSQNPRMAYLN |
| | | YRDLDLGKN*FRES**LHTSTYLG*KVFW*KF**VSKSKNQG*SR*FL*KRTKHP |
| | | TSSPASSL |
| 50 | >QKVJ02000019.1_709260_711882 | MKYSTFCFWYVCKIIFFFLSFNIQISIANPQENFLKCFSQYIPTNVTNAKLVYTQ |
| | chr:nan | HDQFYMSILNSTIQNLRFTSEQPQNHLLSSLL*MSPISKALFYAPRKLACRFELE |
| | | AVVMMLRACPTYLKSHLL**T*ETCIRSK*MFIAKLHGLKPELPLEKFIIGSMRT |
| | | MRILVFLLGTALLLARVDTLVEEAMEH*CEIMASRLIISLMRT*SMLMEKF*IEN |
| | | PWGKICFGLYVVVEEKTLESLQRGKLDLMLSHQCLLYSVLKRTWRYMSLSS*L |
| | | TNGKILLTCMKKNYYSLLTL*PGILQIIKGRIRQQYTVTSPPFSMVEWIV*ST**T |
| | | RAFLNWVLKKQIANS*AGLILSSSTVVL*ITTQLILKKKFCLIDQVGGRRLSRLS* |
| | | TMLRNRFQKPQWSQFWKNYMKKM*ELGCLCFTLMVV*WMRFQNQQFHSLIE |
| | | LESCMKFGT*LHGRSKKIMKSI*TGFGMFIISRLLMCPKIQEWRISIIGTLI*EKLIS |
| | | RVLIITHKHVFGVKSILVKILIG**K*KPRLITIISLETNKASHLFPCVII |
| 51 | >QKVJ02000019.1_709488_711894 | MKYSTFCFWYVCKIIFFFLSFNIQISIANPQENFLKCFSQYIPTNVTNAKLVYTQ |
| | chr:nan | HDQFYMSILNSTIQNLRFTSEQPQNHLLSSLL*MSPISKALFYAPRKLACRFELE |
| | CBDAS | AVVMMLRACPTYLKSHLL**T*ETCIRSK*MFIAKLHGLKPELPLEKFIIGSMRT |
| | (709988) | MRILVFLLGTALLLARVDTLVEEAMEH*CEIMASRLIISLMRT*SMLMEKF*IEN |
| | | PWGKICFGLYVVVEEKTLESLQRGKLDLMLSHQCLLYSVLKRTWRYMSLSS*L |
| | | TNGKILLTCMKKNYYSLLTL*PGILQIIKGRIRQQYTVTSPPFSMVEWIV***T |
| | | RAFLNWVLKKQIANS*AGLILSSSTVVL*ITTQLILKKKFCLIDQVGGRRLSRLS* |
| | | TMLRNRFQKPQWSQFWKNYMKKM*ELGCLCFTLMVV*WMRFQNQQFHSLIE |
| | | LESCMKFGT*LHGRSKKIMKSI*TGFGMFIISRLLMCPKIQEWRISIIGTLI*EKLIS |
| | | RVLIITHKHVFGVKSILVKILIG**K*KPRLITIISLETNKASHLFPCVII |

### Example 5

### Bioinformatic Analysis of CBDAS in Purple Kush

CBDAS analysis in purple kush was performed to identify sequences of interest to design gRNA. Sequence alignments were performed to identify regions of interest in purple kush, Table 20 and FIG. 6.

TABLE 20
CBDAS in purple kush (using 80% stringency)

| | | | | | | | BLAST | BLASTx |
|---|---|---|---|---|---|---|---|---|
| | | | | | | | search of | search |
| Hit | | | | | | | nucleotide | (nucleotide |
| number | chromosome | end | homology | length | scaffold | start | sequence | to aa search) |
| 1 | 2 | 58202370 | 90.257353 | 1631 | CM010792.2 | 58200739 | CBDAS | CBDAS 91.34% |
| | | | | | | | (CBDA3 top | identity with |
| | | | | | | | hit Accession | AYW35112.1 |
| | | | | | | | KJ469376.1, | |
| | | | | | | | 99.63% | |
| | | | | | | | identity) ~ | |
| | | | | | | | top 30 | |
| | | | | | | | named hits | |
| | | | | | | | are CBDAS | |
| 2 | 2 | 58109265 | 86.213235 | 1622 | CM010792.2 | 58107643 | CBDAS | CBDAS 69.69% |
| | | | | | | | (CBDA2 top | identity with |
| | | | | | | | hit Accession | AKC34414.1 |
| | | | | | | | KJ469375.1, | |
| | | | | | | | 98.8% | |
| | | | | | | | identity, | |
| | | | | | | | second hit | |
| | | | | | | | CBDA3 | |
| | | | | | | | Accession | |
| | | | | | | | KJ469376.1, | |
| | | | | | | | 6^{th} hit | |
| | | | | | | | CBDA1 | |
| | | | | | | | Accession | |
| | | | | | | | KJ469374.1) | |
| 3 | 6 | 62091076 | 83.823529 | 1623 | CM010796.2 | 62089453 | CBDAS | CBDAS 98.71% |
| | | | | | | | (CBDAS2 top | identity with |
| | | | | | | | hit at 99.32% | A6P6W0.1 |
| | | | | | | | Accession | |
| | | | | | | | AB292683.1, | |
| | | | | | | | CBDAS3 | |
| | | | | | | | second hit | |
| | | | | | | | at 98.95% | |
| | | | | | | | identity | |
| | | | | | | | Accession | |
| | | | | | | | AB292684.1) | |
| 4 | 7 | 28651687 | 83.823529 | 1635 | CM010797.2 | 28650052 | (Appeared in | THCAS 100% |
| | | | | | | | the THCAS | identity with |
| | | | | | | | search as | AMQ48600.1 |
| | | | | | | | top hit) | |
| 5 | NaN | 2191 | 83.272059 | 1622 | AGQN03001397.1 | 569 | BLAST | CBDAS |
| | | | | | | | search = | 97.69% |
| | | | | | | | CBDAS2 (99.2% | identity with |
| | | | | | | | identity to | A6P6W1.1 |
| | | | | | | | AB292683.1) | |
| | | | | | | | and 2^{nd} hit | |
| | | | | | | | CBDAS3 (99.08% | |
| | | | | | | | identity to | |
| | | | | | | | AB292684.1), | |
| | | | | | | | Lower hits | |
| | | | | | | | are THCAS | |
| 6 | NaN | 89742 | 82.625 | 1550 | AGQN03001397.1 | 88192 | BLAST | CBDAS 96.91% |
| | | | | | | | search = | identity with |
| | | | | | | | CBDAS2 (99.52% | A6P6W0.1 |
| | | | | | | | identity to | |
| | | | | | | | AB292683.1) | |
| | | | | | | | and 2^{nd} hit | |
| | | | | | | | CBDAS3 (99.13% | |
| | | | | | | | identity to | |
| | | | | | | | AB292684.1), | |
| | | | | | | | Lower hits | |
| | | | | | | | are THCAS | |
| 7 | NaN | 4620 | 82.169118 | 1623 | AGQN03005496.1 | 2997 | THCAS (top | THCAS 100% |
| | | | | | | | hit Accession | identity with |
| | | | | | | | MG996405.1 | AYW35091.1 |
| | | | | | | | and all hits | |
| | | | | | | | THCAS) | |
| 8 | 7 | 46551515 | 81.433824 | 1622 | CM010797.2 | 46549893 | THCAS (top | THCAS 82.69% |
| | | | | | | | hit Accession | identity with |
| | | | | | | | MG996405.1 | AMQ46804.1 |
| | | | | | | | and all hits | |
| | | | | | | | THCAS) | |
| 9 | NaN | 4605 | 81.25 | 1617 | AGQN03010271.1 | 2988 | THCAS (top | THCAS 97.35% |
| | | | | | | | hit Accession | identity with |
| | | | | | | | MG996405.1 | AYW35096.1 |
| | | | | | | | and all hits | |
| | | | | | | | THCAS) | |
| 10 | NaN | 37400 | 81.066176 | 1617 | AGQN03001586.1 | 35783 | THCAS (later | THCAS 89.11% |
| | | | | | | | down in the | identity with |
| | | | | | | | hits, no | AF124256.1 |
| | | | | | | | annotated | |
| | | | | | | | top hits) | |
| 11 | NaN | 15918 | 80.514706 | 1619 | AGQN03006963.1 | 14299 | THCAS (top | THCAS 99.78% |
| | | | | | | | hit Accession | identity with |
| | | | | | | | MG996405.1 | AYW35096.1 |
| | | | | | | | and all hits | |
| | | | | | | | THCAS) | |

CBDAS hits in purple kush were translated to amino acid sequences using BlastX Amino acid sequences are shown in Table 21.

TABLE 21
CBDAS amino acid sequences translated directly from the nucleotide sequences of
purple kush. Sequences described in Table 21.

| SEQ | | |
|---|---|---|
| ID | | |
| NO | Name | Sequence |
| 52 | >CM010792.2_58200739_58202370 | SKKIGLQIRTRSGGHDSEDMSYISQVPFVIVDLRNMHSINIDVHSQIARVEAGAT |
| | chr:2.0 | LGEVYYWVNEKNENLSLAAGYCPTVSAAGHFGGGGYGPLMQNYGLAADNIV |
| | CBDAS | DAHLVNVDAKVLDRKSMGEDLFWAIRGGGGESFGIIVAWKIRLVAVPTKSTM |
| | | FSVKKIMEIHELVK*VNKWQNIAYKYDKDLLLMTHFITRNITNNHGKNKTTIH |
| | | TYFSSVFLGGVDSLVDLMNKSFPELGIKKTDCKQLS*IDIIIFYSGVVNYGTDNF |
| | | NKEILLDRSAGQNGSLKIKLDYVKKPIPESAFVKILEKLYEEDEGAGMYALYPY |
| | | GGIMDEISESAIPFPH*AGIMYELWYICSWEKHEDNEK |
| 53 | >CM010792.2_58107643_58109265 | MKYSTFSFWFVCKIIFFFLSFNIQPSIANPRENFLKCFSQYIPTNVTNLKLTPKTT |
| | chr:2.0 | LYMPVQNSTIHNLRFTSNTTPKLLVIVTLHMSLISKALFYVQENWFANSNSKR |
| | CBDAS | WS*F*RHVPHISSPICYSRLEKHAFNQKMFIAKSQGLKPELPLEKFIIGLMRKMR |
| | | S*FGCWYCPTVSAAGHFGGGGYGPLM*NYGLADDNIVDAHLVNVDGKVLDR |
| | | KSMGQDLFWAIRGGGRESFRIIVAWKIRLVAVPTKSTMFSVKKIKEIHELVKLV |
| | | NKWQNISYKYDIDLLLMTHFITRNITDNQGKNKTTIHTYFSLVFLGGVDSLVDL |
| | | MNKSFPEFGIKKIDCKQLSWIDTIIFYSGVVNYGTDNFNNQISLVRSAGQNGAF |
| | | KIKLDYVKKPIPESAFVKILEKLYEEDKGVGMYALYPYGCLMDEISESAIPFPH |
| | | RVGIMYELWYICSWEKHEDKEKYLNWIRNVDNFMTPYVSQNPRLTYLNYRHL |
| | | DIGINDPKSQNNYTEACIWGEK |
| 54 | >CM010796.2_62089453_62091076 | MKYSTFCFWYVCKIIFFFLSFNIQISIANPQENFLKCFSQYIPTNVTNAKLVYTQ |
| | chr:6.0 | HDQFYMSILNSTIQNLRFTSDTTPKPLVIITPLNVSHIQGTILCSKKVGLQIRTRS |
| | CBDAS | GGHDAEGMSYISQVPFVIVDLRNMHSVKIDVHSQTAWVEAGATLGEVYYWIN |
| | | ENNENLSFPAGYCPTVGAGGHFSGGGYGALMRNYGLAADNIIDAHLVNVDGK |
| | | VLDRKSMGEDLFWAIRGGGGENFGIIAAWKIRLVAVPSMSTIFSVKKNMEIHE |
| | | LVKLVNKWQNIAYMYEKELLLFTHFITRNITDNQGKNKTTIHCYFSSIFHGGLD |
| | | SLVDLMNKSFPELGIKKTDCKQLSWIDTIIFNSGLVNYNTTNFKKEILL*RSGGR |
| | | KAAFSIKLDYVKKPIPETAMVTILEKLYEEDVGVGMFVFYPYGGIMDEISESAIP |
| | | FPHRAGIMYEIWYIASWEKQEDNEKHINWIRNVYNFTTPYVSQNPRMAYLNY |
| | | RDLDLGKTNFESPNNYTQARIWGEKYFGKNFNRLVKVKTKVDPDNFFRNEQSI |
| | | PPLP |
| 55 | >CM010797.2_28650052_28651687 | MNCSAFSFWFVCKIIFFFLSFHIQISIANPRENFLKCFSKHIPNNVANPKLVYTQH |
| | chr:7.0 | DQLYMSILNSTIQNLRFISDTTPKPLVIVTPSNNSHIQATILCSKKVGLQIRTRSG |
| | THCAS | GHDAEGMSYISQVPFVVVDLRNMHSIKIDVHSQTAWVEAGATLGEVYYWINE |
| | | KNENLSFPGGYCPTVGVGGHFSGGGYGALMRNYGLAADNIIDAHLVNVDGK |
| | | VLDRKSMGEDLFWAIRGGGGENFGHAAWKIKLVAVPSKSTIFSVKKNMEIHG |
| | | LVKLFNKWQNIAYKYDKDLVLMTHFITKNITDNHGKNKTTVHGYFSSIFHGG |
| | | VDSLVDLMNKSFRELGIKKTDCKEFSWIDTTIFYSGVVNFNTANFKKEILLDRS |
| | | AGKKTAFSIKLDYVKKPIPETAMVKILEKLYEEDVGAGMYVLYPYGGIMEEIS |
| | | ESAIPFPHRAGIMYELWYTASWEKQEDNEKHINWVRSVYNFTTPYVSQNPRLA |
| | | YLNYRDLDLGKTNHASPNNYTQARIWGEKYFGKNFNRLVKVKTKVDPNNFF |
| | | RNEQSIPPLPPHHH |
| 56 | >AGQN03001397.1_569_2191 | MKYSTFCFWYVCKIIFFFLSFNIQISIANPQENFLKCLSQYIPTNVTNAKLVYTQ |
| | chr:nan | HDQFYMSILNSTIQNLRFTSDTTPKPLVIITPLNVSHIQGTILCSKKFGLQIRTRSG |
| | CBDAS | GHDAEGMSYISQVPFVIVDLRNMHSVKIDVHSQNAWVEAGATLGEVYYWINE |
| | | NNENLSFPAGYCPTVGACGHFSGGGYGALMRNYGLAADNIIDAHLVNVDGK |
| | | VLDRKSMGEDLFWAIRGGGGENFGIIAAWKIRLVAVPSMSTIFSVKKNMEIHE |
| | | LVKLVNKWQNIAYMYEKELLLFTHFITRNITDNQGKNKTTIHSYFSSIFHGGVD |
| | | SLVDLMNKSFPELGIKKRDCKQLSWIDTIIFYSGLVNYNTTNFKKEILLDRSGG |
| | | RKAAFSIKLDYVKKPIPETAMVTILEKLYEEDVGVGMFVFYPYGGIMDEISESA |
| | | IPF |
| 57 | >AGQN03001397.1_569_2191 | MKYSTFCFWYVCKIIFFFLSFNIQISIANPQENFLKCLSQYIPTNVTNAKLVYTQ |
| | chr:nan | HDQFYMSILNSTIQNLRFTSDTTPKPLVIITPLNVSHIQGTILCSKKFGLQIRTRSG |
| | CBDAS | GHDAEGMSYISQVPFVIVDLRNMHSVKIDVHSQNAWVEAGATLGEVYYWINE |
| | | NNENLSFPAGYCPTVGACGHFSGGGYGALMRNYGLAADNIIDAHLVNVDGK |
| | | VLDRKSMGEDLFWAIRGGGGENFGIIAAWKIRLVAVPSMSTIFSVKKNMEIHE |
| | | LVKLVNKWQNIAYMYEKELLLFTHFITRNITDNQGKNKTTIHSYFSSIFHGGVD |
| | | SLVDLMNKSFPELGIKKRDCKQLSWIDTIIFYSGLVNYNTTNFKKEILLDRSGG |
| | | RKAAFSIKLDYVKKPIPETAMVTILEKLYEEDVGVGMFVFYPYGGIMDEISESA |
| | | IPF |
| 58 | >AGQN03001397.1_88192_89742 | NPEGNFLKCFSQYIPTNVTNAKLVYTQHDQFYMSILNSTIQNLRFTFDTTPKPL |
| | chr:nan | VIITPLNVSHIQGTILCSKKVGL*IRTRSGGHDAEGMSYISQVPFVIVNLRNMHS |
| | THCAS | VKIDVHSETAWVEAGATLGEVYYWINENNENLSFLAGYCPTVGAGGHFSGGG |
| | | YGALMRNYGLAANNIIDAHENFGHAAWKIRFVAVPSMSTIFSVKKNMEIHELV |
| | | KLVNKWQNIAYMYEKE*LLFTHFITRNITDNQGKNKTTIHSYFSSIFYGGVDSL |
| | | VDLMNKSFPELGIKKTDCKQLSWIDTIIFYSGLVNYNTTNFKKELLLDRSGGRK |
| | | AAFSIKLD*VKKPIPETAMVTILEKLYEEDVGVGMFVFYPYGGIMDEISESAIPF |
| | | PHRAGIMYEIWYIASWEKQEDNEKHINWIRNVYNFTTPYVSQNPRMAYLNYR |
| | | DLDLGKTNFESPNNYTQARIWGEKYFGKNFNRLVKVKTKVDPDNFFRNEQSIP |
| | | PLPLRHH |
| 59 | >AGQN03005496.1_2997_4620 | MNCSTFSFWFVCKIIFFFLSFNIQISIANPQENFLKCFSEYIPNNPANPKFIYTQHD |
| | chr:nan | QLYMSVLNSTIQNLRFTSDTTPKPLVIVTPSNVSHIQASILCSKKVGLQIRTRSG |
| | THCAS | GHDAEGLSYISQVPFAIVDLRNMHTVKVDIHSQTAWVEAGATLGEVYYWINE |
| | | MNENFSFPGGYCPTVGVGGHFSGGGYGALMRNYGLAADNIIDAHLVNVDGK |
| | | VLDRKSMGEDLFWAIRGGGGENFGHAAWKIKLVVVPSKATIFSVKKNMEIHG |
| | | LVKLFNKWQNIAYKYDKDLMLTTHFRTRNITDNHGKNKTTVHGYFSSIFLGG |
| | | VDSLVDLMNKSFPELGIKKTDCKELSWIDTTIFYSGVVNYNTANFKKEILLDRS |
| | | AGKKTAFSIKLDYVKKLIPETAMVKILEKLYEEEVGVGMYVLYPYGGIMDEIS |
| | | ESAIPFPHRAGIMYELWYTATWEKQEDNEKHINWVRSVYNFTTPYVSQNPRLA |
| | | YLNYRDLDLGKTNPESPNNYTQARIWGEKYFGKNFNRLVKVKTKADPNNFFR |
| | | NEQSIPPLP |
| 60 | >CM010797.2_46549893_46551515 | PICYSRLENMHTVKVDIHSQTAWVEAGATLGEVYYWINEMNENFSFPGGYCP |
| | chr:7.0THCAS | TVGVGGHFSGGGYGALMRNYGLAADNIIDAHLVNVDGKVLDRKSMEKIYFG |
| | | LYVVEEEKTLESLQHGKSNLLLSHQRLLYSVLKRTWRYMGLSSYLTNGKILLT |
| | | SMTKI*CSRLTSETRNITDNHGKNKTTVHGYFSSIFLGGVDSLVDLMNKSFPEL |
| | | GIKKTDCKELSWIDTTIFYSGVVNYNTANFKKEILLDRSAGKKTAFSIKLDYVK |
| | | KLIPETVMVKILEKLYEEEVGVGMYVLYPYGGIMDEISESAIPFPHRAGIMYEL |
| | | WYTATWEKQEDNEKHINWVRSVYNFTTPYVSQNPRLAYLNYRDLDLGKTNP |
| | | ESPNNYTQARIWGEKYFGKNFNRLVKVKTKADPNNFFRNEQSIPPLP |
| 61 | >AGQN03010271.1_2988_4605 | MNCSTFSFWFVCKIIFFFLSFNIQISIANPQENFLKCFSEYIPNNPANPKFIYTQHD |
| | chr:nan | QLYMSVLNSTIQNLRFTSDTTPKPLVIVTPSNVSHIQASILCSKKVGLQIRTRSG |
| | THCAS | GHDAEGLSYISQVPFAIVDLRNMHTVKVDIHSQTAWVEAGATLGEVYYWIKM |
| | | NENFSFPGGYCPTVGVGGHFSGGGYGALMRNYGLAADNIIDAHLVNVDGKVL |
| | | DRKSMGEDLFWAIRGGGGENFGIIAAWKIKLVVVPSKATIFSVKKNMEIHGLV |
| | | KLFNKWQNIAYKYDKDLMLTTHFRTRNITDNHGKNKTTVHGYFSSIFLGGVD |
| | | SLVDLMNKSFPELGIKKTDCKELSWIDTTIFYSGVVNYNTANFKKEIFLIDQLG |
| | | RR |
| 62 | >AGQN03001586.1_35783_37400 | STFSFRFVYKIIFFFLSFNIKISIANPQENFLKCFSQYIHNNPANLKLVYTQHDQL |
| | chr:nan | YMSVLNLTIQNLRFTSDTTPKPLVIVTPSNVSHIQATILCSKKVGLQIRTRSGGH |
| | THCAS | DAEGLSYTSQVPFVIVDLRNMHSVKIDIRSQIAWVEAGATLGEVYYWINENLS |
| | | FPGGYCPTVGVGGHFSGGGYRALMRNYGLAADNIIDAHLVNVDGKVLDRKS |
| | | MGEDLFWAIRGGGGENFGHAAWKIRLVAVPSRATIFSVKRNMEIHGLVKLFN |
| | | KWQNIAYKYDKDLLLMTHFITRNIIDNQGKNKTTVHGYFSCIFHGGVDSLVNL |
| | | MNKSFPELGIKKTDCKELSWIDTTIFYSGVVNYNTTNFQKEILLDRSAGQKVAF |
| | | SVKLDYVKKPIPETAIVKILEKLYEEDVGVGVYVLYPYGGIMDKISESTIPFPHR |
| | | AGIMYEV*YAATWEKQEDNEKHINWV*SVYNFMTPYVSQNPRMAYLNYRDL |
| | | DLGKTDPKSPNNYTQARIWGEKYFGKNFDKLVKVKTKVDPNNFFRNEQSIPPL |
| | | PPRRH |
| 63 | >AGQN03006963.114299_15918 | MNCSTFSFWFVCKIIFFFLSFNIQISIANPQENFLKCFSEYIPNNPANPKFIYTQHD |
| | chr:nan | QLYMSVLNSTIQNLRFTSDTTPKPLVIVTPSNVSHIQASILCSKKVGLQIRTRSG |
| | THCAS | GHDAEGLSYISQVPFAIVDLRNMHTVKVDIHSQTAWVEAGATLGEVYYWINE |
| | | MNENFSFPGGYCPTVGVGGHFSGGGYGALMRNYGLAADNIIDAHLVNVDGK |
| | | VLDRKSMGEDLFWAIRGGGGENFGHAAWKIKLVVVPSKATIFSVKKNMEIHG |
| | | LVKLFNKWQNIAYKYDKDLMLTTHFRTRNITDNHGKNKTTVHGYFSSIFLGG |
| | | VDSLVDLMNKSFPELGIKKTDCKELSWIDTTIFYSGVVNYNTANFKKEILLDRS |
| | | AGKKTAFSIKLDYVKKLIPETAMVKILEKLYEEEVGVGMYVLYPYGGIMDEIS |
| | | ESAIPFPHRAGIMYELWYTATWE |

### Example 6

### Transformation of Cannabis and/or Hemp

Seeds were disinfected using ethanol 70% for 30 sec and 5% bleach for 5-10 min. Seeds were then washed using sterile water 4 times. Subsequently seeds were germinated on half-strength 1/2 MS medium supplemented with 10 g·L-1sucrose, 5.5 g·L-lagar (pH 6.8) or 0.05% diluted agar at 25+/−2C under 16/8 photoperiod and 36-52 uM×m−1×s−1 intensity. Young leaves were selected at about 0.5-10 mm for initiation of shoot culture. Explants were disinfected using 0.5% NaOCL (15% v/v bleach) and 0.1% tween 20 for 20 min (Optional as plantlets were growing in an aseptic environment). Additionally, a different tissue was tested, for example young cotyledons 2-3 days old.

### Callus Induction/Inoculation

Leaves were cultivated on MS media supplemented with 3% sucrose and 0.8% Bacteriological agar (PH 5. 8). Autoclave after measuring pH). Add filtered sterilized 0.5 uM NAA*+1 uM TDZ* and plates kept at 25+/−2C in the dark. NAA/TDZ was replaced with 2-4D and Kinetin at different concentrations. Copper sulphate and additional myo-inositol and proline were tested for callus quality. In addition, Glutamine was added to MS media prior pH measurement to increase callus generation and quality. The callus was broken in smaller pieces and allowed to grow as in for 2-3 days before inoculation.

Callus were generated using leaf tissue from 1 month old in-vitro Finola plants. The protocol disclosed below are focused on the transformation of callus in conditions that promote healthy tissue formation without hyperhydricity (excessive hydration, low lignification, impaired stomatal function and reduced mechanical strength of tissue culture-generated plants). Prior to CRISPR delivery and genome modification in the callus tissue, protocols disclosed below are being modified using the GUS (beta-glucuronidase) reporter gene system to identify conditions for maximal expression of transgenes and successful regeneration of plants. FIGS. 7A and 7B show that Hemp callus inoculated with agrobacterial carrying the GUS expressing vector pCambia1301 following staining with X-Gluc to visualize the cells that have been successfully transformed with the DNA. In some embodiments, a skilled artisan may be able to use the protocols disclosed herein to regenerate plants with CRISPR mediated THCAS gene over-expressing in suitable vector.

### Callus Generation Protocol was Performed as Outlined Below

Disinfect seeds using ethanol 70% for 30 sec and 5% bleach for 5-10 min. Wash seeds using abundant sterile water 4 times. Germinate seeds on half-strength 1/2 MS medium supplemented with 15 g·L-1sucrose, 5.5 g·L-lagar (pH 6.8) at 25+/−2C under 16/8 photoperiod.

Select young leaves 0.5-10 mm for initiation of shoot culture. Disinfect explants using 0.5% NaOCL (15% v/v bleach) and 0.1% tween 20 for 20 min (Optional as plantlets are growing in an aseptic environment).

Callus induction: Cultivate leaves on MS media+3% sucrose and 0.8% TYPE E agar (Sigma)+0.15mg/l IAA+0.1mg/l TDZ+0.001mg/l Pyridoxine+10 mg/l myo-inositol+0.001 mg/l nicotinic acid+0.01 mg/l Thiamine+0.5 mg/l AgNO3 (CI.1.98.3) and place them at 25C+/−2 and 16H photoperiod and 52 uM/m/s light intensity for 4 weeks.

Break the callus in smaller pieces and let them grow as in 4 for one week before inoculation.

| | | | | | | | Nicotinic | | |
|---|---|---|---|---|---|---|---|---|---|
| | | Sucrose | IAA | TDZ | Pyridoxine | Myo- | acid | Thiamine | AgNO3 |
| | MSg/l | g/l | mg/l | mg/l | mg/l | inositolmg/l | mg/l | mg/l | mg/l |
| CI.1.98.3 | 4.92 | 30 | 0.15 | 0.1 | 0.001 | 10 | 0.001 | 0.01 | 0.5 |

### Callus Inoculation and Regeneration Protocol was Performed as Outlined Below

Grow LBA4404/AGL1:desired vector to 10 in LB+Rif and Spec media at 28C 24 Hrs.

Transfer 200 ul for previous culture into 100 ml MGL without antibiotic and incubate at 28C 24 Hr.

Spin culture at 3000 rpm and 4C and resuspend it in cells in MS+10 g/l glucose+15 g/l sucrose and pH 5.8) to obtain OD600≈0.6-0.8. Agrobacterium cells were activated by treating with 200 μM acetosyringone (AS) for 45-60 min in dark before infection.

Calli were added into the agrobacterium for 15-20 min with continuous shaking at 28C.

Transfer infected calli to sterile filter paper and dry. Transfer to co-culture media at 25C for 48 Hrs.

After 2-3 days of co-cultivation, the infected calli were washed 3 times in sterile water and then washed once in sterile water containing 400 mg/l Timentine and again in sterile water containing 200 mg/l Timentine to remove Agrobacterium.

The washed calli were dried on sterile filter papers and cultured on callus selection medium containing 160 mg/l Timentine and 50 mg/l Hyg). Kept in dark for selecting transgenic calli for 15 days.

After first round of selection for 20 days, brownish or black coloured calli were discarded and white calli were transferred to fresh selection medium for second selection cycle for 15 days.

This step allowed the proliferation of micro calli and when small micro calli started growing on the mother calli, each micro callus was gently separated from the mother calli and transferred to fresh selection medium for the third selection 15 days. Healthy calli were selected for regeneration and PCR analysis.

Shoot regeneration: After three selection cycles, healthy callus were transferred to MS+3% sucrose and 0.8% TYPE E agar (Sigma)+0.5 uMTDZ plus selective antibiotic (depending on vector used) and 160 mg/l of Timentin for shoot regeneration. Healthy callus were placed at 25C+/−2 and 16H photoperiod and 52 uM/m/s light intensity (Acclimation process could be used by placing tissue paper on top to avoid excessive light for at least 1-2 weeks).

Once shoots were observed to be well stablished, 2-3 weeks, plantlets were transferred to Rooting media containing: half MS media+3% sucrose, 0.8% TYPE E agar (Sigma), auxins 2.5 uM IBA and selective antibiotic (depending on vector used) and 160 mg/l of Timentin. Place them at 25+/−2C, 16 h photoperiod and 52 uM×m−1×s−1 intensity.

Transfer stablished plants to soil. Explants had the roots cleaned from any rest of agar. Plantlets were preincubated in coco natural growth medium (Canna Continental) in thermocups (Walmart store, Inc) for 10 days. The cups were covered with polythene bags to maintain humidity, kept in a growth room and later acclimatized in sterile potting mix (fertilome; Canna Continental) in large pots. All the plants were kept under strict controlled environmental conditions (25±3° C. temperature and 55±5% RH). Initially, plants were kept under cool fluorescent light for 10 days and later exposed to full spectrum grow lights (18-hour photoperiod, ˜700±24 μmol·m−2·s−1 at plant canopy level

### Callus Transformation

Agrobacterium culture was prepared from glycerol stock/single colony on agar plate transfer Agrobacterium colonies carrying the vector of interest into liquid LB media*+15 uM acetoseryngone (plus selection antibiotic: this will depend on vector and Agrobacterium strain used). Shook culture overnight at 28° C. Additionally, different Agrobacterium inoculation media will be tested. Once Agrobacterium liquid culture containing antibiotic reaches an OD600=0.5 approx., Agrobacterium liquid culture was centrifuged at 4000 rpm maximum for 15 min at 4° C. The Agrobacterium pellet was collected and resuspended it in inoculation media comprising LB media adjusting OD600 to approximately 0.3 without antibiotics. After pellet resuspension, the culture is left for 1-2 hours before inoculation. The calli were mixed into the culture and incubated in a shaker, 150 rpm, for 15-30 min. The reaction mixture was monitored, as excessive OD can generate contamination. Inoculation media is tested to increase efficiency of Agrobacterium infection. Calli were collected in sterilized filter paper and allowed to dry and placed on a single sterile filter paper which is placed on a petri dish containing callus induction media (MS media containing 3% sucrose and 0.8% Bacteriological agar (pH 5.8, autoclave). Afterwards, it was filtered and sterilized (0.5 uM NAA and 1 uM TDZ) and placed at 25C+/−2 in the dark for 2-3 days. Excessive Agrobacterium Contamination was monitored during the incubation. Additionally, replace NAA/TDZ with 2-4D and Kinetin at different concentrations. In some cases, copper sulphate, myo-inositol, and proline were tested for callus quality. In addition, Glutamine was added to MS media prior to pH measurement to increase callus generation and quality.

The callus MS media+3% sucrose and 0.8% bacteriological agar (pH 5.8) was transferred and autoclaved. Filtered, sterilized 0.5 uM NAA+1 uM TDZ (Replace NAA/TDZ with 2-4D and Kinetin at different concentrations. In this step, Copper sulphate and additional myo-inositol and proline were tested for callus quality. In addition, Glutamine may be added to MS media prior pH measurement to increase callus generation and quality. If Agrobacterium overgrow and threaten to overwhelm calli, calli (disinfection may be conducted before continuing callus induction) was added along with a selective antibiotic (depending on vector used) and 160-200 mg/l of Timentin to inhibit Agrobacterium growth. The reaction mixture was placed at 25C+/−2 in the dark. The selection media was renewed every week. Growth of callus was monitored as well as health. Two weeks after selection started, callus was transferred to shooting media (This step is tested for different selection time.)

### Cotyledon Inoculation

Cotyledon is the embryonic leaf in seed-bearing plants and represent the first leaves to appear from a germinating seed. Protocols disclosed below have been developed for the excision of cotyledon from 5 to 7-day old plantlets prior to submerging into a suspension of agrobacterium carrying the GUS reporter vector pCambia1301. After 7 days on Hygromycin selection agar plates, the tissue was stained with X-Gluc and GUS expression visualized. The blue staining indicated by black arrows shown in FIGS. 8A-8C was observed in callus forming areas, areas where plant regeneration is expected to occur (ongoing evaluation).

### Cotyledon and Hypocotyls Inoculation

Grow AGL1:desired vector (from glycerol stock/colony) in LB+Rifampicin (Rif) and Kanamycin (Kan) media at 28C 48 Hrs.

Transfer 200 ul for previous culture into 100 ml LB+Rif and Kan media at 28C for 24 Hrs.

Spin down culture at 4 C and resuspend cells in MS+10 g/l glucose+15 g/l sucrose and pH 5.8) to obtain OD₆₀₀≈0.6-0.8. Agrobacterium cells were activated by treating with 200 μM acetosyringone (AS) for 45-60 min in dark before infection.

Add cotyledon/hypocotyl into the agrobacterium for 15-20 min with continuous shaking at 28C.

Transfer infected explants to sterile filter paper and dry. Transfer to co-culture media* at 25C for 48 Hrs.

After 2-3 days of co-cultivation, the infected explants were washed 3 times in sterile water and then washed once in sterile water containing 400 mg/l Timentine (Tim) and again in sterile water containing 200 mg/l Timentine to remove Agrobacterium.

The washed explants were dried on sterile filter papers and cultured on Regeneration-selection containing 160mg/l Timentine and 5 mg/l Hygromycin (Hyg). Kept under 16 hr photoperiod for 15 days and 25C.

After first round of selection for 15 days, brownish or black coloured explants were discarded.

For hypocotyls, shooting/rooting will occur during the first 15 days on selection media.

For Cotyledon, callus will be formed in the proximal side and shoots will be already visible.

Healthy explants were transferred to fresh regeneration-selection media* for second selection cycle for 15 days (A third cycle may be needed depending explant appearance and development).

After selection:

Hypocotyl: Those explants generating shoots and roots can be transferred to compost for acclimatization.

Cotyledon: Shoots formed from callus may be transferred to rooting media*.

*Cotyledon Co-culture/Regeneration-Selection media (Tim 160mg/l+Hyg 5 mg/L).

| | | | | TDZ | NAA | AgNO3 |
|---|---|---|---|---|---|---|
| Cultivars | MS | Agar | Sucrose | mg/l | mg/l | mg/l |
| Co-cultivation/ | 4.93 g/l | 8 g/l | 30 g/l | 0.6 | 0.3 | 5 |
| Regeneration | | | | | | |

| | | | | | AgNO3 |
|---|---|---|---|---|---|
| | MS | Agar | Sucrose | IBA mg/l | mg/l |
| Rooting | 2.46 | 8 g/l | 30 g/l | 1 | 5 |

*Hypocotyl Co-culture/Regeneration-Selection media (Tim 160 mg/l + Hyg 5 mg/L).

| | | | | | | Nicotinic | Myo- |
|---|---|---|---|---|---|---|---|
| Cultivars | ½MS | Gelrite | Sucrose | Thiamine | Pyridoxine | acid | inositol |
| Co-cultivation**/ | 2.46 | 3.5 | 1.5% | 0.01 | 0.001 | 0.001 | 10 |
| Regeneration**/rooting | g/l | g/l | | mg/l | mg/l | mg/l | mg/l |

**Add 3 mM MES and 5 mg/l AgNO3 to avoid browning and enhance shoot proliferation.

### Hypocotyl Inoculation

The hypocotyl is part of the stem of an embryonic plant, beneath the stalks of the seed leaves or cotyledons, and directly above the root. Hypocotyls were excised from 5-7 days old plantlets and submerged into a suspension of agrobacterium carrying the GUS reporter vector pCambia1301. After 3 days on Timentine growth-media, inoculated hypocotyls were transferred to Hygromycin selection plates for 5 days. Then the tissue was stained with X-Gluc and GUS expression visualized. The blue staining was observed in regenerated explants (indicated by white arrows shown in FIGS. 9A and 9C) and regenerative tissue (indicated by white arrows shown in FIGS. 9B and 9D).

### Protoplast Isolation and Transformation

Protocols have been developed for the successful isolation of healthy viable protoplasts from Hemp and Cannabis leaves. The Isolated protoplast transfection conditions have been developed using PEG-transfection of plasmid DNA. Initial evaluation of transformation efficiencies have been performed with the GUS reporter gene vector and conditions identified for successful introduction and expression of the plasmids.

### Floral Dipping

Floral dipping has been used successfully in model plant systems such as Arabidopsis Thaliana, as a method for direct introduction of Agrobacterium into the flowers of growing plantlets. The immature female flowers, containing the sexual organs are immersed into an Agrobacterium suspension carrying the desired vector (either GUS reporter or CRISPR gRNA). After two rounds of dipping, female flowers are crossed with male pollen to obtain seeds in an attempt to produce seeds carrying the transformed DNA in the germline. Seeds may be grown on selective media to confirm transformation and integration of the drug selection marker and transmission of the CRISPR modified genome.

### Callus Regeneration

Multiple experiments have been conducted to identify growth conditions to obtain Cannabis and Hemp callus tissue with the quality and viability to enable regeneration of mature plants. Table 22. showing the different growth factors and nutrients test in various combinations

| MS | Sugar | Agar | | | | | |
|---|---|---|---|---|---|---|---|
| source | source | Type | Cytokinins | Auxins | Nitrogen | Vitamins | Additives |
| basal | Sucrose | Agar | BAP | NAA | Glutamine | Thiamine | CuSO4 |
| MSB5 | Maltose | Type E Agar | Kin | IAA | Caseine | Pyridoxine | AgNO3 |
| | | BactoAgar | Zea | IBA | | Nicotinic acid | |
| | | Gelrite | TDZ | 2-4D | | Myo-Inositol | |
| | | | | Dicamba | | | |

Two callus generation protocols and media compositions showed promising looking callus with the ideal characteristics for regeneration: Granular, breakable and dry.

From first protocol 1.31 listed below performed the best and was expanded to protocols 1.97 to 1.104, and from this method, 1.97 and 1.98 enabled the generation of callus with the ideal characteristics.

| | | | | | | | | | | | |
|---|---|---|---|---|---|---|---|---|---|---|---|
| | | Agar | | | | | | Myo- | | | |
| MS | Sucrose | type E | IAA | IBA | NAA | TDZ | Caseine | inos | Prolien | Thiamine | CuSO4 |
| g/L | g/L | g/L | mg/L | mg/L | mg/L | mg/L | mg/L | mg/L | mg/L | mg/L | mg/L |

| | | | | | | | | | | | |
|---|---|---|---|---|---|---|---|---|---|---|---|
| Cl.1.31 | 4.92 | 30 | 8 | 0.09 | | 0.22 | | | | | |
| Cl.1.32 | 4.92 | 30 | 8 | 0.18 | | 0.22 | | | | | |
| Cl.1.33 | 4.92 | 30 | 8 | 0.26 | | 0.22 | | | | | |
| Cl.1.34 | 4.92 | 30 | 8 | 0.36 | | 0.22 | | | | | |
| Cl.1.35 | 4.92 | 30 | 8 | | 0.1 mg/l | 0.22 | | | | | |
| Cl.1.36 | 4.92 | 30 | 8 | | 0.2 mg/l | 0.22 | | | | | |
| Cl.1.37 | 4.92 | 30 | 8 | | 0.3 mg/l | 0.22 | | | | | |
| Cl.1.38 | 4.92 | 30 | 8 | | 0.4 mg/l | 0.22 | | | | | |
| Cl.1.97 | 4.92 | 30 | 8 | 0.09 | | 0.05 | | | | | |
| Cl.1.98 | 4.92 | 30 | 8 | 0.09 | | 0.1 | | | | | |
| Cl.1.99 | 4.92 | 30 | 8 | 0.09 | | 0.22 | | | | | |
| Cl.1.100 | 4.92 | 30 | 8 | 0.09 | | 0.44 | | | | | |
| Cl.1.101 | 4.92 | 30 | 8 | 0.09 | | 0.05 | 1 | 350 | 690 | 1 | 1.25 |
| Cl.1.102 | 4.92 | 30 | 8 | 0.09 | | 0.1 | 1 | 350 | 690 | 1 | 1.25 |
| Cl.1.103 | 4.92 | 30 | 8 | 0.09 | | 0.22 | 1 | 350 | 690 | 1 | 1.25 |
| Cl.1.104 | 4.92 | 30 | 8 | 0.09 | | 0.44 | 1 | 350 | 690 | 1 | 1.25 |

Two callus generation protocols and media compositions showed promising looking callus with the ideal characteristics for regeneration: Granular, breakable and dry. From first protocol 1.31 performed the best and was expanded to protocols 1.97 to 1.104, and from this method, 1.97 and 1.98 enabled the generation of callus with the ideal characteristics.

| | | | | | | | | Nicotinic | | |
|---|---|---|---|---|---|---|---|---|---|---|
| | MS | Sucrose | Gelrite | IAA | TDZ | Pyridoxine | Myo-inos | acid | Thiamine | AgNO3 |
| | g/l | g/L | g/L | mg/L | mg/L | mg/L | mg/L | mg/L | mg/L | mg/L |
| Cl.1.98.1 | 4.92 | 30 | 3.5 | 0.05 mg/l | 0.1 mg/l | 0.001 | 10 | 0.001 | 0.01 | 0.5 mg/l |
| | | | | | | (25 ul) | (14.28 ul) | (25 ul) | (50 ul) | |
| Cl.1.98.2 | 4.92 | 30 | 3.5 | 0.05 mg/l | 0.1 mg/l | | | | | 0.5 mg/l |
| Cl.1.98.3 | 4.92 | 30 | 3.5 | 0.05 mg/l | 0.1 mg/l | 0.001 | 10 | 0.001 | 0.01 | 0.5 mg/l |
| | | | | | | (25 ul) | (14.28 ul) | (25 ul) | (50 ul) | |
| Cl.1.98.4 | 4.92 | 30 | 3.5 | 0.05 mg/l | 0.1 mg/l | | | | | 0.5 mg/l |

### Cotyledon Regeneration

Regeneration of mature plants from cotyledon tissue is a proven method for fast regeneration when compared to callus formation in other plants. Regeneration was observed from two distinct sources: direct from meristem and indirect from small callus formation.

Protocols have now been developed that have demonstrated early regeneration capacities as shown in FIGS. 12A-12C.

### Hypocotyl Regeneration

Regeneration protocols have been developed to now show Hypocotyl to be highly regenerative, forming adult plants without vitrification problems. Hypocotyl excised from 5-7 days old plantlets regenerated roots and small shoots in the first 5-7 days. Once shoots and roots were regenerated, plantlets were transferred to bigger pots where they remain for 3-4 weeks before transferring them to compost.

| Cultivar | MS | Sucrose | Gelrite | Myo-inositol | Pyridoxine | Nicotinic acid | Thiamine |
|---|---|---|---|---|---|---|---|
| Finola (Hemp) | ½ | 1.5% | 3.5 g/L | 10 mg/L | 0.001 gl/L | 0.001 mg/L | 0.01 mg/L |

### Example 7

### Shoot Regeneration and Plant Growth

### Shoot Regeneration

Agrobacterium treated callus are transferred to MS+3% sucrose and 0.8% Bacteriological agar (pH 5.8. Autoclaved at this point. Filtered sterilized 0.5 uM TDZ is added along with a selective antibiotic (depending on vector used) and 160-200 mg/l of Timentin for shoot regeneration. The reaction mixture is placed at 25C+/−2 and 16/8H photoperiod and 36-52 uM/m/s light intensity (Acclimation process could be used by placing tissue paper on top to avoid excessive light for at least 1-2 weeks).

Once shoots are observed and established, approximately 2-3 weeks, plantlets are transferred to Rooting media containing: half MS media+3% sucrose, 0.8% Bacteriological agar (ph 5.8. and autoclave). Filtered sterilized 2.5uM IBA and selective antibiotic are added (depending on vector used) along with 160-200 mg/l of Timentin. The reaction mixture is placed at 25+/−2C, 16/8 h photoperiod and 36-52 uM×m−1×s−1 intensity. Established plants are planted in soil. Explant's roots are cleaned from agar. Plantlets are covered once in the pot using a plastic sleeve to maintain humidity. Plants are kept under controlled environmental conditions (25±3° C. temperature and 36-55±5% RH).

### Method 1: Protoplast Extraction Transfection and Regeneration in Cannabis

### Reagents

Enzyme solution: 20 mM MES (pH 5.7) containing 1.5% (wt/vol) cellulase R10, 0.4% (wt/vol) macerozyme R10, 0.4 M mannitol and 20 mM KC1 is prepared. The solution is warmed at 55° C. for 10 min to inactivate DNAse and proteases and enhance enzyme solubility. Cool it to room temperature (25° C.) and add 10 mM CaCl₂, 1-5 mM β-mercaptoethanol (optional) and 0.1% BSA. Addition of 1-5 mM β-mercaptoethanol is optional, and its use should be determined according to the experimental purpose. Additionally, before the enzyme powder is added, the MES solution is preheated at 70° C. for 3-5 min. The final enzyme solution should be clear light brown. Filter the final enzyme solution through a 0.45-μm syringe filter device into a Petri dish (100×25 mm² for 10 ml enzyme solution).

WI solution: 4 mM MES (pH 5.7) containing 0.5 M mannitol and 20 mM KCl is prepared. The prepared WI solution can be stored at room temperature (22-25° C.).

W5 solution: 2 mM MES (pH 5.7) containing 154 mM NaCl, 125 mM CaCl₂ and 5 mM KCl is prepared. The prepared W5 solution can be stored at room temperature.

MMG solution: 4 mM MES (pH 5.7) containing 0.4 M mannitol and 15 mM MgCl₂. The prepared MMG solution can be stored at room temperature.

PEG—calcium transfection solution 20-40% (wt/vol) PEG4000 in ddH₂O containing 0.2 M mannitol and 100 mM CaCl₂. PEG solution is prepared at least 1 h before transfection to completely dissolve PEG. The PEG solution can be stored at room temperature and used within 5 d. However, freshly prepared PEG solution gives relatively better protoplast transfection efficiency. PEG solution may not be autoclaved.

Protoplast lysis buffer: 25 mM Tris—phosphate (pH 7.8) containing 1 mM DTT, 2 mM DACTAA, 10% (vol/vol) glycerol and 1% (vol/vol) Triton X-100. The lysis buffer is prepared fresh.

MUG substrate mix for GUS assay 10 mM Tris-HCl (pH 8) containing 1 mM MUG and 2 mM MgCl₂. The prepared GUS assay substrate can be stored at −20° C.

Following the protoplast transfection, gDNA is extracted from the protoplasts, the THCAS target region amplified by PCR, sequenced and analyzed using an analysis tool such as Tide analysis which will compare the cut site to the WT sequencing result. This procedure will provide cutting efficiencies and show indel patterns.

### Plant Growth

Plant growth can take from about 3-4 weeks. In brief, seeds are disinfected using ethanol 70% for 30 sec and 5% bleach for 5-10 min. Seeds are washed using sterile water 4 times. Seeds are germinated on half-strength 1/2 MS medium supplemented with 10 g·L-1sucrose, 5.5 g·L-lagar (pH 6.8) at 25+/−2C under 16/8 photoperiod or 0.05% diluted agar. Media can also be prepared as: MS media, 3% sucrose, 0.8% agar, at pH 5.8. Young leaves are selected, 0.5-10 mm (Additionally, other tissues may be considered such as cotyledons, petioles) for initiation of shoot culture. Explants are disinfected using 0.5% NaOCL (15% v/v bleach) and 0.1% tween 20 for 20 min (Optional as plantlets are growing in an aseptic environment). Plant growth was monitored for contamination. Additionally, different tissues such as young leaves or coleoptiles can be tested.

### Protoplast Isolation

Protoplast isolation is performed utilizing healthy leaves from 3-4week-old plants grown in sterile tissue culture before flowering occurs. Protoplasts prepared from leaves recovered from stress conditions such as: drought, flooding, extreme temperature, and mechanical assault may look similar to those from healthy leaves. However, low transfection efficiency may occur with the protoplasts from stressed leaves.

Protoplast are isolated from healthy leaves, and 0.5-1-mm leaf strips are cut from the middle part of a leaf using a fresh sharp razor blade. Approximately 10⁷ protoplasts per gram fresh weight (approximately 100-150 leaves digested in 40-60 ml of enzyme solution) are obtained. For routine experiments, 10-20 leaves digested in 5-10 ml enzyme solution will give 0.5-1×10⁶ protoplasts, enough for more than 25-100 samples (1-2×10⁴protoplasts per sample). The blade is changed after cutting four to five leaves. Leaves are cut on a piece of clean white paper (8″ x 11″) on top of the solid and clean laboratory bench, which provides for good support and easy inspection of wounded/crushed tissue (juicy and dark green stain).

Leaf strips are transferred quickly into the prepared enzyme solution (10-20 leaves in 5-10 ml.) by dipping both sides of the strips (completely submerged) using a pair of flat-tip forceps. In some cases, immediate dipping and submerging of leaf strips is a factor considered for protoplast yield. When leaf strips are dried out on the paper during cutting, the enzyme solution cannot penetrate, and protoplast yield can be decreased. Afterwards, infiltrate leaf strips are vacuumed for 30 min in the dark using a desiccator. The digestion is continued, without shaking, in the dark for at least 3 h at room temperature. The release of protoplasts is observed when the enzyme solutions turns green after mixing. Digestion time depends on the experimental goals, desirable responses and materials used, and can be optimized empirically. After 3 h digestion, most protoplasts are released from leaf strips in case of Col-0. The digesting time is optimized for each ecotype and genotype of plants being modified. The release of protoplasts in the solution is monitored under the microscope; the size of Arabidopsis mesophyll protoplasts is approximately 30-50 μm.

The enzyme/protoplast solution is diluted with an equal volume of W5 solution before filtration to remove undigested leaf tissues. A clean 75-μm nylon mesh with water is used to remove ethanol (the mesh is normally kept in 95% ethanol) then excess water is removed before protoplast filtration. Filter the enzyme solution containing protoplasts after wetting the 75-μm nylon mesh with W5 solution. The solution is centrifuged, the flow-through at 100 g-200 g, to pellet the protoplasts in a 30-ml round-bottomed tube for 1-2 min. Supernatant is removed. The protoplast pellet is resuspended by gentle swirling. A higher speed (200g) of centrifugation may help to increase protoplast recovery. Protoplasts are resuspended at 2×10⁵ in (2×10⁵ per ml of W5) W5 solution after counting cells under the microscope (x 100) using a hemocytometer. The protoplasts are kept on ice for 30 minutes at room temperature. Although the protoplasts can be kept on ice for at least 24 h, freshly prepared protoplasts should be used for the study of gene expression regulation, signal transduction and protein trafficking, processing and localization.

### DNA-PEG—Calcium Transfection

A transfection is performed by adding 10 μl DNA (10-20 μg of plasmid DNA of 5-10 kb in size) to a 2-ml microfuge tube. 100 μl MMG/protoplasts is added (2×10⁴ protoplasts) and mixed gently. 110 μl of PEG solution is added, and then mixed completely by gently tapping the tube. The transfection mixture is maintained at room temperature for up to 15 min (5 min is sufficient). The transfection mixture is maintained in 400-440 μl W5 solution at room temperature and well mixed by gently rocking or inverting to stop the transfection process. The reaction mixture is centrifuged at 100 g for 2 min at room temperature using a bench-top centrifuge and supernatant removed. Protoplasts are resuspended gently with 1 ml WI in each well of a 6-well tissue culture plate.

Additionally, high transfection efficiency can be achieved using 10-20% PEG final concentration. The optimal PEG concentration is determined empirically for each experimental purpose. Visual reporters such as GFP are used to determine optimal DNA transfection conditions. If protoplasts are derived from healthy leaf materials, most protoplasts should remain intact throughout the isolation, transfection, culture and harvesting procedures.

### Protoplast Culture and Harvest

Protoplasts are incubated at room temperature (20-25° C.) for the desired period of time and then subjected to method 2.

### Method 2: Protoplast Regeneration After Transfection

### Reagents

0.2 M 4-morpholineethanesulfonic acid (MES, pH 5.7; Sigma, cat. no. M8250), sterilize using a 0.45-μm filter

0.8 M mannitol (Sigma, cat. no.M4125), sterilize using a 0.45-μm filter

1 M CaCl₂ (Sigma, cat. no. C7902), sterilize using a 0.45-μm filter

2 M KCl (Sigma, cat. no. P3911), sterilize using a 0.45-μm filter

2 M MgCl₂ (Sigma, cat. no. M9272), sterilize using a 0.45-μm filter

β-Mercaptoethanol (Sigma, cat. no. M6250)

10% (wt/vol) BSA (Sigma, cat. no. A-6793), sterilize using a 0.45-μm filter

Cellulase R10 (Yakult Pharmaceutical Ind. Co., Ltd., Japan)

Macerozyme R10 (Yakult Pharmaceutical Ind. Co., Ltd., Japan)

1 M Tris phosphate (pH 7.8), sterilize using a 0.45-μm filter

100 mM trans-1,2-diaminocyclo-hexane-N,N,N′,N′-tetraacetic acid (DACTAA; Sigma, cat. no. D-1383)

50% (vol/vol) glycerol (Fisher, cat. no. 15892), sterilize using a 0.45-μm filter

20% (vol/vol) Triton X-100 (Sigma, cat. no. T-8787)

1 M DTT (Sigma, cat. no. D-9779)

LUC assay system (Promega, cat. no. E1501)

1 M Tris-HCl (pH 8.0) (US Biological, cat. no. T8650), sterilize using a 0.45-μm filter

0.1 M 4-methylumbelliferyl glucuronide (MUG; Gold BioTechnology, Inc., cat. no. MUG-1G)

0.2 M Na₂CO₃ (Sigma, cat. no. 57795)

1 M methylumbelliferone (MU; Fluka, cat. no. 69580)

Metro-Mix 360 (Sun Gro Horticulture, Inc.)

Jiffy 7 (Jiffy Products Ltd., Canada)

Arabidopsis accessions: Col-0 and Ler (ABRC)

After transfection, protoplast is transfered into a 5 cm diameter petri dish containing liquid callus medium (1/2MS medium supplemented with 0.4 M mannitol, 30 g/L sucrose, 1 mg/L NAA and 3 mg/L kinetin (pH5.8) and incubate 2-3 weeks in the dark at room temperature. After this time the proliferating calli form dust-like calli). Calli are embedded in solid callus medium (1/2MS medium supplemented with 0.4 M mannitol, 30 g/L sucrose, 1 mg/L NAA and 3 mg/L kinetin+0.4% agar, pH 5.8) in a 9 cm diameter petri dish for 3-4 weeks at 25C. In the callus stage, the explants are incubated in the dark (gray background). Calli larger than 3 mm are embedded in solid shooting medium (MS medium supplemented with 2 mg/L kinetin, 0.3 mg/L IAA, 0.4 M mannitol, and 30 g/L sucrose+0.4% Agar, pH 5.8) for shoot induction at 25C and 16/8 photoperiod (3000 lux) for a month. After one month, the multiple shoots which contain leaves or are of a size larger than 5 mm are transferred to fresh shooting medium (pH 5.8) for 2-3 weeks for shoot proliferation at 25C and 16/8 photoperiod (30001ux). After this time multiple shoots with leaves are transferred to solidified rooting medium (MS medium supplemented with 0.1 mg/L IAA, and 30 g/L sucrose+0.4% agar, pH 5.8) 25C and 16/8 photoperiod (3000 lux).

### Agroinfiltration

Agroinfiltration is a fast method to test Agrobacterium reagents in plant tissue. Protocols are developed to test the GUS reporter and CRISPR vectors in Agrobacterium in Cannabis and Hemp leaf tissue to demonstrate the agrobacterium can deliver the desired vector and that the vector expressed, enabling reporter gene expression and/or gene editing. The protocol comprises of infiltrating the Agrobacterium with a syringe into the adaxial part of the leave as shown in FIG. 14.

Disclosed below are protocols for agroinfiltration:

For plant growth conditions, first, sow Cannabis seeds in water-soaked soil mix in a plant pot or in agar plate. Cover the pot with cling film and place it in a growth chamber with 16 h photoperiod cycle at 25/22° C. day and night respectively. Grow until the seedlings have two true leaves (around 7-10 days). Carefully transplant seedlings to the final destination in seed trays. Grow plants for approximately 3-4 more weeks inside the growth chamber. After this, plants are ready for infiltration.

With respect to agrobacterium cultures, this protocol can be used with, at least, three different commonly used strains of Agrobacterium: LBA4404, GV3101 and AGL1. For example, AGL1 has proven to be the most efficient. First, using a glycerol stock and a sterile toothpick, streak the Agrobacterium clone(s) to be used in LB solid plates supplemented with the appropriate antibiotics. Place the plates inside a 28° C. incubator for 48 h to obtain fresh and single colonies. The day before starting the infiltration, start liquid Agrobacterium cultures in LB liquid medium using the fresh colonies on the plates. Pick Agrobacterium biomass from a single colony, using a sterile toothpick, place it inside a sterile Erlenmeyer flask with 100 ml LB liquid media supplemented with the appropriate antibiotics, and culture them at 28° C. and 180 rpm overnight.

For the step of infiltration, pour saturated cultures into 50 ml Falcon tubes to prepare agrobacterium. Spin down cells at 4,000×g for 10 min. Discard LB medium supernatant by decanting. Eliminate as much supernatant as possible and resuspend with vortex the cell pellets using 1 volume of freshly prepared infiltration buffer. After resuspension, leave cultures for 2-4 h in darkness at room temperature. Subsequently, prepare a 1/20 dilution of the saturated culture, measure OD600 and calculate necessary volume to have a final OD600 of 0.05. Dilute using infiltration buffer.

Once the agrobacterium is prepared, fill a 1 or 2 ml needleless syringe with the resuspended culture at a final OD600 of 0.05. Perform the infiltration by pressing the syringe (without needle) on the abaxial side of the leaf while exerting counter-pressure with a fingertip on the adaxial side. Observe how the liquid spreads within the leaf if the infiltration is successful. Infiltrate whole leaves (ca. 100 μl of bacterial suspension/leave). Dry the excess of culture from the leaf surface using tissue paper. Two to four days after infiltration, observe fluorescence of infiltrated proteins or harvest infiltrated leaves to do a protein extraction.

Infiltration Solution (100 ml)

| | | |
|---|---|---|
| Reagent | Volume | Final concentration |

| | | | | |
|---|---|---|---|---|
| 1M MES | 1 | ml | 10 | mM |
| 1M MgCl₂ | 1 | ml | 10 | mM |
| 0.1M acetosyringone | 100 | μl | 0.1 | mM |

The MES solution can be prepared with sterile deionized water by adding 17.5 g MES to sterile deionized water. Then adjust the pH of the solution to 5.6 and sterilize the solution by filtration. The infiltration solution can be stored at room temperature. The MgCl₂ solution can be prepared by adding 20.3 g MgCl₂ to sterile deionized water. The MgCl₂ solution may be sterilized by autoclaving and stored at room temperature. The acetosyringone solution can be prepared by adding 0.196 g acetosyringone to 10 ml DMSO. The acetosyringone solution can be prepared as 1 ml aliquots and stored at −20° C.

For Cannabis protoplasting, BSA (10mg/ml): 0.1 g in 10 ml H₂O (need to be frozen), MgCl₂ 500 mM, CaCl₂ 1M, KCL 1M, KOH 1M, NaCl 5M are solutions needed for needed for protoplast extraction in Cannabis. MES-KOH 100 mM (50 ml-pH 5.6) is prepared by adding 0.976 g MES to about 1 ml 1M KOH. Mannitol 1M (50 ml) may be prepared in multiple stocks by adding 9.11 g Mannitol to water (heat to 55C to dissolve), which may be stored frozen. Plasmolysis buffer (0.6 M Mannitol-10 ml) may be made fresh by adding 6 ml Mannitol 1M (0.6 M final conc.) to 4 ml water. Enzyme solution (20 ml) comprising 0.3g Cellulase RS (sigma C0615) (1.5% final), 0.15g Macerozyme R10 (Calbiochem) (0.75% final), 1 ml KCL 1M (10 mM final concentration), 0.8 ml water, 12 ml 1M Mannitol (0.6 M final conc.), 4 ml MES-KOH 100 (20 mM final conc.) may be made up fresh before each protoplasting and can be sterilized by filtration. The enzyme solution may be incubated for 10 mins at 55 C (water bath) to inactivate proteases and enhance enzyme solubility. After the enzyme solution is cooled then add 200 μl 1M CaCl₂ (10 mM final conc.) and 2 ml 10 mg/ml BSA (0.1% BSA final). For W5 solution (50 ml): make 2×50 ml 40.5 ml water, 6.25 ml CaCl₂ 1M (125 mM final), 1.54 ml NaCl 5M (154 mM final), 1 ml MES-KOH 100 (2 mM final), and 0.25 ml KCL 1M (5 mM final). For W1 Solution (50 ml): prepare 4 mM MES (pH 5.7) containing 0.5 M mannitol and 20 mM KCl. The prepared W1 solution can be stored at room temperature (22-25° C.). Prepare MMG solution (50 ml) by mixing 26.5 ml water, 20 ml Mannitol 1M (0.4 M Final), 1.5 ml MgCl₂ 500 mM (15 mM final), 2 ml MES-KOH (4 mM final), and PEG-CTS (5 ml). The PEG-CTS (5 ml) solution can be made 30 mins before by adding in order of 1 ml Mannitol 1M (0.2 M final conc.), 0.5 ml CaCl₂ 1M (100 mM final conc), 2 g PEG 4000 (40% wt/vol final conc.), and water (up to 5 ml). Vortex can be used to mix the solution without heat.

For protoplast isolation protocols, switch on 55° C. incubator, then thaw 1 M Mannitol (55° C.), and make up fresh enzyme solution. Cut 10-20 shoots from 9-12 day old plants into big beaker with distilled water and swirl. Bunch up leaves in petri dish and cut 0.5-1 mm leaf strips with fresh razor blade. Pour in 10 ml of Plasmolysis buffer (0.6 M Mannitol) and incubate for 10 mins (dark). Remove Plasmolysis buffer with 5 ml pipette without sucking up leaf strips and discard. Transfer tissue to 125 ml glass beaker using the razor blade and add all 20 ml of enzyme solution. Gently swirl to mix then wrap in foil. Place beaker in dessicator (dark). Turn on pump and incubate for 30 minutes. Incubate in dark for 4 hours at 23° C. with gentle shaking (60 RPM). Add 20 ml of room temp W5 to enzyme solution and swirl for 10 s to release protoplasts. Place a 40 μm nylon mesh in a non-skirted 50 ml tube. Swirl enzyme solution round and gently pour slowly through mesh (keep tube on a slight angle to limit fall of liquid). With the remaining 30 ml of W5, wash the leaf strips in the mesh 3-5 times with W5 solution and catch in a fresh non-skirted 50 ml tube. Balance and centrifuge both tubes 3 mins at 80×G—discard supernatant carefully. Resuspend both pellets in 10 ml W5 solution (Combine into one tube then swirl and remove a drop for the haemocytometer). Count protoplasts with haemocytometer (10×mag). (Place cover slip on slide and add protoplast drop to top and bottom to be drawn in by capillary action). Spin down again 3 mins at 80×G. Make the PEG-CTS solution. This should be dissolved and vortexed 30 mins before use. It may require 10 mins or vortexing but it needs to be as fresh as possible. Remove supernatant from protoplasts—Intact protoplasts will have settled by gravity in 30 mins. Try and remove as much liquid as possible without sucking up all the protoplasts. Resuspend protoplasts from second spin (11) to ˜1×10⁶ cell per ml in MMG Transformation. Pipette 10-20 μl plasmid (10-20 μg) into 2 ml Eppendorf. Add 100 μl protoplast (˜100,000 cells) to DNA, mix gently but well by moving tube nearly horizontal and tapping tube. Add 110 μl PEG-CTS. Mix gently as before by tapping tube. Incubate at 23 C for 10 mins in dark. Add 880 μl W5 solution to stop the transformation and mix by inverting tube. Spin at 80×G (1100 RPM in a minispin) for 3 mins and remove supernatant. Resuspend gently in 2 ml of W1 solution. Incubate in the dark at 23 C for 48 hours and remove most of supernatant to leave 200 μl of settled protoplasts.

### Example 8

### Identification of Transgenic Plants

β-glucuronidase Assay

GUS activity was demonstrated by histochemical staining as described by Jefferson (1987 Jefferson, R A. 1987. Assaying chimeric genes in plants: the GUS gene fusion system. Root tissues were incubated in 5-bromo-4-chloro-3-indolyl β-D-glucuronic acid (X-Gluc) for 12 h at 37° C. The appearance of a dark blue color was taken as an indicator of GUS activity.

### Genotyping

Cannabis and/or hemp protoplasts transfected with the anti THCA synthase CRISPR system are cultivated for 48 hours and then collected after removal of the alginate. Total genomic DNA is isolated from the samples using the DNeasy Plant Mini Kit (Qiagen) and used as a template for the amplification of the THCA synthase target site using gene specific primers. The PCR fragment is then purified using the DNeasy PCR purification kit and is ligated into a plasmid using the Zero Blunt PCR Cloning Kit (Invitrogen). The ligation is transformed to chemically competent E. coli cells which are plated on solid LB medium containing kanamycin (50 pg/ml). PCR is performed on 96 individual colonies using the M13 forward and M 13 reverse primers and these PCR products are then directly digested with the restriction enzyme Xho. The gRNA induces indels at the Xho site and thus the loss of this site, as scored by lack of digestion, is a simple method of genotyping a large number of clones to determine the efficiency of indel formation. The PCR products that are resistant to Xho digestion are sequenced to confirm the presence of an indel. Calli are genotyped directly using the direct PCR kit (Phire Plant Direct PCR kit, Thermo Scientific) and the THCA synthase gene specific primers. The resulting PCR products were then directly digested with Xho and analyzed on an agarose gel.

### Tracking of Indels by Decomposition (Tide) Analysis

Cannabis and/or hemp protoplasts transfected with the anti THCA synthase CRISPR system are cultivated for 48 hours and then collected after removal of the alginate. Total genomic DNA is isolated from the samples using the DNeasy Plant Mini Kit (Qiagen) and used as a template for the amplification of the THCA synthase target site using gene specific primers. A control PCR on WT plants is also obtained and both WT and edited PCR products are purified and sent for sequencing. The sequencing products are used for analysis using the online Tide analysis tool (or similar tools for example ICE, Synthego).

### Example 9

### Analysis of THCA Synthase Disruption

After regeneration of multiple transformed Cannabis and/or hemp plants, polynucleotide analysis is performed to confirm gene integration and to determine RNA expression levels. In addition, mRNA and protein levels of THCA synthase is determined. The content of one or more bioactive metabolites, such as terpenes or cannabinoids in plant tissues can also be determined. For example, the content of one or more of THC, CBD, and/or Cannabichromene can be determined with well-established procedures, such as the methods described in US Patent Publication 20160139055, which is hereby incorporated in its entirety. Plants in which THCA synthase activity is disrupted and which have reduced THC and/or increased CBD content are selected.

TABLE 23
Cannabis sativa gene for tetrahydrocannabinolic acid synthase, partial cds

| SEQ | | |
|---|---|---|
| ID | | |
| NO | Strain | Sequence |
| 64 | AB212829 | atgaattgctcagcattaccttaggtagtagcaaaataatatttactactctcattccatatccaaatttcaatagctaat |
| | | cctcgagaaaacttccttaaatgcttctcaaaacatattcccaacaatgtagcaaatccaaaactcgtatacactcaac |
| | | acgaccaattgtatatgtctctcctgaattcgacaatacaaaatcttagattcatctctgatacaaccccaaaaccactc |
| | | gttattgtcactccttcaaataactcccatatccaagcaactattttatgctctaagaaagttggcttgcagattcgaactc |
| | | gaagcggtggccatgatgctgagggtatgtcctacatttctcaagtcccatttgttgtagtagacttgaggaacatgca |
| | | ttcgatcaaaatagatgttcatagccaaactgcgtgggttgaagccggagctacccttggagaagtttattattggatc |
| | | aatgagaagaatgagaatcttagttttcctggtgggtattgccctactgttggcgtaggtggacactttagtggaggag |
| | | gctatggagcattgatgcgaaattatggccttgcggctgataatattattgatgcacacttagtcaatgttgatggaaaa |
| | | gttctagatcgaaaatccatgggagaagatctgttttgggctatacgtggtggtggaggagaaaactttggaatcattg |
| | | cagcatggaaaatcaaactggttgctgtcccatcaaagtctactatattcagtgttaaaaagaacatggagatacatgg |
| | | gcttgtcaagttatttaacaaatggcaaaatattgcttacaagtatgacaaagatttagtactcatgactcacttcataac |
| | | aaagaatattacagataatcatgggaagaataagactacagtacatggttacttctcttcaattatcatggtggagtgg |
| | | atagtctagtcgacttgatgaacaagagctttcctgagttgggtattaaaaaaactgattgcaaagaatttagctggatt |
| | | gatacaaccatcttctacagtggtgagtaaattttaacactgctaattttaaaaaggaaattagcttgatagatcagctg |
| | | ggaagaagacggctttctcaattaagttagactatgttaagaaaccaattcctgaaactgcaatggtcaaaattttgga |
| | | aaaattatatgaagaagatgtaggagctgggatgtatgtgttgtacccttacggtggtataatggaggagatttcagaa |
| | | tcagcaattccattccctcatcgagctggaataatgtatgaactttggtacactgcttcctgggagaagcaagaagata |
| | | atgaaaagcatataaactgggttcgaagtgtttataattttacgactccttatgtgtcccaaaatccaagattggcgtatc |
| | | tcaattatagggaccttgatttaggaaaaactaatcatgcgagtcctaataattacacacaagcacgtatttggggtga |
| | | aaagtattaggtaaaaattttaacaggttagttaaggtgaaaactaaagttgatcccaataatttattagaaacgaacaa |
| | | agtatcccacctcttccaccgcatcatcat |
| 65 | AB212830 | atgaattgctcagcattaccttaggtagtagcaaaataatatttactactctcattcaatatccaaatttcaatagctaat |
| | | cctcaagaaaacttccttaaatgcttctcggaatatattcctaacaatccagcaaatccaaaattcatatacactcaaca |
| | | cgaccaattgtatatgtctgtcctgaattcgacaatacaaaatcttagattcacctctgatacaaccccaaaaccactcg |
| | | ttattgtcactccttcaaatgtctcccatatccaggccagtattctctgctccaagaaagttggtttgcagattcgaactc |
| | | gaagcggtggccatgatgctgagggtttgtcctacatatctcaagtcccatttgctatagtagacttgagaaacatgca |
| | | tacggtcaaagtagatattcatagccaaactgcgtgggttgaagccggagctacccttggagaagtttattattggatc |
| | | aatgagatgaatgagaattttagttttcctggtgggtattgccctactgttggcgtaggtggacactttagtggaggag |
| | | gctatggagcattgatgcgaaattatggccttgcggctgataatatcattgatgcacacttagtcaatgttgatggaaaa |
| | | gttctagatcgaaaatccatgggagaagatctattttgggctatacgtggtggaggaggagaaaactttggaatcatt |
| | | gcagcatggaaaatcaaacttgttgttgtcccatcaaaggctactatattcagtgttaaaaagaacatggagatacatg |
| | | ggcttgtcaagttatttaacaaatggcaaaatattgcttacaagtatgacaaagatttaatgctcacgactcacttcaga |
| | | actaggaatattacagataatcatgggaagaataagactacagtacatggttacttctcttccatttttcttggtggagtg |
| | | gatagtctagttgacttgatgaacaagagctttcctgagttgggtattaaaaaaactgattgcaaagaattgagctggat |
| | | tgatacaaccatcttctacagtggtgttgtaaattacaacactgctaattttaaaaaggaaattagcttgatagatcagct |
| | | gggaagaagacggctttctcaattaagttagactatgttaagaaactaatacctgaaactgcaatggtcaaaattttgg |
| | | aaaaattatatgaagaagaggtaggagttgggatgtatgtgagtacccttacggtggtataatggatgagatttcaga |
| | | atcagcaattccattccctcatcgagctggaataatgtatgaactttggtacactgctacctgggagaagcaagaaga |
| | | taacgaaaagcatataaactgggttcgaagtgtttataatttcacaactccttatgtgtcccaaaatccaagattggcgt |
| | | atctcaattatagggaccttgatttaggaaaaactaatcctgagagtcctaataattacacacaagcacgtatttggggt |
| | | gaaaagtattaggtaaaaattttaacaggttagttaaggtgaaaaccaaagctgatcccaataatttattagaaacgaa |
| | | caaagtatcccacctcttccaccgcatcatcat |
| 66 | AB212831 | atgaattgctcagcattaccttaggtagtagcaaaataatatttttctactctcattcaatatccaaatttcaatagctaat |
| | | cctcaagaaaacttccttaaatgcttctcggaatatattcctaacaatccagcaaatccaaaattcatatacactcaaca |
| | | cgaccaattgtatatgtctgtcctgaattcaacaatacaaaatcttagattcacctctgatacaaccccaaaaccactcg |
| | | ttattgtcactccttcaaatgtctcccatatccaggccagtattctctgctccaagaaagttggtagcagattcgaactc |
| | | gaagcggtggccatgatgctgagggtttgtcctacatatctcaagtcccatttgctatagtagacttgagaaacatgca |
| | | tacggtcaaagtagatattcatagccaaactgcgtgggttgaagccggagctacccttggagaagtttattattggatc |
| | | aatgagatgaatgagaattttagttttcctggtgggtattgccctactgttggcgtaggtggacactttagtggaggag |
| | | gctatggagcattgatgcgaaattatggccttgcggctgataatatcattgatgcacacttagtcaatgttgatggaaaa |
| | | gttctagatcgaaaatccatgggagaagatctattagggctatacgtggtggaggaggagaaaactttggaatcatt |
| | | gcagcatggaaaatcaaacttgttgttgtcccatcaaaggctactatattcagtgttaaaaagaacatggagatacatg |
| | | ggcttgtcaagttatttaacaaatggcaaaatattgcttacaagtatgacaaagatttaatgctcacgactcacttcaga |
| | | actaggaatattacagataatcatgggaagaataagactacagtacatggttacttctcttccatttttcttggtggagtg |
| | | gatagtctagttgacttgatgaacaagagctttcctgagttgggtattaaaaaaactgattgcaaagaattgagctggat |
| | | tgatacaaccatcttctacagtggtgttgtaaattacaacactgctaattttaaaaaggaaattagcttgatagatcagct |
| | | gggaagaagacggctttctcaattaagttagactatgttaagaaactaatacctgaaactgcaatggtcaaaattttgg |
| | | aaaaattatatgaagaagaggtaggagttgggatgtatgtgagtacccttacggtggtataatggatgagatttcaga |
| | | atcagcaattccattccctcatcgagctggaataatgtatgaactttggtacactgctacctgggagaagcaagaaga |
| | | taacgaaaagcatataaactgggttcgaagtgtttataatttcacaactccttatgtgtcccaaaatccaagattggcgt |
| | | atctcaattatagggaccttgatttaggaaaaactaatcctgagagtcctaataattacacacaagcacgtatttggggt |
| | | gaaaagtattaggtaaaaattttaacaggttagttaaggtgaaaaccaaagctgatcccaataatttttttagaaacgaa |
| | | caaagtatcccacctcttccaccgcatcatcat |
| 67 | AB212832 | atgaattgctcagcattaccttaggtagtagcaaaataatatttttctactctcattccatatccaaatttcaatagctaat |
| | | cctcgagaaaacttccttaaatgcttctcaaaacatattcccaacaatgtagcaaatccaaaactcgtatacactcaac |
| | | acgaccaattgtatatgtctatcctgaattcgacaatacaaaatcttagattcatctctgatacaaccccaaaaccactc |
| | | gttattgtcactccttcaaataactcccatatccaagcaactattttatgctctaagaaagttggcttgcagattcgaactc |
| | | gaagcggtggccatgatgctgagggtatgtcctacatatctcaagtcccatttgttgtagtagacttgagaaacatgca |
| | | ttcgatcaaaatagatgttcatagccaaactgcgtgggttgaagccggagctacccttggagaagtttattattggatc |
| | | aatgagaagaatgagaatcttagttttcctggtgggtattgccctactgttggcgtaggtggacactttagtggaggag |
| | | gctatggagcattgatgcgaaattatggccttgcggctgataatattattgatgcacacttagtcaatgttgatggaaaa |
| | | gttctagatcgaaaatccatgggagaagatctgttttgggctatacgtggtggtggaggagaaaactttggaatcattg |
| | | cagcatggaaaatcaaactggttgctgtcccatcaaagtctactatattcagtgttaaaaagaacatggagatacatgg |
| | | gcttgtcaagttatttaacaaatggcaaaatattgcttacaagtatgacaaagatttagtactcatgactcacttcataac |
| | | aaagaatattacagataatcatgggaagaataagactacagtacatggttacttctcttcaatttttcatggtggagtgg |
| | | atagtctagtcgacttgatgaacaagagctttcctgagttgggtattaaaaaaactgattgcaaagaatttagctggatt |
| | | gatacaaccatcttctacagtggtgagtaaattttaacactgctaattttaaaaaggaaattagcttgatagatcagctg |
| | | ggaagaagacggctttctcaattaagttagactatgttaagaaaccaattccagaaactgcaatggtcaaaattttgga |
| | | aaaattatatgaagaagatgtaggagctgggatgtatgtgttgtacccttacggtggtataatggaggagatttcagaa |
| | | tcagcaattccattccctcatcgagctggaataatgtatgaactttggtacactgcttcctgggagaagcaagaagata |
| | | atgaaaagcatataaactgggttcgaagtgtttataattttacgactccttatgtgtcccaaaatccaagattggcgtatc |
| | | tcaattatagggaccttgatttaggaaaaactaatcatgcgagtcctaataattacacacaagcacgtatttggggtga |
| | | aaagtattaggtaaaaattttaacaggttagttaaggtgaaaactaaagttgatcccaataatttttttagaaacgaacaa |
| | | agtatcccacctcttccaccgcatcatcat |
| 68 | AB212833 | atgaattgctcagcattaccttaggtagtagcaaaataatatttttctactctcattcaatatccaaatttcaatagctaat |
| | | cctcaagaaaacttccttaaatgcttctcggaatatattcctaacaatccagcaaatccaaaattcatatacactcaaca |
| | | cgaccaattgtatatgtctgtcctgaattcgacaatacaaaatcttagattcacctctgatacaaccccaaaaccactcg |
| | | ttattgtcactccttcaaatgtctcccatatccaggccagtattctctgctccaagaaagttggtagcagattcgaactc |
| | | gaagcggtggccatgatgctgagggtttgtcctacatatctcaagtcccatttgctatagtagacttgagaaacatgca |
| | | tacggtcaaagtagatattcatagccaaactgcgtgggttgaagccggagctacccttggagaagtttattattggatc |
| | | aatgagatgaatgagaattttagttttcctggtgggtattgccctactgttggcgtaggtggacactttagtggaggag |
| | | gctatggagcattgatgcgaaattatggccttgcggctgataatatcattgattcacacttagtcaatgagatggaaaa |
| | | gttctagatcgaaaatccatgggagaagatctattagggctatacgtggtggaggaggagaaaactttggaatcatt |
| | | gcagcatggaaaatcaaacttgttgttgtcccatcaaaggctactatattcagtgttaaaaagaacatggagatacatg |
| | | ggcttgtcaagttatttaacaaatggcaaaatattgcttacaagtatgacaaagatttaatgctcacgactcacttcaga |
| | | actaggaatattacagataatcatgggaagaataagactacagtacatggttacttctcttccatttttcttggtggagtg |
| | | gatagtctagttgacttgatgaacaagagctttcctgagttgggtattaaaaaaactgattgcaaagaattgagctggat |
| | | tgatacaaccatcttctacagtggtgttgtcaattacaacactgctaattttaaaaaggaaattagcttgatagatcagct |
| | | gggaagaagacggctttctcaattaagttagactatgttaagaaactaatacctgaaactgcaatggtcaaaattttgg |
| | | aaaaattatatgaagaagaggtaggagttgggatgtatgtgagtacccttacggtggtataatggatgagatttcaga |
| | | atcagcaattccattccctcatcgagctggaataatgtatgaactttggtacactgctacctgggagaagcaagaaga |
| | | taacgaaaagcatataaactgggttcgaagtgtttataatttcacaactccttatgtgtcccaaaatccaagattggcgt |
| | | atctcaattatagggaccttgatttaggaaaaactaatcctgagagtcctaataattacacacaagcacgtatttggggt |
| | | gaaaagtattaggtaaaaattttaacaggttagttaaagtgaaaaccaaagctgatcccaataatttttttagaaacgaa |
| | | caaagtatcccacctcttccaccgcatcatcat |
| 69 | AB212834 | atgaattgctcagcattaccttaggtagtagcaaaataatatttttctactctcattccatatccaaatttcaatagctaat |
| | | cctcgagaaaacttccttaaatgcttctcaaaacatattcccaacaatgtagcaaatccaaaactcgtatacactcaac |
| | | acgaccaattgtatatgtctatcctgaattcgacaatacaaaatcttagattcatctctgatacaaccccaaaaccactc |
| | | gttattgtcactccttcaaataactcccatatccaagcaactattttatgctctaagaaagttggcttgcagattcgaactc |
| | | gaagcggtggccatgatgctgagggtatgtcctacatatctcaagtcccatttgttgtagtagacttgagaaacatgca |
| | | ttcgatcaaaatagatgttcatagccaaactgcgtgggttgaagccggagctacccttggagaagtttattattggatc |
| | | aatgagaagaatgagaatcttagttttcctggtgggtattgccctactgttggcgtaggtggacactttagtggaggag |
| | | gctatggagcattgatgcgaaattatggccttgcggctgataatattattgatgcacacttagtcaatgttgatggaaaa |
| | | gttctagatcgaaaatccatgggagaagatctgttttgggctatacgtggtggtggaggagaaaactttggaatcattg |
| | | cagcatggaaaatcaaactggttgctgtcccatcaaagtctactatattcagtgttaaaaagaacatggagatacatgg |
| | | gcttgtcaagttatttaacaaatggcaaaatattgcttacaagtatgacaaagatttagtactcatgactcacttcataac |
| | | aaagaatattacagataatcatgggaagaataagactacagtacatggttacttctcttcaatttttcatggtggagtgg |
| | | atagtctagtcgacttgatgaacaagagctttcctgagttgggtattaaaaaaactgattgcaaagaatttagctggatt |
| | | gatacaaccatcttctacagtggtgagtaaattttaacactgctaattttaaaaaggaaattagcttgatagatcagctg |
| | | ggaagaagacggctttctcaattaagttagactatgttaagaaaccaattccagaaactgcaatggtcaaaattttgga |
| | | aaaattatatgaagaagatgtaggagctgggatgtatgtgttgtacccttacggtggtataatggaggagatttcagaa |
| | | tcagcaattccattccctcatcgagctggaataatgtatgaactttggtacactgcttcctgggagaagcaagaagata |
| | | atgaaaagcatataaactgggttcgaagtgtttataattttacgactccttatgtgtcccaaaatccaagattggcgtatc |
| | | tcaattatagggaccttgatttaggaaaaactaatcatgcgagtcctaataattacacacaagcacgtatttggggtga |
| | | aaagtattaggtaaaaattttaacaggttagttaaggtgaaaactaaagttgatcccaataatttttttagaaacgaacaa |
| | | agtatcccacctcttccaccgcatcatcat |
| 70 | AB212835 | atgaattgctcagcattaccttaggtagtagcaaaataatatttttctactctcattccatatccaaatttcaatagctaat |
| | | cctcgagaaaacttccttaaatgcttctcaaaacatattcccaacaatgtagcaaatccaaaactcgtatacactcaac |
| | | acgaccaattgtatatgtctatcctgaattcgacaatacaaaatcttagattcatctctgatacaaccccaaaaccactc |
| | | gttattgtcactccttcaaataactcccatatccaagcaactattttatgctctaagaaagttggcttgcagattcgaactc |
| | | gaagcggtggccatgatgctgagggtatgtcctacatatctcaagtcccatttgttgtagtagacttgagaaacatgca |
| | | ttcgatcaaaatagatgttcatagccaaactgcgtgggttgaagccggagctacccttggagaagtttattattggatc |
| | | aatgagaagaatgagaatcttagttttcctggtgggtattgccctactgttggcgtaggtggacactttagtggaggag |
| | | gctatggagcattgatgcgaaattatggccttgcggctgataatattattgatgcacacttagtcaatgttgatggaaaa |
| | | gttctagatcgaaaatccatgggagaagatctgttttgggctatacgtggtggtggaggagaaaactttggaatcattg |
| | | cagcatggaaaatcaaactggttgctgtcccatcaaagtctactatattcagtgttaaaaagaacatggagatacatgg |
| | | gcttgtcaagttatttaacaaatggcaaaatattgcttacaagtatgacaaagatttagtactcatgactcacttcataac |
| | | aaagaatattacagataatcatgggaagaataagactacagtacatggttacttctcttcaatttttcatggtggagtgg |
| | | atagtctagtcgacttgatgaacaagagctttcctgagttgggtattaaaaaaactgattgcaaagaatttagctggatt |
| | | gatacaaccatcttctacagtggtgagtaaattttaacactgctaattttaaaaaggaaattagcttgatagatcagctg |
| | | ggaagaagacggctttctcaattaagttagactatgttaagaaaccaattccagaaactgcaatggtcaaaattttgga |
| | | aaaattatatgaagaagatgtaggagctgggatgtatgtgttgtacccttacggtggtataatggaggagatttcagaa |
| | | tcagcaattccattccctcatcgagctggaataatgtatgaactttggtacactgcttcctgggagaagcaagaagata |
| | | atgaaaagcatataaactgggttcgaagtgtttataattttacgactccttatgtgtcccaaaatccaagattggcgtatc |
| | | tcaattatagggaccttgatttaggaaaaactaatcatgcgagtcctaataattacacacaagcacgtatttggggtga |
| | | aaagtattaggtaaaaattttaacaggttagttaaggtgaaaactaaagttgatcccaataatttttttagaaacgaacaa |
| | | agtatcccacctcttccaccgcatcatcat |
| 71 | AB212836 | atgaattgctcagcattaccttaggtagtagcaaaataatatttttctactctcattcaatatccaaatttcattagctaat |
| | | cctcaagaaaacttccttaaatgcttctcggaatatattcctaacaatccagcaaatccaaaattcatatacactcaaca |
| | | cgaccaattgtatatgtctgtcctgaattcgacaatacaaaatcttagattcacctctgatacaaccccaaaaccactcg |
| | | ttattgtcactccttcaaatgtctcccatatccaggccagtattctctgctccaagaaagttggtagcagattcgaactc |
| | | gaagcggtggccatgatgctgagggtttgtcctacatatctcaagtcccatttgctatagtagacttgagaaacatgca |
| | | tacggtcaaagtagatattcatagccaaactgcgtgggttgaagccggagctacccttggagaagtttattattggatc |
| | | aatgagatgaatgagaattttagttttcctggtgggtattgccctactgttggcgtaggtggacactttagtggaggag |
| | | gctatggagcattgatgcgaaattatggccttgcggctgataatatcattgatgcacacttagtcaatgttgatggaaaa |
| | | gttctagatcgaaaatccatgggagaagatctattttgggctatacgtggtggaggaggagaaaactttggaatcatt |
| | | gcagcatggaaaatcaaacttgttgttgtcccatcaaaggctactatattcagtgttaaaaagaacatggagatacgtg |
| | | ggcttgtcaagttatttaacaaatggcaaaatattgcttacaagtatgacaaagatttaatgctcacgactcacttcaga |
| | | actaggaatattacagataatcatgggaagaataagactacagtacatggttacttctcttccatttttcttggtggagtg |
| | | gatagtctagttgacttgatgaacaagagctttcctgagttgggtattaaaaaaactgattgcaaagaattgagctggat |
| | | tgatacaaccatcttctacagtggtgttgtaaattacaacactgctaattttaaaaaggaaattagcttgatagatcagct |
| | | gggaagaagacggctttctcaattaagttagactatgttaagaaactaatacctgaaactgcaatggtcaaaattttgg |
| | | aaaaattatatgaagaagaggtaggagttgggatgtatgtgagtacccttacggtggtataatggatgagatttcaga |
| | | atcagcaattccattccctcatcgagctggaataatgtatgaactttggtacactgctacctgggagaagcaagaaga |
| | | taacgaaaagcatataaactgggttcgaagtgtttacaatttcacaactccttatgtgtcccaaaatccaagattggcgt |
| | | atctcaattatagggaccttgatttaggaaaaactaatcctgagagtcctaataattacacacaagcacgtatttggggt |
| | | gaaaagtattaggtaaaaattttaacaggttagttaaggtgaaaaccaaagctgatcccaataatttttttagaaacgaa |
| | | caaagtatcccacctcttccaccgcatcatcat |
| 72 | AB212837 | atgaattgctcagcattaccttaggtagtagcaaaataatatttttctactctcattccatatccaaatttcaatagctaat |
| | | cctcgagaaaacttccttaaatgcttctcaaaacatattcccaacaatgtagcaaatccaaaactcgtatacactcaac |
| | | acgaccaattgtatatgtctatcctgaattcgacaatacaaaatcttagattcatctctgatacaaccccaaaaccactc |
| | | gttattgtcactccttcaaataactcccatatccaagcaactattttatgctctaagaaagttggcttgcagattcgaactc |
| | | gaagcggtggccatgatgctgagggtatgtcctacatatctcaagtcccatttgttgtagtagacttgagaaacatgca |
| | | ttcgatcaaaatagatgttcatagccaaactgcgtgggttgaagccggagctacccttggagaagtttattattggatc |
| | | aatgagaagaatgagaatcttagttttcctggtgggtattgccctactgttggcgtaggtggacactttagtggaggag |
| | | gctatggagcattgatgcgaaattatggccttgcggctgataatattattgatgcacacttagtcaatgttgatggaaaa |
| | | gttctagatcgaaaatccatgggagaagatctgttttgggctatacgtggtggtggaggagaaaactttggaatcattg |
| | | cagcatggaaaatcaaactggttgctgtcccatcaaagtctactatattcagtgttaaaaagaacatggagatacatgg |
| | | gcttgtcaagttatttaacaaatggcaaaatattgcttacaagtatgacaaagatttagtactcatgactcacttcataac |
| | | aaagaatattacagataatcatgggaagaataagactacagtacatggttacttctcttcaatttttcatggtggagtgg |
| | | atagtctagtcgacttgatgaacaagagctttcctgagttgggtattaaaaaaactgattgcaaagaatttagctggatt |
| | | gatacaaccatcttctacagtggtgagtaaattttaacactgctaattttaaaaaggaaattagcttgatagatcagctg |
| | | ggaagaagacggctttctcaattaagttagactatgttaagaaaccaattccagaaactgcaatggtcaaaattttgga |
| | | aaaattatatgaagaagatgtaggagctgggatgtatgtgttgtacccttacggtggtataatggaggagatttcagaa |
| | | tcagcaattccattccctcatcgagctggaataatgtatgaactttggtacactgcttcctgggagaagcaagaagata |
| | | atgaaaagcatataaactgggttcgaagtgtttataattttacgactccttatgtgtcccaaaatccaagattggcgtatc |
| | | tcaattatagggaccttgatttaggaaaaactaatcatgcgagtcctaataattacacacaagcacgtatttggggtga |
| | | aaagtattaggtaaaaattttaacaggttagttaaggtgaaaactaaagttgatcccaataatttttttagaaacgaacaa |
| | | agtatcccacctcttccaccgcatcatcat |
| 73 | AB212838 | atgaattgctcagcattaccttaggtagtagcaaaataatatttttctactctcattccatatccaaatttcaatagctaat |
| | | cctcgagaaaacttccttaaatgcttctcaaaacatattcccaacaatgtagcaaatccaaaactcgtatacactcaac |
| | | acgaccaattgtatatgtctatcctgaattcgacaatacaaaatcttagattcatctctgatacaaccccaaaaccactc |
| | | gttattgtcactccttcaaataactcccatatccaagcaactattttatgctctaagaaagttggcttgcagattcgaactc |
| | | gaagcggtggccatgatgctgagggtatgtcctacatatctcaagtcccatttgttgtagtagacttgagaaacatgca |
| | | ttcgatcaaaatagatgttcatagccaaactgcgtgggttgaagccggagctacccttggagaagtttattattggatc |
| | | aatgagaagaatgagaatcttagttttcctggtgggtattgccctactgttggcgtaggtggacactttagtggaggag |
| | | gctatggagcattgatgcgaaattatggccttgcggctgataatattattgatgcacacttagtcaatgttgatggaaaa |
| | | gttctagatcgaaaatccatgggagaagatctgttttgggctatacgtggtggtggaggagaaaactttggaatcattg |
| | | cagcatggaaaatcaaactggttgctgtcccatcaaagtctactatattcagtgttaaaaagaacatggagatacatgg |
| | | gcttgtcaagttatttaacaaatggcaaaatattgcttacaagtatgacaaagatttagtactcatgactcacttcataac |
| | | aaagaatattacagataatcatgggaagaataagactacagtacatggttacttctcttcaatttttcatggtggagtgg |
| | | atagtctagtcgacttgatgaacaagagctttcctgagttgggtattaaaaaaactgattgcaaagaatttagctggatt |
| | | gatacaaccatcttctacagtggtgagtaaattttaacactgctaattttaaaaaggaaattagcttgatagatcagctg |
| | | ggaagaagacggctttctcaattaagttagactatgttaagaaaccaattccagaaactgcaatggtcaaaattttgga |
| | | aaaattatatgaagaagatgtaggagctgggatgtatgtgttgtacccttacggtggtataatggaggagatttcagaa |
| | | tcagcaattccattccctcatcgagctggaataatgtatgaactttggtacactgcttcctgggagaagcaagaagata |
| | | atgaaaagcatataaactgggttcgaagtgtttataattttacgactccttatgtgtcccaaaatccaagattggcgtatc |
| | | tcaattatagggaccttgatttaggaaaaactaatcatgcgagtcctaataattacacacaagcacgtatttggggtga |
| | | aaagtattaggtaaaaattttaacaggttagttaaggtgaaaactaaagttgatcccaataatttttttagaaacgaacaa |
| | | agtatcccacctcttccaccgcatcatcat |
| 74 | AB212839 | atgaattgctcagcattttccttttggtttgtttgcaaaataataattttctttctctcattcaatatccaaatttcaatagctaat |
| | | cctcaagaaaacttccttaaatgcttctcggaatatattcctaacaatccagcaaatccaaaattcatatacactcaaca |
| | | cgaccaattgtatatgtctgtcctgaattcgacaatacaaaatcttagattcacctctgatacaaccccaaaaccactcg |
| | | ttattgtcactccttcaaatgtctcccatatccaggccagtattctctgctccaagaaagttggtagcagattcgaactc |
| | | gaagcggtggccatgatgctgagggtagtcctacatatctcaagtcccatttgctatagtagacttgagaaacatgca |
| | | tacggtcaaagtagatattcatagccaaactgcgtgggttgaagccggagctacccttggagaagtttattattggatc |
| | | aatgagatgaatgagaattttagttttcctggtgggtattgccctactgttggcgtaggtggacactttagtggaggag |
| | | gctatggagcattgatgcgaaattatggccttgcggctgataatatcattgatgcacacttagtcaatgttgatggaaaa |
| | | gttctagatcgaaaatccatgggagaagatctattttgggctatacgtggtggaggaggagaaaactttggaatcatt |
| | | gcagcatggaaaatcaaacttgttgttgtcccatcaaaggctactatattcagtgttaaaaagaacatggagatacatg |
| | | ggcttgtcaagttatttaacaaatggcaaaatattgcttacaagtatgacaaagatttaatgctcacgactcacttcaga |
| | | actaggaatattacagataatcatgggaagaataagactacagtacatggttacttctcttccatttttcttggtggagtg |
| | | gatagtctagttgacttgatgaacaagagctttcctgagttgggtattaaaaaaactgattgcaaagaattgagctggat |
| | | tgatacaaccatcttctacagtggtgttgtaaattacaacactgctaattttaaaaaggaaattagcttgatagatcagct |
| | | gggaagaagacggctttctcaattaagttagactatgttaagaaactaatacctgaaactgcaatggtcaaaattttgg |
| | | aaaaattatatgaagaagaggtaggagttgggatgtatgtgagtacccttacggtggtataatggatgagatttcaga |
| | | atcagcaattccattccctcatcgagctggaataatgtatgaactttggtacactgctacctgggagaagcaagaaga |
| | | taacgaaaagcatataaactgggttcgaagtgtttataatttcacaactccttatgtgtcccaaaatccaagattggcgt |
| | | atctcaattatagggaccttgatttaggaaaaactaatcctgagagtcctaataattacacacaagcacgtatttggggt |
| | | gaaaagtattaggtaaaaattttaacaggttagttaaggtgaaaaccaaagctgatcccaataatttttttagaaacgaa |
| | | caaagtatcccacctcttccaccgcatcatcat |
| 75 | AB212840 | atgaattgctcagcattaccttaggtagtagcaaaataatatttttctactctcattcaatatccaaatttcaatagctaat |
| | | cctcaagaaaacttccttaaatgcttctcggaatatattcctaacaatccagcaaatccaaaattcatatacactcaaca |
| | | cgaccaattgtatatgtctgtcctgaattcgacaatacaaaatcttagattcacctctgatgcaaccccaaaaccactcg |
| | | ttattgtcactccttcaaatgtctcccatatccaggccagtattctctgctccaagaaagttggtagcagattcgaactc |
| | | gaagcggtggccatgatgctgagggtttgtcctacatatctcaagtcccatttgctatagtagacttgagaaacatgca |
| | | tacggtcaaagtagatattcatagccaaactgcgtgggttgaagccggagctacccttggagaagtttattattggatc |
| | | aatgagatgaatgagaattttagttttcctggtgggtattgccctactgttggcgtaggtggacactttagtggaggag |
| | | gctatggagcattgatgcgaaattatggccttgcggctgataatatcattgatgcacacttagtcaatgttgatggaaaa |
| | | gttctagatcgaaaatccatgggagaagatctattttgggctatacgtggtggaggaggagaaaactttggaatcatt |
| | | gcagcatggaaaatcaaacttgttgttgtcccatcaaaggctactatattcagtgttaaaaagaacatggagatacatg |
| | | ggcttgtcaagttatttaacaaatggcaaaatattgcttacaagtatgacaaagatttaatgctcacgactcacttcaga |
| | | actaggaatattacagataatcatgggaagaataagactacagtacatggttacttctcttccatttttcttggtggagtg |
| | | gatagtctagttgacttgatgaacaagagctttcctgagttgggtattaaaaaaactgattgcaaagaattgagctggat |
| | | tgatacaaccatcttctacagtggtgttgtaaattacaacactgctaattttaaaaaggaaattagcttgatagatcagct |
| | | gggaagaagacggctttctcaattaagttagactatgttaagaaactaatacctgaaactgcaatggtcaaaattttgg |
| | | aaaaattatatgaagaagaggtaggagttgggatgtatgtgagtacccttacggtggtataatggatgagatttcaga |
| | | atcagcaattccattccctcatcgagctggaataatgtatgaactttggtacactgctacctgggagaagcaagaaga |
| | | taacgaaaagcatataaactgggttcgaagtgtttataatttcacaactccttatgtgtcccaaaatccaagattggcgt |
| | | atctcaattatagggaccttgatttaggaaaaactaatcctgagagtcctaataattacacacaagcacgtatttggggt |
| | | gaaaagtattaggtaaaaattttaacaggttagttaaggtgaaaaccaaagctgatcccaataatttttttagaaacgaa |
| | | caaagtatcccacctcttccaccgcatcatcat |
| 76 | AB212841 | atgaattgctcagcattaccttaggtagtagcaaaataatatttttctactctcattcaatatccaaatttcattagctaat |
| | | cctcaagaaaacttccttaaatgcttctcggaatatattcctaacaatccagcaaatccaaaattcatatacactcaaca |
| | | cgaccaattgtatatgtctgtcctgaattcgacaatacaaaatcttagattcacctctgatacaaccccaaaaccactcg |
| | | ttattgtcactccttcaaatgtctcccatatccaggccagtattctctgctccaagaaagttggtagcagattcgaactc |
| | | gaagcggtggccatgatgctgagggtttgtcctacatatctcaagtcccatttgctatagtagacttgagaaacatgca |
| | | tacggtcaaagtagatattcatagccaaactgcgtgggttgaagccggagctacccttggagaagtttattattggatc |
| | | aatgagatgaatgagaattttagttttcctggtgggtattgccctactgttggcgtaggtggacactttagtggaggag |
| | | gctatggagcattgatgcgaaattatggccttgcggctgataatatcattgatgcacacttagtcaatgttgatggaaaa |
| | | gttctagatcgaaaatccatgggagaagatctattttgggctatacgtggtggaggaggagaaaactttggaatcatt |
| | | gcagcatggaaaatcaaacttgttgttgtcccatcaaaggctactatattcagtgttaaaaagaacatggagatacatg |
| | | ggcttgtcaagttatttaacaaatggcaaaatattgcttacaagtatgacaaagatttaatgctcacgactcacttcaga |
| | | actaggaatattacagataatcatgggaagaataagactacagtacatggttacttctcttccatttttcttggtggagtg |
| | | gatagtctagttgacttgatgaacaagagctttcctgagttgggtattaaaaaaactgattgcaaagaattgagctggat |
| | | tgatacaaccatcttctacagtggtgttgtaaattacaacactgctaattttaaaaaggaaattagcttgatagatcagct |
| | | gggaagaagacggctttctcaattaagttagactatgttaagaaactaatacctgaaactgcaatggtcaaaattttgg |
| | | aaaaattatatgaagaagaggtaggagttgggatgtatgtgagtacccttacggtggtataatggatgagatttcaga |
| | | atcagcaattccattccctcatcgagctggaataatgtatgaactttggtacactgctacctgggagaagcaagaaga |
| | | taacgaaaagcatataaactgggttcgaagtgtttataatttcacaacgccttatgtgtcccaaaatccaagattggcgt |
| | | atctcaattatagggaccttgatttaggaaaaactaatcctgagagtcctaataattacacacaagcacgtatttggggt |
| | | gaaaagtattaggtaaaaattttaacaggttagttaaggtgaaaaccaaagctgatcccaataatttttttagaaacgaa |
| | | caaagtatcccacctcttccaccgcatcatcat |

### Example 10

### Target THCA Synthase Sequences for Gene Disruption

Several different regions of the THCAS/CBCAS gene maybe targeted for genetic modification. Table 24 lists gRNA target sequences of the THCAS/CBCAS gene for genetic disruption of the THCAS/CBCAS gene, leading to down regulation of the THCAS/CBCAS expression level. In some cases, the target sites of the THCAS/CBCAS gene are at least about 10, 15, 20, 25, 30, 35, 40, 45, 50, 55, 60, 65, 70, 75, 80, 85, 90, 95, 100, 120, 140, 160, 180, 200, 250, 300, 350, 400, 450, 500, 550, 600, 650, or 700 bases apart. In some cases, the target sites of the THCAS/CBCAS gene are at most about 700, 650, 600, 550, 500, 450, 400, 350, 300, 250, 200, 180, 160, 140, 120, 100, 95, 90, 85, 80, 75, 70, 65, 60, 55, 50, 45, 40, 35, 30, 25, 20, 15, or 10 bases.

TABLE 24
THCAS/CBCAS gene Target Sequences

| SEQ | | |
|---|---|---|
| ID NO | Strand | Guide Target sequence |
| 77 | Positive | CGAGAAAACTTCCTTAAATG |
| 78 | Positive | CAAAACCACTCGTTATTGTC |
| 79 | Positive | CTCGTTATTGTCACTCCTTC |
| 80 | Negative | AACGTCTAAGCTTGAGCTTC |
| 81 | Negative | GTCTAAGCTTGAGCTTCGCC |
| 82 | Positive | TGATGCTGAGGGTATGTCCT |
| 83 | Negative | TCGCCACCGGTACTACGACT |
| 84 | Negative | ACAAGTATCGGTTTGACGCA |
| 85 | Positive | GGTGGGTATTGCCCTACTGT |
| 86 | Negative | CATCCACCTGTGAAATCACC |

Guide polynucleotide sequences may be designed to be hybridizable to the target sequences listed in Table 24. In some cases, the gRNA has a guide space sequence that has a length of about 15 to 45 bases. In some cases, the guide space sequence has a length of about 20 bases. Table 25 lists a plurality of guide polynucleotide sequences that may be utilized to disrupt the THCAS gene and Table 25 is not meant to be limiting.

TABLE 25
Anti-THCAS/CBCAS specific guide polynucleotide
sequences and relevant protospacer
sequences (underlined) of the same

| SEQ ID | |
|---|---|
| NO | GUIDE SEQUENCE |
| 87 | CAUUUAAGGAAGUUUUCUCGGUUUUAGAGCUAGAAA |
| | UAGCAAGUUAAAAUAAGGCUAGUCCGUUAUCAACUU |
| | GAAAAAGUGGCACCGAGUCGGUGC |
| 88 | GACAAUAACGAGUGGUUUUGGUUUUAGAGCUAGAAA |
| | UAGCAAGUUAAAAUAAGGCUAGUCCGUUAUCAACUU |
| | GAAAAAGUGGCACCGAGUCGGUGC |
| 89 | GAAGGAGUGACAAUAACGAGGUUUUAGAGCUAGAAA |
| | UAGCAAGUUAAAAUAAGGCUAGUCCGUUAUCAACUU |
| | GAAAAAGUGGCACCGAGUCGGUGC |
| 90 | CAGAUUCGAACUCGAAGCGGGUUUUAGAGCUAGAAA |
| | UAGCAAGUUAAAAUAAGGCUAGUCCGUUAUCAACUU |
| | GAAAAAGUGGCACCGAGUCGGUGC |
| 91 | AGGACAUACCCUCAGCAUCAGUUUUAGAGCUAGAAA |
| | UAGCAAGUUAAAAUAAGGCUAGUCCGUUAUCAACUU |
| | GAAAAAGUGGCACCGAGUCGGUGC |
| 92 | AGCGGUGGCCAUGAUGCUGAGUUUUAGAGCUAGAAA |
| | UAGCAAGUUAAAAUAAGGCUAGUCCGUUAUCAACUU |
| | GAAAAAGUGGCACCGAGUCGGUGC |
| 93 | UGUUCAUAGCCAAACUGCGUGUUUUAGAGCUAGAAA |
| | UAGCAAGUUAAAAUAAGGCUAGUCCGUUAUCAACUU |
| | GAAAAAGUGGCACCGAGUCGGUGC |
| 94 | ACAGUAGGGCAAUACCCACCGUUUUAGAGCUAGAAA |
| | UAGCAAGUUAAAAUAAGGCUAGUCCGUUAUCAACUU |
| | GAAAAAGUGGCACCGAGUCGGUGC |
| 95 | GUAGGUGGACACUUUAGUGGGUUUUAGAGCUAGAAA |
| | UAGCAAGUUAAAAUAAGGCUAGUCCGUUAUCAACUU |
| | GAAAAAGUGGCACCGAGUCGGUGC |

Table 26 lists vector sequences.

| SEQ | | |
|---|---|---|
| ID | | |
| NO | Name | Sequence |
| 96 | pAGM8031:AtU3promoter: | AGCCTGAACTCACCGCGACGTCTGTCGAGAAGTTTCT |
| | gRNA::Cassava | GATCGAAAAGTTCGACAGCGTCTCCGACCTGATGCA |
| | promoter:CAS9:3xTHCAS/ | GCTCTCGGAGGGCGAAGAATCTCGTGCTTTCAGCTTC |
| | CBCAS targets | GATGTAGGAGGGCGTGGATATGTCCTGCGGGTAAAT |
| | | AGCTGCGCCGATGGTTTCTACAAAGATCGTTATGTTT |
| | | ATCGGCACTTTGCATCGGCCGCGCTCCCGATTCCGGA |
| | | AGTGCTTGACATTGGGGAATTCAGCGAGAGCCTGAC |
| | | CTATTGCATCTCCCGCCGTGCACAGGGTGTCACGTTG |
| | | CAAGACCTGCCTGAAACCGAACTGCCCGCTGTTCTGC |
| | | AGGTAAATTTCTAGTTTTTCTCCTTCATTTTCTTGGTT |
| | | AGGACCCTTTTCTCTTTTTATTTTTTTGAGCTTTGATC |
| | | TTTCTTTAAACTGATCTATTTTTTAATTGATTGGTTAT |
| | | GGTGTAAATATTACATAGCTTTAACTGATAATCTGAT |
| | | TACTTTATTTCGTGTGTCTATGATGATGATGATAACT |
| | | GCAGCCGGTCGCGGAGGCCATGGATGCGATCGCTGC |
| | | GGCCGATCTTAGCCAGACGAGCGGGTTCGGCCCATT |
| | | CGGACCGCAAGGAATCGGTCAATACACTACATGGCG |
| | | TGATTTCATATGCGCGATTGCTGATCCCCATGTGTAT |
| | | CACTGGCAAACTGTGATGGACGACACCGTCAGTGCG |
| | | TCCGTCGCGCAGGCTCTCGATGAGCTGATGCTTTGGG |
| | | CCGAGGACTGCCCCGAAGTCCGGCACCTCGTGCACG |
| | | CGGATTTCGGCTCCAACAATGTCCTGACGGACAATG |
| | | GCCGCATAACAGCGGTCATTGACTGGAGCGAGGCGA |
| | | TGTTCGGGGATTCCCAATACGAGGTCGCCAACATCTT |
| | | CTTCTGGAGGCCGTGGTTGGCTTGTATGGAGCAGCA |
| | | GACGCGCTACTTCGAGCGGAGGCATCCGGAGCTTGC |
| | | AGGATCGCCGCGGCTCCGGGCGTATATGCTCCGCATT |
| | | GGTCTTGACCAACTCTATCAGAGCTTGGTTGACGGCA |
| | | ATTTCGATGATGCAGCTTGGGCGCAGGGTCGATGCG |
| | | ACGCAATCGTCCGATCCGGAGCCGGGACTGTCGGGC |
| | | GTACACAAATCGCCCGCAGAAGCGCGGCCGTCTGGA |
| | | CCGATGGCTGTGTAGAAGTACTCGCCGATAGTGGAA |
| | | ACCGACGCCCCAGCACTCGTCCGAGGGCAAAGGAAT |
| | | AGGCTTCTCTAGCTAGAGTCGATCGACAAGCTCGAG |
| | | TTTCTCCATAATAATGTGTGAGTAGTTCCCAGATAAG |
| | | GGAATTAGGGTTCCTATAGGGTTTCGCTCATGTGTTG |
| | | AGCATATAAGAAACCCTTAGTATGTATTTGTATTTGT |
| | | AAAATACTTCTATCAATAAAATTTCTAATTCCTAAAA |
| | | CCAAAATCCAGTACTAAAATCCAGATCGCTGCAAgcaa |
| | | gaattcaagcttggagccagaaggtaattatccaagatgtagcatcaagaatccaatgttt |
| | | acgggaaaaactatggaagtattatgtaagctcagcaagaagcagatcaatatgcggc |
| | | acatatgcaacctatgttcaaaaatgaagaatgtacagatacaagatcctatactgccag |
| | | aatacgaagaagaatacgtagaaattgaaaaagaagaaccaggcgaagaaaagaatc |
| | | ttgatgacgtaagcactgacgacaacaatgaaaagaagaagataaggtcggtgattgtg |
| | | aaagagacatagaggacacatgtaaggtggaaaatgtaagggcggaaagtaaccttat |
| | | cacaaaggaatcttatcccccactacttatccttttatatttttccgtgtcatttttgcccttga |
| | | gttttcctatataaggaaccaagttcggcatttgtgaaaacaagaaaaaatttggtgtaag |
| | | ctattttctttgaagtactgaggatacaacttcagagaaatttgtaagtttgtaatggacaag |
| | | aagtactccattgggctcgatatcggcacaaacagcgtcggctgggccgtcattacgga |
| | | cgagtacaaggtgccgagcaaaaaattcaaagttctgggcaataccgatcgccacagc |
| | | ataaagaagaacctcattggcgccctcctgttcgactccggggagacggccgaagcca |
| | | cgcggctcaaaagaacagcacggcgcagatatacccgcagaaagaatcggatctgct |
| | | acctgcaggagatctttagtaatgagatggctaaggtggatgactctttcttccataggct |
| | | ggaggagtcctttttggtggaggaggataaaaagcacgagcgccacccaatctttggc |
| | | aatatcgtggacgaggtggcgtaccatgaaaagtacccaaccatatatcatctgaggaa |
| | | gaagcttgtagacagtactgataaggctgacttgcggttgatctatctcgcgctggcgca |
| | | tatgatcaaatttcggggacacttcctcatcgagggggacctgaacccagacaacagc |
| | | gatgtcgacaaactctttatccaactggttcagacttacaatcagcttttcgaagagaacc |
| | | cgatcaacgcatccggagttgacgccaaagcaatcctgagcgctaggctgtccaaatc |
| | | ccggcggctcgaaaacctcatcgcacagctccctggggagaagaagaacggcctgtt |
| | | tggtaatcttatcgccctgtcactcgggctgacccccaactttaaatctaacttcgacctgg |
| | | ccgaagatgccaagcttcaactgagcaaagacacctacgatgatgatctcgacaatctg |
| | | ctggcccagatcggcgaccagtacgcagacctttttttggcggcaaagaacctgtcaga |
| | | cgccattctgctgagtgatattctgcgagtgaacacggagatcaccaaagctccgctga |
| | | gcgctagtatgatcaagcgctatgatgagcaccaccaagacttgactttgctgaaggcc |
| | | cttgtcagacagcaactgcctgagaagtacaaggaaattttcttcgatcagtctaaaaatg |
| | | gctacgccggatacattgacggcggagcaagccaggaggaattttacaaatttattaag |
| | | cccatcttggaaaaaatggacggcaccgaggagctgctggtaaagcttaacagagaa |
| | | gatctgttgcgcaaacagcgcactttcgacaatggaagcatcccccaccagattcacct |
| | | gggcgaactgcacgctatcctcaggcggcaagaggatactacccctttttgaaagataa |
| | | cagggaaaagattgagaaaatcctcacatttcggataccctactatgtaggccccctcgc |
| | | ccggggaaattccagattcgcgtggatgactcgcaaatcagaagagactatcactccct |
| | | ggaacttcgaggaagtcgtggataagggggcctctgcccagtccttcatcgaaaggat |
| | | gactaactttgataaaaatctgcctaacgaaaaggtgcttcctaaacactctctgctgtac |
| | | gagtacttcacagtttataacgagctcaccaaggtcaaatacgtcacagaagggatgag |
| | | aaagccagcattcctgtctggagagcagaagaaagctatcgtggacctcctcttcaaga |
| | | cgaaccggaaagttaccgtgaaacagctcaaagaagattatttcaaaaagattgaatgtt |
| | | tcgactctgttgaaatcagcggagtggaggatcgcttcaacgcatccctgggaacgtat |
| | | cacgatctcctgaaaatcattaaagacaaggacttcctggacaatgaggagaacgagg |
| | | acattcttgaggacattgtcctcacccttacgttgtttgaagatagggagatgattgaaga |
| | | acgcttgaaaacttacgctcatctcttcgacgacaaagtcatgaaacagctcaagaggc |
| | | gccgatatacaggatgggggcggctgtcaagaaaactgatcaatgggatccgagaca |
| | | agcagagtggaaagacaatcctggattttcttaagtccgatggatttgccaaccggaact |
| | | tcatgcagttgatccatgatgactctctcacctttaaggaggacatccagaaagcacaag |
| | | tttctggccagggggacagtctccacgagcacatcgctaatcttgcaggtagcccagct |
| | | atcaaaaagggaatactgcagaccgttaaggtcgtggatgaactcgtcaaagtaatggg |
| | | aaggcataagcccgagaatatcgttatcgagatggcccgagagaaccaaactaccca |
| | | gaagggacagaagaacagtagggaaaggatgaagaggattgaagagggtataaaag |
| | | aactggggtcccaaatccttaaggaacacccagttgaaaacacccagcttcagaatga |
| | | gaagctctacctgtactacctgcagaacggcagggacatgtacgtggatcaggaactg |
| | | gacatcaatcggctctccgactacgacgtggatcatatcgtgccccagtcttttctcaaag |
| | | atgattctattgataataaagtgttgacaagatccgataaaaatagagggaagagtgata |
| | | acgtcccctcagaagaagttgtcaagaaaatgaaaaattattggcggcagctgctgaac |
| | | gccaaactgatcacacaacggaagttcgataatctgactaaggctgaacgaggtggcc |
| | | tgtctgagttggataaagccggcttcatcaaaaggcagcttgagagacacgccagatc |
| | | accaagcacgtggcccaaattctcgattcacgcatgaacaccaagtacgatgaaaatga |
| | | caaactgattcgagaggtgaaagttattactctgaagtctaagctggtttcagatttcagaa |
| | | aggactttcagttttataaggtgagagagatcaacaattaccaccatgcgcatgatgccta |
| | | cctgaatgcagtggtaggcactgcacttatcaaaaaatatcccaagcttgaatctgaattt |
| | | gtttacggagactataaagtgtacgatgttaggaaaatgatcgcaaagtctgagcagga |
| | | aataggcaaggccaccgctaagtacttcttttacagcaatattatgaattttttcaagaccg |
| | | agattacactggccaatggagagattcggaagcgaccacttatcgaaacaaacggaga |
| | | aacaggagaaatcgtgtgggacaagggtagggatttcgcgacagtccggaaggtcct |
| | | gtccatgccgcaggtgaacatcgttaaaaagaccgaagtacagaccggaggcttctcc |
| | | aaggaaagtatcctcccgaaaaggaacagcgacaagctgatcgcacgcaaaaaagat |
| | | tgggaccccaagaaatacggcggattcgattctcctacagtcgcttacagtgtactggtt |
| | | gtggccaaagtggagaaagggaagtctaaaaaactcaaaagcgtcaaggaactgctg |
| | | ggcatcacaatcatggagcgatcaagcttcgaaaaaaaccccatcgactttctcgaggc |
| | | gaaaggatataaagaggtcaaaaaagacctcatcattaagcttcccaagtactctctcttt |
| | | gagcttgaaaacggccggaaacgaatgctcgctagtgcgggcgagctgcagaaaggt |
| | | aacgagctggcactgccctctaaatacgttaatttcttgtatctggccagccactatgaaa |
| | | agctcaaaggatctcccgaagataatgagcagaagcagctgttcgtggaacaacacaa |
| | | acactaccttgatgagatcatcgagcaaataagcgaattctccaaaagagtgatcctcgc |
| | | cgacgctaacctcgataaggtgctttctgcttacaataagcacagggataagcccatca |
| | | gggagcaggcagaaaacattatccacttgtttactctgaccaacttgggcgcgcctgca |
| | | gccttcaagtacttcgacaccaccatagacagaaagcggtacacctctacaaaggagg |
| | | tcctggacgccacactgattcatcagtcaattacggggctctatgaaacaagaatcgacc |
| | | tctctcagctcggtggagacagcagggctgaccccaagaagaagaggaaggtgtgag |
| | | cttctctagctagagtcgatcgacaagctcgagtactccataataatgtgtgagtagttcc |
| | | cagataagggaattagggttcctatagggtttcgctcatgtgttgagcatataagaaaccc |
| | | ttagtatgtatttgtatttgtaaaatacttctatcaataaaatttctaattcctaaaaccaaaatc |
| | | cagtactaaaatccagatcgctactaggagcatcttcattcttaagatatgaagataatctt |
| | | caaaaggcccctgggaatctgaaagaagagaagcaggcccatttatatgggaaagaa |
| | | caatagtatttcttatataggcccatttaagttgaaaacaatcttcaaaagtcccacatcgct |
| | | tagataagaaaacgaagctgagtttatatacagctagagtcgaagtagtgcttgCCTC |
| | | TGTTCCCCAGAGGGCAgttttagagctagaaatagcaagttaaaataagg |
| | | ctagtccgttatcaacttgaaaaagtggcaccgagtcggtgctattactagacccagctt |
| | | tcttgtacaaagttggcattacgctttacgaattcccatggggagcatcttcattcttaagat |
| | | atgaagataatcttcaaaaggcccctgggaatctgaaagaagagaagcaggcccattta |
| | | tatgggaaagaacaatagtatttcttatataggcccatttaagttgaaaacaatcttcaaaa |
| | | gtcccacatcgcttagataagaaaacgaagctgagtttatatacagctagagtcgaagta |
| | | gtgcttgCTGTTCCCCAGAGGGCAGGGgttttagagctagaaatagca |
| | | agttaaaataaggctagtccgttatcaacttgaaaaagtggcaccgagtcggtgcttttttt |
| | | ctagacccagctacttgtacaaagaggcattacgctcagagaattcgcatgcggagca |
| | | tcttcattcttaagatatgaagataatcttcaaaaggcccctgggaatctgaaagaagaga |
| | | agcaggcccatttatatgggaaagaacaatagtatttcttatataggcccatttaagttgaa |
| | | aacaatcttcaaaagtcccacatcgcttagataagaaaacgaagctgagtttatatacag |
| | | ctagagtcgaagtagtgcttgAACCTCAAGCACGAGAACTTgttttag |
| | | agctagaaatagcaagttaaaataaggctagtccgttatcaacttgaaaaagtggcacc |
| | | gagtcggtgctttttttctagacccagctacttgtacaaagttggcattacgcttgtgtgag |
| | | accgaggatgcacatgtgaccgagggacacgaagtgatccgtttaaactatcagtgttt |
| | | gacaggatatattggcgggtaaacctaagagaaaagagcgtttattagaataatcggat |
| | | atttaaaagggcgtgaaaaggtttatccgttcgtccatttgtatgtgccagccgcctttgcg |
| | | acgctcaccgggctggttgccctcgccgctgggctggcggccgtctatggccctgcaa |
| | | acgcgccagaaacgccgtcgaagccgtgtgcgagacaccgcggccgccggcgttgt |
| | | ggatacctcgcggaaaacttggccctcactgacagatgaggggcggacgttgacactt |
| | | gaggggccgactcacccggcgcggcgttgacagatgaggggcaggctcgatttcgg |
| | | ccggcgacgtggagctggccagcctcgcaaatcggcgaaaacgcctgattttacgcg |
| | | agtttcccacagatgatgtggacaagcctggggataagtgccctgcggtattgacacttg |
| | | aggggcgcgactactgacagatgaggggcgcgatccttgacacttgaggggcagagt |
| | | gctgacagatgaggggcgcacctattgacatttgaggggctgtccacaggcagaaaat |
| | | ccagcatttgcaagggtttccgcccgtttttcggccaccgctaacctgtatttaacctgctt |
| | | ttaaaccaatatttataaaccttgtattaaccagggctgcgccctgtgcgcgtgaccgcg |
| | | cacgccgaaggggggtgcccccccttctcgaaccctcccggcccgctaacgcgggc |
| | | ctcccatccccccaggggctgcgcccctcggccgcgaacggcctcaccccaaaaatg |
| | | gcagcgctggccaattcccgaggcacgaacccagtggacataagcctgttcggttcgt |
| | | aagctgtaatgcaagtagcgtatgcgctcacgcaactggtccagaaccttgaccgaac |
| | | gcagcggtggtaacggcgcagtggcggttacatggcttgttatgactgtttttttggggta |
| | | cagtctatgcctcgggcatccaagcagcaagcgcgttacgccgtgggtcgatgtttgat |
| | | gttatggagcagcaacgatgttacgcagcagggcagtcgccctaaaacaaagttaaac |
| | | atcatgggggaagcggtgatcgccgaagtatcgactcaactatcagaggtagttggcgt |
| | | catcgagcgccatctcgaaccgacgttgctggccgtacatttgtacggctccgcagtgg |
| | | atggcggcctgaagccacacagcgatattgatttgctggttacggtgaccgtaaggat |
| | | gatgaaacaacgcggcgagctttgatcaacgaccttttggaaacttcggcttcccctgga |
| | | gagagcgagattctccgcgctgtagaagtcaccattgttgtgcacgacgacatcattcc |
| | | gtggcgttatccagctaagcgcgaactgcaatttggagaatggcagcgcaatgacattc |
| | | ttgcaggtatcttcgagccagccacgatcgacattgatctggctatcttgctgacaaaag |
| | | caagagaacatagcgttgccttggtaggtccagcggcggaggaactctttgatccggtt |
| | | cctgaacaggatctatttgaggcgctaaatgaaaccttaacgctatggaactcgccgcc |
| | | cgactgggctggcgatgagcgaaatgtagtgcttacgttgtcccgcatttggtacagcg |
| | | cagtaaccggcaaaatcgcgccgaaggatgtcgctgccgactgggcaatggagcgc |
| | | ctgccggcccagtatcagcccgtcatacttgaagctagacaggcttatcttggacaaga |
| | | agaagatcgcttggcctcgcgcgcagatcagttggaagaatttgtccattacgtgaaag |
| | | gcgagatcaccaaggtagtcggcaaataatgtctagctagaaattcgttcaagccgacg |
| | | ccgcttcgcggcgcggcttaactcaagcgttagatgcactaagcacataattgctcaca |
| | | gccaaactatcaggtcaagtctgcttttattatttttaagcgtgcataataagccctacacaa |
| | | attgggagatatatcatgctgtcagaccaagtttactcatatatactttagattgatttaaaac |
| | | ttcatttttaatttaaaaggatctaggtgaagatcctttttgataatctcatgaccaaaatccct |
| | | taacgtgagttttcgttccactgagcgtcagaccccgtagaaaagatcaaaggatcttctt |
| | | gagatcctttttttctgcgcgtaatctgctgcttgcaaacaaaaaaaccaccgctaccagc |
| | | ggtggtttgtttgccggatcaagagctaccaactctttttccgaaggtaactggcttcagc |
| | | agagcgcagataccaaatactgtccttctagtgtagccgtagttaggccaccacttcaag |
| | | aactctgtagcaccgcctacatacctcgctctgctaatcctgttaccagtggctgctgcca |
| | | gtggcgataagtcgtgtcttaccgggttggactcaagacgatagttaccggataaggcg |
| | | cagcggtcgggctgaacggggggttcgtgcacacagcccagcttggagcgaacgac |
| | | ctacaccgaactgagatacctacagcgtgagctatgagaaagcgccacgcttcccgaa |
| | | gggagaaaggcggacaggtatccggtaagcggcagggtcggaacaggagagcgca |
| | | cgagggagcttccagggggaaacgcctggtatctttatagtcctgtcgggtttcgccac |
| | | ctctgacttgagcgtcgatttttgtgatgctcgtcaggggggcggagcctatggaaaaac |
| | | gccagcaacgcggcctttttacggttcctggcagatcctagatgtggcgcaacgatgcc |
| | | ggcgacaagcaggagcgcaccgacttcttccgcatcaagtgttttggctctcaggccga |
| | | ggcccacggcaagtatttgggcaaggggtcgctggtattcgtgcagggcaagattcgg |
| | | aataccaagtacgagaaggacggccagacggtctacgggaccgacttcattgccgata |
| | | aggtggattatctggacaccaaggcaccaggcgggtcaaatcaggaataagggcaca |
| | | ttgccccggcgtgagtcggggcaatcccgcaaggagggtgaatgaatcggacgtttga |
| | | ccggaaggcatacaggcaagaactgatcgacgcggggttttccgccgaggatgccga |
| | | aaccatcgcaagccgcaccgtcatgcgtgcgccccgcgaaaccttccagtccgtcgg |
| | | ctcgatggtccagcaagctacggccaagatcgagcgcgacagcgtgcaactggctcc |
| | | ccctgccctgcccgcgccatcggccgccgtggagcgttcgcgtcgtcttgaacaggag |
| | | gcggcaggtttggcgaagtcgatgaccatcgacacgcgaggaactatgacgaccaag |
| | | aagcgaaaaaccgccggcgaggacctggcaaaacaggtcagcgaggccaagcagg |
| | | ccgcgttgctgaaacacacgaagcagcagatcaaggaaatgcagctttccttgttcgat |
| | | attgcgccgtggccggacacgatgcgagcgatgccaaacgacacggcccgctctgcc |
| | | ctgttcaccacgcgcaacaagaaaatcccgcgcgaggcgctgcaaaacaaggtcattt |
| | | tccacgtcaacaaggacgtgaagatcacctacaccggcgtcgagctgcgggccgacg |
| | | atgacgaactggtgtggcagcaggtgttggagtacgcgaagcgcacccctatcggcg |
| | | agccgatcaccttcacgttctacgagctttgccaggacctgggctggtcgatcaatggc |
| | | cggtattacacgaaggccgaggaatgcctgtcgcgcctacaggcgacggcgatgggc |
| | | ttcacgtccgaccgcgttgggcacctggaatcggtgtcgctgctgcaccgcttccgcgt |
| | | cctggaccgtggcaagaaaacgtcccgttgccaggtcctgatcgacgaggaaatcgtc |
| | | gtgctgtttgctggcgaccactacacgaaattcatatgggagaagtaccgcaagctgtc |
| | | gccgacggcccgacggatgacgactatttcagctcgcaccgggagccgtacccgctc |
| | | aagctggaaaccttccgcctcatgtgcggatcggattccacccgcgtgaagaagtggc |
| | | gcgagcaggtcggcgaagcctgcgaagagttgcgaggcagcggcctggtggaaca |
| | | cgcctgggtcaatgatgacctggtgcattgcaaacgctagggccttgtggggtcagttc |
| | | cggctgggggttcagcagcccctgctcggatctgttggaccggacagtagtcatggttg |
| | | atgggctgcctgtatcgagtggtgattttgtgccgagctgccggtcggggagctgttgg |
| | | ctggctggtggcaggatatattgtggtgtaaacaaattgacgcttagacaacttaataaca |
| | | cattgcggacgtttttaatgtactggggttgaacactctgtgggtctcaTGCCGAAT |
| | | TCGGATCCGGAGGAATTCCAATCCCACAAAAATCTG |
| | | AGCTTAACAGCACAGTTGCTCCTCTCAGAGCAGAAT |
| | | CGGGTATTCAACACCCTCATATCAACTACTACGTTGT |
| | | GTATAACGGTCCACATGCCGGTATATACGATGACTG |
| | | GGGTTGTACAAAGGCGGCAACAAACGGCGTTCCCGG |
| | | AGTTGCACACAAGAAATTTGCCACTATTACAGAGGC |
| | | AAGAGCAGCAGCTGACGCGTACACAACAAGTCAGCA |
| | | AACAGACAGGTTGAACTTCATCCCCAAAGGAGAAGC |
| | | TCAACTCAAGCCCAAGAGCTTTGCTAAGGCCCTAAC |
| | | AAGCCCACCAAAGCAAAAAGCCCACTGGCTCACGCT |
| | | AGGAACCAAAAGGCCCAGCAGTGATCCAGCCCCAAA |
| | | AGAGATCTCCTTTGCCCCGGAGATTACAATGGACGA |
| | | TTTCCTCTATCTTTACGATCTAGGAAGGAAGTTCGAA |
| | | GGTGAAGGTGACGACACTATGTTCACCACTGATAAT |
| | | GAGAAGGTTAGCCTCTTCAATTTCAGAAAGAATGCT |
| | | GACCCACAGATGGTTAGAGAGGCCTACGCAGCAAGT |
| | | CTCATCAAGACGATCTACCCGAGTAACAATCTCCAG |
| | | GAGATCAAATACCTTCCCAAGAAGGTTAAAGATGCA |
| | | GTCAAAAGATTCAGGACTAATTGCATCAAGAACACA |
| | | GAGAAAGACATATTTCTCAAGATCAGAAGTACTATT |
| | | CCAGTATGGACGATTCAAGGCTTGCTTCATAAACCA |
| | | AGGCAAGTAATAGAGATTGGAGTCTCTAAAAAGGTA |
| | | GTTCCTACTGAATCTAAGGCCATGCATGGAGTCTAAG |
| | | ATTCAAATCGAGGATCTAACAGAACTCGCCGTCAAG |
| | | ACTGGCGAACAGTTCATACAGAGTCTTTTACGACTCA |
| | | ATGACAAGAAGAAAATCTTCGTCAACATGGTGGAGC |
| | | ACGACACTCTGGTCTACTCCAAAAATGTCAAAGATA |
| | | CAGTCTCAGAAGATCAAAGGGCTATTGAGACTTTTC |
| | | AACAAAGGATAATTTCGGGAAACCTCCTCGGATTCC |
| | | ATTGCCCAGCTATCTGTCACTTCATCGAAAGGACAGT |
| | | AGAAAAGGAAGGTGGCTCCTACAAATGCCATCATTG |
| | | CGATAAAGGAAAGGCTATCATTCAAGATCTCTCTGC |
| | | CGACAGTGGTCCCAAAGATGGACCCCCACCCACGAG |
| | | GAGCATCGTGGAAAAAGAAGAGGTTCCAACCACGTC |
| | | TACAAAGCAAGTGGATTGATGTGACATCTCCACTGA |
| | | CGTAAGGGATGACGCACAATCCCACTATCCTTCGCA |
| | | AGACCCTTCCTCTATATAAGGAAGTTCATTTCATTTG |
| | | GAGAGGACACGCTCGAGTATAAGAGCTCATTTTTAC |
| | | AACAATTACCAACAACAACAAACAACAAACAACATT |
| | | ACAATTACATTTACAATTATCGATACAATGAAAA |
| 97 | U6:gRNA::35S:CAS9::Neomycin | ctcgagcttctactgggcggttttatggacagcaagcgaaccggaattgccagctgggg |
| | | cgccctctggtaaggagggaagccctgcaaagtaaactggatggctactcgccgcca |
| | | aggatctgatggcgcaggggatcaagctctgatcaagagacaggatgaggatcgtttc |
| | | gcatgattgaacaagatggattgcacgcaggttctccggccgcttgggtggagaggct |
| | | attcggctatgactgggcacaacagacaatcggctgctctgatgccgccgtgaccggc |
| | | tgtcagcgcaggggcgcccggactttttgtcaagaccgacctgtccggtgccctgaat |
| | | gaactgcaagacgaggcagcgcggctatcgtggctggccacgacgggcgttccttgc |
| | | gcagctgtgctcgacgttgtcactgaagcgggaagggactggctgctattgggcgaag |
| | | tgccggggcaggatctcctgtcatctcaccttgctcctgccgagaaagtatccatcatgg |
| | | ctgatgcaatgcggcggctgcatacgcttgatccggctacctgcccattcgaccaccaa |
| | | gcgaaacatcgcatcgagcgagcacgtactcggatggaagccggtcttgtcgatcagg |
| | | atgatctggacgaagagcatcaggggctcgcgccagccgaactgttcgccaggctca |
| | | aggcgagcatgcccgacggcgaggatctcgtcgtgacccatggcgatgcctgcttgc |
| | | cgaatatcatggtggaaaatggccgcttttctggattcatcgactgtggccggctgggtg |
| | | tggcggaccgctatcaggacatagcgttggctacccgtgatattgctgaagagcttggc |
| | | ggcgaatgggctgaccgcttcctcgtgctttacggtatcgccgctcccgattcgcagcg |
| | | catcgccttctatcgccttcttgacgagttcttctgaattattaacgcttacaatttcctgatg |
| | | cggtattttctccttacgcatctgtgcggtatttcacaccgcatacaggtggcacttttcgg |
| | | ggaaatgtgcgcggaacccctatagtttatttttctaaatacattcaaatatgtatccgctca |
| | | tgagacaataaccctgataaatgcttcaataatagcacgtgctaaaacttcatttttaattta |
| | | aaaggatctaggtgaagatcctttttgataatctcatgaccaaaatcccttaacgtgagtttt |
| | | cgttccactgagcgtcagaccccgtagaaaagatcaaaggatcttcttgagatccttttttt |
| | | ctgcgcgtaatctgctgcttgcaaacaaaaaaaccaccgctaccagcggtggtttgtttg |
| | | ccggatcaagagctaccaactctttttccgaaggtaactggcttcagcagagcgcagat |
| | | accaaatactgtccttctagtgtagccgtagttaggccaccacttcaagaactctgtagca |
| | | ccgcctacatacctcgctctgctaatcctgttaccagtggctgctgccagtggcgataag |
| | | tcgtgtcttaccgggttggactcaagacgatagttaccggataaggcgcagcggtcgg |
| | | gctgaacggggggttcgtgcacacagcccagcttggagcgaacgacctacaccgaac |
| | | tgagatacctacagcgtgagctatgagaaagcgccacgcttcccgaagggagaaagg |
| | | cggacaggtatccggtaagcggcagggtcggaacaggagagcgcacgagggagct |
| | | tccagggggaaacgcctggtatctttatagtcctgtcgggtttcgccacctctgacttgag |
| | | cgtcgatttttgtgatgctcgtcaggggggcggagcctatggaaaaacgccagcaacg |
| | | cggcctttttacggacctgggcttagctggccttttgctcacatgttcttgactcttcgcga |
| | | tgtacgggccagatatgtcgaccgacatgtcgcacaagtcctaagttacgcgacaggct |
| | | gccgccctgcccttttcctggcgttttcttgtcgcgtgttttagtcgcataaagtagaatact |
| | | tgcgactagaaccggagacattacgccatgaacaagagcgccgccgctggcctgctg |
| | | ggctatgcccgcgtcagcaccgacgaccaggacttgaccaaccaacgggccgaact |
| | | gcacgcggccggctgcaccaagctgttaccgagaagatcaccggcaccaggcgcga |
| | | ccgcccggagctggccaggatgcttgaccacctacgccctggcgacgttgtgacagtg |
| | | accaggctagaccgcctggcccgcagcacccgcgacctactggacattgccgagcg |
| | | catccaggaggccggcgcgggcctgcgtagcctggcagagccgtgggccgacacc |
| | | accacgccggccggccgcatggtgttgaccgtgttcgccggcattgccgagttcgagc |
| | | gttccctaatcatcgaccgcacccggagcgggcgcgaggccgccaaggcgcgaggc |
| | | gtgaagtttggcccccgccctaccctcaccccggcacagatcgcgcacgcccgcgag |
| | | ctgatcgaccaggaaggccgcaccgtgaaagaggcggctgcactgcttggcgtgcat |
| | | cgctcgaccctgtaccgcgcacttgagcgcagcgaggaagtgacgcccaccgaggc |
| | | caggcggcgcggtgccttccgtgaggacgcattgaccgaggccgacgccctggcgg |
| | | ccgccgagaatgaacgccaagaggaacaagcatgaaaccgcaccaggacggccag |
| | | gacgaaccgtttttcattaccgaagagatcgaggcggagatgatcgcggccgggtacg |
| | | tgttcgagccgcccgcgcacgtctcaaccgtgcggctgcatgaaatcctggccggtttg |
| | | tctgatgccaagctcgcggcctggccggcgagcttggccgctgaagaaaccgagcgc |
| | | cgccgtctaaaaaggtgatgtgtatttgagtaaaacagcttgcgtcatgcggtcgctgcg |
| | | tatatgatgcgatgagtaaataaacaaatacgcaaggggaacgcatgaaggttatcgct |
| | | gtacttaaccagaaaggcgggtcaggcaagacgaccatcgcaacccatctagcccgc |
| | | gccctgcaactcgccggggccgatgttctgttagtcgattccgatccccagggcagtgc |
| | | ccgcgattgggcggccgtgcgggaagatcaaccgctaaccgttgtcggcatcgaccg |
| | | cccgacgattgaccgcgacgtgaaggccatcggccggcgcgacttcgtagtgatcga |
| | | cggagcgccccaggcggcggacttggctgtgtccgcgatcaaggcagccgacttcgt |
| | | gctgattccggtgcagccaagcccttacgacatatgggccaccgccgacctggtggag |
| | | ctggttaagcagcgcattgaggtcacggatggaaggctacaagcggcctttgtcgtgtc |
| | | gcgggcgatcaaaggcacgcgcatcggcggtgaggttgccgaggcgctggccgggt |
| | | acgagctgcccattcttgagtcccgtatcacgcagcgcgtgagctacccaggcactgc |
| | | cgccgccggcacaaccgttcttgaatcagaacccgagggcgacgctgcccgcgaggt |
| | | ccaggcgctggccgctgaaattaaatcaaaactcatttgagttaatgaggtaaagagaa |
| | | aatgagcaaaagcacaaacacgctaagtgccggccgtccgagcgcacgcagcagca |
| | | aggctgcaacgttggccagcctggcagacacgccagccatgaagcgggtcaactttc |
| | | agttgccggcggaggatcacaccaagctgaagatgtacgcggtacgccaaggcaag |
| | | accattaccgagctgctatctgaatacatcgcgcagctaccagagtaaatgagcaaatg |
| | | aataaatgagtagatgaattttagcggctaaaggaggcggcatggaaaatcaagaaca |
| | | accaggcaccgacgccgtggaatgccccatgtgtggaggaacgggcggttggccag |
| | | gcgtaagcggctgggttgtctgccggccctgcaatggcactggaacccccaagcccg |
| | | aggaatcggcgtgagcggtcgcaaaccatccggcccggtacaaatcggcgcggcgc |
| | | tgggtgatgacctggtggagaagttgaaggcggcgcaggccgcccagcggcaacgc |
| | | atcgaggcagaagcacgccccggtgaatcgtggcaagcggccgctgatcgaatccg |
| | | caaagaatcccggcaaccgccggcagccggtgcgccgtcgattaggaagccgccca |
| | | agggcgacgagcaaccagattttttcgttccgatgctctatgacgtgggcacccgcgat |
| | | agtcgcagcatcatggacgtggccgttttccgtctgtcgaagcgtgaccgacgagctgg |
| | | cgaggtgatccgctacgagcttccagacgggcacgtagaggtttccgcagggccggc |
| | | cggcatggcgagtgtgtgggattacgacctggtactgatggcggtttcccatctaaccg |
| | | aatccatgaaccgataccgggaagggaagggagacaagcccggccgcgtgttccgt |
| | | ccacacgttgcggacgtactcaagttctgccggcgagccgatggcggaaagcagaaa |
| | | gacgacctggtagaaacctgcattcggttaaacaccacgcacgttgccatgcagcgtac |
| | | gaagaaggccaagaacggccgcctggtgacggtatccgagggtgaagccttgattag |
| | | ccgctacaagatcgtaaagagcgaaaccgggcggccggagtacatcgagatcgagtt |
| | | agctgattggatgtaccgcgagatcacagaaggcaagaacccggacgtgctgacggtt |
| | | caccccgattactttttgatcgatcccggcatcggccgttttctctaccgcctggcacgcc |
| | | gcgccgcaggcaaggcagaagccagatggttgttcaagacgatctacgaacgcagtg |
| | | gcagcgccggagagttcaagaagttctgtttcaccgtgcgcaagctgatcgggtcaaat |
| | | gacctgccggagtacgatttgaaggaggaggcggggcaggctggcccgatcctagtc |
| | | atgcgctaccgcaacctgatcgagggcgaagcatccgccggttcctaatgtacggagc |
| | | agatgctagggcaaattgccctagcaggggaaaaaggtcgaaaaggtctctttcctgtg |
| | | gatagcacgtacattgggaacccaaagccgtacattgggaaccggaacccgtacattg |
| | | ggaacccaaagccgtacattgggaaccggtcacacatgtaagtgactgatataaaaga |
| | | gaaaaaaggcgatttttccgcctaaaactctttaaaacttattaaaactcttaaaacccgcc |
| | | tggcctgtgcataactgtctggccagcgcacagccgaagagctgcaaaaagcgcctac |
| | | ccttcggtcgctgcgctccctacgccccgccgcttcgcgtcggcctatcgcggccgctg |
| | | gccgctcaaaaatggctggcctacggccaggcaatctaccagggcgcggacaagcc |
| | | gcgccgtcgccactcgaccgccggcgcccacatcaaggcacctctagatggcaggat |
| | | atattgtggtgtaaacagtttaaacagtgttttactcctcatattaacttcggtcattagaggc |
| | | cacgatttgacacatttttactcaaaacaaaatgtagcatatctcttataatttcaaattcaac |
| | | acacaacaaataagagaaaaaacaaataatattaatttgagaatgaacaaaaggaccat |
| | | atcattcattaactcttctccatccataccatttcacagttcgatagcgaaaaccgaataaa |
| | | aaacacagtaaattacaagcacaacaaatggtacaagaaaaacagttttcccaatgccat |
| | | aatactcgaacgtccggagttatcagaagaactcgtcaagaaggcgatagaaggcgat |
| | | gcgctgcgaatcgggagcggcgataccgtaaagcacgaggaagcggtcagcccatt |
| | | cgccgccaagctcttcagcaatatcacgggtagccaacgctatgtcctgatagcggtcc |
| | | gccacacccagccggccacagtcgatgaatccagaaaagcggccattttccaccatga |
| | | tattcggcaagcaggcatcgccatgggtcacgacgagatcctcgccgtcgggcatgcg |
| | | cgccttgagcctggcgaacagttcggctggcgcgagcccctgatgctcttcgtccagat |
| | | catcctgatcgacaagaccggcttccatccgagtacgtgctcgctcgatgcgatgtttcg |
| | | cttggtggtcgaatgggcaggtagccggatcaagcgtatgcagccgccgcattgcatc |
| | | agccatgatggatactttctcggcaggagcaaggtgagatgacaggagatcctgcccc |
| | | ggcacttcgcccaatagcagccagtcccttcccgcttcagtgacaacgtcgagcacag |
| | | ctgcgcaaggaacgcccgtcgtggccagccacgatagccgcgctgcctcgtcctgca |
| | | gttcattcagggcaccggacaggtcggtcttgacaaaaagaaccgggcgcccctgcg |
| | | ctgacagccggaacacggcggcatcagagcagccgattgtctgttgtgcccagtcata |
| | | gccgaatagcctctccacccaagcggccggagaacctgcgtgcaatccatcttgttcaa |
| | | tccaagctcccattgttggtacccagcttgggtctagtcgtattaagagatagatttgtaga |
| | | gagagactggtgatttcagcgtgtcctctccaaatgaaatgaacttccttatatagaggaa |
| | | ggtcttgcgaaggatagtgggattgtgcgtcatcccttacgtcagtggagatatcacatc |
| | | aatccacttgctttgaagacgtggaggaacgtcttctttttccacgatgctcctcgtgggt |
| | | gggggtccatctttgggaccactgtcggcagaggcatcttgaacgatagcctttcctttat |
| | | cgcaatgatggcatttgtaggtgccaccttccttttctactgtccttttgatgaagtgacaga |
| | | tagctgggcaatggaatccgaggaggtttcccgatattaccctttgttgaaaagtctcaat |
| | | agccctaggtcttctgagactgtatctagatattcttggagtagacgagagtgtcgtgctc |
| | | caccatgttatcacatcaatccacttgctttgaagacgtggaggaacgtcttctttttccac |
| | | gatgctcctcgtgggtgggggtccatctttgggaccactgtcggcagaggcatcttgaa |
| | | cgatagcctttcctttatcgcaatgatggcatttgtaggtgccaccttccttttctactgtcctt |
| | | ttgatgaagtgacagatagctgggcaatggaatccgaggaggtttcccgatattaccctt |
| | | tgttgaaaagtctcaatagccctaggtcttctgagactgtatctagatattcttggagtaga |
| | | cgagagtgtcgtgctccaccattacataggcccatcggagctaacgcagtgaattcaga |
| | | aatctcaaaattccggcagaacaattttgaatctcgatccgtagaaacgagacggtcatt |
| | | gttttagttccaccacgattatatttgaaatttacgtgagtgtgagtgagacttgcataagaa |
| | | aataaaatctttagttgggaaaaaattcaataatataaatgggcttgagaaggaagcgag |
| | | ggataggcctttttctaaaataggcccatttaagctattaacaatcttcaaaagtaccacag |
| | | cgcttaggtaaagaaagcagctgagtttatatatggttagagacgaagtagtgattggat |
| | | ggcaggtggaagaatggacacctgcgagagttttagagctagaaatagcaagttaaaa |
| | | taaggctagtccgttatcaacttgaaaaagtggcaccgagtcggtgctttttttacagtga |
| | | aagcttactgcgttagctccgatgggcctatgtaatggtggagcacgacactctcgtcta |
| | | ctccaagaatatcaaagatacagtctcagaagaccaaagggctattgagacttttcaaca |
| | | aagggtaatatcgggaaacctcctcggattccattgcccagctatctgtcacttcatcaaa |
| | | aggacagtagaaaaggaaggtggcacctacaaatgccatcattgcgataaaggaaag |
| | | gctatcgttcaagatgcctctgccgacagtggtcccaaagatggacccccacccacga |
| | | ggagcatcgtggaaaaagaagacgttccaaccacgtcttcaaagcaagtggattgatgt |
| | | gataacatggtggagcacgacactctcgtctactccaagaatatcaaagatacagtctca |
| | | gaagaccaaagggctattgagacttacaacaaagggtaatatcgggaaacctcctcgg |
| | | attccattgcccagctatctgtcacttcatcaaaaggacagtagaaaaggaaggtggca |
| | | cctacaaatgccatcattgcgataaaggaaaggctatcgttcaagatgcctctgccgaca |
| | | gtggtcccaaagatggacccccacccacgaggagcatcgtggaaaaagaagacgttc |
| | | caaccacgtcttcaaagcaagtggattgatgtgatatctccactgacgtaagggatgacg |
| | | cacaatcccactatccttcgcaagaccttcctctatataaggaagttcatttcatttggaga |
| | | ggacacgctgaaatcaccagtctctctctacaaatctatctcttaatacgactcactatagg |
| | | gagacccaagctggctagcaacaatggataagaagtactctatcggactcgatatcgg |
| | | aactaactctgttggatgggctgtgatcaccgatgagtacaaggtgccatctaagaagtt |
| | | caaggttctcggaaacaccgataggcactctatcaagaaaaaccttatcggtgctctcct |
| | | cttcgattctggtgaaactgctgaggctaccagactcaagagaaccgctagaagaaggt |
| | | acaccagaagaaagaacaggatctgctacctccaagagattttctctaacgagatggct |
| | | aaagtggatgattcattcttccacaggctcgaagagtcattcctcgtggaagaagataag |
| | | aagcacgagaggcaccctatcttcggaaacatcgttgatgaggtggcataccacgaga |
| | | agtaccctactatctaccacctcagaaagaagctcgttgattctactgataaggctgatct |
| | | caggctcatctacctcgctctcgctcacatgatcaagttcagaggacacttcctcatcga |
| | | gggtgatctcaaccctgataactctgatgtggataagttgttcatccagctcgtgcagacc |
| | | tacaaccagcttttcgaagagaaccctatcaacgcttcaggtgtggatgctaaggctatc |
| | | ctctctgctaggctctctaagtcaagaaggcttgagaacctcattgctcagctccctggtg |
| | | agaagaagaacggacttttcggaaacttgatcgctctctctctcggactcacccctaactt |
| | | caagtctaacttcgatctcgctgaggatgcaaagctccagctctcaaaggatacctacga |
| | | tgatgatctcgataacctcctcgctcagatcggagatcagtacgctgatttgttcctcgctg |
| | | ctaagaacctctctgatgctatcctcctcagtgatatcctcagggtgaacaccgagatca |
| | | ccaaggctccactttctgcttctatgatcaagagatacgatgagcaccaccaggatctca |
| | | cacttctcaaggctcttgttagacagcagctcccagagaagtacaaagaaatcttcttcg |
| | | atcagtctaagaacggatacgctggttacatcgatggtggtgcatctcaagaagagttct |
| | | acaagttcatcaagccaatcttggagaagatggatggaaccgaggaactcctcgtgaa |
| | | gctcaatagagaggatctccttaggaagcagaggaccttcgataacggatctatccctc |
| | | atcagatccacctcggagagttgcacgctatccttagaaggcaagaggatttctacccat |
| | | tcctcaaggataacagagagaagattgagaagatcctcaccttcagaatcccttactacg |
| | | tgggacctctcgctagaggaaactcaagattcgcttggatgaccagaaagtctgaggaa |
| | | accatcaccccttggaacttcgaagaggtggtggataagggtgctagtgctcagtctttc |
| | | atcgagaggatgaccaacttcgataagaaccttcctaacgagaaggtgctccctaagca |
| | | ctctttgctctacgagtacttcaccgtgtacaacgagttgaccaaggttaagtacgtgacc |
| | | gagggaatgaggaagcctgcttttttgtcaggtgagcaaaagaaggctatcgttgatctc |
| | | ttgttcaagaccaacagaaaggtgaccgtgaagcagctcaaagaggattacttcaagaa |
| | | aatcgagtgcttcgattcagtggaaatctctggtgttgaggataggttcaacgcatctctc |
| | | ggaacctaccacgatctcctcaagatcattaaggataaggatttcttggataacgaggaa |
| | | aacgaggatatcttggaggatatcgttcttaccctcaccctcttcgaggatagagagatg |
| | | atagaagaaaggctcaagacctacgctcatctcttcgatgataaggtgatgaagcagttg |
| | | aagagaagaagatacactggttggggaaggctctcaagaaagctcattaacggaatca |
| | | gggataagcagtctggaaagacaatccttgatttcctcaagtctgatggattcgctaaca |
| | | gaaacttcatgcagctcatccacgatgattctctcacctttaaagaggatatccagaagg |
| | | ctcaggtttcaggacagggtgatagtctccatgagcatatcgctaacctcgctggatccc |
| | | ctgcaatcaagaagggaatcctccagactgtgaagattgtggatgagttggtgaaggtg |
| | | atgggacacaagcctgagaacatcgtgatcgaaatggctagagagaaccagaccact |
| | | cagaagggacagaagaactctagggaaaggatgaagaggatcgaggaaggtatcaa |
| | | agagcttggatctcagatcctcaaagagcaccctgagagaacactcagctccagaacg |
| | | agaagctctacctctactacttgcagaacggaagggatatgtatgtggatcaagagcttg |
| | | atattaacaggctctctgattacgatgttgatcatatcgtgccacagtcttttatcaaagatg |
| | | attctatcgataacaaggtgctcactaggtctgataagaacaggggtaagagtgataac |
| | | gtgccaagtgaagaggttgtgaagaaaatgaagaactattggaggcagctcctcaacg |
| | | ctaagctcatcactcagagaaagttcgataacttgaccaaggctgagaggggaggact |
| | | ctctgaattggataaggcaggattcatcaagagacagctcgtggaaaccaggcagatc |
| | | accaaacatgtggcacagatcctcgattctaggatgaacaccaagtacgatgagaacg |
| | | ataagttgatcagggaagtgaaggttatcaccctcaagtcaaagctcgtgtctgatttcag |
| | | aaaggatttccaattctacaaggtgagggaaatcaacaactaccaccacgctcacgatg |
| | | cttaccttaacgctgttgttggaaccgctctcatcaagaagtatccaaagttggagtctga |
| | | gttcgtgtacggtgattataaggtgtacgatgtgaggaagatgatcgctaagtctgagca |
| | | agagatcggaaaggctaccgctaagtatttcttctactctaacatcatgaatacttcaaga |
| | | ccgagatcactctcgctaacggtgagatcagaaagaggccactcatcgagacaaacg |
| | | gtgaaacaggtgagatcgtgtgggataagggaagggatttcgctaccgttagaaaggt |
| | | gctctctatgcctcaggtgaacatcgttaagaaaaccgaggtgcagaccggtggattct |
| | | ctaaagagtctatcctccctaagaggaactctgataagctcattgctaggaagaaggatt |
| | | gggaccctaagaaatacggtggtttcgattctcctaccgtggcttactctgttctcgttgtg |
| | | gctaaggttgagaagggaaagagtaagaagctcaagtctgttaaggaacttctcggaat |
| | | cactatcatggaaaggtcatctttcgagaagaacccaatcgatttccttgaggctaaggg |
| | | atacaaagaggttaagaaggatctcatcatcaagctcccaaagtactcacttttcgagttg |
| | | gagaacggtagaaagaggatgctcgcttctgctggtgagcttcaaaagggaaacgag |
| | | cttgctctcccatctaagtacgttaactttctttacctcgcttctcactacgagaagttgaag |
| | | ggatctccagaagataacgagcagaagcaacttttcgttgagcagcacaagcactactt |
| | | ggatgagatcatcgagcagatcagtgagttctctaaaagggtgatcctcgctgatgcaa |
| | | acctcgataaggtgttgtctgcttacaacaagcacagagataagcctatcagggaacag |
| | | gcagagaacatcatccatctcttcacccttaccaacctcggtgctcctgctgctttcaagt |
| | | acttcgatacaaccatcgataggaagagatacacctctaccaaagaagtgctcgatgct |
| | | accctcatccatcagtctatcactggactctacgagactaggatcgatctctcacagcttg |
| | | gaggtgatcctaagaagaaaagaaaggttagatcttgatgacccgggtctccataataat |
| | | gtgtgagtagacccagataagggaattagggacctatagggtacgctcatgtgttgag |
| | | catataagaaacccttagtatgtatttgtatttgtaaaatacttctatcaataaaatttctaattc |
| | | ctaaaaccaaaatccagtactaaaatccagatcccccgaattaaggccttgacaggatat |
| | | attggcgggtaaacctaagagaaaagagcgtttattagaataacggatatttaaaactcg |
| | | ag |
| 98 | pCambia1301:35S:GUS | GATCTGAGGGTAAATTTCTAGTTTTTCTCCTTCATTTT |
| | | CTTGGTTAGGACCCTTTTCTCTTTTTATTTTTTTGAGC |
| | | TTTGATCTTTCTTTAAACTGATCTATTTTTTAATTGAT |
| | | TGGTTATGGTGTAAATATTACATAGCTTTAACTGATA |
| | | ATCTGATTACTTTATTTCGTGTGTCTATGATGATGAT |
| | | GATAGTTACAGAACCGACGACTCGTCCGTCCTGTAG |
| | | AAACCCCAACCCGTGAAATCAAAAAACTCGACGGCC |
| | | TGTGGGCATTCAGTCTGGATCGCGAAAACTGTGGAA |
| | | TTGATCAGCGTTGGTGGGAAAGCGCGTTACAAGAAA |
| | | GCCGGGCAATTGCTGTGCCAGGCAGTTTTAACGATC |
| | | AGTTCGCCGATGCAGATATTCGTAATTATGCGGGCA |
| | | ACGTCTGGTATCAGCGCGAAGTCTTTATACCGAAAG |
| | | GTTGGGCAGGCCAGCGTATCGTGCTGCGTTTCGATGC |
| | | GGTCACTCATTACGGCAAAGTGTGGGTCAATAATCA |
| | | GGAAGTGATGGAGCATCAGGGCGGCTATACGCCATT |
| | | TGAAGCCGATGTCACGCCGTATGTTATTGCCGGGAA |
| | | AAGTGTACGTATCACCGTTTGTGTGAACAACGAACT |
| | | GAACTGGCAGACTATCCCGCCGGGAATGGTGATTAC |
| | | CGACGAAAACGGCAAGAAAAAGCAGTCTTACTTCCA |
| | | TGATTTCTTTAACTATGCCGGAATCCATCGCAGCGTA |
| | | ATGCTCTACACCACGCCGAACACCTGGGTGGACGAT |
| | | ATCACCGTGGTGACGCATGTCGCGCAAGACTGTAAC |
| | | CACGCGTCTGTTGACTGGCAGGTGGTGGCCAATGGT |
| | | GATGTCAGCGTTGAACTGCGTGATGCGGATCAACAG |
| | | GTGGTTGCAACTGGACAAGGCACTAGCGGGACTTTG |
| | | CAAGTGGTGAATCCGCACCTCTGGCAACCGGGTGAA |
| | | GGTTATCTCTATGAACTCGAAGTCACAGCCAAAAGC |
| | | CAGACAGAGTCTGATATCTACCCGCTTCGCGTCGGCA |
| | | TCCGGTCAGTGGCAGTGAAGGGCCAACAGTTCCTGA |
| | | TTAACCACAAACCGTTCTACTTTACTGGCTTTGGTCG |
| | | TCATGAAGATGCGGACTTACGTGGCAAAGGATTCGA |
| | | TAACGTGCTGATGGTGCACGACCACGCATTAATGGA |
| | | CTGGATTGGGGCCAACTCCTACCGTACCTCGCATTAC |
| | | CCTTACGCTGAAGAGATGCTCGACTGGGCAGATGAA |
| | | CATGGCATCGTGGTGATTGATGAAACTGCTGCTGTCG |
| | | GCTTTCAGCTGTCTTTAGGCATTGGTTTCGAAGCGGG |
| | | CAACAAGCCGAAAGAACTGTACAGCGAAGAGGCAG |
| | | TCAACGGGGAAACTCAGCAAGCGCACTTACAGGCGA |
| | | TTAAAGAGCTGATAGCGCGTGACAAAAACCACCCAA |
| | | GCGTGGTGATGTGGAGTATTGCCAACGAACCGGATA |
| | | CCCGTCCGCAAGGTGCACGGGAATATTTCGCGCCAC |
| | | TGGCGGAAGCAACGCGTAAACTCGACCCGACGCGTC |
| | | CGATCACCTGCGTCAATGTAATGTTCTGCGACGCTCA |
| | | CACCGATACCATCAGCGATCTCTTTGATGTGCTGTGC |
| | | CTGAACCGTTATTACGGATGGTATGTCCAAAGCGGC |
| | | GATTTGGAAACGGCAGAGAAGGTACTGGAAAAAGA |
| | | ACTTCTGGCCTGGCAGGAGAAACTGCATCAGCCGAT |
| | | TATCATCACCGAATACGGCGTGGATACGTTAGCCGG |
| | | GCTGCACTCAATGTACACCGACATGTGGAGTGAAGA |
| | | GTATCAGTGTGCATGGCTGGATATGTATCACCGCGTC |
| | | TTTGATCGCGTCAGCGCCGTCGTCGGTGAACAGGTAT |
| | | GGAATTTCGCCGATTTTGCGACCTCGCAAGGCATATT |
| | | GCGCGTTGGCGGTAACAAGAAAGGGATCTTCACTCG |
| | | CGACCGCAAACCGAAGTCGGCGGCTTTTCTGCTGCA |
| | | AAAACGCTGGACTGGCATGAACTTCGGTGAAAAACC |
| | | GCAGCAGGGAGGCAAACAAGCTAGCCACCACCACCA |
| | | CCACCACGTGTGAATTACAGGTGACCAGCTCGAATTT |
| | | CCCCGATCGTTCAAACATTTGGCAATAAAGTTTCTTA |
| | | AGATTGAATCCTGTTGCCGGTCTTGCGATGATTATCA |
| | | TATAATTTCTGTTGAATTACGTTAAGCATGTAATAAT |
| | | TAACATGTAATGCATGACGTTATTTATGAGATGGGTT |
| | | TTTATGATTAGAGTCCCGCAATTATACATTTAATACG |
| | | CGATAGAAAACAAAATATAGCGCGCAAACTAGGATA |
| | | AATTATCGCGCGCGGTGTCATCTATGTTACTAGATCG |
| | | GGAATTAAACTATCAGTGTTTGACAGGATATATTGGC |
| | | GGGTAAACCTAAGAGAAAAGAGCGTTTATTAGAATA |
| | | ACGGATATTTAAAAGGGCGTGAAAAGGTTTATCCGT |
| | | TCGTCCATTTGTATGTGCATGCCAACCACAGGGTTCC |
| | | CCTCGGGATCAAAGTACTTTGATCCAACCCCTCCGCT |
| | | GCTATAGTGCAGTCGGCTTCTGACGTTCAGTGCAGCC |
| | | GTCTTCTGAAAACGACATGTCGCACAAGTCCTAAGTT |
| | | ACGCGACAGGCTGCCGCCCTGCCCTTTTCCTGGCGTT |
| | | TTCTTGTCGCGTGTTTTAGTCGCATAAAGTAGAATAC |
| | | TTGCGACTAGAACCGGAGACATTACGCCATGAACAA |
| | | GAGCGCCGCCGCTGGCCTGCTGGGCTATGCCCGCGT |
| | | CAGCACCGACGACCAGGACTTGACCAACCAACGGGC |
| | | CGAACTGCACGCGGCCGGCTGCACCAAGCTGTTTTCC |
| | | GAGAAGATCACCGGCACCAGGCGCGACCGCCCGGAG |
| | | CTGGCCAGGATGCTTGACCACCTACGCCCTGGCGAC |
| | | GTTGTGACAGTGACCAGGCTAGACCGCCTGGCCCGC |
| | | AGCACCCGCGACCTACTGGACATTGCCGAGCGCATC |
| | | CAGGAGGCCGGCGCGGGCCTGCGTAGCCTGGCAGAG |
| | | CCGTGGGCCGACACCACCACGCCGGCCGGCCGCATG |
| | | GTGTTGACCGTGTTCGCCGGCATTGCCGAGTTCGAGC |
| | | GTTCCCTAATCATCGACCGCACCCGGAGCGGGCGCG |
| | | AGGCCGCCAAGGCCCGAGGCGTGAAGTTTGGCCCCC |
| | | GCCCTACCCTCACCCCGGCACAGATCGCGCACGCCC |
| | | GCGAGCTGATCGACCAGGAAGGCCGCACCGTGAAAG |
| | | AGGCGGCTGCACTGCTTGGCGTGCATCGCTCGACCCT |
| | | GTACCGCGCACTTGAGCGCAGCGAGGAAGTGACGCC |
| | | CACCGAGGCCAGGCGGCGCGGTGCCTTCCGTGAGGA |
| | | CGCATTGACCGAGGCCGACGCCCTGGCGGCCGCCGA |
| | | GAATGAACGCCAAGAGGAACAAGCATGAAACCGCA |
| | | CCAGGACGGCCAGGACGAACCGTTTTTCATTACCGA |
| | | AGAGATCGAGGCGGAGATGATCGCGGCCGGGTACGT |
| | | GTTCGAGCCGCCCGCGCACGTCTCAACCGTGCGGCT |
| | | GCATGAAATCCTGGCCGGTTTGTCTGATGCCAAGCTG |
| | | GCGGCCTGGCCGGCCAGCTTGGCCGCTGAAGAAACC |
| | | GAGCGCCGCCGTCTAAAAAGGTGATGTGTATTTGAG |
| | | TAAAACAGCTTGCGTCATGCGGTCGCTGCGTATATGA |
| | | TGCGATGAGTAAATAAACAAATACGCAAGGGGAACG |
| | | CATGAAGGTTATCGCTGTACTTAACCAGAAAGGCGG |
| | | GTCAGGCAAGACGACCATCGCAACCCATCTAGCCCG |
| | | CGCCCTGCAACTCGCCGGGGCCGATGTTCTGTTAGTC |
| | | GATTCCGATCCCCAGGGCAGTGCCCGCGATTGGGCG |
| | | GCCGTGCGGGAAGATCAACCGCTAACCGTTGTCGGC |
| | | ATCGACCGCCCGACGATTGACCGCGACGTGAAGGCC |
| | | ATCGGCCGGCGCGACTTCGTAGTGATCGACGGAGCG |
| | | CCCCAGGCGGCGGACTTGGCTGTGTCCGCGATCAAG |
| | | GCAGCCGACTTCGTGCTGATTCCGGTGCAGCCAAGC |
| | | CCTTACGACATATGGGCCACCGCCGACCTGGTGGAG |
| | | CTGGTTAAGCAGCGCATTGAGGTCACGGATGGAAGG |
| | | CTACAAGCGGCCTTTGTCGTGTCGCGGGCGATCAAA |
| | | GGCACGCGCATCGGCGGTGAGGTTGCCGAGGCGCTG |
| | | GCCGGGTACGAGCTGCCCATTCTTGAGTCCCGTATCA |
| | | CGCAGCGCGTGAGCTACCCAGGCACTGCCGCCGCCG |
| | | GCACAACCGTTCTTGAATCAGAACCCGAGGGCGACG |
| | | CTGCCCGCGAGGTCCAGGCGCTGGCCGCTGAAATTA |
| | | AATCAAAACTCATTTGAGTTAATGAGGTAAAGAGAA |
| | | AATGAGCAAAAGCACAAACACGCTAAGTGCCGGCCG |
| | | TCCGAGCGCACGCAGCAGCAAGGCTGCAACGTTGGC |
| | | CAGCCTGGCAGACACGCCAGCCATGAAGCGGGTCAA |
| | | CTTTCAGTTGCCGGCGGAGGATCACACCAAGCTGAA |
| | | GATGTACGCGGTACGCCAAGGCAAGACCATTACCGA |
| | | GCTGCTATCTGAATACATCGCGCAGCTACCAGAGTA |
| | | AATGAGCAAATGAATAAATGAGTAGATGAATTTTAG |
| | | CGGCTAAAGGAGGCGGCATGGAAAATCAAGAACAA |
| | | CCAGGCACCGACGCCGTGGAATGCCCCATGTGTGGA |
| | | GGAACGGGCGGTTGGCCAGGCGTAAGCGGCTGGGTT |
| | | GTCTGCCGGCCCTGCAATGGCACTGGAACCCCCAAG |
| | | CCCGAGGAATCGGCGTGAGCGGTCGCAAACCATCCG |
| | | GCCCGGTACAAATCGGCGCGGCGCTGGGTGATGACC |
| | | TGGTGGAGAAGTTGAAGGCCGCGCAGGCCGCCCAGC |
| | | GGCAACGCATCGAGGCAGAAGCACGCCCCGGTGAAT |
| | | CGTGGCAAGCGGCCGCTGATCGAATCCGCAAAGAAT |
| | | CCCGGCAACCGCCGGCAGCCGGTGCGCCGTCGATTA |
| | | GGAAGCCGCCCAAGGGCGACGAGCAACCAGATTTTT |
| | | TCGTTCCGATGCTCTATGACGTGGGCACCCGCGATAG |
| | | TCGCAGCATCATGGACGTGGCCGTTTTCCGTCTGTCG |
| | | AAGCGTGACCGACGAGCTGGCGAGGTGATCCGCTAC |
| | | GAGCTTCCAGACGGGCACGTAGAGGTTTCCGCAGGG |
| | | CCGGCCGGCATGGCCAGTGTGTGGGATTACGACCTG |
| | | GTACTGATGGCGGTTTCCCATCTAACCGAATCCATGA |
| | | ACCGATACCGGGAAGGGAAGGGAGACAAGCCCGGC |
| | | CGCGTGTTCCGTCCACACGTTGCGGACGTACTCAAGT |
| | | TCTGCCGGCGAGCCGATGGCGGAAAGCAGAAAGACG |
| | | ACCTGGTAGAAACCTGCATTCGGTTAAACACCACGC |
| | | ACGTTGCCATGCAGCGTACGAAGAAGGCCAAGAACG |
| | | GCCGCCTGGTGACGGTATCCGAGGGTGAAGCCTTGA |
| | | TTAGCCGCTACAAGATCGTAAAGAGCGAAACCGGGC |
| | | GGCCGGAGTACATCGAGATCGAGCTAGCTGATTGGA |
| | | TGTACCGCGAGATCACAGAAGGCAAGAACCCGGACG |
| | | TGCTGACGGTTCACCCCGATTACTTTTTGATCGATCC |
| | | CGGCATCGGCCGTTTTCTCTACCGCCTGGCACGCCGC |
| | | GCCGCAGGCAAGGCAGAAGCCAGATGGTTGTTCAAG |
| | | ACGATCTACGAACGCAGTGGCAGCGCCGGAGAGTTC |
| | | AAGAAGTTCTGTTTCACCGTGCGCAAGCTGATCGGGT |
| | | CAAATGACCTGCCGGAGTACGATTTGAAGGAGGAGG |
| | | CGGGGCAGGCTGGCCCGATCCTAGTCATGCGCTACC |
| | | GCAACCTGATCGAGGGCGAAGCATCCGCCGGTTCCT |
| | | AATGTACGGAGCAGATGCTAGGGCAAATTGCCCTAG |
| | | CAGGGGAAAAAGGTCGAAAAGGTCTCTTTCCTGTGG |
| | | ATAGCACGTACATTGGGAACCCAAAGCCGTACATTG |
| | | GGAACCGGAACCCGTACATTGGGAACCCAAAGCCGT |
| | | ACATTGGGAACCGGTCACACATGTAAGTGACTGATA |
| | | TAAAAGAGAAAAAAGGCGATTTTTCCGCCTAAAACT |
| | | CTTTAAAACTTATTAAAACTCTTAAAACCCGCCTGGC |
| | | CTGTGCATAACTGTCTGGCCAGCGCACAGCCGAAGA |
| | | GCTGCAAAAAGCGCCTACCCTTCGGTCGCTGCGCTCC |
| | | CTACGCCCCGCCGCTTCGCGTCGGCCTATCGCGGCCG |
| | | CTGGCCGCTCAAAAATGGCTGGCCTACGGCCAGGCA |
| | | ATCTACCAGGGCGCGGACAAGCCGCGCCGTCGCCAC |
| | | TCGACCGCCGGCGCCCACATCAAGGCACCCTGCCTC |
| | | GCGCGTTTCGGTGATGACGGTGAAAACCTCTGACAC |
| | | ATGCAGCTCCCGGAGACGGTCACAGCTTGTCTGTAA |
| | | GCGGATGCCGGGAGCAGACAAGCCCGTCAGGGCGCG |
| | | TCAGCGGGTGTTGGCGGGTGTCGGGGCGCAGCCATG |
| | | ACCCAGTCACGTAGCGATAGCGGAGTGTATACTGGC |
| | | TTAACTATGCGGCATCAGAGCAGATTGTACTGAGAG |
| | | TGCACCATATGCGGTGTGAAATACCGCACAGATGCG |
| | | TAAGGAGAAAATACCGCATCAGGCGCTCTTCCGCTT |
| | | CCTCGCTCACTGACTCGCTGCGCTCGGTCGTTCGGCT |
| | | GCGGCGAGCGGTATCAGCTCACTCAAAGGCGGTAAT |
| | | ACGGTTATCCACAGAATCAGGGGATAACGCAGGAAA |
| | | GAACATGTGAGCAAAAGGCCAGCAAAAGGCCAGGA |
| | | ACCGTAAAAAGGCCGCGTTGCTGGCGTTTTTCCATAG |
| | | GCTCCGCCCCCCTGACGAGCATCACAAAAATCGACG |
| | | CTCAAGTCAGAGGTGGCGAAACCCGACAGGACTATA |
| | | AAGATACCAGGCGTTTCCCCCTGGAAGCTCCCTCGTG |
| | | CGCTCTCCTGTTCCGACCCTGCCGCTTACCGGATACC |
| | | TGTCCGCCTTTCTCCCTTCGGGAAGCGTGGCGCTTTC |
| | | TCATAGCTCACGCTGTAGGTATCTCAGTTCGGTGTAG |
| | | GTCGTTCGCTCCAAGCTGGGCTGTGTGCACGAACCCC |
| | | CCGTTCAGCCCGACCGCTGCGCCTTATCCGGTAACTA |
| | | TCGTCTTGAGTCCAACCCGGTAAGACACGACTTATCG |
| | | CCACTGGCAGCAGCCACTGGTAACAGGATTAGCAGA |
| | | GCGAGGTATGTAGGCGGTGCTACAGAGTTCTTGAAG |
| | | TGGTGGCCTAACTACGGCTACACTAGAAGGACAGTA |
| | | TTTGGTATCTGCGCTCTGCTGAAGCCAGTTACCTTCG |
| | | GAAAAAGAGTTGGTAGCTCTTGATCCGGCAAACAAA |
| | | CCACCGCTGGTAGCGGTGGTTTTTTTGTTTGCAAGCA |
| | | GCAGATTACGCGCAGAAAAAAAGGATCTCAAGAAG |
| | | ATCCTTTGATCTTTTCTACGGGGTCTGACGCTCAGTG |
| | | GAACGAAAACTCACGTTAAGGGATTTTGGTCATGCA |
| | | TTCTAGGTACTAAAACAATTCATCCAGTAAAATATAA |
| | | TATTTTATTTTCTCCCAATCAGGCTTGATCCCCAGTA |
| | | AGTCAAAAAATAGCTCGACATACTGTTCTTCCCCGAT |
| | | ATCCTCCCTGATCGACCGGACGCAGAAGGCAATGTC |
| | | ATACCACTTGTCCGCCCTGCCGCTTCTCCCAAGATCA |
| | | ATAAAGCCACTTACTTTGCCATCTTTCACAAAGATGT |
| | | TGCTGTCTCCCAGGTCGCCGTGGGAAAAGACAAGTT |
| | | CCTCTTCGGGCTTTTCCGTCTTTAAAAAATCATACAG |
| | | CTCGCGCGGATCTTTAAATGGAGTGTCTTCTTCCCAG |
| | | TTTTCGCAATCCACATCGGCCAGATCGTTATTCAGTA |
| | | AGTAATCCAATTCGGCTAAGCGGCTGTCTAAGCTATT |
| | | CGTATAGGGACAATCCGATATGTCGATGGAGTGAAA |
| | | GAGCCTGATGCACTCCGCATACAGCTCGATAATCTTT |
| | | TCAGGGCTTTGTTCATCTTCATACTCTTCCGAGCAAA |
| | | GGACGCCATCGGCCTCACTCATGAGCAGATTGCTCC |
| | | AGCCATCATGCCGTTCAAAGTGCAGGACCTTTGGAA |
| | | CAGGCAGCTTTCCTTCCAGCCATAGCATCATGTCCTT |
| | | TTCCCGTTCCACATCATAGGTGGTCCCTTTATACCGG |
| | | CTGTCCGTCATTTTTAAATATAGGTTTTCATTTTCTCC |
| | | CACCAGCTTATATACCTTAGCAGGAGACATTCCTTCC |
| | | GTATCTTTTACGCAGCGGTATTTTTCGATCAGTTTTTT |
| | | CAATTCCGGTGATATTCTCATTTTAGCCATTTATTATT |
| | | TCCTTCCTCTTTTCTACAGTATTTAAAGATACCCCAA |
| | | GAAGCTAATTATAACAAGACGAACTCCAATTCACTG |
| | | TTCCTTGCATTCTAAAACCTTAAATACCAGAAAACAG |
| | | CTTTTTCAAAGTTGTTTTCAAAGTTGGCGTATAACAT |
| | | AGTATCGACGGAGCCGATTTTGAAACCGCGGTGATC |
| | | ACAGGCAGCAACGCTCTGTCATCGTTACAATCAACA |
| | | TGCTACCCTCCGCGAGATCATCCGTGTTTCAAACCCG |
| | | GCAGCTTAGTTGCCGTTCTTCCGAATAGCATCGGTAA |
| | | CATGAGCAAAGTCTGCCGCCTTACAACGGCTCTCCCG |
| | | CTGACGCCGTCCCGGACTGATGGGCTGCCTGTATCGA |
| | | GTGGTGATTTTGTGCCGAGCTGCCGGTCGGGGAGCT |
| | | GTTGGCTGGCTGGTGGCAGGATATATTGTGGTGTAA |
| | | ACAAATTGACGCTTAGACAACTTAATAACACATTGC |
| | | GGACGTTTTTAATGTACTGAATTAACGCCGAATTAAT |
| | | TCGGGGGATCTGGATTTTAGTACTGGATTTTGGTTTT |
| | | AGGAATTAGAAATTTTATTGATAGAAGTATTTTACAA |
| | | ATACAAATACATACTAAGGGTTTCTTATATGCTCAAC |
| | | ACATGAGCGAAACCCTATAGGAACCCTAATTCCCTT |
| | | ATCTGGGAACTACTCACACATTATTATGGAGAAACTC |
| | | GAGCTTGTCGATCGACAGATCCGGTCGGCATCTACTC |
| | | TATTTCTTTGCCCTCGGACGAGTGCTGGGGCGTCGGT |
| | | TTCCACTATCGGCGAGTACTTCTACACAGCCATCGGT |
| | | CCAGACGGCCGCGCTTCTGCGGGCGATTTGTGTACGC |
| | | CCGACAGTCCCGGCTCCGGATCGGACGATTGCGTCG |
| | | CATCGACCCTGCGCCCAAGCTGCATCATCGAAATTGC |
| | | CGTCAACCAAGCTCTGATAGAGTTGGTCAAGACCAA |
| | | TGCGGAGCATATACGCCCGGAGTCGTGGCGATCCTG |
| | | CAAGCTCCGGATGCCTCCGCTCGAAGTAGCGCGTCT |
| | | GCTGCTCCATACAAGCCAACCACGGCCTCCAGAAGA |
| | | AGATGTTGGCGACCTCGTATTGGGAATCCCCGAACA |
| | | TCGCCTCGCTCCAGTCAATGACCGCTGTTATGCGGCC |
| | | ATTGTCCGTCAGGACATTGTTGGAGCCGAAATCCGC |
| | | GTGCACGAGGTGCCGGACTTCGGGGCAGTCCTCGGC |
| | | CCAAAGCATCAGCTCATCGAGAGCCTGCGCGACGGA |
| | | CGCACTGACGGTGTCGTCCATCACAGTTTGCCAGTGA |
| | | TACACATGGGGATCAGCAATCGCGCATATGAAATCA |
| | | CGCCATGTAGTGTATTGACCGATTCCTTGCGGTCCGA |
| | | ATGGGCCGAACCCGCTCGTCTGGCTAAGATCGGCCG |
| | | CAGCGATCGCATCCATAGCCTCCGCGACCGGTTGTA |
| | | GAACAGCGGGCAGTTCGGTTTCAGGCAGGTCTTGCA |
| | | ACGTGACACCCTGTGCACGGCGGGAGATGCAATAGG |
| | | TCAGGCTCTCGCTAAACTCCCCAATGTCAAGCACTTC |
| | | CGGAATCGGGAGCGCGGCCGATGCAAAGTGCCGATA |
| | | AACATAACGATCTTTGTAGAAACCATCGGCGCAGCT |
| | | ATTTACCCGCAGGACATATCCACGCCCTCCTACATCG |
| | | AAGCTGAAAGCACGAGATTCTTCGCCCTCCGAGAGC |
| | | TGCATCAGGTCGGAGACGCTGTCGAACTTTTCGATCA |
| | | GAAACTTCTCGACAGACGTCGCGGTGAGTTCAGGCT |
| | | TTTTCATATCTCATTGCCCCCCGGGATCTGCGAAAGC |
| | | TCGAGAGAGATAGATTTGTAGAGAGAGACTGGTGAT |
| | | TTCAGCGTGTCCTCTCCAAATGAAATGAACTTCCTTA |
| | | TATAGAGGAAGGTCTTGCGAAGGATAGTGGGATTGT |
| | | GCGTCATCCCTTACGTCAGTGGAGATATCACATCAAT |
| | | CCACTTGCTTTGAAGACGTGGTTGGAACGTCTTCTTT |
| | | TTCCACGATGCTCCTCGTGGGTGGGGGTCCATCTTTG |
| | | GGACCACTGTCGGCAGAGGCATCTTGAACGATAGCC |
| | | TTTCCTTTATCGCAATGATGGCATTTGTAGGTGCCAC |
| | | CTTCCTTTTCTACTGTCCTTTTGATGAAGTGACAGAT |
| | | AGCTGGGCAATGGAATCCGAGGAGGTTTCCCGATAT |
| | | TACCCTTTGTTGAAAAGTCTCAATAGCCCTTTGGTCT |
| | | TCTGAGACTGTATCTTTGATATTCTTGGAGTAGACGA |
| | | GAGTGTCGTGCTCCACCATGTTATCACATCAATCCAC |
| | | TTGCTTTGAAGACGTGGTTGGAACGTCTTCTTTTTCC |
| | | ACGATGCTCCTCGTGGGTGGGGGTCCATCTTTGGGAC |
| | | CACTGTCGGCAGAGGCATCTTGAACGATAGCCTTTCC |
| | | TTTATCGCAATGATGGCATTTGTAGGTGCCACCTTCC |
| | | TTTTCTACTGTCCTTTTGATGAAGTGACAGATAGCTG |
| | | GGCAATGGAATCCGAGGAGGTTTCCCGATATTACCC |
| | | TTTGTTGAAAAGTCTCAATAGCCCTTTGGTCTTCTGA |
| | | GACTGTATCTTTGATATTCTTGGAGTAGACGAGAGTG |
| | | TCGTGCTCCACCATGTTGGCAAGCTGCTCTAGCCAAT |
| | | ACGCAAACCGCCTCTCCCCGCGCGTTGGCCGATTCAT |
| | | TAATGCAGCTGGCACGACAGGTTTCCCGACTGGAAA |
| | | GCGGGCAGTGAGCGCAACGCAATTAATGTGAGTTAG |
| | | CTCACTCATTAGGCACCCCAGGCTTTACACTTTATGC |
| | | TTCCGGCTCGTATGTTGTGTGGAATTGTGAGCGGATA |
| | | ACAATTTCACACAGGAAACAGCTATGACCATGATTA |
| | | CGAATTCGAGCTCGGTACCCGGGGATCCTCTAGAGT |
| | | CGACCTGCAGGCATGCAAGCTTGGCACTGGCCGTCG |
| | | TTTTACAACGTCGTGACTGGGAAAACCCTGGCGTTAC |
| | | CCAACTTAATCGCCTTGCAGCACATCCCCCTTTCGCC |
| | | AGCTGGCGTAATAGCGAAGAGGCCCGCACCGATCGC |
| | | CCTTCCCAACAGTTGCGCAGCCTGAATGGCGAATGCT |
| | | AGAGCAGCTTGAGCTTGGATCAGATTGTCGTTTCCCG |
| | | CCTTCAGTTTAGCTTCATGGAGTCAAAGATTCAAATA |
| | | GAGGACCTAACAGAACTCGCCGTAAAGACTGGCGAA |
| | | CAGTTCATACAGAGTCTCTTACGACTCAATGACAAG |
| | | AAGAAAATCTTCGTCAACATGGTGGAGCACGACACA |
| | | CTTGTCTACTCCAAAAATATCAAAGATACAGTCTCAG |
| | | AAGACCAAAGGGCAATTGAGACTTTTCAACAAAGGG |
| | | TAATATCCGGAAACCTCCTCGGATTCCATTGCCCAGC |
| | | TATCTGTCACTTTATTGTGAAGATAGTGGAAAAGGA |
| | | AGGTGGCTCCTACAAATGCCATCATTGCGATAAAGG |
| | | AAAGGCCATCGTTGAAGATGCCTCTGCCGACAGTGG |
| | | TCCCAAAGATGGACCCCCACCCACGAGGAGCATCGT |
| | | GGAAAAAGAAGACGTTCCAACCACGTCTTCAAAGCA |
| | | AGTGGATTGATGTGATATCTCCACTGACGTAAGGGA |
| | | TGACGCACAATCCCACTATCCTTCGCAAGACCCTTCC |
| | | TCTATATAAGGAAGTTCATTTCATTTGGAGAGAACAC |
| | | GGGGGACTCTTGACCATGGTA |
| 99 | pGWB5:35S:CBCAScds:stop | tgagcgtcgcaaaggcgctcggtcttgccttgctcgtcggtgatgtacttcaccagctcc |
| | | gcgaagtcgctcttcttgatggagcgcatggggacgtgcttggcaatcacgcgcaccc |
| | | cccggccgttttagcggctaaaaaagtcatggctctgccctcgggcggaccacgccca |
| | | tcatgaccttgccaagctcgtcctgcttctcttcgatcttcgccagcagggcgaggatcgt |
| | | ggcatcaccgaaccgcgccgtgcgcgggtcgtcggtgagccagagtttcagcaggcc |
| | | gcccaggcggcccaggtcgccattgatgcgggccagctcgcggacgtgctcatagtc |
| | | cacgacgcccgtgattttgtagccctggccgacggccagcaggtaggccgacaggct |
| | | catgccggccgccgccgccttttcctcaatcgctcttcgttcgtctggaaggcagtacac |
| | | cttgataggtgggctgcccttcctggttggcttggtttcatcagccatccgcttgccctcat |
| | | ctgttacgccggcggtagccggccagcctcgcagagcaggattcccgttgagcaccg |
| | | ccaggtgcgaataagggacagtgaagaaggaacacccgctcgcgggtgggcctactt |
| | | cacctatcctgcccggctgacgccgttggatacaccaaggaaagtctacacgaaccctt |
| | | tggcaaaatcctgtatatcgtgcgaaaaaggatggatataccgaaaaaatcgctataatg |
| | | accccgaagcagggttatgcagcggaaaagcgccacgcttcccgaagggagaaagg |
| | | cggacaggtatccggtaagcggcagggtcggaacaggagagcgcacgagggagct |
| | | tccagggggaaacgcctggtatctttatagtcctgtcgggtttcgccacctctgacttgag |
| | | cgtcgatttttgtgatgctcgtcaggggggcggagcctatggaaaaacgccagcaacg |
| | | cggcctttttacggttcctggccttttgctggccttttgctcacatgttctttcctgcgttatcc |
| | | cctgattctgtggataaccgtattaccgcctttgagtgagctgataccgctcgccgcagc |
| | | cgaacgaccgagcgcagcgagtcagtgagcgaggaagcggaagagcgccagaag |
| | | gccgccagagaggccgagcgcggccgtgaggcttggacgctagggcagggcatga |
| | | aaaagcccgtagcgggctgctacgggcgtctgacgcggtggaaagggggaggggat |
| | | gttgtctacatggctctgctgtagtgagtgggttgcgctccggcagcggtcctgatcaat |
| | | cgtcaccctttctcggtccacaacgttcctgacaacgagcctccttttcgccaatccatcg |
| | | acaatcaccgcgagtccctgctcgaacgctgcgtccggaccggcttcgtcgaaggcgt |
| | | ctatcgcggcccgcaacagcggcgagagcggagcctgttcaacggtgccgccgcgc |
| | | tcgccggcatcgctgtcgccggcctgctcctcaagcacggccccaacagtgaagtagc |
| | | tgattgtcatcagcgcattgacggcgtccccggccgaaaaacccgcctcgcagaggaa |
| | | gcgaagctgcgcgtcggccgtttccatctgcggtgcgcccggtcgcgtgccggcatg |
| | | gatgcgcgcgccatcgcggtaggcgagcagcgcctgcctgaagctgcgggcattccc |
| | | gatcagaaatgagcgccagtcgtcgtcggctctcggcaccgaatgcgtatgattctccg |
| | | ccagcatggcttcggccagtgcgtcgagcagcgcccgcttgttcctgaagtgccagta |
| | | aagcgccggctgctgaacccccaaccgttccgccagtttgcgtgtcgtcagaccgtcta |
| | | cgccgacctcgttcaacaggtccagggcggcacggatcactgtattcggctgcaacttt |
| | | gtcatgcttgacactttatcactgataaacataatatgtccaccaacttatcagtgataaag |
| | | aatccgcgcgttcaatcggaccagcggaggctggtccggaggccagacgtgaaacc |
| | | caacatacccctgatcgtaattctgagcactgtcgcgctcgacgctgtcggcatcggcct |
| | | gattatgccggtgctgccgggcctcctgcgcgatctggttcactcgaacgacgtcaccg |
| | | cccactatggcattctgctggcgctgtatgcgaggtgcaatttgcctgcgcacctgtgct |
| | | gggcgcgctgtcggatcgtttcgggcggcggccaatcttgctcgtctcgctggccggc |
| | | gccagatctggggaaccctgtggttggcatgcacatacaaatggacgaacggataaac |
| | | cttttcacgcccttttaaatatccgattattctaataaacgctcttatttcttaggatacccgcc |
| | | aatatatcctgtcaaacactgatagtttaaactgaaggcgggaaacgacaatctgatcat |
| | | gagcggagaattaagggagtcacgttatgacccccgccgatgacgcgggacaagcc |
| | | gttttacgtttggaactgacagaaccgcaacgttgaaggagccactcagccgcgggttt |
| | | ctggagtttaatgagctaagcacatacgtcagaaaccattattgcgcgttcaaaagtcgc |
| | | ctaaggtcactatcagctagcaaatatttcttgtcaaaaatgctccactgacgttccataaa |
| | | ttcccctcggtatccaattagagtctcatattcactctcaatccaaataatctgcaccggat |
| | | ctggatcgtttcgcatgattgaacaagatggattgcacgcaggactccggccgcaggg |
| | | tggagaggctattcggctatgactgggcacaacagacaatcggctgctctgatgccgc |
| | | cgtgaccggctgtcagcgcaggggcgcccggactttttgtcaagaccgacctgtccg |
| | | gtgccctgaatgaactgcaggacgaggcagcgcggctatcgtggctggccacgacg |
| | | ggcgttccttgcgcagctgtgctcgacgttgtcactgaagcgggaagggactggctgct |
| | | attgggcgaagtgccggggcaggatctcctgtcatctcaccttgctcctgccgagaaag |
| | | tatccatcatggctgatgcaatgcggcggctgcatacgcttgatccggctacctgcccat |
| | | tcgaccaccaagcgaaacatcgcatcgagcgagcacgtactcggatggaagccggtc |
| | | ttgtcgatcaggatgatctggacgaagagcatcaggggctcgcgccagccgaactgtt |
| | | cgccaggctcaaggcgcgcatgcccgacggcgatgatctcgtcgtgacccatggcga |
| | | tgcctgcttgccgaatatcatggtggaaaatggccgcttttctggattcatcgactgtggc |
| | | cggctgggtgtggcggaccgctatcaggacatagcgttggctacccgtgatattgctga |
| | | agagcttggcggcgaatgggctgaccgcttcctcgtgctttacggtatcgccgctcccg |
| | | attcgcagcgcatcgccttctatcgccttcttgacgagttcttctgagcgggactctgggg |
| | | ttcgaaatgaccgaccaagcgacgcccaacctgccatcacgagatttcgattccaccgc |
| | | cgccactatgaaaggttgggcttcggaatcgttttccgggacgccggctggatgatcct |
| | | ccagcgcggggatctcatgctggagttcttcgcccacgggatctctgcggaacaggcg |
| | | gtcgaaggtgccgatatcattacgacagcaacggccgacaagcacaacgccacgatc |
| | | ctgagcgacaatatgatcgggcccggcgtccacatcaacggcgtcggcggcgactgc |
| | | ccaggcaagaccgagatgcaccgcgatatcttgctgcgttcggatattttcgtggagttc |
| | | ccgccacagacccggatgatccccgatcgttcaaacatttggcaataaagtttcttaaga |
| | | ttgaatcctgttgccggtcttgcgatgattatcatataatttctgttgaattacgttaagcatgt |
| | | aataattaacatgtaatgcatgacgttatttatgagatgggtttttatgattagagtcccgca |
| | | attatacatttaatacgcgatagaaaacaaaatatagcgcgcaaactaggataaattatcg |
| | | cgcgcggtgtcatctatgttactagatcgggcctcctgtcaatgctggcggcggctctgg |
| | | tggtggttctggtggcggctctgagggtggtggctctgagggtggcggttctgagggtg |
| | | gcggctctgagggaggcggttccggtggtggctctggttccggtgattttgattatgaaa |
| | | agatggcaaacgctaataagggggctatgaccgaaaatgccgatgaaaacgcgctac |
| | | agtctgacgctaaaggcaaacttgattctgtcgctactgattacggtgctgctatcgatgg |
| | | tttcattggtgacgtttccggccagctaatggtaatggtgctactggtgattttgctggctct |
| | | aattcccaaatggctcaagtcggtgacggtgataattcaccataatgaataatttccgtca |
| | | atatttaccttccctccctcaatcggttgaatgtcgcccttttgtctttggcccaatacgcaa |
| | | accgcctctccccgcgcgttggccgattcattaatgcagctggcacgacaggtttcccg |
| | | actggaaagcgggcagtgagcgcaacgcaattaatgtgagttagctcactcattaggca |
| | | ccccaggctttacactttatgcttccggctcgtatgttgtgtggaattgtgagcggataaca |
| | | atttcacacaggaaacagctatgaccatgattacgccaagcttgcatgcctgcaggtccc |
| | | cagattagccttttcaatttcagaaagaatgctaacccacagatggttagagaggcttacg |
| | | cagcaggtctcatcaagacgatctacccgagcaataatctccaggaaatcaaataccttc |
| | | ccaagaaggttaaagatgcagtcaaaagattcaggactaactgcatcaagaacacaga |
| | | gaaagatatatttctcaagatcagaagtactattccagtatggacgattcaaggcttgcttc |
| | | acaaaccaaggcaagtaatagagattggagtctctaaaaaggtagttcccactgaatca |
| | | aaggccatggagtcaaagattcaaatagaggacctaacagaactcgccgtaaagactg |
| | | gcgaacagttcatacagagtctcttacgactcaatgacaagaagaaaatcttcgtcaaca |
| | | tggtggagcacgacacacttgtctactccaaaaatatcaaagatacagtctcagaagac |
| | | caaagggcaattgagacttttcaacaaagggtaatatccggaaacctcctcggattccat |
| | | tgcccagctatctgtcactttattgtgaagatagtggaaaaggaaggtggctcctacaaat |
| | | gccatcattgcgataaaggaaaggccatcgttgaagatgcctctgccgacagtggtccc |
| | | aaagatggacccccacccacgaggagcatcgtggaaaaagaagacgttccaaccac |
| | | gtcttcaaagcaagtggattgatgtgatatctccactgacgtaagggatgacgcacaatc |
| | | ccactatccttcgcaagacccttcctctatataaggaagttcatttcatttggagagaacac |
| | | gggggactctaatcaaacaagtttgtacaaaaaagctgaacgagaaacgtaaaatgatg |
| | | aattgctcaacattctccttttggtttgtttgcaaaataattttttactactctcattcaatatcca |
| | | aatttcaatagctaatcctcaagaaaacttccttaaatgcttctcggaatatattcctaacaa |
| | | tccagcaaatccaaaattcatatacactcaacacgaccaattgtatatgtctgtcctgaatt |
| | | cgacaatacaaaatcttagattcacctctgatacaaccccaaaaccactcgttattgtcact |
| | | ccttcaaatgtctcccatatccaggccagtattctctgctccaagaaagttggtttgcagat |
| | | tcgaactcgaagcggtggccatgatgctgagggtttgtcctacatatctcaagtcccattt |
| | | gctatagtagacttgagaaacatgcatacggtcaaagtagatattcatagccaaactgcg |
| | | tgggttgaagccggagctacccttggagaagtttattattggatcaatgagatgaatgag |
| | | aattttagttttcctggtgggtattgccctactgttggcgtaggtggacactttagtggagg |
| | | aggctatggagcattgatgcgaaattatggccttgcggctgataatatcattgatgcacac |
| | | ttagtcaatgttgatggaaaagttctagatcgaaaatccatgggagaagatctattttggg |
| | | ctatacgtggtggaggaggagaaaactttggaatcattgcagcatggaaaatcaaactt |
| | | gttgttgtcccatcaaaggctactatattcagtgttaaaaagaacatggagatacatgggc |
| | | ttgtcaagttatttaacaaatggcaaaatattgcttacaagtatgacaaagatttaatgctca |
| | | cgactcacttcagaactaggaatattacagataatcatgggaagaataagactacagtac |
| | | atggttacttctcttccatttttcttggtggagtggatagtctagttgacttgatgaacaagag |
| | | ctttcctgagttgggtattaaaaaaactgattgcaaagaattgagctggattgatacaacc |
| | | atcttctacagtggtgttgtaaattacaacactgctaattttaaaaaggaaattttgcttgata |
| | | gatcagctgggaagaagacggctttctcaattaagttagactatgttaagaaactaatacc |
| | | tgaaactgcaatggtcaaaattttggaaaaattatatgaagaagaggtaggagttgggat |
| | | gtatgtgttgtacccttacggtggtataatggatgagatttcagaatcagcaattccattcc |
| | | ctcatcgagctggaataatgtatgaactttggtacactgctacctgggagaagcaagaa |
| | | gataacgaaaagcatataaactgggttcgaagtgtttataatttcacaactccttatgtgtc |
| | | ccaaaatccaagattggcgtatctcaattatagggaccttgatttaggaaaaactaatcct |
| | | gagagtcctaataattacacacaagcacgtatttggggtgaaaagtattttggtaaaaattt |
| | | taacaggttagttaaggtgaaaaccaaagctgatcccaataatttttttagaaacgaacaa |
| | | agtatcccacctcttccaccgcgtcatcattaaaatatattgatatttatatcattttacgtttct |
| | | cgttcagctttcttgtacaaagtggttcgatctagaggatccatggtgagcaagggcgag |
| | | gagctgttcaccggggtggtgcccatcctggtcgagctggacggcgacgtgaacggc |
| | | cacaagttcagcgtgtccggcgagggcgagggcgatgccacctacggcaagctgac |
| | | cctgaagttcatctgcaccaccggcaagctgcccgtgccctggcccaccctcgtgacc |
| | | accttcacctacggcgtgcagtgcttcagccgctaccccgaccacatgaagcagcacg |
| | | acttcttcaagtccgccatgcccgaaggctacgtccaggagcgcaccatcttcttcaag |
| | | gacgacggcaactacaagacccgcgccgaggtgaagttcgagggcgacaccctggt |
| | | gaaccgcatcgagctgaagggcatcgacttcaaggaggacggcaacatcctggggc |
| | | acaagctggagtacaactacaacagccacaacgtctatatcatggccgacaagcagaa |
| | | gaacggcatcaaggtgaacttcaagatccgccacaacatcgaggacggcagcgtgca |
| | | gctcgccgaccactaccagcagaacacccccatcggcgacggccccgtgctgctgcc |
| | | cgacaaccactacctgagcacccagtccgccctgagcaaagaccccaacgagaagc |
| | | gcgatcacatggtcctgctggagttcgtgaccgccgccgggatcactcacggcatgga |
| | | cgagctgtacaagtaaagcggcccgagctcgaatttccccgatcgttcaaacatttggc |
| | | aataaagtttcttaagattgaatcctgttgccggtcttgcgatgattatcatataatttctgttg |
| | | aattacgttaagcatgtaataattaacatgtaatgcatgacgttatttatgagatgggttttta |
| | | tgattagagtcccgcaattatacatttaatacgcgatagaaaacaaaatatagcgcgcaa |
| | | actaggataaattatcgcgcgcggtgtcatctatgttactagatcgggaattagcttcatca |
| | | acgcaagacatgcgcacgaccgtctgacaggagaggaataccgacgagcacagaa |
| | | aggacttgctcttggacgtaggcctatttctcaggcacatgtatcaagtgttcggacgtgg |
| | | gttttcgatggtgtatcagccgccgccaactgggagatgaggaggctttcttggggggc |
| | | agtcagcagttcatttcacaagacagaggaacttgtaaggagatgcactgatttatcttgg |
| | | cgcaaaccagcaggacgaattagtgggaatagcccgcgaatatctaagttatgcctgtc |
| | | ggcatgagcagaaacttccaattcgaaacagtttggagaggttgtttttgggcatacctttt |
| | | gttagtcagcctctcgattgctcatcgtcattacacagtaccgaagtttgatcgatctagta |
| | | acatagatgacaccgcgcgcgataatttatcctagtttgcgcgctatattttgttttctatcg |
| | | cgtattaaatgtataattgcgggactctaatcataaaaacccatctcataaataacgtcatg |
| | | cattacatgttaattattacatgcttaacgtaattcaacagaaattatatgataatcatcgcaa |
| | | gaccggcaacaggattcaatcttaagaaactttattgccaaatgtttgaacgatctgcttc |
| | | gacgcactccttctttactccaccatctcgtccttattgaaaacgtgggtagcaccaaaac |
| | | gaatcaagtcgctggaactgaagttaccaatcacgctggatgatttgccagttggattaat |
| | | cttgcctttccccgcatgaataatattgatgaatgcatgcgtgaggggtatttcgattttgg |
| | | caatagctgcaattgccgcgacatcctccaacgagcataattcttcagaaaaatagcgat |
| | | gttccatgttgtcagggcatgcatgatgcacgttatgaggtgacggtgctaggcagtatt |
| | | ccctcaaagtttcatagtcagtatcatattcatcattgcattcctgcaagagagaattgaga |
| | | cgcaatccacacgctgcggcaaccttccggcgttcgtggtctatttgctcttggacgttgc |
| | | aaacgtaagtgttggatcccggtcggcatctactctattcctagccctcggacgagtgct |
| | | ggggcgtcggtttccactatcggcgagtacttctacacagccatcggtccagacggcc |
| | | gcgcttctgcgggcgatttgtgtacgcccgacagtcccggctccggatcggacgattgc |
| | | gtcgcatcgaccctgcgcccaagctgcatcatcgaaattgccgtcaaccaagctctgat |
| | | agagttggtcaagaccaatgcggagcatatacgcccggagccgcggcgatcctgcaa |
| | | gctccggatgcctccgctcgaagtagcgcgtctgctgctccatacaagccaaccacgg |
| | | cctccagaagaagatgttggcgacctcgtattgggaatccccgaacatcgcctcgctcc |
| | | agtcaatgaccgctgttatgcggccattgtccgtcaggacattgttggagccgaaatccg |
| | | cgtgcacgaggtgccggacttcggggcagtcctcggcccaaagcatcagctcatcga |
| | | gagcctgcgcgacggacgcactgacggtgtcgtccatcacagtttgccagtgatacac |
| | | atggggatcagcaatcgcgcatatgaaatcacgccatgtagtgtattgaccgattccttg |
| | | cggtccgaatgggccgaacccgctcgtctggctaagatcggccgcagcgatcgcatc |
| | | catggcctccgcgaccggctgcagaacagcgggcagttcggtttcaggcaggtcttgc |
| | | aacgtgacaccctgtgcacggcgggagatgcaataggtcaggctctcgctgaattccc |
| | | caatgtcaagcacttccggaatcgggagcgcggccgatgcaaagtgccgataaacata |
| | | acgatctttgtagaaaccatcggcgcagctatttacccgcaggacatatccacgccctcc |
| | | tacatcgaagctgaaagcacgagattcttcgccctccgagagctgcatcaggtcggag |
| | | acgctgtcgaacttttcgatcagaaacttctcgacagacgtcgcggtgagttcaggctttt |
| | | tcatatcggggtcgtcctctccaaatgaaatgaacttccttatatagaggaagggtcttgc |
| | | gaaggatagtgggattgtgcgtcatcccttacgtcagtggagatatcacatcaatccactt |
| | | gctttgaagacgtggaggaacgtcttctttttccacgatgctcctcgtgggtgggggtcc |
| | | atctttgggaccactgtcggcagaggcatcttgaacgatagcctttcctttatcgcaatgat |
| | | ggcatttgtaggtgccaccttccttttctactgtccttttgatgaagtgacagatagctggg |
| | | caatggaatccgaggaggtttcccgatattaccctttgttgaaaagtctcaatagccctttg |
| | | gtcttctgagactgtatctttgatattcttggagtagacgagagtgtcgtgctccaccatgtt |
| | | gacggatctctaggacgcgtcctagaagctaattcactggccgtcgttttacaacgtcgt |
| | | gactgggaaaaccctggcgttacccaacttaatcgccttgcagcacatccccctttcgcc |
| | | agctggcgtaatagcgaagaggcccgcaccgatcgcccttcccaacagttgcgcagc |
| | | ctgaatggcgcccgctcctttcgctacttcccttcctactcgccacgttcgccggctttcc |
| | | ccgtcaagctctaaatcgggggctccctttagggttccgatttagtgctttacggcacctc |
| | | gaccccaaaaaacttgatttgggtgatggttcacgtagtgggccatcgccctgatagac |
| | | ggtttttcgccctttgacgttggagtccacgttctttaatagtggactcttgttccaaactgg |
| | | aacaacactcaaccctatctcgggctattcttttgatttataagggattttgccgatttcgga |
| | | accaccatcaaacaggattttcgcctgctggggcaaaccagcgtggaccgcttgctgc |
| | | aactctctcagggccaggcggtgaagggcaatcagctgttgcccgtctcactggtgaa |
| | | aagaaaaaccaccccagtacattaaaaacgtccgcaatgtgttattaagttgtctaagcgt |
| | | caatttgtttacaccacaatatatcctgccaccagccagccaacagctccccgaccggc |
| | | agctcggcacaaaatcaccactcgatacaggcagcccatcagtccgggacggcgtca |
| | | gcgggagagccgttgtaaggcggcagactttgctcatgttaccgatgctattcggaaga |
| | | acggcaactaagctgccgggtttgaaacacggatgatctcgcggagggtagcatgttg |
| | | attgtaacgatgacagagcgttgctgcctgtgatcaaatatcatctccctcgcagagatcc |
| | | gaattatcagccttcttattcatttctcgcttaaccgtgacaggctgtcgatcttgagaacta |
| | | tgccgacataataggaaatcgctggataaagccgctgaggaagctgagtggcgctattt |
| | | ctttagaagtgaacgttgacgatatcaactcccctatccattgctcaccgaatggtacagg |
| | | tcggggacccgaagttccgactgtcggcctgatgcatccccggctgatcgaccccaga |
| | | tctggggctgagaaagcccagtaaggaaacaactgtaggttcgagtcgcgagatcccc |
| | | cggaaccaaaggaagtaggttaaacccgctccgatcaggccgagccacgccaggcc |
| | | gagaacattggttcctgtaggcatcgggattggcggatcaaacactaaagctactggaa |
| | | cgagcagaagtcctccggccgccagttgccaggcggtaaaggtgagcagaggcacg |
| | | ggaggttgccacttgcgggtcagcacggttccgaacgccatggaaaccgcccccgcc |
| | | aggcccgctgcgacgccgacaggatctagcgctgcgtttggtgtcaacaccaacagc |
| | | gccacgcccgcagttccgcaaatagcccccaggaccgccatcaatcgtatcgggctac |
| | | ctagcagagcggcagagatgaacacgaccatcagcggctgcacagcgcctaccgtc |
| | | gccgcgaccccgcccggcaggcggtagaccgaaataaacaacaagctccagaatag |
| | | cgaaatattaagtgcgccgaggatgaagatgcgcatccaccagattcccgttggaatct |
| | | gtcggacgatcatcacgagcaataaacccgccggcaacgcccgcagcagcataccg |
| | | gcgacccctcggcctcgctgttcgggctccacgaaaacgccggacagatgcgccttgt |
| | | gagcgtccttggggccgtcctcctgtttgaagaccgacagcccaatgatctcgccgtcg |
| | | atgtaggcgccgaatgccacggcatctcgcaaccgttcagcgaacgcctccatgggct |
| | | ttttctcctcgtgctcgtaaacggacccgaacatctctggagctttcttcagggccgacaa |
| | | tcggatctcgcggaaatcctgcacgtcggccgctccaagccgtcgaatctgagccttaa |
| | | tcacaattgtcaattttaatcctctgtttatcggcagttcgtagagcgcgccgtgcgtcccg |
| | | agcgatactgagcgaagcaagtgcgtcgagcagtgcccgcttgttcctgaaatgccag |
| | | taaagcgctggctgctgaacccccagccggaactgaccccacaaggccctagcgtttg |
| | | caatgcaccaggtcatcattgacccaggcgtgttccaccaggccgctgcctcgcaactc |
| | | ttcgcaggcttcgccgacctgctcgcgccacttcttcacgcgggtggaatccgatccgc |
| | | acatgaggcggaaggtttccagcttgagcgggtacggctcccggtgcgagctgaaata |
| | | gtcgaacatccgtcgggccgtcggcgacagcttgcggtacttctcccatatgaatttcgt |
| | | gtagtggtcgccagcaaacagcacgacgatttcctcgtcgatcaggacctggcaacgg |
| | | gacgttttcttgccacggtccaggacgcggaagcggtgcagcagcgacaccgattcca |
| | | ggtgcccaacgcggtcggacgtgaagcccatcgccgtcgcctgtaggcgcgacagg |
| | | cattcctcggccttcgtgtaataccggccattgatcgaccagcccaggtcctggcaaag |
| | | ctcgtagaacgtgaaggtgatcggctcgccgataggggtgcgcttcgcgtactccaac |
| | | acctgctgccacaccagttcgtcatcgtcggcccgcagctcgacgccggtgtaggtgat |
| | | cttcacgtccttgttgacgtggaaaatgaccttgttttgcagcgcctcgcgcgggattttct |
| | | tgttgcgcgtggtgaacagggcagagcgggccgtgtcgtttggcatcgctcgcatcgt |
| | | gtccggccacggcgcaatatcgaacaaggaaagctgcatttccttgatctgctgcttcgt |
| | | gtgtttcagcaacgcggcctgcttggcctcgctgacctgttttgccaggtcctcgccggc |
| | | ggtttttcgcttcttggtcgtcatagttcctcgcgtgtcgatggtcatcgacttcgccaaacc |
| | | tgccgcctcctgttcgagacgacgcgaacgctccacggcggccgatggcgcgggca |
| | | gggcagggggagccagttgcacgctgtcgcgctcgatcttggccgtagcttgctggac |
| | | catcgagccgacggactggaaggtttcgcggggcgcacgcatgacggtgcggcttgc |
| | | gatggtacggcatcctcggcggaaaaccccgcgtcgatcagacttgcctgtatgccttc |
| | | cggtcaaacgtccgattcattcaccctccttgcgggattgccccgactcacgccggggc |
| | | aatgtgcccttattcctgatttgacccgcctggtgccttggtgtccagataatccaccttatc |
| | | ggcaatgaagtcggtcccgtagaccgtctggccgtccttctcgtacttggtattccgaatc |
| | | ttgccctgcacgaataccagcgaccccttgcccaaatacttgccgtgggcctcggcctg |
| | | agagccaaaacacttgatgcggaagaagtcggtgcgctcctgcttgtcgccggcatcg |
| | | ttgcgccacatctaggtactaaaacaattcatccagtaaaatataatattttattttctcccaa |
| | | tcaggcttgatccccagtaagtcaaaaaatagctcgacatactgttcaccccgatatcct |
| | | ccctgatcgaccggacgcagaaggcaatgtcataccacttgtccgccctgccgcttctc |
| | | ccaagatcaataaagccacttactttgccatctttcacaaagatgttgctgtctcccaggtc |
| | | gccgtgggaaaagacaagttcctcttcgggcttttccgtcttaaaaaatcatacagctcg |
| | | cgcggatctttaaatggagtgtcttcttcccagttttcgcaatccacatcggccagatcgtt |
| | | attcagtaagtaatccaattcggctaagcggctgtctaagctattcgtatagggacaatcc |
| | | gatatgtcgatggagtgaaagagcctgatgcactccgcatacagctcgataatcttttca |
| | | gggctttgttcatcttcatactcttccgagcaaaggacgccatcggcctcactcatgagca |
| | | gattgctccagccatcatgccgttcaaagtgcaggacctttggaacaggcagctttcctt |
| | | ccagccatagcatcatgtccttttcccgttccacatcataggtggtccctttataccggctg |
| | | tccgtcattataaatataggttttcattttctcccaccagcttatataccttagcaggagacat |
| | | tccttccgtatcttttacgcagcggtatttttcgatcagttttacaattccggtgatattctcatt |
| | | ttagccatttattatttccttcctcttttctacagtatttaaagataccccaagaagctaattata |
| | | acaagacgaactccaattcactgttccttgcattctaaaaccttaaataccagaaaacagc |
| | | tttttcaaagttgttttcaaagttggcgtataacatagtatcgacggagccgattttgaaacc |
| | | acaattatgggtgatgctgccaacttactgatttagtgtatgatggtgtttttgaggtgctcc |
| | | agtggcttctgtgtctatcagctgtccctcctgttcagctactgacggggtggtgcgtaac |
| | | ggcaaaagcaccgccggacatcagcgctatctctgctctcactgccgtaaaacatggc |
| | | aactgcagttcacttacaccgcttctcaacccggtacgcaccagaaaatcattgatatgg |
| | | ccatgaatggcgttggatgccgggcaacagcccgcattatgggcgttggcctcaacac |
| | | gattttacgtcacttaaaaaactcaggccgcagtcggtaacctcgcgcatacagccggg |
| | | cagtgacgtcatcgtctgcgcggaaatggacgaacagtggggctatgtcggggctaaa |
| | | tcgcgccagcgctggctgttttacgcgtatgacagtctccggaagacggttgttgcgca |
| | | cgtattcggtgaacgcactatggcgacgctggggcgtcttatgagcctgctgtcaccctt |
| | | tgacgtggtgatatggatgacggatggctggccgctgtatgaatcccgcctgaaggga |
| | | aagctgcacgtaatcagcaagcgatatacgcagcgaattgagcggcataacctgaatct |
| | | gaggcagcacctggcacggctgggacggaagtcgctgtcgttctcaaaatcggtgga |
| | | gctgcatgacaaagtcatcgggcattatctgaacataaaacactatcaataagttggagt |
| | | cattacccaattatgatagaatttacaagctataaggttattgtcctgggtacaagcattag |
| | | tccatgcaagtttttatgctagcccattctatagatatattgataagcgcgctgcctatgcct |
| | | tgccccctgaaatccttacatacggcgatatcttctatataaaagatatattatcttatcagta |
| | | ttgtcaatatattcaaggcaatctgcctcctcatcctcttcatcctcttcgtcttggtagcttttt |
| | | aaatatggcgcttcatagagtaattctgtaaaggtccaattctcgttttcatacctcggtata |
| | | atcttacctatcacctcaaatggttcgctgggtttatcgcacccccgaacacgagcacgg |
| | | cacccgcgaccactatgccaagaatgcccaaggtaaaaattgccggccccgccatga |
| | | agtccgtgaatgccccgacggccgaagtgaagggcaggccgccacccaggccgcc |
| | | gccctcactgcccggcacctggtcgctgaatgtcgatgccagcacctgcggcacgtca |
| | | atgcttccgggcgtcgcgctcgggctgatcgcccatcccgttactgccccgatcccggc |
| | | aatggcaaggactgccagcgctgccatttttggggtgaggccgttcgcggccgaggg |
| | | gcgcagcccctggggggatgggaggcccgcgttagcgggccgggagggttcgaga |
| | | agggggggcaccccccttcggcgtgcgcggtcacgcgcacagggcgcagccctggt |
| | | taaaaacaaggtttataaatattggtaaaaagcaggttaaaagacaggttagcggtggc |
| | | cgaaaaacgggcggaaacccttgcaaatgctggattttctgcctgtggacagcccctca |
| | | aatgtcaataggtgcgcccctcatctgtcagcactctgcccctcaagtgtcaaggatcgc |
| | | gcccctcatctgtcagtagtcgcgcccctcaagtgtcaataccgcagggcacttatcccc |
| | | aggcttgtccacatcatctgtgggaaactcgcgtaaaatcaggcgttttcgccgatttgcg |
| | | aggctggccagctccacgtcgccggccgaaatcgagcctgcccctcatctgtcaacgc |
| | | cgcgccgggtgagtcggcccctcaagtgtcaacgtccgcccctcatctgtcagtgagg |
| | | gccaagttttccgcgaggtatccacaacgccggcggccgcggtgtctcgcacacggct |
| | | tcgacggcgtttctggcgcgtttgcagggccatagacggccgccagcccagcggcga |
| | | gggcaaccagcccgg |
| 100 | pGWB5:35S:CBDAScds:Stop | tgagcgtcgcaaaggcgctcggtcttgccttgctcgtcggtgatgtacttcaccagctcc |
| | | gcgaagtcgctcttcttgatggagcgcatggggacgtgcttggcaatcacgcgcaccc |
| | | cccggccgttttagcggctaaaaaagtcatggctctgccctcgggcggaccacgccca |
| | | tcatgaccttgccaagctcgtcctgcttctcttcgatcttcgccagcagggcgaggatcgt |
| | | ggcatcaccgaaccgcgccgtgcgcgggtcgtcggtgagccagagtttcagcaggcc |
| | | gcccaggcggcccaggtcgccattgatgcgggccagctcgcggacgtgctcatagtc |
| | | cacgacgcccgtgattttgtagccctggccgacggccagcaggtaggccgacaggct |
| | | catgccggccgccgccgccttttcctcaatcgctcttcgttcgtctggaaggcagtacac |
| | | cttgataggtgggctgcccttcctggttggcttggtttcatcagccatccgcttgccctcat |
| | | ctgttacgccggcggtagccggccagcctcgcagagcaggattcccgttgagcaccg |
| | | ccaggtgcgaataagggacagtgaagaaggaacacccgctcgcgggtgggcctactt |
| | | cacctatcctgcccggctgacgccgttggatacaccaaggaaagtctacacgaaccctt |
| | | tggcaaaatcctgtatatcgtgcgaaaaaggatggatataccgaaaaaatcgctataatg |
| | | accccgaagcagggttatgcagcggaaaagcgccacgcttcccgaagggagaaagg |
| | | cggacaggtatccggtaagcggcagggtcggaacaggagagcgcacgagggagct |
| | | tccagggggaaacgcctggtatctttatagtcctgtcgggtttcgccacctctgacttgag |
| | | cgtcgatttttgtgatgctcgtcaggggggcggagcctatggaaaaacgccagcaacg |
| | | cggcctttttacggacctggccttttgctggccttttgctcacatgttctttcctgcgttatcc |
| | | cctgattctgtggataaccgtattaccgcctttgagtgagctgataccgctcgccgcagc |
| | | cgaacgaccgagcgcagcgagtcagtgagcgaggaagcggaagagcgccagaag |
| | | gccgccagagaggccgagcgcggccgtgaggcttggacgctagggcagggcatga |
| | | aaaagcccgtagcgggctgctacgggcgtctgacgcggtggaaagggggaggggat |
| | | gttgtctacatggctctgctgtagtgagtgggttgcgctccggcagcggtcctgatcaat |
| | | cgtcaccctttctcggtccttcaacgttcctgacaacgagcctccttttcgccaatccatcg |
| | | acaatcaccgcgagtccctgctcgaacgctgcgtccggaccggcttcgtcgaaggcgt |
| | | ctatcgcggcccgcaacagcggcgagagcggagcctgttcaacggtgccgccgcgc |
| | | tcgccggcatcgctgtcgccggcctgctcctcaagcacggccccaacagtgaagtagc |
| | | tgattgtcatcagcgcattgacggcgtccccggccgaaaaacccgcctcgcagaggaa |
| | | gcgaagctgcgcgtcggccgtttccatctgcggtgcgcccggtcgcgtgccggcatg |
| | | gatgcgcgcgccatcgcggtaggcgagcagcgcctgcctgaagctgcgggcattccc |
| | | gatcagaaatgagcgccagtcgtcgtcggctctcggcaccgaatgcgtatgattctccg |
| | | ccagcatggcttcggccagtgcgtcgagcagcgcccgcttgttcctgaagtgccagta |
| | | aagcgccggctgctgaacccccaaccgttccgccagtttgcgtgtcgtcagaccgtcta |
| | | cgccgacctcgttcaacaggtccagggcggcacggatcactgtattcggctgcaacttt |
| | | gtcatgcttgacactttatcactgataaacataatatgtccaccaacttatcagtgataaag |
| | | aatccgcgcgttcaatcggaccagcggaggctggtccggaggccagacgtgaaacc |
| | | caacatacccctgatcgtaattctgagcactgtcgcgctcgacgctgtcggcatcggcct |
| | | gattatgccggtgctgccgggcctcctgcgcgatctggttcactcgaacgacgtcaccg |
| | | cccactatggcattctgctggcgctgtatgcgaggtgcaatttgcctgcgcacctgtgct |
| | | gggcgcgctgtcggatcgtttcgggcggcggccaatcttgctcgtctcgctggccggc |
| | | gccagatctggggaaccctgtggttggcatgcacatacaaatggacgaacggataaac |
| | | cttttcacgcccttttaaatatccgattattctaataaacgctcttttctcttaggtttacccgcc |
| | | aatatatcctgtcaaacactgatagtttaaactgaaggcgggaaacgacaatctgatcat |
| | | gagcggagaattaagggagtcacgttatgacccccgccgatgacgcgggacaagcc |
| | | gttttacgtttggaactgacagaaccgcaacgttgaaggagccactcagccgcgggttt |
| | | ctggagtttaatgagctaagcacatacgtcagaaaccattattgcgcgttcaaaagtcgc |
| | | ctaaggtcactatcagctagcaaatatttcttgtcaaaaatgctccactgacgttccataaa |
| | | ttcccctcggtatccaattagagtctcatattcactctcaatccaaataatctgcaccggat |
| | | ctggatcgtttcgcatgattgaacaagatggattgcacgcaggttctccggccgcttggg |
| | | tggagaggctattcggctatgactgggcacaacagacaatcggctgctctgatgccgc |
| | | cgtgttccggctgtcagcgcaggggcgcccggttctttttgtcaagaccgacctgtccg |
| | | gtgccctgaatgaactgcaggacgaggcagcgcggctatcgtggctggccacgacg |
| | | ggcgttccttgcgcagctgtgctcgacgttgtcactgaagcgggaagggactggctgct |
| | | attgggcgaagtgccggggcaggatctcctgtcatctcaccttgctcctgccgagaaag |
| | | tatccatcatggctgatgcaatgcggcggctgcatacgcttgatccggctacctgcccat |
| | | tcgaccaccaagcgaaacatcgcatcgagcgagcacgtactcggatggaagccggtc |
| | | ttgtcgatcaggatgatctggacgaagagcatcaggggctcgcgccagccgaactgtt |
| | | cgccaggctcaaggcgcgcatgcccgacggcgatgatctcgtcgtgacccatggcga |
| | | tgcctgcttgccgaatatcatggtggaaaatggccgcttttctggattcatcgactgtggc |
| | | cggctgggtgtggcggaccgctatcaggacatagcgttggctacccgtgatattgctga |
| | | agagcttggcggcgaatgggctgaccgcttcctcgtgctttacggtatcgccgctcccg |
| | | attcgcagcgcatcgccttctatcgccttcttgacgagttcttctgagcgggactctgggg |
| | | ttcgaaatgaccgaccaagcgacgcccaacctgccatcacgagatttcgattccaccgc |
| | | cgccttctatgaaaggttgggcttcggaatcgttttccgggacgccggctggatgatcct |
| | | ccagcgcggggatctcatgctggagttcttcgcccacgggatctctgcggaacaggcg |
| | | gtcgaaggtgccgatatcattacgacagcaacggccgacaagcacaacgccacgatc |
| | | ctgagcgacaatatgatcgggcccggcgtccacatcaacggcgtcggcggcgactgc |
| | | ccaggcaagaccgagatgcaccgcgatatcttgctgcgttcggatattttcgtggagttc |
| | | ccgccacagacccggatgatccccgatcgttcaaacatttggcaataaagtttcttaaga |
| | | ttgaatcctgttgccggtcttgcgatgattatcatataatttctgttgaattacgttaagcatgt |
| | | aataattaacatgtaatgcatgacgttatttatgagatgggtattatgattagagtcccgca |
| | | attatacatttaatacgcgatagaaaacaaaatatagcgcgcaaactaggataaattatcg |
| | | cgcgcggtgtcatctatgttactagatcgggcctcctgtcaatgctggcggcggctctgg |
| | | tggtggttctggtggcggctctgagggtggtggctctgagggtggcggttctgagggtg |
| | | gcggctctgagggaggcggttccggtggtggctctggttccggtgattttgattatgaaa |
| | | agatggcaaacgctaataagggggctatgaccgaaaatgccgatgaaaacgcgctac |
| | | agtctgacgctaaaggcaaacttgattctgtcgctactgattacggtgctgctatcgatgg |
| | | tttcattggtgacgtttccggccttgctaatggtaatggtgctactggtgattttgctggctct |
| | | aattcccaaatggctcaagtcggtgacggtgataattcacctttaatgaataataccgtca |
| | | atatttaccttccctccctcaatcggttgaatgtcgcccttttgtctttggcccaatacgcaa |
| | | accgcctctccccgcgcgttggccgattcattaatgcagctggcacgacaggtttcccg |
| | | actggaaagcgggcagtgagcgcaacgcaattaatgtgagttagctcactcattaggca |
| | | ccccaggctttacactttatgcttccggctcgtatgagtgtggaattgtgagcggataaca |
| | | atttcacacaggaaacagctatgaccatgattacgccaagcttgcatgcctgcaggtccc |
| | | cagattagccttttcaatttcagaaagaatgctaacccacagatggttagagaggcttacg |
| | | cagcaggtctcatcaagacgatctacccgagcaataatctccaggaaatcaaataccttc |
| | | ccaagaaggttaaagatgcagtcaaaagattcaggactaactgcatcaagaacacaga |
| | | gaaagatatatttctcaagatcagaagtactattccagtatggacgattcaaggcttgcttc |
| | | acaaaccaaggcaagtaatagagattggagtctctaaaaaggtagttcccactgaatca |
| | | aaggccatggagtcaaagattcaaatagaggacctaacagaactcgccgtaaagactg |
| | | gcgaacagttcatacagagtctcttacgactcaatgacaagaagaaaatcttcgtcaaca |
| | | tggtggagcacgacacacttgtctactccaaaaatatcaaagatacagtctcagaagac |
| | | caaagggcaattgagacttttcaacaaagggtaatatccggaaacctcctcggattccat |
| | | tgcccagctatctgtcactttattgtgaagatagtggaaaaggaaggtggctcctacaaat |
| | | gccatcattgcgataaaggaaaggccatcgttgaagatgcctctgccgacagtggtccc |
| | | aaagatggacccccacccacgaggagcatcgtggaaaaagaagacgttccaaccac |
| | | gtcttcaaagcaagtggattgatgtgatatctccactgacgtaagggatgacgcacaatc |
| | | ccactatccttcgcaagacccttcctctatataaggaagttcatttcatttggagagaacac |
| | | gggggactctaatcaaacaagtttgtacaaaaaagctgaacgagaaacgtaaaatgA |
| | | TGAAGTACTCAACATTCTCCTTTTGGTTTGTTTGCAA |
| | | GATAATATTTTTCTTTTTCTCATTCAATATCCAAACTT |
| | | CCATTGCTAATCCTCGAGAAAACTTCCTTAAATGCTT |
| | | CTCGCAATATATTCCCAATAATGCAACAAATCTAAA |
| | | ACTCGTATACACTCAAAACAACCCATTGTATATGTCT |
| | | GTCCTAAATTCGACAATACACAATCTTAGATTCAGCT |
| | | CTGACACAACCCCAAAACCACTTGTTATCGTCACTCC |
| | | TTCACATGTCTCTCATATCCAAGGCACTATTCTATGC |
| | | TCCAAGAAAGTTGGCTTGCAGATTCGAACTCGAAGT |
| | | GGTGGTCATGATTCTGAGGGCATGTCCTACATATCTC |
| | | AAGTCCCATTTGTTATAGTAGACTTGAGAAACATGCG |
| | | TTCAATCAAAATAGATGTTCATAGCCAAACTGCATG |
| | | GGTTGAAGCCGGAGCTACCCTTGGAGAAGTTTATTAT |
| | | TGGGTTAATGAGAAAAATGAGAGTCTTAGTTTGGCT |
| | | GCTGGGTATTGCCCTACTGTTTGCGCAGGTGGACACT |
| | | TTGGTGGAGGAGGCTATGGACCATTGATGAGAAGCT |
| | | ATGGCCTCGCGGCTGATAATATCATTGATGCACACTT |
| | | AGTCAACGTTCATGGAAAAGTGCTAGATCGAAAATC |
| | | TATGGGGGAAGATCTCTTTTGGGCTTTACGTGGTGGT |
| | | GGAGCAGAAAGCTTCGGAATCATTGTAGCATGGAAA |
| | | ATTAGACTGGTTGCTGTCCCAAAGTCTACTATGTTTA |
| | | GTGTTAAAAAGATCATGGAGATACATGAGCTTGTCA |
| | | AGTTAGTTAACAAATGGCAAAATATTGCTTACAAGT |
| | | ATGACAAAGATTTATTACTCATGACTCACTTCATAAC |
| | | TAGGAACATTACAGATAATCAAGGGAAGAATAAGAC |
| | | AGCAATACACACTTACTTCTCTTCAGTTTTCCTTGGT |
| | | GGAGTGGATAGTCTAGTCGACTTGATGAACAAGAGT |
| | | TTTCCTGAGTTGGGTATTAAAAAAACGGATTGCAGA |
| | | CAATTGAGCTGGATTGATACTATCATCTTCTATAGTG |
| | | GTGTTGTAAATTACGACACTGATAATTTTAACAAGGA |
| | | AATTTTGCTTGATAGATCCGCTGGGCAGAACGGTGCT |
| | | TTCAAGATTAAGTTAGACTACGTTAAGAAACCAATTC |
| | | CAGAATCTGTATTTGTCCAAATTTTGGAAAAATTATA |
| | | TGAAGAAGATATAGGAGCTGGGATGTATGCGTTGTA |
| | | CCCTTACGGTGGTATAATGGATGAGATTTCTGAATCA |
| | | GCAATTCCATTCCCTCATCGAGCTGGAATCTTGTATG |
| | | AGTTATGGTACATATGTAGCTGGGAGAAGCAAGAAG |
| | | ATAACGAAAAGCATCTAAACTGGATTAGAAATATTT |
| | | ATAACTTCATGACTCCTTATGTGTCCCAAAATCCAAG |
| | | ATTGGCATATCTCAATTATAGAGACCTTGATATAGGA |
| | | ATAAATGATCCCAAGAATCCAAATAATTACACACAA |
| | | GCACGTATTTGGGGTGAGAAGTATTTTGGTAAAAATT |
| | | TTGACAGGCTAGTAAAAGTGAAAACCCTGGTTGATC |
| | | CCAATAATTTTTTTAGAAACGAACAAAGCATCCCACC |
| | | TCTTCCACGGCATCATCATTAAaatatattgatatttatatcattttacg |
| | | tttctcgttcagctttcttgtacaaagtggttcgatctagaggatccatggtgagcaagggc |
| | | gaggagctgttcaccggggtggtgcccatcctggtcgagctggacggcgacgtgaac |
| | | ggccacaagttcagcgtgtccggcgagggcgagggcgatgccacctacggcaagct |
| | | gaccctgaagttcatctgcaccaccggcaagctgcccgtgccctggcccaccctcgtg |
| | | accaccttcacctacggcgtgcagtgcttcagccgctaccccgaccacatgaagcagc |
| | | acgacttcttcaagtccgccatgcccgaaggctacgtccaggagcgcaccatcttcttca |
| | | aggacgacggcaactacaagacccgcgccgaggtgaagttcgagggcgacaccctg |
| | | gtgaaccgcatcgagctgaagggcatcgacttcaaggaggacggcaacatcctgggg |
| | | cacaagctggagtacaactacaacagccacaacgtctatatcatggccgacaagcaga |
| | | agaacggcatcaaggtgaacttcaagatccgccacaacatcgaggacggcagcgtgc |
| | | agctcgccgaccactaccagcagaacacccccatcggcgacggccccgtgctgctgc |
| | | ccgacaaccactacctgagcacccagtccgccctgagcaaagaccccaacgagaag |
| | | cgcgatcacatggtcctgctggagttcgtgaccgccgccgggatcactcacggcatgg |
| | | acgagctgtacaagtaaagcggcccgagctcgaatttccccgatcgttcaaacatttgg |
| | | caataaagtttcttaagattgaatcctgttgccggtcttgcgatgattatcatataatttctgtt |
| | | gaattacgttaagcatgtaataattaacatgtaatgcatgacgttatttatgagatgggttttt |
| | | atgattagagtcccgcaattatacatttaatacgcgatagaaaacaaaatatagcgcgca |
| | | aactaggataaattatcgcgcgcggtgtcatctatgttactagatcgggaattagcttcatc |
| | | aacgcaagacatgcgcacgaccgtctgacaggagaggaatttccgacgagcacaga |
| | | aaggacttgctcttggacgtaggcctatttctcaggcacatgtatcaagtgttcggacgtg |
| | | ggttttcgatggtgtatcagccgccgccaactgggagatgaggaggctttcttgggggg |
| | | cagtcagcagttcatttcacaagacagaggaacttgtaaggagatgcactgatttatcttg |
| | | gcgcaaaccagcaggacgaattagtgggaatagcccgcgaatatctaagttatgcctgt |
| | | cggcatgagcagaaacttccaattcgaaacagtttggagaggttgtttttgggcatacctt |
| | | ttgttagtcagcctctcgattgctcatcgtcattacacagtaccgaagtttgatcgatctagt |
| | | aacatagatgacaccgcgcgcgataatttatcctagtttgcgcgctatattttgttttctatc |
| | | gcgtattaaatgtataattgcgggactctaatcataaaaacccatctcataaataacgtcat |
| | | gcattacatgttaattattacatgcttaacgtaattcaacagaaattatatgataatcatcgca |
| | | agaccggcaacaggattcaatcttaagaaactttattgccaaatgtttgaacgatctgcttc |
| | | gacgcactccttctttactccaccatctcgtccttattgaaaacgtgggtagcaccaaaac |
| | | gaatcaagtcgctggaactgaagttaccaatcacgctggatgatttgccagttggattaat |
| | | cttgcctttccccgcatgaataatattgatgaatgcatgcgtgaggggtatttcgattttgg |
| | | caatagctgcaattgccgcgacatcctccaacgagcataattcttcagaaaaatagcgat |
| | | gttccatgttgtcagggcatgcatgatgcacgttatgaggtgacggtgctaggcagtatt |
| | | ccctcaaagtttcatagtcagtatcatattcatcattgcattcctgcaagagagaattgaga |
| | | cgcaatccacacgctgcggcaaccttccggcgttcgtggtctatttgctcttggacgttgc |
| | | aaacgtaagtgttggatcccggtcggcatctactctattcctagccctcggacgagtgct |
| | | ggggcgtcggtttccactatcggcgagtacttctacacagccatcggtccagacggcc |
| | | gcgcttctgcgggcgatttgtgtacgcccgacagtcccggctccggatcggacgattgc |
| | | gtcgcatcgaccctgcgcccaagctgcatcatcgaaattgccgtcaaccaagctctgat |
| | | agagttggtcaagaccaatgcggagcatatacgcccggagccgcggcgatcctgcaa |
| | | gctccggatgcctccgctcgaagtagcgcgtctgctgctccatacaagccaaccacgg |
| | | cctccagaagaagatgttggcgacctcgtattgggaatccccgaacatcgcctcgctcc |
| | | agtcaatgaccgctgttatgcggccattgtccgtcaggacattgttggagccgaaatccg |
| | | cgtgcacgaggtgccggacttcggggcagtcctcggcccaaagcatcagctcatcga |
| | | gagcctgcgcgacggacgcactgacggtgtcgtccatcacagtttgccagtgatacac |
| | | atggggatcagcaatcgcgcatatgaaatcacgccatgtagtgtattgaccgattccttg |
| | | cggtccgaatgggccgaacccgctcgtctggctaagatcggccgcagcgatcgcatc |
| | | catggcctccgcgaccggctgcagaacagcgggcagttcggtttcaggcaggtcttgc |
| | | aacgtgacaccctgtgcacggcgggagatgcaataggtcaggctctcgctgaattccc |
| | | caatgtcaagcacttccggaatcgggagcgcggccgatgcaaagtgccgataaacata |
| | | acgatctttgtagaaaccatcggcgcagctatttacccgcaggacatatccacgccctcc |
| | | tacatcgaagctgaaagcacgagattcttcgccctccgagagctgcatcaggtcggag |
| | | acgctgtcgaacttttcgatcagaaacttctcgacagacgtcgcggtgagttcaggctttt |
| | | tcatatcggggtcgtcctctccaaatgaaatgaacttccttatatagaggaagggtcttgc |
| | | gaaggatagtgggattgtgcgtcatcccttacgtcagtggagatatcacatcaatccactt |
| | | gctttgaagacgtggaggaacgtcttctttttccacgatgctcctcgtgggtgggggtcc |
| | | atctttgggaccactgtcggcagaggcatcttgaacgatagcctttcctttatcgcaatgat |
| | | ggcatttgtaggtgccaccttccttttctactgtccttttgatgaagtgacagatagctggg |
| | | caatggaatccgaggaggtttcccgatattaccctagttgaaaagtctcaatagccctttg |
| | | gtcttctgagactgtatctttgatattcttggagtagacgagagtgtcgtgctccaccatgtt |
| | | gacggatctctaggacgcgtcctagaagctaattcactggccgtcgttttacaacgtcgt |
| | | gactgggaaaaccctggcgttacccaacttaatcgccttgcagcacatccccctttcgcc |
| | | agctggcgtaatagcgaagaggcccgcaccgatcgcccttcccaacagttgcgcagc |
| | | ctgaatggcgcccgctcctttcgctacttcccttcctactcgccacgttcgccggctttcc |
| | | ccgtcaagctctaaatcgggggctccctttagggttccgatttagtgctttacggcacctc |
| | | gaccccaaaaaacttgatagggtgatggttcacgtagtgggccatcgccctgatagac |
| | | ggtttttcgccctttgacgttggagtccacgttctttaatagtggactcttgttccaaactgg |
| | | aacaacactcaaccctatctcgggctattcttttgatttataagggattttgccgatttcgga |
| | | accaccatcaaacaggattttcgcctgctggggcaaaccagcgtggaccgcttgctgc |
| | | aactctctcagggccaggcggtgaagggcaatcagctgttgcccgtctcactggtgaa |
| | | aagaaaaaccaccccagtacattaaaaacgtccgcaatgtgttattaagagtctaagcgt |
| | | caatttgtttacaccacaatatatcctgccaccagccagccaacagctccccgaccggc |
| | | agctcggcacaaaatcaccactcgatacaggcagcccatcagtccgggacggcgtca |
| | | gcgggagagccgttgtaaggcggcagactttgctcatgttaccgatgctattcggaaga |
| | | acggcaactaagctgccgggtttgaaacacggatgatctcgcggagggtagcatgttg |
| | | attgtaacgatgacagagcgttgctgcctgtgatcaaatatcatctccctcgcagagatcc |
| | | gaattatcagccttcttattcatttctcgcttaaccgtgacaggctgtcgatcttgagaacta |
| | | tgccgacataataggaaatcgctggataaagccgctgaggaagctgagtggcgctattt |
| | | ctttagaagtgaacgttgacgatatcaactcccctatccattgctcaccgaatggtacagg |
| | | tcggggacccgaagttccgactgtcggcctgatgcatccccggctgatcgaccccaga |
| | | tctggggctgagaaagcccagtaaggaaacaactgtaggttcgagtcgcgagatcccc |
| | | cggaaccaaaggaagtaggttaaacccgctccgatcaggccgagccacgccaggcc |
| | | gagaacattggttcctgtaggcatcgggattggcggatcaaacactaaagctactggaa |
| | | cgagcagaagtcctccggccgccagttgccaggcggtaaaggtgagcagaggcacg |
| | | ggaggttgccacttgcgggtcagcacggttccgaacgccatggaaaccgcccccgcc |
| | | aggcccgctgcgacgccgacaggatctagcgctgcgtttggtgtcaacaccaacagc |
| | | gccacgcccgcagttccgcaaatagcccccaggaccgccatcaatcgtatcgggctac |
| | | ctagcagagcggcagagatgaacacgaccatcagcggctgcacagcgcctaccgtc |
| | | gccgcgaccccgcccggcaggcggtagaccgaaataaacaacaagctccagaatag |
| | | cgaaatattaagtgcgccgaggatgaagatgcgcatccaccagattcccgttggaatct |
| | | gtcggacgatcatcacgagcaataaacccgccggcaacgcccgcagcagcataccg |
| | | gcgacccctcggcctcgctgttcgggctccacgaaaacgccggacagatgcgccttgt |
| | | gagcgtccttggggccgtcctcctgtttgaagaccgacagcccaatgatctcgccgtcg |
| | | atgtaggcgccgaatgccacggcatctcgcaaccgttcagcgaacgcctccatgggct |
| | | ttttctcctcgtgctcgtaaacggacccgaacatctctggagctttcttcagggccgacaa |
| | | tcggatctcgcggaaatcctgcacgtcggccgctccaagccgtcgaatctgagccttaa |
| | | tcacaattgtcaattaaatcctctgatatcggcagttcgtagagcgcgccgtgcgtcccg |
| | | agcgatactgagcgaagcaagtgcgtcgagcagtgcccgcttgttcctgaaatgccag |
| | | taaagcgctggctgctgaacccccagccggaactgaccccacaaggccctagcgtttg |
| | | caatgcaccaggtcatcattgacccaggcgtgttccaccaggccgctgcctcgcaactc |
| | | ttcgcaggcttcgccgacctgctcgcgccacttcttcacgcgggtggaatccgatccgc |
| | | acatgaggcggaaggtttccagcttgagcgggtacggctcccggtgcgagctgaaata |
| | | gtcgaacatccgtcgggccgtcggcgacagcttgcggtacttctcccatatgaatttcgt |
| | | gtagtggtcgccagcaaacagcacgacgatttcctcgtcgatcaggacctggcaacgg |
| | | gacgttttcttgccacggtccaggacgcggaagcggtgcagcagcgacaccgattcca |
| | | ggtgcccaacgcggtcggacgtgaagcccatcgccgtcgcctgtaggcgcgacagg |
| | | cattcctcggccttcgtgtaataccggccattgatcgaccagcccaggtcctggcaaag |
| | | ctcgtagaacgtgaaggtgatcggctcgccgataggggtgcgcttcgcgtactccaac |
| | | acctgctgccacaccagttcgtcatcgtcggcccgcagctcgacgccggtgtaggtgat |
| | | cttcacgtccttgttgacgtggaaaatgaccttgttttgcagcgcctcgcgcgggattttct |
| | | tgttgcgcgtggtgaacagggcagagcgggccgtgtcgtttggcatcgctcgcatcgt |
| | | gtccggccacggcgcaatatcgaacaaggaaagctgcataccttgatctgctgcttcgt |
| | | gtgtttcagcaacgcggcctgcttggcctcgctgacctgttttgccaggtcctcgccggc |
| | | ggtttttcgcttcaggtcgtcatagacctcgcgtgtcgatggtcatcgacttcgccaaacc |
| | | tgccgcctcctgttcgagacgacgcgaacgctccacggcggccgatggcgcgggca |
| | | gggcagggggagccagttgcacgctgtcgcgctcgatcttggccgtagcttgctggac |
| | | catcgagccgacggactggaaggtttcgcggggcgcacgcatgacggtgcggcttgc |
| | | gatggtttcggcatcctcggcggaaaaccccgcgtcgatcagacttgcctgtatgccttc |
| | | cggtcaaacgtccgattcattcaccctccttgcgggattgccccgactcacgccggggc |
| | | aatgtgcccttattcctgatttgacccgcctggtgccttggtgtccagataatccaccttatc |
| | | ggcaatgaagtcggtcccgtagaccgtctggccgtccttctcgtacttggtattccgaatc |
| | | ttgccctgcacgaataccagcgaccccttgcccaaatacttgccgtgggcctcggcctg |
| | | agagccaaaacacttgatgcggaagaagtcggtgcgctcctgcttgtcgccggcatcg |
| | | ttgcgccacatctaggtactaaaacaattcatccagtaaaatataatattttattttctcccaa |
| | | tcaggcttgatccccagtaagtcaaaaaatagctcgacatactgacaccccgatatcct |
| | | ccctgatcgaccggacgcagaaggcaatgtcataccacttgtccgccctgccgcttctc |
| | | ccaagatcaataaagccacttactttgccatctttcacaaagatgttgctgtctcccaggtc |
| | | gccgtgggaaaagacaagttcctcttcgggcttttccgtctttaaaaaatcatacagctcg |
| | | cgcggatctttaaatggagtgtcttcttcccagttttcgcaatccacatcggccagatcgtt |
| | | attcagtaagtaatccaattcggctaagcggctgtctaagctattcgtatagggacaatcc |
| | | gatatgtcgatggagtgaaagagcctgatgcactccgcatacagctcgataatcttttca |
| | | gggctttgttcatcttcatactcttccgagcaaaggacgccatcggcctcactcatgagca |
| | | gattgctccagccatcatgccgttcaaagtgcaggacctttggaacaggcagctttcctt |
| | | ccagccatagcatcatgtccttttcccgttccacatcataggtggtccctttataccggctg |
| | | tccgtcatttttaaatataggttttcattttctcccaccagcttatataccttagcaggagacat |
| | | tccttccgtatcttttacgcagcggtatttttcgatcagttttttcaattccggtgatattctcatt |
| | | ttagccatttattatttccttcctcttttctacagtatttaaagataccccaagaagctaattata |
| | | acaagacgaactccaattcactgttccttgcattctaaaaccttaaataccagaaaacagc |
| | | tttttcaaagttgttttcaaagttggcgtataacatagtatcgacggagccgattttgaaacc |
| | | acaattatgggtgatgctgccaacttactgatttagtgtatgatggtgtttttgaggtgctcc |
| | | agtggcttctgtgtctatcagctgtccctcctgttcagctactgacggggtggtgcgtaac |
| | | ggcaaaagcaccgccggacatcagcgctatctctgctctcactgccgtaaaacatggc |
| | | aactgcagttcacttacaccgcttctcaacccggtacgcaccagaaaatcattgatatgg |
| | | ccatgaatggcgttggatgccgggcaacagcccgcattatgggcgttggcctcaacac |
| | | gattttacgtcacttaaaaaactcaggccgcagtcggtaacctcgcgcatacagccggg |
| | | cagtgacgtcatcgtctgcgcggaaatggacgaacagtggggctatgtcggggctaaa |
| | | tcgcgccagcgctggctgttttacgcgtatgacagtctccggaagacggagttgcgca |
| | | cgtattcggtgaacgcactatggcgacgctggggcgtcttatgagcctgctgtcaccctt |
| | | tgacgtggtgatatggatgacggatggctggccgctgtatgaatcccgcctgaaggga |
| | | aagctgcacgtaatcagcaagcgatatacgcagcgaattgagcggcataacctgaatct |
| | | gaggcagcacctggcacggctgggacggaagtcgctgtcgttctcaaaatcggtgga |
| | | gctgcatgacaaagtcatcgggcattatctgaacataaaacactatcaataagttggagt |
| | | cattacccaattatgatagaatttacaagctataaggttattgtcctgggtttcaagcattag |
| | | tccatgcaagtttttatgctttgcccattctatagatatattgataagcgcgctgcctatgcct |
| | | tgccccctgaaatccttacatacggcgatatcttctatataaaagatatattatcttatcagta |
| | | ttgtcaatatattcaaggcaatctgcctcctcatcctcttcatcctcttcgtcttggtagcttttt |
| | | aaatatggcgcttcatagagtaattctgtaaaggtccaattctcgttttcatacctcggtata |
| | | atcttacctatcacctcaaatggttcgctgggtttatcgcacccccgaacacgagcacgg |
| | | cacccgcgaccactatgccaagaatgcccaaggtaaaaattgccggccccgccatga |
| | | agtccgtgaatgccccgacggccgaagtgaagggcaggccgccacccaggccgcc |
| | | gccctcactgcccggcacctggtcgctgaatgtcgatgccagcacctgcggcacgtca |
| | | atgcttccgggcgtcgcgctcgggctgatcgcccatcccgttactgccccgatcccggc |
| | | aatggcaaggactgccagcgctgccatttttggggtgaggccgttcgcggccgaggg |
| | | gcgcagcccctggggggatgggaggcccgcgttagcgggccgggagggttcgaga |
| | | agggggggcaccccccttcggcgtgcgcggtcacgcgcacagggcgcagccctggt |
| | | taaaaacaaggtttataaatattggtttaaaagcaggttaaaagacaggttagcggtggc |
| | | cgaaaaacgggcggaaacccttgcaaatgctggattttctgcctgtggacagcccctca |
| | | aatgtcaataggtgcgcccctcatctgtcagcactctgcccctcaagtgtcaaggatcgc |
| | | gcccctcatctgtcagtagtcgcgcccctcaagtgtcaataccgcagggcacttatcccc |
| | | aggcttgtccacatcatctgtgggaaactcgcgtaaaatcaggcgttacgccgatttgcg |
| | | aggctggccagctccacgtcgccggccgaaatcgagcctgcccctcatctgtcaacgc |
| | | cgcgccgggtgagtcggcccctcaagtgtcaacgtccgcccctcatctgtcagtgagg |
| | | gccaagttaccgcgaggtatccacaacgccggcggccgcggtgtctcgcacacggct |
| | | tcgacggcgtttctggcgcgtttgcagggccatagacggccgccagcccagcggcga |
| | | gggcaaccagcccgg |

1-109. (canceled)

110. A transgenic plant that comprises an endonuclease-mediated stably inherited genomic modification of a tetrahydrocannabinol acid synthase (THCAS) gene, the modification resulting in the at least one of the following a-d:
a. increased cannabidiol (CBD) in the transgenic plant as compared to a comparable control plant without the modification,
b. less than 0.5% by weight of tetrahydrocannabinol (THC) in the transgenic plant as measured by dry weight of the transgenic plant,
c. a CBD to THC ratio in the transgenic plant of at least about 25:1,
d. reduced or suppressed expression of the THCAS gene in the transgenic plant; wherein the transgenic plant comprises an unmodified endogenous cannabidiolic acid synthase (CBDAS) gene.

111. The transgenic plant of claim 110, comprising at least 25%, or at least 50% more CBD as measured by dry weight of the transgenic plant as compared to an amount of CBD as measured by dry weight of a comparable control plant without the endonuclease-mediated stably inherited genomic modification of the THCAS gene.

112. The transgenic plant of claim 110, comprising a CBD to THC ratio of at least about: 25:1, 26:1, 27:1, 28:1, 29:1, 30:1, 31:1, 32:1, 33:1, 34:1, 35:1, 40:1, 45:1; or up to about 50:1.

113. The transgenic plant of claim 110, containing 0% THC or an untraceable amount thereof as measured by dry weight of the transgenic plant.

114. The transgenic plant of claim 110, wherein the endonuclease comprises a clustered regularly interspaced short palindromic repeats (CRISPR) enzyme.

115. The transgenic plant of claim 114, wherein the endonuclease comprises Cas9.

116. The transgenic plant of claim 110, wherein the endonuclease-mediated stably inherited genomic modification of the THCAS gene comprises an insertion.

117. The transgenic plant of claim 110, wherein the endonuclease-mediated stably inherited genomic modification of the THCAS gene comprises a deletion.

118. The transgenic plant of claim 110, wherein the endonuclease-mediated stably inherited genomic modification of the THCAS gene comprises a substitution.

119. The transgenic plant of claim 110, wherein the endonuclease-mediated stably inherited genomic modification of the THCAS gene comprises a frame shift.

120. The transgenic plant of claim 110, wherein the endonuclease-mediated stably inherited genomic modification of the THCAS gene is in a coding region of the THCAS gene.

121. The transgenic plant of claim 110, wherein the endonuclease-mediated stably inherited genomic modification of the THCAS gene is in a regulatory region of the THCAS gene.

122. The transgenic plant of claim 110, wherein the endonuclease-mediated stably inherited genomic modification of the THCAS gene comprises an introduced stop codon.

123. The transgenic plant of claim 110, that is a Cannabis plant.

124. The transgenic plant of claim 110, further comprising a barcode.

125. The transgenic plant of claim 110, further comprising a reporter.

126. The transgenic plant of claim 110, further comprising a selection marker.

127. The transgenic plant of claim 110, wherein the endonuclease-mediated stably inherited genomic modification of the THCAS gene modification results in a.

128. The transgenic plant of claim 110, wherein the endonuclease-mediated stably inherited genomic modification of the THCAS gene modification results in a and b.

129. The transgenic plant of claim 110, wherein the endonuclease-mediated stably inherited genomic modification of the THCAS gene modification results in a, b, and c.

130. The transgenic plant of claim 110, wherein the endonuclease-mediated stably inherited genomic modification of the THCAS gene modification results in a, b, c, and d.

131. The transgenic plant of claim 110, wherein the endonuclease-mediated stably inherited genomic modification of the THCAS gene modification results in a and c.

132. The transgenic plant of claim 110, wherein the endonuclease-mediated stably inherited genomic modification of the THCAS gene modification results in a and d.

133. The transgenic plant of claim 110, wherein the endonuclease-mediated stably inherited genomic modification of the THCAS gene modification results in a, c and d.

134. The transgenic plant of claim 110, wherein the endonuclease-mediated stably inherited genomic modification of the THCAS gene modification results in c and d.

135. The transgenic plant of claim 110, wherein the endonuclease-mediated stably inherited genomic modification of the THCAS gene modification results in b and c.

136. The transgenic plant of claim 110, wherein the endonuclease-mediated stably inherited genomic modification of the THCAS gene modification results in b and d.

137. The transgenic plant of claim 110, wherein the endonuclease-mediated stably inherited genomic modification of the THCAS gene modification results in b, c and d.

138. The transgenic plant of claim 110, wherein the endonuclease-mediated stably inherited genomic modification of the THCAS gene results in d.

139. The transgenic plant of claim 110, which has increased cannabidiolic acid (CBDA) in the transgenic plant relative to a comparable control plant without the endonuclease-mediated stably inherited genomic modification of the THCAS gene.
